(19) United States
(12) Patent Application Publication
(10) Pub. No.: US 2023/0107941 A1
(43) Pub. Date: Apr. 6, 2023

(54) HEPATITIS B CAPSID ASSEMBLY MODULATORS

(71) Applicant: Venatorx Pharmaceuticals, Inc., Malvern, PA (US)

(72) Inventors: Christopher J. BURNS, Malvern, PA (US); Glen COBURN, Bethel, CT (US); Bin LIU, Plainsboro, NJ (US); Jiangchao YAO, Princeton, NJ (US); Christopher BENETATOS, Chester Springs, PA (US); Steven A. BOYD, Chester Springs, PA (US); Stephen M. CONDON, Glenmoore, PA (US); Thomas HAIMOWITZ, Newtown, PA (US)

(21) Appl. No.: 17/783,836

(22) PCT Filed: Dec. 9, 2020
(86) PCT No.: PCT/US20/63936

§ 371 Date: Jun. 9, 2022

Related U.S. Application Data
(60) Provisional application No. 62/946,362, filed on Dec. 10, 2019.

(51) Int. Cl. C07D 207/34; A61K 45/06; A61K 31/40; C07D 401/12; A61K 31/4439; A61K 31/454; A61K 31/4025; C07D 403/12; C07D 471/04; A61K 31/437; A61K 31/5377; C07D 401/14; C07D 417/14; A61K 31/501; A61P 31/20; C07D 417/04; A61K 31/427

(52) U.S. Cl. CPC C07D 207/34; A61K 45/06; A61K 31/40; C07D 401/12; A61K 31/4439; A61K 31/454; A61K 31/4025; C07D 403/12; C07D 471/04; A61K 31/437; A61K 31/5377; C07D 401/14; C07D 417/14; A61K 31/501; A61P 31/20; C07D 417/04; A61K 31/427

(57) ABSTRACT

Described herein are hepatitis B capsid assembly modulators and pharmaceutical compositions comprising said compounds. The subject compounds and compositions are useful for the treatment of hepatitis B.

### CROSS-REFERENCE

This application claims the benefit of U.S. Provisional Application Ser. No. 62/946,362 filed Dec. 10, 2019 which is hereby incorporated by reference in its entirety.

### BACKGROUND OF THE INVENTION

The present invention relates to small-molecule compounds that modulate capsid assembly and block hepatitis B virus (HBV) replication with the potential to be used as a monotherapy or in combination with other antivirals for the treatment of chronic HBV infection.

HBV is a small enveloped DNA virus belonging to the Hepadnaviridae family that is distributed worldwide as ten geographically distinct genotypes. Infection with HBV is typically self-limiting in otherwise healthy adults; however, vertical transmission or exposure during early childhood often results in a chronic lifelong infection. Worldwide there are an estimated >400 million individuals chronically infected with HBV that are at risk for complications due to liver disease, including cirrhosis, fibrosis, hepatocellular carcinoma and death. Each year 500,000 to 1 million people die from end stage liver disease as a consequence of HBV infection

The compact HBV genome utilizes four overlapping reading frames to encode the major structural and non-structural proteins: polymerase (F), envelope (S), core (C) and the X protein (X). HBV enters human hepatocytes via receptor mediated endocytosis, following binding of the envelope glycoprotein to its primary receptor, the bile acid transporter sodium taurocholate co-transporting polypeptide (NTCP). Following fusion with the endosome membrane, the capsid is ejected into the cytoplasm and translocated to the nucleus. The partially double-stranded, relaxed, circular HBV genome (RC DNA) is converted to a covalently closed circular DNA form (cccDNA) by host cellular DNA repair mechanisms. The HBV cccDNA serves as the template for RNA polymerase II-dependent transcription of multiple RNA species, including viral mRNAs and the 3.2-kbp pre-genomic RNA (pgRNA). During the maturation process, pgRNA is packaged into capsids along with the HBV polymerase. The pgRNA is then reverse transcribed into a negative-stranded DNA template that is subsequently converted into the partially double-stranded RC DNA species by the polymerase. Mature, enveloped HBV particles containing the RC DNA genome are secreted from the surface of the infected hepatocyte ready to initiate new cycles of infection.

The capsid is composed of 240 copies of the core protein that spontaneously self-assemble through a network of weak inter-subunit interactions. In vitro evidence suggests that a trimer of core dimers initiates the nucleation event that rapidly recruits additional dimers to form the icosahedral core structure (T=4). In addition to its structural role, encapsidation of the pgRNA is an essential step required for HBV DNA synthesis and formation of the mature capsid particle. The core protein also plays an important role in shuttling the RC DNA into the nucleus to initiate and maintain the cccDNA pools and may also play a role in regulating interferon sensitive gene expression. Thus, capsid modulators may have the unique ability to intervene at multiple points in the HBV lifecycle.

Several chemotype series of HBV capsid assembly modulators have been reported in the literature including: phenylpropenamides (PP) (e.g., AT-130), heteroarylpyrimidines (HAP) (e.g. Bay 41-4109), and sulfamoylbenzamides (SBA) (e.g. NVR 3-778). Capsid modulators exert their effects on the assembly process through one of two different mechanisms of action. The HAP series induces the aberrant assembly of large capsid aggregates that subsequently triggers the degradation of the core protein. The PP and SBA series, on the other hand, appear to accelerate capsid assembly resulting in the production of authentic empty capsid particles that have failed to incorporate pgRNA. Assembly modulators representing both mechanisms have demonstrated the ability to reduce HBV DNA levels in mouse models of infection. More recently, NVR 3-778 (SBA) demonstrated clinical proof-of-concept in a Phase 1b clinical trial, resulting in a −1.7 log 10 reduction in HBV DNA following 600 mg bid dosing for 29 days.

### SUMMARY OF THE INVENTION

Described herein are compounds of Formula (I) that modulate the normal capsid assembly of hepatitis B core proteins to inhibit the hepatitis B lifecycle, and thus act as antiviral agents toward HBV.

Disclosed herein are compounds of Formula (I), or a pharmaceutically acceptable salt, solvate, or stereoisomer thereof:

Disclosed herein is a compound of Formula (I), or a pharmaceutically acceptable salt, solvate, or stereoisomer thereof:

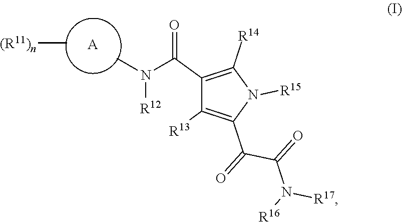

wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
R¹² is hydrogen or C₁-C₆alkyl;
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³;
R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
provided that one of R¹³ or R¹⁴ is not —CH₃;
R¹⁵ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁵;
R¹⁶ and R¹⁷ are each independently hydrogen, —CN, —OR²⁰, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, cycloalkenyl, heterocycloalkyl, heterocycloalkenyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁶;
or R¹⁶ and R¹⁷ are taken together with the nitrogen atom to which they are attached to form a heterocycloalkyl or a heterocycloalkenyl; each optionally substituted with one, two, or three R⁷;
each R²⁰ is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R⁶;
n is 0-4;
each R¹, R², R³, R⁴, R⁵, and R⁷ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R⁶ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —B(OR^{b})(OR^{c}), —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R^{6a};
each R^{6a} is independently oxo, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{b} and R^{c} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{b} and R^{c} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

Also disclosed herein are pharmaceutical compositions comprising a compound disclosed herein, or a pharmaceutically acceptable salt, solvate, or stereoisomer thereof, and a pharmaceutically acceptable excipient.

Also disclosed herein are methods of treating an infection in a subject, comprising administering to the subject a compound disclosed herein, or a pharmaceutically acceptable salt, solvate, or stereoisomer thereof or a pharmaceutical composition disclosed herein. In some embodiments of a method of treating an infection; the infection is a viral infection. In some embodiments of a method of treating an infection; the infection is caused by the hepatitis B virus. In some embodiments of a method of treating an infection; the infection is hepatitis B. In some embodiments, the method further comprises administering an additional therapeutic agent useful for treating a chronic HBV infection. In some embodiments, the additional therapeutic agent useful for treating a chronic HBV infection is a reverse transcriptase inhibitor; an HBV polymerase inhibitor, a capsid inhibitor; a cccDNA formation inhibitor; an RNA destabilizer; a checkpoint inhibitor (PD-1/PD-L1 inhibitor); a therapeutic vaccine; an RNA interference (RNAi) therapeutic; an antisense-based therapeutic, an HBV entry inhibitor; a TLR agonist; an RIG-I agonist, or an interferon.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Chronic hepatitis B infection (CHB) is currently managed with interferon-alpha or nucleoside(tide) analog-based therapies that target the HBV encoded polymerase/reverse transcriptase. The effectiveness of interferon-alpha is limited by inadequate long term responses and severe side effects, while entecavir and tenofovir, are generally well-tolerated, possess a high barrier to resistance and potently suppress viral replication. None of the aforementioned frontline therapies are curative, however, and expensive lifelong therapy is required to maintain a virologic response and prevent the complications associated with liver disease. Novel therapies representing different treatment classes are therefore urgently required to improve functional cure rates (i.e. defined as the loss of HBsAg expression) and shorten treatment durations. Modulators of HBV capsid assembly represent one such class of antivirals with the potential to improve outcomes for chronically infected individuals.

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“oxo” refers to ═O.

“Alkyl” refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkyl is optionally substituted with oxo, halogen, —CN, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkenyl” refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), 1-propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated. Unless stated otherwise specifically in the specification, an alkenyl group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkenyl is optionally substituted with oxo, halogen, —CN, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to an optionally substituted straight-chain or optionally substituted branched-chain hydrocarbon monoradical having one or more carbon-carbon triple-bonds and having from two to about ten carbon atoms, more preferably from two to about six carbon atoms. Examples include, but are not limited to ethynyl, 2-propynyl, 2-butynyl, 1,3-butadiynyl and the like. Whenever it appears herein, a numerical range such as “C₂-C₆ alkynyl” or “C₂₋₆alkynyl”, means that the alkynyl group may consist of 2 carbon atoms, 3 carbon atoms, 4 carbon atoms, 5 carbon atoms or 6 carbon atoms, although the present definition also covers the occurrence of the term “alkynyl” where no numerical range is designated. Unless stated otherwise specifically in the specification, an alkynyl group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkynyl is optionally substituted with oxo, halogen, —CN, —OH, —OMe, —NH₂, or —NO₂. In some embodiments, the alkynyl is optionally substituted with halogen, —CN, —OH, or —OMe. In some embodiments, the alkynyl is optionally substituted with halogen.

“Alkylene” refers to a straight or branched divalent hydrocarbon chain. Unless stated otherwise specifically in the specification, an alkylene group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkylene is optionally substituted with oxo, halogen, —CN, —OH, —OMe, —NH₂, or —NO₂. In some embodiments, the alkylene is optionally substituted with halogen, —CN, —OH, or —OMe. In some embodiments, the alkylene is optionally substituted with halogen.

“Alkoxy” refers to a radical of the formula —ORₐ where Rₐ is an alkyl radical as defined. Unless stated otherwise specifically in the specification, an alkoxy group may be optionally substituted as described below, for example, with oxo, halogen, amino, nitrile, nitro, hydroxyl, haloalkyl, alkoxy, aryl, cycloalkyl, heterocycloalkyl, heteroaryl, and the like. In some embodiments, the alkoxy is optionally substituted with halogen, —CN, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from a hydrocarbon ring system comprising hydrogen, 6 to 30 carbon atoms and at least one aromatic ring. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the aryl is optionally substituted with halogen, methyl, ethyl, —CN, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a stable, partially or fully saturated, monocyclic or polycyclic carbocyclic ring, which may include fused (when fused with an aryl or a heteroaryl ring, the cycloalkyl is bonded through a non-aromatic ring atom) or bridged ring systems. Representative cycloalkyls include, but are not limited to, cycloalkyls having from three to fifteen carbon atoms (C₃-C₁₅ cycloalkyl), from three to ten carbon atoms (C₃-C₁₀ cycloalkyl), from three to eight carbon atoms (C₃-C₈ cycloalkyl), from three to six carbon atoms (C₃-C₆ cycloalkyl), from three to five carbon atoms (C₃-C₅ cycloalkyl), or three to four carbon atoms (C₃-C₄ cycloalkyl). In some embodiments, the cycloalkyl is a 3- to 10-membered cycloalkyl. In some embodiments, the cycloalkyl is a 3- to 6-membered cycloalkyl. In some embodiments, the cycloalkyl is a 5- to 6-membered cycloalkyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a cycloalkyl is optionally substituted with oxo, halogen, methyl, ethyl, —CN, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Cycloalkenyl” refers to a partially unsaturated, monocyclic or polycyclic carbocyclic ring, which may include fused (when fused with an aryl or a heteroaryl ring, the cycloalkenyl is bonded through a non-aromatic ring atom) or bridged ring systems. Representative cycloalkenyl include, but are not limited to, cycloalkenyls having from three to fifteen carbon atoms (C₃-C₁₅ cycloalkenyl), from three to ten carbon atoms (C₃-C₁₀ cycloalkenyl), from three to eight carbon atoms (C₃-C₈ cycloalkenyl), from three to six carbon atoms (C₃-C₆ cycloalkenyl), from three to five carbon atoms (C₃-C₅ cycloalkenyl), four to six carbon atoms (C₄-C₆ cycloalkenyl), four to eight carbon atoms (C₄-C₈ cycloalkenyl), or four to ten carbon atoms (C₄-C₁₀ cycloalkenyl). Monocyclic cycloalkenyl include, for example, cyclopentene, cyclohexene, cycloheptene, cyclopentadiene, cyclohexadiene, cycloheptadiene, and cycloheptatriene. Unless stated otherwise specifically in the specification, a cycloalkenyl may be optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the cycloalkenyl is optionally substituted with oxo, halogen, methyl, ethyl, —CN, —CF₃, —OH, —OMe, —NH₂, or —NO₂. In some embodiments, the cycloalkenyl is optionally substituted with halogen, methyl, ethyl, —CN, —CF₃, —OH, or —OMe. In some embodiments, the cycloalkenyl is optionally substituted with halogen.

“Halo” or “halogen” refers to bromo, chloro, fluoro or iodo. In some embodiments, halogen is fluoro or chloro. In some embodiments, halogen is fluoro.

“Haloalkyl” refers to an alkyl radical, as defined above, that is substituted by one or more halo radicals, as defined above, e.g., trifluoromethyl, difluoromethyl, fluoromethyl, trichloromethyl, 2,2,2-trifluoroethyl, 1,2-difluoroethyl, 3-bromo-2-fluoropropyl, 1,2-dibromoethyl, and the like.

“Heterocycloalkyl” refers to a stable 3- to 24-membered fully saturated ring radical comprising 2 to 23 carbon atoms and from one to 8 heteroatoms selected from the group consisting of nitrogen, oxygen, phosphorous and sulfur. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl), from two to ten carbon atoms (C₂-C₁₀ heterocycloalkyl), from two to eight carbon atoms (C₂-C₈ heterocycloalkyl), from two to seven carbon atoms (C₂-C₇ heterocycloalkyl), from two to six carbon atoms (C₂-C₆ heterocycloalkyl), from two to five carbon atoms (C₂-C₅ heterocycloalkyl), or two to four carbon atoms (C₂-C₄ heterocycloalkyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. Unless otherwise noted, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e. skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the heterocycloalkyl is optionally substituted with oxo, halogen, methyl, ethyl, —CN, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heterocycloalkenyl” refers to a stable 3- to 24-membered partially unsaturated ring radical comprising 2 to 23 carbon atoms and from one to 8 heteroatoms selected from the group consisting of nitrogen, oxygen, phosphorous and sulfur. In some embodiments, the heterocycloalkenyl comprises one to three heteroatoms selected from the group consisting of nitrogen, oxygen, and sulfur. In some embodiments, the heterocycloalkenyl comprises one to three heteroatoms selected from the group consisting of nitrogen and oxygen. In some embodiments, the heterocycloalkenyl comprises one to three nitrogens. In some embodiments, the heterocycloalkenyl comprises one or two nitrogens. In some embodiments, the heterocycloalkenyl comprises one nitrogen. Unless stated otherwise specifically in the specification, the heterocycloalkenyl may be a monocyclic, bicyclic, tricyclic or tetracyclic ring system, which may include fused (when fused with an aryl or a heteroaryl ring, the heterocycloalkyl is bonded through a non-aromatic ring atom) or bridged ring systems; and the nitrogen, carbon or sulfur atoms in the heterocycloalkenyl radical may be optionally oxidized; the nitrogen atom may be optionally quaternized. Representative heterocycloalkenyls include, but are not limited to, heterocycloalkenyls having from two to ten carbon atoms (C₂-C₁₀ heterocycloalkenyl), from two to eight carbon atoms (C₂-C₈ heterocycloalkenyl), from two to seven carbon atoms (C₂-C₇ heterocycloalkenyl), from two to six carbon atoms (C₂-C₆ heterocycloalkenyl), from two to five carbon atoms (C₂-C₅ heterocycloalkenyl), or two to four carbon atoms (C₂-C₄ heterocycloalkenyl). Examples of such heterocycloalkenyls include, but are not limited to, 2,3-dihydro-1H-pyrrole, 1,2,3,6-tetrahydropyridine, 1,2-dihydropyridine, 1,2,3,4-tetrahydropyrazine, and 3,4-dihydro-2H-1,4-oxazine. Unless otherwise noted, heterocycloalkenyls have from 2 to 10 carbons in the ring. It is understood that when referring to the number of carbon atoms in a heterocycloalkenyl, the number of carbon atoms in the heterocycloalkenyl is not the same as the total number of atoms (including the heteroatoms) that make up the heterocycloalkenyl (i.e. skeletal atoms of the heterocycloalkenyl ring). In some embodiments, the heterocycloalkenyl is a 3- to 8-membered heterocycloalkenyl. In some embodiments, the heterocycloalkenyl is a 3- to 7-membered heterocycloalkenyl. In some embodiments, the heterocycloalkenyl is a 3- to 6-membered heterocycloalkenyl. In some embodiments, the heterocycloalkenyl is a 4- to 6-membered heterocycloalkenyl. In some embodiments, the heterocycloalkenyl is a 5- to 6-membered heterocycloalkenyl. Unless stated otherwise specifically in the specification, a heterocycloalkenyl may be optionally substituted as described below, for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the heterocycloalkenyl is optionally substituted with oxo, halogen, methyl, ethyl, —CN, —CF₃, —OH, —OMe, —NH₂, or —NO₂. In some embodiments, the heterocycloalkenyl is optionally substituted with halogen, methyl, ethyl, —CN, —CF₃, —OH, or —OMe. In some embodiments, the heterocycloalkenyl is optionally substituted with halogen.

“Heteroaryl” refers to a 5- to 14-membered ring system radical comprising hydrogen atoms, one to thirteen carbon atoms, one to six heteroatoms selected from the group consisting of nitrogen, oxygen, phosphorous and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted as described below, for example, with halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, the heteroaryl is optionally substituted with halogen, methyl, ethyl, —CN, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “optional” or “optionally” means that the subsequently described event or circumstance may or may not occur, and that the description includes instances where said event or circumstance occurs and instances in which it does not. For example, “optionally substituted alkyl” means either “alkyl” or “substituted alkyl” as defined above. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc.). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 1,000 daltons, and more typically, up to about 500 daltons.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition. In some embodiments, treatment also includes prophylactic treatment (e.g., administration of a composition described herein when an individual is suspected to be suffering from a viral infection, e.g., hepatitis B).

### Compounds

Described herein are compounds of Formula (I), or a pharmaceutically acceptable salt, solvate, or stereoisomer thereof useful in the treatment of viral infections. In some embodiments, the viral infection is a chronic hepatitis B infection.

Disclosed herein is a compound of Formula (I), or a pharmaceutically acceptable salt, solvate, or stereoisomer thereof:

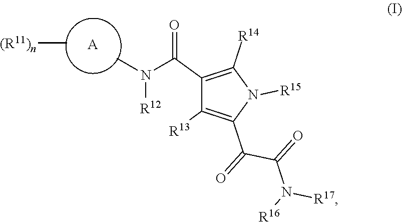

wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
R¹² is hydrogen or C₁-C₆alkyl;
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³;
R¹⁴ is —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
provided that one of R¹³ or R¹⁴ is not —CH₃;
R¹⁵ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁵;
R¹⁶ and R¹⁷ are each independently hydrogen, —CN, —OR²⁰, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, cycloalkenyl, heterocycloalkyl, heterocycloalkenyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁶;
or R¹⁶ and R¹⁷ are taken together with the nitrogen atom to which they are attached to form a heterocycloalkyl or a heterocycloalkenyl; each optionally substituted with one, two, or three R⁷;
each R²⁰ is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R⁶;
n is 0-4;
each R¹, R², R³, R⁴, R⁵, and R⁷ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R⁶ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —B(OR^{b})(OR^{c}), —C(═O)R^{a}, —OC(═O)R^{a}, —C(═O)OR^{b}, —OC(═O)OR, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R^{6a};
each R^{6a} is independently oxo, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{b} and R^{c} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{b} and R^{c} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

In some embodiment of a compound of Formula (I), Ring A is cycloalkyl or heterocycloalkyl. In some embodiment of a compound of Formula (I), Ring A is cycloalkyl, aryl or heteroaryl. In some embodiment of a compound of Formula (I), Ring A is aryl or heteroaryl. In some embodiment of a compound of Formula (I), Ring A is phenyl or 5- or 6-membered heteroaryl. In some embodiment of a compound of Formula (I), Ring A is phenyl or 6-membered heteroaryl. In some embodiment of a compound of Formula (I), Ring A is phenyl or pyridyl. In some embodiment of a compound of Formula (I), Ring A is phenyl.

In some embodiment of a compound of Formula (I), n is 0-3. In some embodiment of a compound of Formula (I), n is 0-2. In some embodiment of a compound of Formula (I), n is 0 or 1. In some embodiment of a compound of Formula (I), n is 1-3. In some embodiment of a compound of Formula (I), n is 1 or 2. In some embodiment of a compound of Formula (I), n is 0. In some embodiment of a compound of Formula (I), n is 1. In some embodiment of a compound of Formula (I), n is 2. In some embodiment of a compound of Formula (I), n is 3. In some embodiment of a compound of Formula (I), n is 4.

In some embodiment of a compound of Formula (I), each R¹¹ is independently halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three R¹. In some embodiment of a compound of Formula (I), each R¹¹ is independently halogen, —CN, C₁-C₆alkyl, C₁-C₆haloalkyl, C₁-C₆hydroxyalkyl, C₁-C₆aminoalkyl, or cycloalkyl; wherein each alkyl and cycloalkyl is independently optionally substituted with one, two, or three R¹. In some embodiment of a compound of Formula (I), each R¹¹ is independently halogen, —CN, C₁-C₆alkyl, C₁-C₆haloalkyl, C₁-C₆hydroxyalkyl, C₁-C₆aminoalkyl; wherein each alkyl is independently optionally substituted with one, two, or three R¹. In some embodiment of a compound of Formula (I), each R¹¹ is independently halogen, —CN, C₁-C₆alkyl, or C₁-C₆haloalkyl; wherein each alkyl is independently optionally substituted with one, two, or three R¹. In some embodiment of a compound of Formula (I), each R¹¹ is independently halogen, —CN, —OH, —OR^{a}, —NR^{b}R^{c}, —C(═O)OR^{b}, —C(═O)NR^{b}R^{c}, C₁-C₆alkyl, C₁-C₆haloalkyl, or cycloalkyl. In some embodiment of a compound of Formula (I), each R¹¹ is independently halogen, —CN, C₁-C₆alkyl, or C₁-C₆haloalkyl. In some embodiment of a compound of Formula (I), each R¹¹ is independently halogen or C₁-C₆alkyl.

In some embodiment of a compound of Formula (I), each alkyl, alkenyl, alkynyl, cycloalkyl, heterocycloalkyl, aryl, or heteroaryl in R¹¹ is optionally substituted with one, two, or three R¹. In some embodiment of a compound of Formula (I), each alkyl, alkenyl, alkynyl, cycloalkyl, heterocycloalkyl, aryl, or heteroaryl in R¹¹ is optionally substituted with one or two R¹. In some embodiment of a compound of Formula (I), each alkyl, alkenyl, alkynyl, cycloalkyl, heterocycloalkyl, aryl, or heteroaryl in R¹¹ is optionally substituted with one R¹. In some embodiment of a compound of Formula (I), each alkyl, alkenyl, alkynyl, cycloalkyl, heterocycloalkyl, aryl, or heteroaryl in R¹¹ is optionally substituted with two R¹. In some embodiment of a compound of Formula (I), each alkyl, alkenyl, alkynyl, cycloalkyl, heterocycloalkyl, aryl, or heteroaryl in R¹¹ is optionally substituted with three R¹.

In some embodiment of a compound of Formula (I), each R¹ is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R¹ is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R¹ is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R¹ is independently oxo, halogen, —CN, —OH, —OR^{a}, —NR^{b}R^{c}, C₁-C₆alkyl, C₁-C₆haloalkyl. In some embodiment of a compound of Formula (I), each R¹ is independently oxo, halogen, —CN, —OH, —OMe, —NH₂, Me, or CF₃. In some embodiment of a compound of Formula (I), each R¹ is independently halogen.

In some embodiment of a compound of Formula (I), two R¹¹ on adjacent atoms are taken together with the atoms to which they are attached to form a cycloalkyl, heterocycloalkyl, aryl, or heteroaryl; each optionally substituted with one, two, or three R². In some embodiment of a compound of Formula (I), two R¹¹ on adjacent atoms are taken together with the atoms to which they are attached to form a cycloalkyl optionally substituted with one, two, or three R². In some embodiment of a compound of Formula (I), two R¹¹ on adjacent atoms are taken together with the atoms to which they are attached to form a heterocycloalkyl optionally substituted with one, two, or three R². In some embodiment of a compound of Formula (I), two R¹¹ on adjacent atoms are taken together with the atoms to which they are attached to form an aryl optionally substituted with one, two, or three R². In some embodiment of a compound of Formula (I), two R¹¹ on adjacent atoms are taken together with the atoms to which they are attached to form a heteroaryl optionally substituted with one, two, or three R².

In some embodiment of a compound of Formula (I), each cycloalkyl, heterocycloalkyl, aryl, or heteroaryl formed when two R¹¹ are taken together is optionally substituted with one, two, or three R². In some embodiment of a compound of Formula (I), each cycloalkyl, heterocycloalkyl, aryl, or heteroaryl formed when two R¹¹ are taken together is optionally substituted with one or two R². In some embodiment of a compound of Formula (I), each cycloalkyl, heterocycloalkyl, aryl, or heteroaryl formed when two R¹¹ are taken together is optionally substituted with one R². In some embodiment of a compound of Formula (I), each cycloalkyl, heterocycloalkyl, aryl, or heteroaryl formed when two R¹¹ are taken together is optionally substituted with two R². In some embodiment of a compound of Formula (I), R¹¹ is optionally substituted with three R².

In some embodiment of a compound of Formula (I), each R² is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R² is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R² is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R² is independently oxo, halogen, —CN, —OH, —OR^{a}, —NR^{b}R^{c}, C₁-C₆alkyl, C₁-C₆haloalkyl. In some embodiment of a compound of Formula (I), each R² is independently oxo, halogen, —CN, —OH, —OMe, —NH₂, Me, or CF₃. In some embodiment of a compound of Formula (I), each R² is independently halogen.

In some embodiment of a compound of Formula (I),

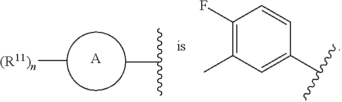

In some embodiment of a compound of Formula (I), R¹² is hydrogen. In some embodiment of a compound of Formula (I), R¹² is C₁-C₆alkyl.

In some embodiment of a compound of Formula (I), R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and

R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
provided that one of R¹³ or R¹⁴ is not —CH₃.

In some embodiment of a compound of Formula (I), R¹³ is —CN, —OH, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and

R¹⁴ is —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴;
provided that one of R¹³ or R¹⁴ is not —CH₃.

In some embodiment of a compound of Formula (I), R¹³ is —CN, —OH, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and

R¹⁴ is —CN, —OH, —OW, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴;
provided that one of R¹³ or R¹⁴ is not —CH₃.

In some embodiment of a compound of Formula (I), R¹³ is —CN, —OH, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R³; and

R¹⁴ is —CN, —OH, —OW, C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R⁴;
provided that one of R¹³ or R¹⁴ is not —CH₃.

In some embodiment of a compound of Formula (I), R¹³ is C₁-C₆alkyl, C₁-C₆haloalkyl, C₁-C₆hydroxyalkyl, cycloalkyl, or heterocycloalkyl; wherein each alkyl, cycloalkyl, and heterocycloalkyl, is optionally substituted with one, two, or three R³; and

R¹⁴ is C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R⁴;
provided that one of R¹³ or R¹⁴ is not —CH₃.

In some embodiment of a compound of Formula (I), R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and

R¹⁴ is —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
or
R¹³ is —CN, —OH, —SH, —SW, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴.

In some embodiment of a compound of Formula (I), R¹³ is —CN, —OH, —NR^{b}R^{c}, —C(═O)R^{a}, —C(═O)OR^{b}, —C(═O)NR^{b}R^{c}, C₂-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R; and

R¹⁴ is —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴;
or
R¹³ is —CN, —OH, —NR^{b}R^{c}, —C(═O)R^{a}, —C(═O)OR, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and
R¹⁴ is —CN, —OH, —OR^{a}, —NR^{b}R^{c}, —C(═O)R^{a}, —C(═O)OR^{b}, —C(═O)NR^{b}R^{c}, C₂-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴.

In some embodiment of a compound of Formula (I), R¹³ is —CN, —OH, —NR^{b}R^{c}, C₂-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R³; and

R¹⁴ is —CN, —OH, —OR^{a}, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R⁴;
or
R¹³ is —CN, —OH, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, or heterocycloalkyl is optionally substituted with one, two, or three R³; and
R¹⁴ is —CN, —OH, —OR^{a}, —NR^{b}R^{c}, C₂-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₂-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R³; and

R¹⁴ is C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R⁴;
or
R¹³ is C₁-C₆alkyl, C₁-C₆haloalkyl, C₁-C₆hydroxyalkyl, cycloalkyl, or heterocycloalkyl; wherein each alkyl, cycloalkyl, or heterocycloalkyl is optionally substituted with one, two, or three R³; and
R¹⁴ is C₂-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₂-C₆alkyl, C₁-C₆haloalkyl, cycloalkyl, or heterocycloalkyl; wherein each alkyl, cycloalkyl, and heterocycloalkyl is optionally substituted with one, two, or three R³; and

R¹⁴ is C₁-C₆alkyl, C₁-C₆haloalkyl, cycloalkyl, or heterocycloalkyl; wherein each alkyl, cycloalkyl, and heterocycloalkyl is optionally substituted with one, two, or three R⁴;
or
R¹³ is C₁-C₆alkyl, C₁-C₆haloalkyl, cycloalkyl, or heterocycloalkyl; wherein each alkyl, cycloalkyl, or heterocycloalkyl is optionally substituted with one, two, or three R³; and
R¹⁴ is C₂-C₆alkyl, C₁-C₆haloalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₂-C₆alkyl or cycloalkyl; wherein each alkyl and cycloalkyl is optionally substituted with one, two, or three R³; and

R¹⁴ is C₁-C₆alkyl or cycloalkyl; wherein each alkyl and cycloalkyl is optionally substituted with one, two, or three R⁴;
or
R¹³ is C₁-C₆alkyl or cycloalkyl; wherein each alkyl and cycloalkyl is optionally substituted with one, two, or three R³; and
R¹⁴ is C₂-C₆alkyl or cycloalkyl; wherein each alkyl and cycloalkyl is optionally substituted with one, two, or three R⁴.

In some embodiment of a compound of Formula (I), R¹³ is C₂-C₆alkyl or cycloalkyl; wherein each alkyl and cycloalkyl is optionally substituted with one, two, or three R³; and

R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
or
R¹³ is —CN, —OH, —SH, —SW, —S(═O)W, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is C₂-C₆alkyl or cycloalkyl; wherein each alkyl and cycloalkyl is optionally substituted with one, two, or three R⁴.

In some embodiment of a compound of Formula (I), R¹³ is C₂-C₆alkyl or cycloalkyl; wherein each alkyl and cycloalkyl is optionally substituted with one, two, or three R³; and

R¹⁴ is —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴;
or
R¹³ is —CN, —OH, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and
R¹⁴ is C₂-C₆alkyl or cycloalkyl; wherein each alkyl and cycloalkyl is optionally substituted with one, two, or three R⁴.

In some embodiment of a compound of Formula (I), R¹³ is C₂-C₆alkyl or cycloalkyl; wherein each alkyl and cycloalkyl is optionally substituted with one, two, or three R³; and

R¹⁴ is —CN, —OH, —OR^{a}, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, or heterocycloalkyl is optionally substituted with one, two, or three R⁴;
or
R¹³ is —CN, —OH, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R³; and
R¹⁴ is C₂-C₆alkyl or cycloalkyl; wherein each alkyl and cycloalkyl is optionally substituted with one, two, or three R⁴.

In some embodiment of a compound of Formula (I), R¹³ is C₂-C₆alkyl or cycloalkyl; wherein each alkyl and cycloalkyl is optionally substituted with one, two, or three R³; and

R¹⁴ is —C₁-C₆alkyl, C₁-C₆haloalkyl, C₁-C₆hydroxyalkyl, C₁-C₆aminoalkyl, or cycloalkyl; wherein each alkyl, and cycloalkyl is optionally substituted with one, two, or three R⁴;
or
R¹³ is C₁-C₆alkyl, C₁-C₆haloalkyl, C₁-C₆hydroxyalkyl, C₁-C₆aminoalkyl, or cycloalkyl; wherein each alkyl, cycloalkyl, and heterocycloalkyl is optionally substituted with one, two, or three R³; and
R¹⁴ is C₂-C₆alkyl or cycloalkyl; wherein each alkyl and cycloalkyl is optionally substituted with one, two, or three R⁴.

In some embodiment of a compound of Formula (I), R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and

R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴.

In some embodiment of a compound of Formula (I), R¹³ is —CN, —OH, —NR^{b}R^{c}, —C(═O)R^{a}, —C(═O)OR^{b}, —C(═O)NR^{b}R^{c}, C₂-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and

R¹⁴ is —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴.

In some embodiment of a compound of Formula (I), R¹³ is —CN, —OH, —NR^{b}R^{c}, C₂-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R³; and

R¹⁴ is —CN, —OH, —OR^{a}, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₂-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R³; and

R¹⁴ is C₁-C₆alkyl, C₁-C₆haloalkyl, C₁-C₆hydroxyalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₂-C₆alkyl, C₁-C₆haloalkyl, or cycloalkyl; wherein each alkyl, and cycloalkyl is optionally substituted with one, two, or three R; and

R¹⁴ is C₁-C₆alkyl, C₁-C₆haloalkyl, or cycloalkyl; wherein each alkyl, and cycloalkyl is optionally substituted with one, two, or three R⁴.

In some embodiment of a compound of Formula (I), R¹³ is —CN, —OH, —NR^{b}R^{c}, —C(═O)R^{a} C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R³; and

R¹⁴ is —CN, —OH, —OR^{a}, —NR^{b}R^{c}, —C(═O)R^{a}, —C(═O)OR^{b}, —C(═O)NR^{b}R^{c}, C₂-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is optionally substituted with one, two, or three R⁴. In some embodiment of a compound of Formula (I), R¹³ is —CN, —OH, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R³; and

R¹⁴ is —CN, —OH, —OR^{a}, —NR^{b}R^{c}, C₂-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is —CN, —OH, —NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R³; and

R¹⁴ is —CN, —OH, —OR^{a}, —NR^{b}R^{c}, C₂-C₆alkyl, C₁-C₆haloalkyl, C₁-C₆hydroxyalkyl, C₁-C₆aminoalkyl, cycloalkyl, heterocycloalkyl, —C₁-C₆alkyl(cycloalkyl), or heteroaryl; wherein each alkyl, cycloalkyl, heterocycloalkyl, and heteroaryl is optionally substituted with one, two, or three R⁴.

In some embodiment of a compound of Formula (I), R¹³ is C₁-C₆alkyl, C₁-C₆haloalkyl, cycloalkyl, or heterocycloalkyl; wherein each alkyl, cycloalkyl, and heterocycloalkyl is optionally substituted with one, two, or three R³; and R¹⁴ is C₂-C₆alkyl, C₁-C₆haloalkyl, cycloalkyl, or heterocycloalkyl; wherein each alkyl, cycloalkyl, and heterocycloalkyl is optionally substituted with one, two, or three R⁴.

In some embodiment of a compound of Formula (I), R¹³ is C₁-C₆alkyl, C₁-C₆haloalkyl, cycloalkyl, or heterocycloalkyl; wherein each alkyl, cycloalkyl, and heterocycloalkyl is optionally substituted with one, two, or three R³; and R¹⁴ is C₂-C₆alkyl, C₁-C₆haloalkyl, cycloalkyl, heterocycloalkyl, —C₁-C₆alkyl(cycloalkyl), or heteroaryl; wherein each alkyl, cycloalkyl, heterocycloalkyl, and heteroaryl is optionally substituted with one, two, or three R⁴.

In some embodiment of a compound of Formula (I), R¹³ is C₂-C₆alkyl, C₁-C₆haloalkyl, or cycloalkyl; and R¹⁴ is C₁-C₆alkyl, C₁-C₆haloalkyl, cycloalkyl, —C₁-C₆alkyl(cycloalkyl), or heteroaryl.

In some embodiment of a compound of Formula (I), R¹³ is C₂-C₆alkyl or cycloalkyl; and R¹⁴ is C₁-C₆alkyl.

In some embodiment of a compound of Formula (I), R¹³ is C₁-C₆alkyl, C₁-C₆haloalkyl, or cycloalkyl; and R¹⁴ is C₂-C₆alkyl, C₁-C₆haloalkyl, cycloalkyl, —C₁-C₆alkyl(cycloalkyl), or heteroaryl.

In some embodiment of a compound of Formula (I), R¹³ is C₁-C₆alkyl; and R¹⁴ is C₂-C₆alkyl, cycloalkyl, —C₁-C₆alkyl(cycloalkyl), or heteroaryl.

In some embodiment of a compound of Formula (I), R¹³ is C₂-C₆alkyl, C₁-C₆haloalkyl, or cycloalkyl; and R¹⁴ is C₁-C₆alkyl, C₁-C₆haloalkyl, or cycloalkyl.

In some embodiment of a compound of Formula (I), R¹³ is C₂-C₆alkyl or cycloalkyl; and R¹⁴ is C₁-C₆alkyl.

In some embodiment of a compound of Formula (I), R¹³ is C₁-C₆alkyl, C₁-C₆haloalkyl, or cycloalkyl; and R¹⁴ is C₂-C₆alkyl, C₁-C₆haloalkyl, or cycloalkyl.

In some embodiment of a compound of Formula (I), R¹³ is C₁-C₆alkyl; and R¹⁴ is C₂-C₆alkyl or cycloalkyl.

In some embodiment of a compound of Formula (I), each alkyl, alkenyl, alkynyl, cycloalkyl, heterocycloalkyl, aryl, or heteroaryl in R¹³ is optionally substituted with one, two, or three R³. In some embodiment of a compound of Formula (I), each alkyl, alkenyl, alkynyl, cycloalkyl, heterocycloalkyl, aryl, or heteroaryl in R¹³ is optionally substituted with one or two R³. In some embodiment of a compound of Formula (I), each alkyl, alkenyl, alkynyl, cycloalkyl, heterocycloalkyl, aryl, or heteroaryl in R¹³ is optionally substituted with one R³. In some embodiment of a compound of Formula (I), each alkyl, alkenyl, alkynyl, cycloalkyl, heterocycloalkyl, aryl, or heteroaryl in R¹³ is optionally substituted with two R³. In some embodiment of a compound of Formula (I), each alkyl, alkenyl, alkynyl, cycloalkyl, heterocycloalkyl, aryl, or heteroaryl in R¹³ is optionally substituted with three R³.

In some embodiment of a compound of Formula (I), each R³ is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R³ is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R³ is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R³ is independently oxo, halogen, —CN, —OH, —OR^{a}, —NR^{b}R^{c}, C₁-C₆alkyl, C₁-C₆haloalkyl. In some embodiment of a compound of Formula (I), each R³ is independently oxo, halogen, —CN, —OH, —OMe, —NH₂, Me, or CF₃. In some embodiment of a compound of Formula (I), each R³ is independently halogen.

In some embodiment of a compound of Formula (I), each alkyl, cycloalkyl, heterocycloalkyl, aryl, and heteroaryl in R¹⁴ is optionally substituted with one, two, or three R⁴. In some embodiment of a compound of Formula (I), each alkyl, cycloalkyl, heterocycloalkyl, aryl, and heteroaryl in R¹⁴ is optionally substituted with one or two R⁴. In some embodiment of a compound of Formula (I), each alkyl, cycloalkyl, heterocycloalkyl, aryl, and heteroaryl in R¹⁴ is optionally substituted with one R⁴. In some embodiment of a compound of Formula (I), each alkyl, cycloalkyl, heterocycloalkyl, aryl, and heteroaryl in R¹⁴ is optionally substituted with two R⁴. In some embodiment of a compound of Formula (I), each alkyl, cycloalkyl, heterocycloalkyl, aryl, and heteroaryl in R¹⁴ is optionally substituted with three R⁴.

In some embodiment of a compound of Formula (I), each R⁴ is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R⁴ is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁴ is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁴ is independently oxo, halogen, —CN, —OH, —OR^{a}, —NR^{b}R^{c}, C₁-C₆alkyl, C₁-C₆haloalkyl. In some embodiment of a compound of Formula (I), each R⁴ is independently oxo, halogen, —CN, —OH, —OMe, —NH₂, Me, or CF₃. In some embodiment of a compound of Formula (I), each R⁴ is independently halogen.

In some embodiment of a compound of Formula (I), R¹⁵ is hydrogen, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three R⁵. In some embodiment of a compound of Formula (I), R¹⁵ is hydrogen, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three R⁵. In some embodiment of a compound of Formula (I), R¹⁵ is hydrogen, C₁-C₆alkyl, C₁-C₆haloalkyl, C₁-C₆hydroxyalkyl, or C₁-C₆aminoalkyl; wherein each alkyl is independently optionally substituted with one, two, or three R⁵. In some embodiment of a compound of Formula (I), R¹⁵ is hydrogen, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, or cycloalkyl. In some embodiment of a compound of Formula (I), R¹⁵ is hydrogen, C₁-C₆alkyl, C₁-C₆haloyalkyl, or C₁-C₆hydroxyalkyl. In some embodiment of a compound of Formula (I), R¹⁵ is hydrogen, C₁-C₆alkyl, or C₁-C₆hydroxyalkyl. In some embodiment of a compound of Formula (I), R¹⁵ is hydrogen or C₁-C₆alkyl. In some embodiment of a compound of Formula (I), R¹⁵ is C₁-C₆alkyl. In some embodiment of a compound of Formula (I), R¹⁵ is hydrogen, C₁-C₆alkyl, C₁-C₆haloalkyl, or C₁-C₆hydroxyalkyl.

In some embodiment of a compound of Formula (I), each alkyl, cycloalkyl, heterocycloalkyl, aryl, and heteroaryl in R¹⁵ is optionally substituted with one, two, or three R⁵. In some embodiment of a compound of Formula (I), each alkyl, cycloalkyl, heterocycloalkyl, aryl, and heteroaryl in R¹⁵ is optionally substituted with one or two R⁵. In some embodiment of a compound of Formula (I), each alkyl, cycloalkyl, heterocycloalkyl, aryl, and heteroaryl is R¹⁵ in optionally substituted with one R⁵. In some embodiment of a compound of Formula (I), each alkyl, cycloalkyl, heterocycloalkyl, aryl, and heteroaryl in R¹⁵ is optionally substituted with two R⁵. In some embodiment of a compound of Formula (I), each alkyl, cycloalkyl, heterocycloalkyl, aryl, and heteroaryl in R¹⁵ is optionally substituted with three R⁵.

In some embodiment of a compound of Formula (I), each R is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R⁵ is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁵ is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁵ is independently oxo, halogen, —CN, —OH, —OR^{a}, —NR^{b}R^{c}, C₁-C₆alkyl, C₁-C₆haloalkyl. In some embodiment of a compound of Formula (I), each R⁵ is independently oxo, halogen, —CN, —OH, —OMe, —NH₂, Me, or CF₃. In some embodiment of a compound of Formula (I), each R⁵ is independently halogen.

In some embodiment of a compound of Formula (I), R¹⁶ is hydrogen, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is optionally substituted with one, two, or three R⁶. In some embodiment of a compound of Formula (I), R¹⁶ is hydrogen, C₁-C₆alkyl, C₁-C₆haloalkyl, C₁-C₆hydroxyalkyl, or C₁-C₆aminoalkyl; wherein each alkyl is optionally substituted with one, two, or three R⁶. In some embodiment of a compound of Formula (I), R¹⁶ is hydrogen, C₁-C₆alkyl, or C₁-C₆haloalkyl; wherein each alkyl is optionally substituted with one, two, or three R⁶. In some embodiment of a compound of Formula (I), R¹⁶ is hydrogen or C₁-C₆alkyl. In some embodiment of a compound of Formula (I), R¹⁶ is hydrogen.

In some embodiment of a compound of Formula (I), R¹⁷ is hydrogen, —CN, —OR²⁰, C₁-C₆alkyl, C₁-C₆haloalkyl, C₁-C₆hydroxyalkyl, C₁-C₆aminoalkyl, C₂-C₆alkenyl, C₂-C₆alkynyl, C₃-C₁₅cycloalkyl, C₂-C₁₅heterocycloalkyl, phenyl, 5- or 6-membered heteroaryl, —C₁-C₆alkyl(phenyl), —C₁-C₆alkyl(5- or 6-membered heteroaryl), —C₁-C₆alkyl(C₃-C₁₅cycloalkyl), or —C₁-C₆alkyl(C₂-C₁₅heterocycloalkyl); wherein each alkyl, alkenyl, alkynyl, cycloalkyl, heterocycloalkyl, aryl, and heteroaryl is optionally substituted with one, two, or three R⁶.

In some embodiment of a compound of Formula (I), R¹⁷ is hydrogen, —CN, —OR²⁰, C₁-C₆alkyl, C₁-C₆haloalkyl, C₁-C₆hydroxyalkyl, C₁-C₆aminoalkyl, C₂-C₆alkenyl, C₂-C₆alkynyl, C₃-C₁₀cycloalkyl, C₂-C₁₀heterocycloalkyl, phenyl, 5- or 6-membered heteroaryl, —C₁-C₆alkyl(phenyl), —C₁-C₆alkyl(5- or 6-membered heteroaryl), —C₁-C₆alkyl(C₃-C₁₀cycloalkyl), or —C₁-C₆alkyl(C₂-C₁₀heterocycloalkyl); wherein each alkyl, alkenyl, alkynyl, cycloalkyl, heterocycloalkyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, C₂-C₆alkynyl, C₃-C₁₀cycloalkyl, C₂-C₁₀cycloalkenyl, C₃-C₁₀heterocycloalkyl, C₂-C₁₀heterocycloalkenyl, phenyl, 5- or 6-membered heteroaryl, —C₁-C₆alkyl(phenyl), —C₁-C₆alkyl(5- or 6-membered heteroaryl), —C₁-C₆alkyl(C₃-C₁₀cycloalkyl), or —C₁-C₆alkyl(C₂-C₁₀heterocycloalkyl); wherein each alkyl, alkynyl, cycloalkyl, cycloalkenyl, heterocycloalkyl, heterocycloalkenyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, C₂-C₆alkynyl, C₃-C₁₀cycloalkyl, C₃-C₁₀cycloalkenyl, C₂-C₁₀heterocycloalkyl, phenyl, 5- or 6-membered heteroaryl, —C₁-C₆alkyl(5- or 6-membered heteroaryl), —C₁-C₆alkyl(C₃-C₁₀cycloalkyl), or —C₁-C₆alkyl(C₂-C₁₀heterocycloalkyl); wherein each alkyl, alkynyl, cycloalkyl, cycloalkenyl, heterocycloalkyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, C₂-C₆alkynyl, cycloalkyl, cycloalkenyl, heterocycloalkyl, heterocycloalkenyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkynyl, cycloalkyl, cycloalkenyl, heterocycloalkyl, heterocycloalkenyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, C₂-C₆alkynyl, cycloalkyl, cycloalkenyl, heterocycloalkyl, aryl, heteroaryl, —C₁-C₆alkyl(heteroaryl), —C₁-C₆alkyl(cycloalkyl), or —C₁-C₆alkyl(heterocycloalkyl); wherein each alkyl, alkynyl, cycloalkyl, cycloalkenyl, heterocycloalkyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, C₁-C₆aminoalkyl, cycloalkyl, heterocycloalkyl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, cycloalkyl, heterocycloalkyl, aryl, and heteroaryl is optionally substituted with one, two, or three R⁶.

In some embodiment of a compound of Formula (I), R¹⁷ is —OR²⁰, C₁-C₆alkyl, C₁-C₆haloalkyl, C₁-C₆hydroxyalkyl, cycloalkyl, heterocycloalkyl, —C₁-C₆alkyl(heteroaryl), or —C₁-C₆alkyl(cycloalkyl); wherein each alkyl, cycloalkyl, heterocycloalkyl, and heteroaryl is optionally substituted with one, two, or three R⁶.

In some embodiment of a compound of Formula (I), R¹⁷ is C₁-C₆alkyl, C₁-C₆haloalkyl, C₁-C₆hydroxyalkyl, or cycloalkyl; each optionally substituted with one, two, or three R⁶.

In some embodiment of a compound of Formula (I), R¹⁷ is C₁-C₆hydroxyalkyl, cycloalkyl, or heterocycloalkyl; each optionally substituted with one, two, or three R⁶.

In some embodiment of a compound of Formula (I), R¹⁷ is C₁-C₆alkyl or cycloalkyl; each optionally substituted with one, two, or three R⁶. In some embodiment of a compound of Formula (I), R¹⁷ is C₁-C₆alkyl optionally substituted with one, two, or three R⁶.

In some embodiment of a compound of Formula (I), R¹⁷ is C₁-C₆haloalkyl, C₁-C₆hydroxyalkyl or cycloalkyl; each optionally substituted with one, two, or three R⁶. In some embodiment of a compound of Formula (I), R¹⁷ is C₁-C₆haloalkyl optionally substituted with one, two, or three R⁶. In some embodiment of a compound of Formula (I), R¹⁷ is C₁-C₆hydroxyalkyl optionally substituted with one, two, or three R⁶. In some embodiment of a compound of Formula (I), R¹⁷ is cycloalkyl optionally substituted with one, two, or three R⁶. In some embodiment of a compound of Formula (I), R¹⁷ is cyclohexyl or cyclopentyl optionally substituted with one, two, or three R⁶. In some embodiment of a compound of Formula (I), R¹⁷ is cyclohexyl optionally substituted with one, two, or three R⁶.

In some embodiment of a compound of Formula (I), R¹⁷ is C₁-C₆alkyl, C₁-C₆haloalkyl, C₁-C₆hydroxyalkyl, cycloalkyl, heterocycloalkyl, —C₁-C₆alkyl(heteroaryl), or —C₁-C₆alkyl(cycloalkyl); wherein each alkyl, cycloalkyl, heterocycloalkyl, and heteroaryl is optionally substituted with one, two, or three R⁶.

In some embodiment of a compound of Formula (I), R¹⁷ is C₁-C₆alkyl, C₁-C₆haloalkyl, C₁-C₆hydroxyalkyl, cycloalkyl, or heterocycloalkyl; wherein each alkyl, cycloalkyl, heterocycloalkyl, and heteroaryl is optionally substituted with one, two, or three R⁶.

In some embodiment of a compound of Formula (I), R¹⁷ is C₁-C₆hydroxyalkyl, cycloalkyl, or heterocycloalkyl; each optionally substituted with one, two, or three R⁶.

In some embodiment of a compound of Formula (I), R¹⁷ is C₁-C₆alkyl or cycloalkyl; each optionally substituted with one, two, or three R⁶.

In some embodiment of a compound of Formula (I), each alkyl, alkenyl, alkynyl, cycloalkyl, cycloalkenyl, heterocycloalkyl, heterocycloalkenyl, aryl, or heteroaryl in R¹⁶ or R¹⁷ is optionally substituted with one, two, or three R⁶. In some embodiment of a compound of Formula (I), each alkyl, alkenyl, alkynyl, cycloalkyl, cycloalkenyl, heterocycloalkyl, heterocycloalkenyl, aryl, or heteroaryl in R¹⁶ or R¹⁷ is optionally substituted with one or two R⁶. In some embodiment of a compound of Formula (I), each alkyl, alkenyl, alkynyl, cycloalkyl, cycloalkenyl, heterocycloalkyl, heterocycloalkenyl, aryl, or heteroaryl in R¹⁶ or R¹⁷ is optionally substituted with one R⁶. In some embodiment of a compound of Formula (I), each alkyl, alkenyl, alkynyl, cycloalkyl, cycloalkenyl, heterocycloalkyl, heterocycloalkenyl, aryl, or heteroaryl in R¹⁶ or R¹⁷ is optionally substituted with two R⁶. In some embodiment of a compound of Formula (I), each alkyl, alkenyl, alkynyl, cycloalkyl, cycloalkenyl, heterocycloalkyl, heterocycloalkenyl, aryl, or heteroaryl in R¹⁶ or R¹⁷ is optionally substituted with three R⁶.

In some embodiment of a compound of Formula (I), each R⁶ is independently oxo, halogen, —CN, —OH, —OR^{a}, —S(═O)₂R^{a}, —NR^{b}R^{c}, —NHS(═O)₂R^{a}, —S(═O)₂NR^{b}R^{c}, —B(OR^{b})(OR^{c}), —C(═O)R^{a}, —C(═O)OR^{b}, —C(═O)NR^{b}R^{c}, —NR^{b}C(═O)R^{a}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three R^{6a}.

In some embodiment of a compound of Formula (I), each R⁶ is independently oxo, halogen, —CN, —OH, —OR^{a}, —S(═O)₂R^{a}, —NR^{b}R^{c}, —NHS(═O)₂R^{a}, —B(OR^{b})(OR^{c}), —C(═O)R^{a}, —C(═O)OR^{b}, —C(═O)NR^{b}R^{c}, —NR^{b}C(═O)R^{a}, C₁-C₆alkyl, C₁-C₆haloalkyl, C₁-C₆hydroxyalkyl, cycloalkyl, heterocycloalkyl, or heteroaryl; wherein each alkyl, alkenyl, alkynyl, cycloalkyl, heterocycloalkyl, aryl, and heteroaryl is independently optionally substituted with one, two, or three R^{6a}.

In some embodiment of a compound of Formula (I), each R⁶ is independently halogen, —CN, —OH, —OR^{a}, —NR^{b}R^{c}, —B(OR^{b})(OR^{c}), —C(═O)R^{a}, —C(═O)OR^{b}, —C(═O)NR^{b}R^{c}, C₁-C₆alkyl, C₁-C₆haloalkyl, C₁-C₆hydroxyalkyl, cycloalkyl, heterocycloalkyl, aryl, or heteroaryl; wherein each alkyl, cycloalkyl, heterocycloalkyl, aryl, and heteroaryl is independently optionally substituted with one, two, or three R^{6a}.

In some embodiment of a compound of Formula (I), each R⁶ is independently halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, cycloalkyl, heterocycloalkyl, aryl, or heteroaryl; wherein each alkyl, cycloalkyl, heterocycloalkyl, aryl, and heteroaryl is independently optionally substituted with one, two, or three R^{6a}.

In some embodiment of a compound of Formula (I), each R⁶ is independently halogen, —CN, —OH, —OR^{a}, —NR^{b}R^{c}, —C(═O)OR^{b}, —C(═O)NR^{b}R^{c}, C₁-C₆alkyl, C₁-C₆haloalkyl, C₁-C₆hydroxyalkyl, cycloalkyl, or heteroaryl; wherein each alkyl, cycloalkyl, heterocycloalkyl, and heteroaryl is independently optionally substituted with one, two, or three R^{6a}. In some embodiment of a compound of Formula (I), each R⁶ is independently halogen, —OH, —OR^{a}, —C(═O)OR^{b}, —C(═O)NR^{b}R^{c}, C₁-C₆alkyl, or heteroaryl; wherein each alkyl, cycloalkyl and heteroaryl is independently optionally substituted with one, two, or three R^{6a}. In some embodiment of a compound of Formula (I), each R⁶ is independently halogen, —C(═O)OR^{b}, —C(═O)NR^{b}R^{c}, C₁-C₆alkyl, or heteroaryl; wherein each alkyl, cycloalkyl and heteroaryl is independently optionally substituted with one, two, or three R^{6a}. In some embodiment of a compound of Formula (I), each R⁶ is independently halogen, —C(═O)OR^{b}, —C(═O)NR^{b}R^{c}, or heteroaryl optionally substituted with one, two, or three R^{6a}. In some embodiment of a compound of Formula (I), each R⁶ is independently halogen, —C(═O)OR^{b}, or —C(═O)NR^{b}R^{c}. In some embodiment of a compound of Formula (I), each R⁶ is independently —C(═O)OR^{b} or —C(═O)NR^{b}R^{c}.

In some embodiment of a compound of Formula (I), each alkyl, alkenyl, alkynyl, cycloalkyl, heterocycloalkyl, aryl, or heteroaryl in R⁶ is optionally substituted with one, two, or three R^{6a}. In some embodiment of a compound of Formula (I), each alkyl, alkenyl, alkynyl, cycloalkyl, heterocycloalkyl, aryl, or heteroaryl in R⁶ is optionally substituted with one or two R^{6a}. In some embodiment of a compound of Formula (I), each alkyl, alkenyl, alkynyl, cycloalkyl, heterocycloalkyl, aryl, or heteroaryl in R⁶ is optionally substituted with one R^{6a}. In some embodiment of a compound of Formula (I), each alkyl, alkenyl, alkynyl, cycloalkyl, heterocycloalkyl, aryl, or heteroaryl in R⁶ is optionally substituted with two R^{6a}. In some embodiment of a compound of Formula (I), each alkyl, alkenyl, alkynyl, cycloalkyl, heterocycloalkyl, aryl, or heteroaryl in R⁶ is optionally substituted with three R^{6a}.

In some embodiment of a compound of Formula (I), each R^{6a} is independently halogen, —CN, —OH, —OR^{a}, —NR^{b}R^{c}, C₁-C₆alkyl, or C₁-C₆haloalkyl. In some embodiment of a compound of Formula (I), each R^{6a} is independently oxo, halogen, —CN, —OH, —OMe, —NH₂, Me, or CF₃. In some embodiment of a compound of Formula (I), each R^{6a} is independently halogen, C₁-C₆alkyl, or C₁-C₆haloalkyl. In some embodiment of a compound of Formula (I), each R^{6a} is independently halogen.

In some embodiment of a compound of Formula (I), R¹⁶ and R¹⁷ are taken together with the nitrogen atom to which they are attached to form a heterocycloalkyl optionally substituted with one, two, or three R⁷. In some embodiment of a compound of Formula (I), R¹⁶ and R¹⁷ are taken together with the nitrogen atom to which they are attached to form a heterocycloalkyl optionally substituted with one, two, or three R⁷; wherein the heterocycloalkyl is pyrrolidine, piperidine, morpholine, or piperazine. In some embodiment of a compound of Formula (I), R¹⁶ and R¹⁷ are taken together with the nitrogen atom to which they are attached to form a heterocycloalkyl optionally substituted with one, two, or three R⁷; wherein the heterocycloalkyl is piperidine.

In some embodiment of a compound of Formula (I), the heterocycloalkyl or heterocycloalkenyl formed when R¹⁶ and R¹⁷ are taken together is optionally substituted with one, two, or three R⁷. In some embodiment of a compound of Formula (I), the heterocycloalkyl or heterocycloalkenyl formed when R¹⁶ and R¹⁷ are taken together is optionally substituted with one or two R⁷. In some embodiment of a compound of Formula (I), the heterocycloalkyl or heterocycloalkenyl formed when R¹⁶ and R¹⁷ are taken together is optionally substituted with one R⁷. In some embodiment of a compound of Formula (I), the heterocycloalkyl or heterocycloalkenyl formed when R¹⁶ and R¹⁷ are taken together is optionally substituted with two R⁷. In some embodiment of a compound of Formula (I), the heterocycloalkyl or heterocycloalkenyl formed when R¹⁶ and R¹⁷ are taken together is optionally substituted with three R⁷.

In some embodiment of a compound of Formula (I), each R⁷ is independently oxo, halogen, —CN, —OH, —OR^{a}, —NR^{b}R^{c}, —C(═O)R^{a}, —C(═O)OR^{b}, C₁-C₆alkyl, C₁-C₆haloalkyl, C₁-C₆hydroxyalkyl, C₁-C₆aminoalkyl, cycloalkyl, or heterocycloalkyl; wherein each alkyl, cycloalkyl, and heterocycloalkyl is independently optionally substituted with one, two, or three oxo, halogen, —CN, —OH, —OMe, —NH₂, C₁-C₆alkyl, C₁-C₆haloalkyl, C₁-C₆hydroxyalkyl, or C₁-C₆aminoalkyl. In some embodiment of a compound of Formula (I), each R⁷ is independently oxo, halogen, —CN, —OH, —OR^{a}, —NR^{b}R^{c}, C₁-C₆alkyl, C₁-C₆haloalkyl, C₁-C₆hydroxyalkyl, or C₁-C₆aminoalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁷ is independently oxo, halogen, —CN, —OH, —OR^{a}, —NR^{b}R^{c}, C₁-C₆alkyl, C₁-C₆haloalkyl; wherein each alkyl is independently optionally substituted with one, two, or three oxo, halogen, —CN, —OH, —OMe, —NH₂, C₁-C₆alkyl, or C₁-C₆haloalkyl. In some embodiment of a compound of Formula (I), each R⁷ is independently oxo, halogen, —CN, —OH, —OR^{a}, —NR^{b}R^{c}, C₁-C₆alkyl, C₁-C₆haloalkyl. In some embodiment of a compound of Formula (I), each R⁷ is independently halogen, —CN, —OH, —OW, —NR^{b}R^{c}, C₁-C₆alkyl, C₁-C₆haloalkyl, or C₁-C₆hydroxyalkyl. In some embodiment of a compound of Formula (I), each R⁷ is independently oxo, halogen, —CN, —OH, —OMe, —NH₂, Me, or CF₃. In some embodiment of a compound of Formula (I), each R⁷ is independently —OH or C₁-C₆alkyl. In some embodiment of a compound of Formula (I), each R⁷ is independently oxo, halogen, —CN, —OH, —OR^{a}, —NR^{b}R^{c}, C₁-C₆alkyl, C₁-C₆haloalkyl, or C₁-C₆hydroxyalkyl.

In some embodiments of a compound of Formula (I), or a pharmaceutically acceptable salt, solvate, or stereoisomer thereof, each R^{a} is independently C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), or a pharmaceutically acceptable salt, solvate, or stereoisomer thereof, each R^{a} is independently C₁-C₆alkyl, C₁-C₆haloalkyl, cycloalkyl, or heterocycloalkyl. In some embodiments of a compound of Formula (I), or a pharmaceutically acceptable salt, solvate, or stereoisomer thereof, each R^{a} is independently C₁-C₆alkyl, C₁-C₆haloalkyl, or cycloalkyl. In some embodiments of a compound of Formula (I), or a pharmaceutically acceptable salt, solvate, or stereoisomer thereof, each R^{a} is independently C₁-C₆alkyl or C₁-C₆haloalkyl. In some embodiments of a compound of Formula (I), or a pharmaceutically acceptable salt, solvate, or stereoisomer thereof, each R^{a} is independently C₁-C₆alkyl.

In some embodiments of a compound of Formula (I), or a pharmaceutically acceptable salt, solvate, or stereoisomer thereof, each R^{b} and R^{c} is independently hydrogen, C₁-C₆alkyl, C₁-C₆haloalkyl, C₁-C₆hydroxyalkyl, C₁-C₆aminoalkyl, cycloalkyl, heterocycloalkyl, aryl, or heteroaryl. In some embodiments of a compound of Formula (I), or a pharmaceutically acceptable salt, solvate, or stereoisomer thereof, each R^{b} and R^{c} is independently hydrogen, C₁-C₆alkyl, C₁-C₆haloalkyl, cycloalkyl, or heterocycloalkyl. In some embodiments of a compound of Formula (I), or a pharmaceutically acceptable salt, solvate, or stereoisomer thereof, each R^{b} and R^{c} is independently hydrogen, C₁-C₆alkyl, C₁-C₆haloalkyl, or cycloalkyl. In some embodiments of a compound of Formula (I), or a pharmaceutically acceptable salt, solvate, or stereoisomer thereof, each R^{b} and R^{c} is independently hydrogen, C₁-C₆alkyl, or C₁-C₆haloalkyl. In some embodiments of a compound of Formula (I), or a pharmaceutically acceptable salt, solvate, or stereoisomer thereof, each R^{b} and R^{c} is independently hydrogen or C₁-C₆alkyl. In some embodiments of a compound of Formula (I), or a pharmaceutically acceptable salt, solvate, or stereoisomer thereof, each R^{b} and R^{c} is hydrogen.

In some embodiments of a compound of Formula (I), or a pharmaceutically acceptable salt, solvate, or stereoisomer thereof, R^{b} and R^{c} are taken together with the atom to which they are attached to form a heterocycloalkyl optionally substituted with one, two, or three halogen, C₁-C₆alkyl, or C₁-C₆haloalkyl.

Any combination of the groups described above for the various variables is contemplated herein. Throughout the specification, groups and substituents thereof are chosen by one skilled in the field to provide stable moieties and compounds.

Described herein is a compound of Formula (I), or a pharmaceutically acceptable salt, solvate, or stereoisomer thereof, selected from a compound in Table 1.

TABLE 1
Exemplary compounds

| | | | ESI-MS |
|---|---|---|---|
| Ex. | Structure | Chemical Name | (M + H)⁺(m/z) |
| 1 | 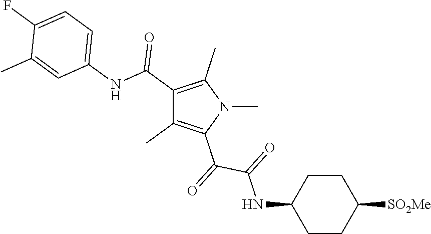 [SMILES: Cc1cc(NC(=O)c2c(C)c(C(=O)C(=O)N[C@H]3CC[C@@H](S(C)(=O)=O)CC3)n(C)c2C)ccc1F] | N-(4-fluoro-3-methylphenyl)- 1,2,4-trimethyl-5-(2-(((1s,4s)-4- (methylsulfonyl)cyclohexyl)amino)- 2-oxoacetyl)-1H-pyrrole-3- carboxamide | 492 |
| 2 | 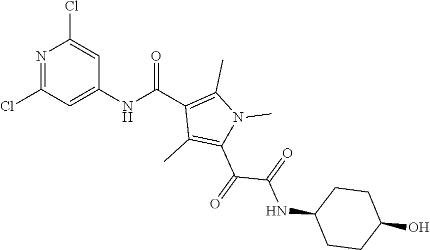 [SMILES: Cc1c(C(=O)Nc2cc(Cl)nc(Cl)c2)c(C)n(C)c1C(=O)C(=O)N[C@H]1CC[C@@H](O)CC1] | N-(2,6-dichloropyridin-4-yl)-5- (2-(((1s,4s)-4- hydroxycyclohexyl)amino)-2- oxoacetyl)-1,2,4-trimethyl-1H- pyrrole-3-carboxamide | 468 |
| 3 | 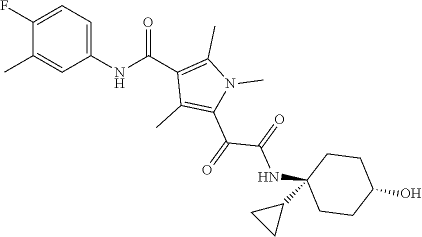 [SMILES: Cc1cc(NC(=O)c2c(C)c(C(=O)C(=O)N[C@]3(C4CC4)CC[C@@H](O)CC3)n(C)c2C)ccc1F] | 5-(2-(((1r,4r)-1-cyclopropyl-4- hydroxycyclohexyl)amino)-2- oxoacetyl)-N-(4-fluoro-3- methylphenyl)-1,2,4-trimethyl- 1H-pyrrole-3-carboxamide | 470 |
| 4 | 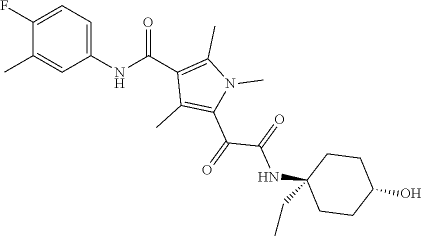 [SMILES: CC[C@]1(NC(=O)C(=O)c2c(C)c(C(=O)Nc3ccc(F)c(C)c3)c(C)n2C)CC[C@H](O)CC1] | 5-(2-(((1r,4r)-1-ethyl-4- hydroxycyclohexyl)amino)-2- oxoacetyl)-N-(4-fluoro-3- methylphenyl)-1,2,4-trimethyl- 1H-pyrrole-3-carboxamide | 458 |
| 5 | 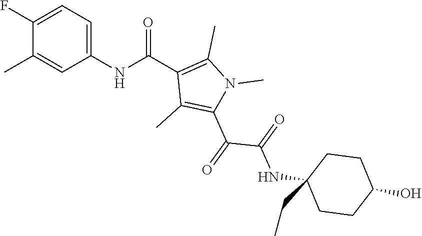 [SMILES: CC[C@]1(NC(=O)C(=O)c2c(C)c(C(=O)Nc3ccc(F)c(C)c3)c(C)n2C)CC[C@@H](O)CC1] | 5-(2-(((1s,4s)-1-ethyl-4- hydroxycyclohexyl)amino)-2- oxoacetyl)-N-(4-fluoro-3- methylphenyl)-1,2,4-trimethyl- 1H-pyrrole-3-carboxamide | 458 |
| 6 | 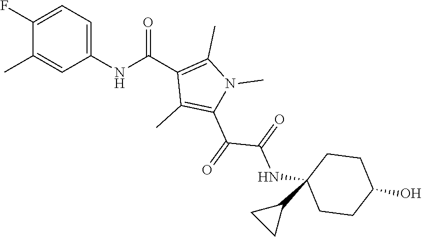 [SMILES: Cc1cc(NC(=O)c2c(C)c(C(=O)C(=O)N[C@]3(C4CC4)CC[C@H](O)CC3)n(C)c2C)ccc1F] | 5-(2-(((1s,4s)-1-cyclopropyl-4- hydroxycyclohexyl)amino)-2- oxoacetyl)-N-(4-fluoro-3- methylphenyl)-1,2,4-trimethyl- 1H-pyrrole-3-carboxamide | 470 |
| 7 | 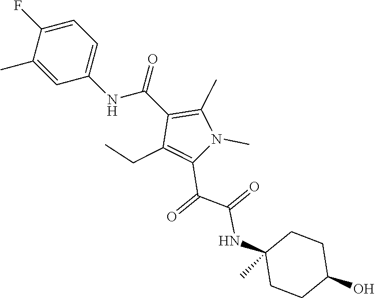 [SMILES: CCc1c(C(=O)Nc2ccc(F)c(C)c2)c(C)n(C)c1C(=O)C(=O)N[C@]1(C)CC[C@H](O)CC1] | 4-ethyl-N-(4-fluoro-3- methylphenyl)-5-(2-(((1s,4s)-4- hydroxy-1- methylcyclohexyl)amino)-2- oxoacetyl)-1,2-dimethyl-1H- pyrrole-3-carboxamide | 458 |
| 8 | 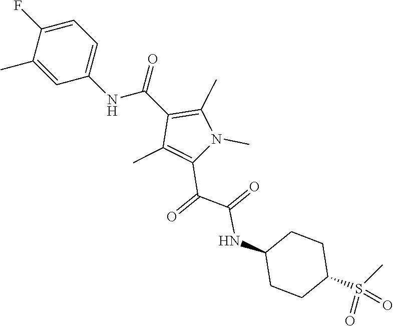 [SMILES: Cc1cc(NC(=O)c2c(C)c(C(=O)C(=O)N[C@H]3CC[C@H](S(C)(=O)=O)CC3)n(C)c2C)ccc1F] | N-(4-fluoro-3-methylphenyl)- 1,2,4-trimethyl-5-(2-(((1r,4r)-4- (methylsulfonyl)cyclohexyl)amino)- 2-oxoacetyl)-1H-pyrrole-3- carboxamide | 492 |
| 9 | 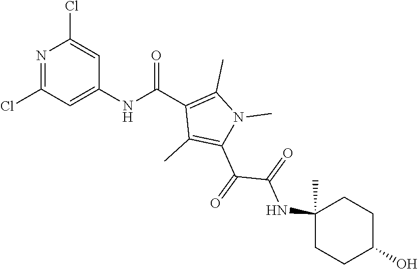 [SMILES: Cc1c(C(=O)Nc2cc(Cl)nc(Cl)c2)c(C)n(C)c1C(=O)C(=O)N[C@]1(C)CC[C@@H](O)CC1] | N-(2,6-dichloropyridin-4-yl)-5- (2-(((1r,4r)-4-hydroxy-1- methylcyclohexyl)amino)-2- oxoacetyl)-1,2,4-trimethyl-1H- pyrrole-3-carboxamide | 482 |
| 10 | 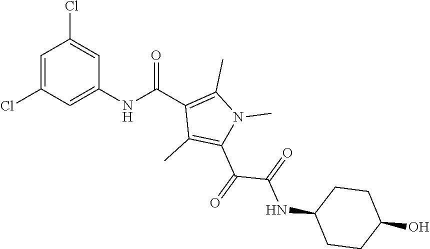 [SMILES: Cc1c(C(=O)Nc2cc(Cl)cc(Cl)c2)c(C)n(C)c1C(=O)C(=O)N[C@H]1CC[C@@H](O)CC1] | N-(3,5-dichlorophenyl)-5-(2- (((1s,4s)-4- hydroxycyclohexyl)amino)-2- oxoacetyl)-1,2,4-trimethyl-1H- pyrrole-3-carboxamide | 467 |
| 11 | 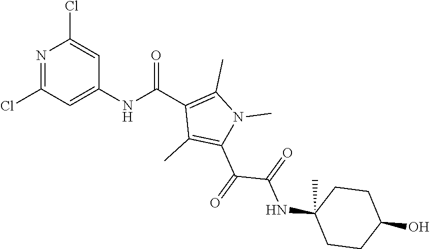 [SMILES: Cc1c(C(=O)Nc2cc(Cl)nc(Cl)c2)c(C)n(C)c1C(=O)C(=O)N[C@]1(C)CC[C@H](O)CC1] | N-(2,6-dichloropyridin-4-yl)-5- (2-(((1s,4s)-4-hydroxy-1- methylcyclohexyl)amino)-2- oxoacetyl)-1,2,4-trimethyl-1H- pyrrole-3-carboxamide | 482 |
| 12 | 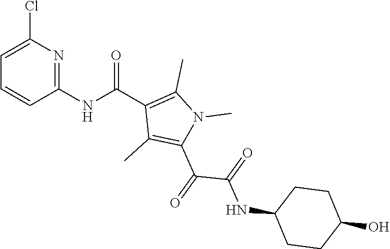 [SMILES: Cc1c(C(=O)Nc2cccc(Cl)n2)c(C)n(C)c1C(=O)C(=O)N[C@H]1CC[C@@H](O)CC1] | N-(6-chloropyridin-2-yl)-5-(2- (((1s,4s)-4- hydroxycyclohexyl)amino)-2- oxoacetyl)-1,2,4-trimethyl-1H- pyrrole-3-carboxamide | 433 |
| 13 | 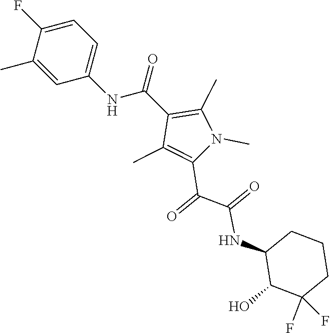 [SMILES: Cc1cc(NC(=O)c2c(C)c(C(=O)C(=O)N[C@H]3CCCC(F)(F)[C@@H]3O)n(C)c2C)ccc1F] | 5-(2-(((1S,2R)-3,3-difluoro-2- hydroxycyclohexyl)amino)-2- oxoacetyl)-N-(4-fluoro-3- methylphenyl)-1,2,4-trimethyl- 1H-pyrrole-3-carboxamide | 466 |
| 14 | 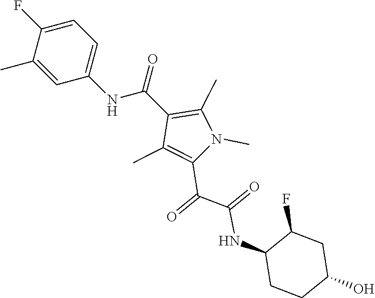 [SMILES: Cc1cc(NC(=O)c2c(C)c(C(=O)C(=O)N[C@@H]3CC[C@@H](O)C[C@@H]3F)n(C)c2C)ccc1F] | N-(4-fluoro-3-methylphenyl)-5- (2-(((1R,2S,4R)-2-fluoro-4- hydroxycyclohexyl)amino)-2- oxoacetyl)-1,2,4-trimethyl-1H- pyrrole-3-carboxamide | 448 |
| 15 | 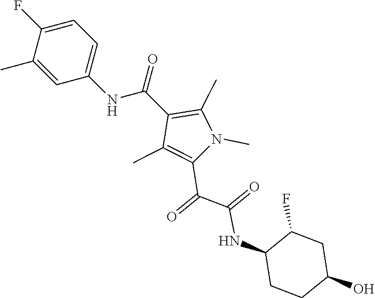 [SMILES: Cc1cc(NC(=O)c2c(C)c(C(=O)C(=O)N[C@@H]3CC[C@H](O)C[C@H]3F)n(C)c2C)ccc1F] | N-(4-fluoro-3-methylphenyl)-5- (2-(((1R,2R,4S)-2-fluoro-4- hydroxycyclohexyl)amino)-2- oxoacetyl)-1,2,4-trimethyl-1H- pyrrole-3-carboxamide | 448 |
| 16 | 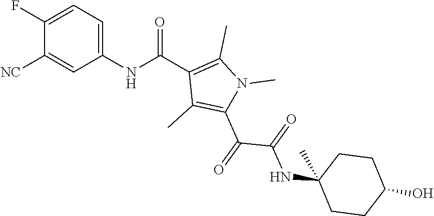 [SMILES: Cc1c(C(=O)Nc2ccc(F)c(C#N)c2)c(C)n(C)c1C(=O)C(=O)N[C@]1(C)CC[C@@H](O)CC1] | N-(3-cyano-4-fluorophenyl)-5- (2-(((1r,4r)-4-hydroxy-1- methylcyclohexyl)amino)-2- oxoacetyl)-1,2,4-trimethyl-1H- pyrrole-3-carboxamide | 455 |
| 17 | 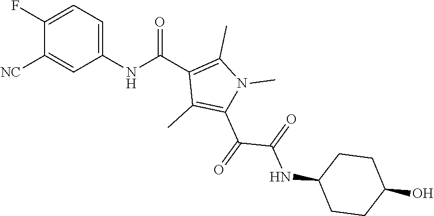 [SMILES: Cc1c(C(=O)Nc2ccc(F)c(C#N)c2)c(C)n(C)c1C(=O)C(=O)N[C@H]1CC[C@@H](O)CC1] | N-(3-cyano-4-fluorophenyl)-5- (2-(((1s,4s)-4- hydroxycyclohexyl)amino)-2- oxoacetyl)-1,2,4-trimethyl-1H- pyrrole-3-carboxamide | 441 |
| 18 | 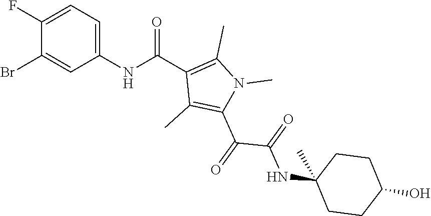 [SMILES: Cc1c(C(=O)Nc2ccc(F)c(Br)c2)c(C)n(C)c1C(=O)C(=O)N[C@]1(C)CC[C@@H](O)CC1] | N-(3-bromo-4-fluorophenyl)-5- (2-(((1r,4r)-4-hydroxy-1- methylcyclohexyl)amino)-2- oxoacetyl)-1,2,4-trimethyl-1H- pyrrole-3-carboxamide | 509 |
| 19 | 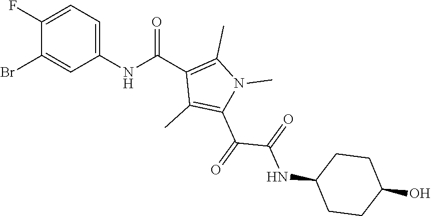 [SMILES: Cc1c(C(=O)Nc2ccc(F)c(Br)c2)c(C)n(C)c1C(=O)C(=O)N[C@H]1CC[C@@H](O)CC1] | N-(3-bromo-4-fluorophenyl)-5- (2-(((1s,4s)-4- hydroxycyclohexyl)amino)-2- oxoacetyl)-1,2,4-trimethyl-1H- pyrrole-3-carboxamide | 495 |
| 20 | 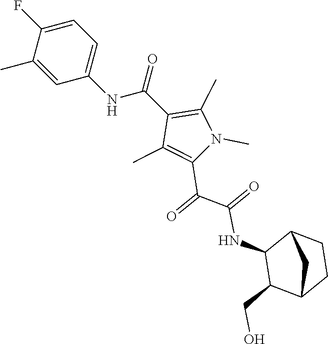 [SMILES: Cc1cc(NC(=O)c2c(C)c(C(=O)C(=O)N[C@H]3[C@@H]4CC[C@@H](C4)[C@H]3CO)n(C)c2C)ccc1F] | N-(4-fluoro-3-methylphenyl)-5- (2-(((1R,2S,3R,4S)-3- (hydroxymethyl)bicyclo[2.2.1] heptan-2-yl)amino)-2-oxoacetyl)- 1,2,4-trimethyl-1H-pyrrole-3- carboxamide | 456 |
| 21 | 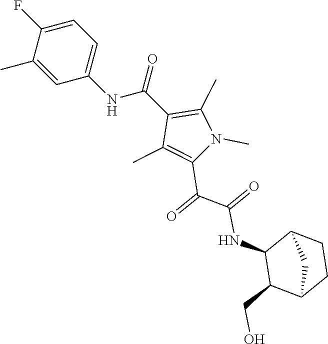 [SMILES: Cc1cc(NC(=O)c2c(C)c(C(=O)C(=O)N[C@H]3[C@H]4CC[C@H](C4)[C@H]3CO)n(C)c2C)ccc1F] | N-(4-fluoro-3-methylphenyl)-5- (2-(((1S,2S,3R,4R)-3- (hydroxymethyl)bicyclo[2.2.1] heptan-2-yl)amino)-2-oxoacetyl)- 1,2,4-trimethyl-1H-pyrrole-3- carboxamide | 456 |
| 22 | 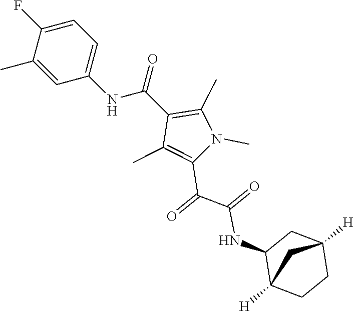 [SMILES: Cc1cc(NC(=O)c2c(C)c(C(=O)C(=O)N[C@H]3C[C@H]4CC[C@@H]3C4)n(C)c2C)ccc1F] | 5-(2-(((1S,2S,4R)- bicyclo[2.2.1]heptan-2- yl)amino)-2-oxoacetyl)-N-(4- fluoro-3-methylphenyl)-1,2,4- trimethyl-1H-pyrrole-3- carboxamide | 426 |
| 23 | 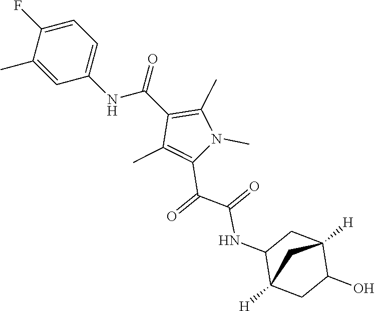 [SMILES: Cc1cc(NC(=O)c2c(C)c(C(=O)C(=O)NC3C[C@@H]4C[C@H]3CC4O)n(C)c2C)ccc1F] | N-(4-fluoro-3-methylphenyl)-5- (2-(((1R,4R)-5- hydroxybicyclo[2.2.1]heptan-2- yl)amino)-2-oxoacetyl)-1,2,4- trimethyl-1H-pyrrole-3- carboxamide | 442 |
| 24 | 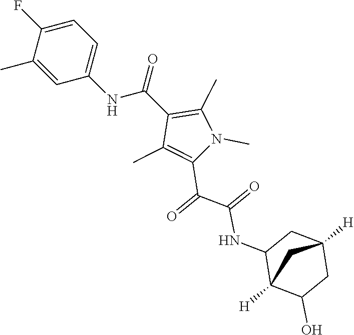 [SMILES: Cc1cc(NC(=O)c2c(C)c(C(=O)C(=O)NC3C[C@H]4CC(O)[C@@H]3C4)n(C)c2C)ccc1F] | N-(4-fluoro-3-methylphenyl)-5- (2-(((1S,4R)-6- hydroxybicyclo[2.2.1]heptan-2- yl)amino)-2-oxoacetyl)-1,2,4- trimethyl-1H-pyrrole-3- carboxamide | 442 |
| 25 | 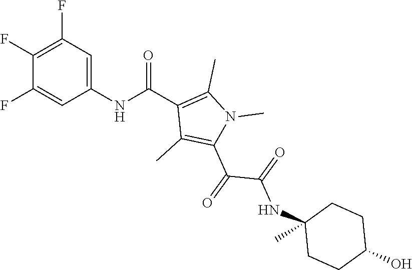 [SMILES: Cc1c(C(=O)Nc2cc(F)c(F)c(F)c2)c(C)n(C)c1C(=O)C(=O)N[C@]1(C)CC[C@@H](O)CC1] | 5-(2-(((1r,4r)-4-hydroxy-1- methylcyclohexyl)amino)-2- oxoacetyl)-1,2,4-trimethyl-N- (3,4,5-trifluorophenyl)-1H- pyrrole-3-carboxamide | 466 |
| 26 | 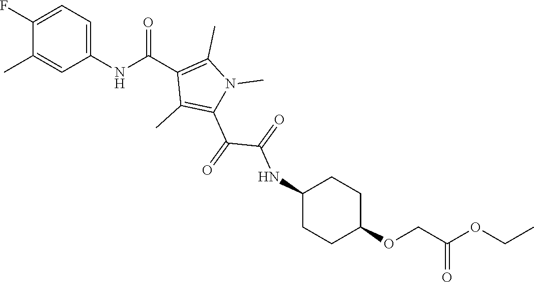 [SMILES: CCOC(=O)CO[C@H]1CC[C@@H](NC(=O)C(=O)c2c(C)c(C(=O)Nc3ccc(F)c(C)c3)c(C)n2C)CC1] | ethyl 2-(((1s,4s)-4-(2-(4-((4- fluoro-3- methylphenyl)carbamoyl)-1,3,5- trimethyl-1H-pyrrol-2-yl)-2- oxoacetamido)cyclohexyl)oxy) acetate | 516 |
| 27 | 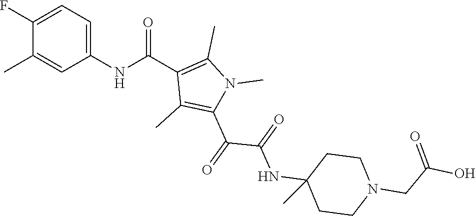 [SMILES: Cc1cc(NC(=O)c2c(C)c(C(=O)C(=O)NC3(C)CCN(CC(=O)O)CC3)n(C)c2C)ccc1F] | 2-(4-(2-(4-((4-fluoro-3- methylphenyl)carbamoyl)-1,3,5- trimethyl-1H-pyrrol-2-yl)-2- oxoacetamido)-4- methylpiperidin-1-yl)acetic acid | 487 |
| 28 | 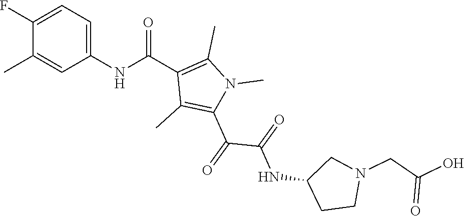 [SMILES: Cc1cc(NC(=O)c2c(C)c(C(=O)C(=O)N[C@H]3CCN(CC(=O)O)C3)n(C)c2C)ccc1F] | (S)-2-(3-(2-(4-((4-fluoro-3- methylphenyl)carbamoyl)-1,3,5- trimethyl-1H-pyrrol-2-yl)-2- oxoacetamido)pyrrolidin-1- yl)acetic acid | 459 |
| 29 | 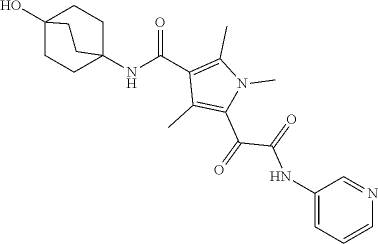 [SMILES: Cc1c(C(=O)NC23CCC(O)(CC2)CC3)c(C)n(C)c1C(=O)C(=O)Nc1cccnc1] | N-(4- hydroxybicyclo[2.2.2]octan-1- yl)-1,2,4-trimethyl-5-(2-oxo-2- (pyridin-3-ylamino)acetyl)-1H- pyrrole-3-carboxamide | 425 |
| 30 | 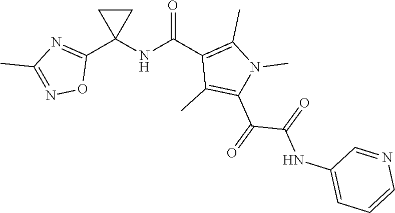 [SMILES: Cc1noc(C2(NC(=O)c3c(C)c(C(=O)C(=O)Nc4cccnc4)n(C)c3C)CC2)n1] | 1,2,4-trimethyl-N-(1-(3-methyl- 1,2,4-oxadiazol-5- yl)cyclopropyl)-5-(2-oxo-2- (pyridin-3-ylamino)acetyl)-1H- pyrrole-3-carboxamide | 423 |
| 31 | 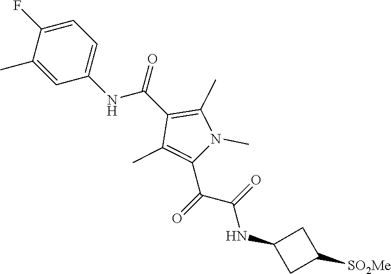 | N-(4-fluoro-3-methylphenyl)- 1,2,4-trimethyl-5-(2-(((1s,3s)-3- (methylsulfonyl)cyclobutyl)amino)- 2-oxoacetyl)-1H-pyrrole-3- carboxamide | 464 |
| 32 | 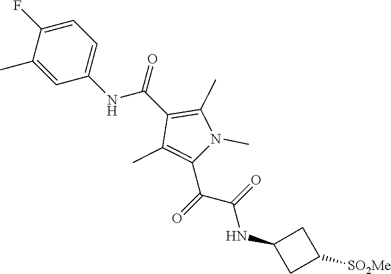 | N-(4-fluoro-3-methylphenyl)- 1,2,4-trimethyl-5-(2-(((1r,3r)-3- (methylsulfonyl)cyclobutyl)amino)- 2-oxoacetyl)-1H-pyrrole-3- carboxamide | 464 |
| 33 | 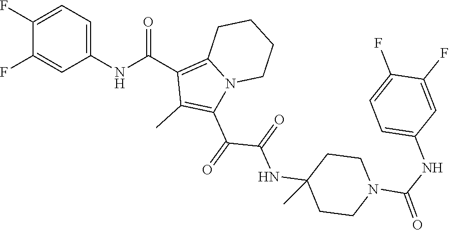 [SMILES: Cc1c(C(=O)Nc2ccc(F)c(F)c2)c2n(c1C(=O)C(=O)NC1(C)CCN(C(=O)Nc3ccc(F)c(F)c3)CC1)CCCC2] | N-(3,4-difluorophenyl)-3-(2-((1- ((3,4- difluorophenyl)carbamoyl)-4- methylpiperidin-4-yl)amino)-2- oxoacetyl)-2-methyl-5,6,7,8- tetrahydroindolizine-1- carboxamide | 614 |
| 35 | 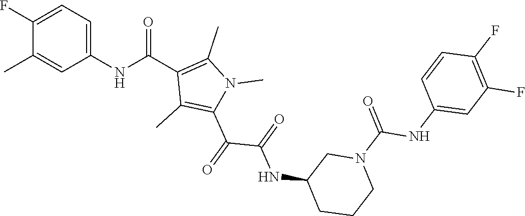 [SMILES: Cc1cc(NC(=O)c2c(C)c(C(=O)C(=O)N[C@@H]3CCCN(C(=O)Nc4ccc(F)c(F)c4)C3)n(C)c2C)ccc1F] | (R)-N-(3,4-difluorophenyl)-3- (2-(4-((4-fluoro-3- methylphenyl)carbamoyl)-1,3,5- trimethyl-1H-pyrrol-2-yl)-2- oxoacetamido)piperidine-1- carboxamide | 570 |
| 36 | 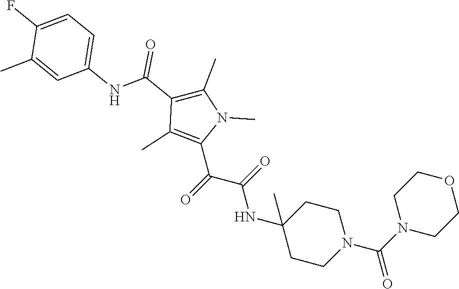 [SMILES: Cc1cc(NC(=O)c2c(C)c(C(=O)C(=O)NC3(C)CCN(C(=O)N4CCOCC4)CC3)n(C)c2C)ccc1F] | N-(4-fluoro-3-methylphenyl)- 1,2,4-trimethyl-5-(2-((4-methyl- 1-(morpholine-4- carbonyl)piperidin-4-yl)amino)- 2-oxoacetyl)-1H-pyrrole-3- carboxamide | 542 |
| 37 | 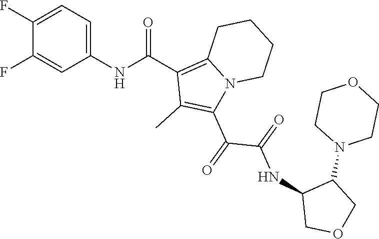 | N-(3,4-difluorophenyl)-2- methyl-3-(2-(((3S,4S)-4- morpholinotetrahydrofuran-3- yl)amino)-2-oxoacetyl)-5,6,7,8- tetrahydroindolizine-1- carboxamide | 517 |
| 38 | 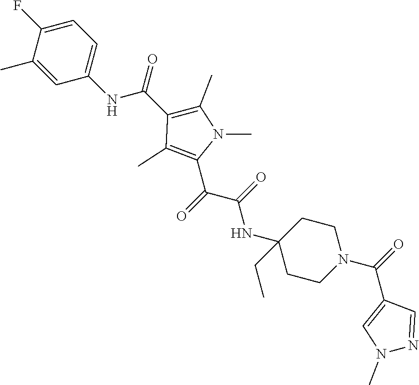 [SMILES: CCC1(NC(=O)C(=O)c2c(C)c(C(=O)Nc3ccc(F)c(C)c3)c(C)n2C)CCN(C(=O)c2cnn(C)c2)CC1] | 5-(2-((4-ethyl-1-(1-methyl-1H- pyrazole-4-carbonyl)piperidin- 4-yl)amino)-2-oxoacetyl)-N-(4- fluoro-3-methylphenyl)-1,2,4- trimethyl-1H-pyrrole-3- carboxamide | 551 |
| 39 | 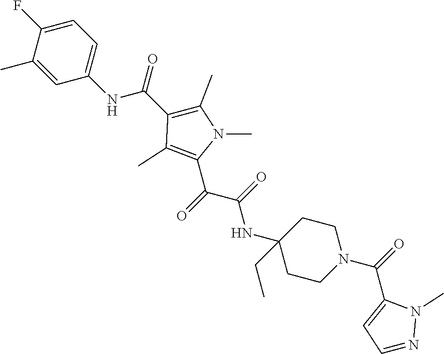 | 5-(2-((4-ethyl-1-(1-methyl-1H- pyrazole-5-carbonyl)piperidin- 4-yl)amino)-2-oxoacetyl)-N-(4- fluoro-3-methylphenyl)-1,2,4- trimethyl-1H-pyrrole-3- carboxamide | 551 |
| 40 | 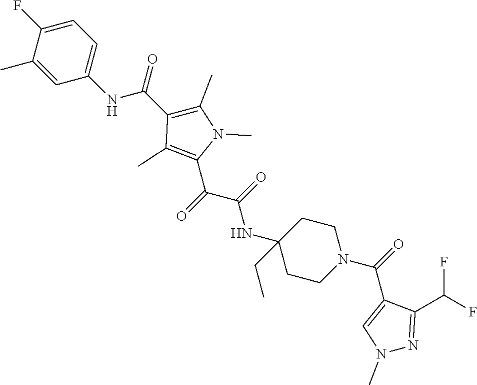 [SMILES: CCC1(NC(=O)C(=O)c2c(C)c(C(=O)Nc3ccc(F)c(C)c3)c(C)n2C)CCN(C(=O)c2cn(C)nc2C(F)F)CC1] | 5-(2-((1-(3-(difluoromethyl)-1- methyl-1H-pyrazole-4- carbonyl)-4-ethylpiperidin-4- yl)amino)-2-oxoacetyl)-N-(4- fluoro-3-methylphenyl)-1,2,4- trimethyl-1H-pyrrole-3- carboxamide | 601 |
| 41 | 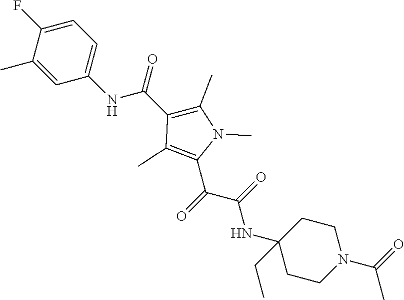 [SMILES: CCC1(NC(=O)C(=O)c2c(C)c(C(=O)Nc3ccc(F)c(C)c3)c(C)n2C)CCN(C(C)=O)CC1] | 5-(2-((1-acetyl-4-ethylpiperidin- 4-yl)amino)-2-oxoacetyl)-N-(4- fluoro-3-methylphenyl)-1,2,4- trimethyl-1H-pyrrole-3- carboxamide | 485 |
| 42 | 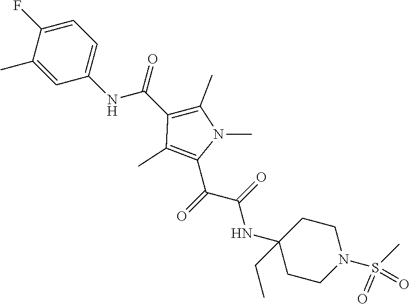 [SMILES: CCC1(NC(=O)C(=O)c2c(C)c(C(=O)Nc3ccc(F)c(C)c3)c(C)n2C)CCN(S(C)(=O)=O)CC1] | 5-(2-((4-ethyl-1- (methylsulfonyl)piperidin-4- yl)amino)-2-oxoacetyl)-N-(4- fluoro-3-methylphenyl)-1,2,4- trimethyl-1H-pyrrole-3- carboxamide | 521 |
| 43 | 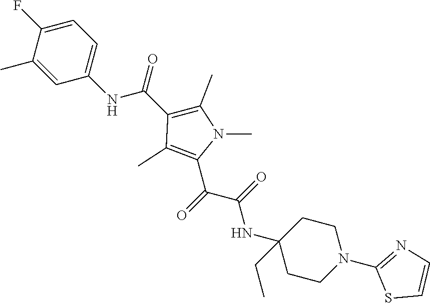 [SMILES: CCC1(NC(=O)C(=O)c2c(C)c(C(=O)Nc3ccc(F)c(C)c3)c(C)n2C)CCN(c2nccs2)CC1] | 5-(2-((4-ethyl-1-(thiazol-2- yl)piperidin-4-yl)amino)-2- oxoacetyl)-N-(4-fluoro-3- methylphenyl)-1,2,4-trimethyl- 1H-pyrrole-3-carboxamide | 526 |
| 44 | 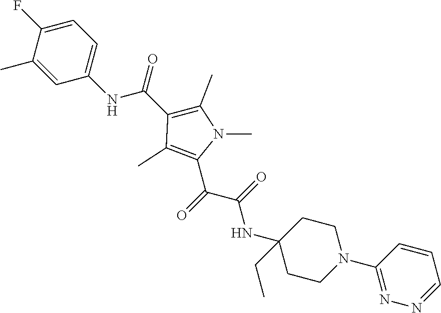 [SMILES: CCC1(NC(=O)C(=O)c2c(C)c(C(=O)Nc3ccc(F)c(C)c3)c(C)n2C)CCN(c2cccnn2)CC1] | 5-(2-((4-ethyl-1-(pyridazin-3- yl)piperidin-4-yl)amino)-2- oxoacetyl)-N-(4-fluoro-3- methylphenyl)-1,2,4-trimethyl- 1H-pyrrole-3-carboxamide | 521 |
| 45 | 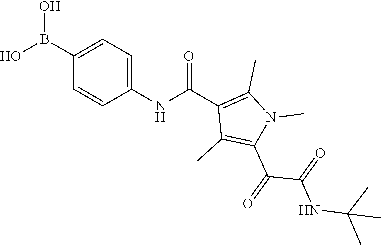 [SMILES: Cc1c(C(=O)Nc2ccc(B(O)O)cc2)c(C)n(C)c1C(=O)C(=O)NC(C)(C)C] | (4-(5-(2-(tert-butylamino)-2- oxoacetyl)-1,2,4-trimethyl-1H- pyrrole-3- carboxamido)phenyl)boronic acid | 400 |
| 46 | 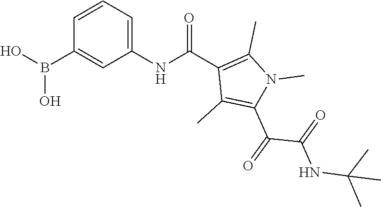 [SMILES: Cc1c(C(=O)Nc2cccc(B(O)O)c2)c(C)n(C)c1C(=O)C(=O)NC(C)(C)C] | (3-(5-(2-(tert-butylamino)-2- oxoacetyl)-1,2,4-trimethyl-1H- pyrrole-3- carboxamido)phenyl)boronic acid | 400 |
| 47 | 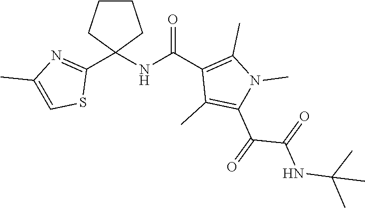 [SMILES: Cc1csc(C2(NC(=O)c3c(C)c(C(=O)C(=O)NC(C)(C)C)n(C)c3C)CCCC2)n1] | 5-(2-(tert-butylamino)-2- oxoacetyl)-1,2,4-trimethyl-N-(1- (4-methylthiazol-2- yl)cyclopentyl)-1H-pyrrole-3- carboxamide | 445 |
| 48 | 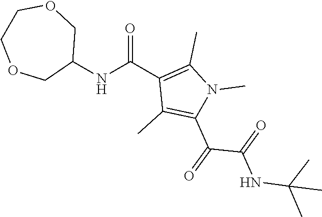 [SMILES: Cc1c(C(=O)NC2COCCOC2)c(C)n(C)c1C(=O)C(=O)NC(C)(C)C] | 5-(2-(tert-butylamino)-2- oxoacetyl)-N-(1,4-dioxepan-6- yl)-1,2,4-trimethyl-1H-pyrrole- 3-carboxamide | 180 |
| 49 | 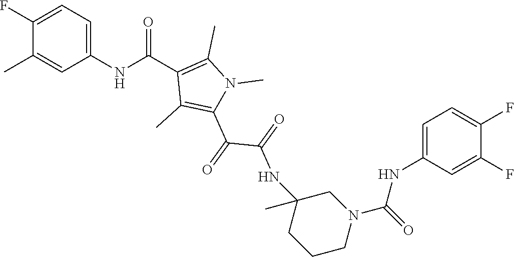 [SMILES: Cc1cc(NC(=O)c2c(C)c(C(=O)C(=O)NC3(C)CCCN(C(=O)Nc4ccc(F)c(F)c4)C3)n(C)c2C)ccc1F] | N-(3,4-difluorophenyl)-3-(2-(4- ((4-fluoro-3- methylphenyl)carbamoyl)-1,3,5- trimethyl-1H-pyrrol-2-yl)-2- oxoacetamido)-3- methylpiperidine-1- carboxamide | 584 |
| 50 | 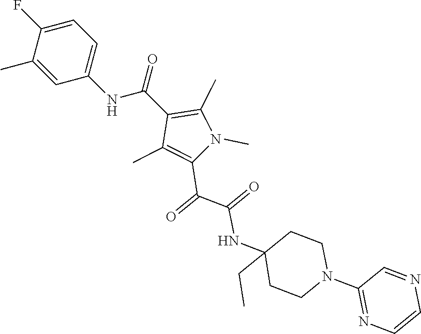 [SMILES: CCC1(NC(=O)C(=O)c2c(C)c(C(=O)Nc3ccc(F)c(C)c3)c(C)n2C)CCN(c2cnccn2)CC1] | 5-(2-((4-ethyl-1-(pyrazin-2- yl)piperidin-4-yl)amino)-2- oxoacetyl)-N-(4-fluoro-3- methylphenyl)-1,2,4-trimethyl- 1H-pyrrole-3-carboxamide | 521 |
| 51 | 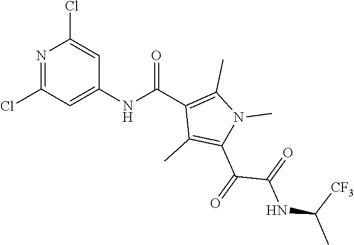 [SMILES: Cc1c(C(=O)Nc2cc(Cl)nc(Cl)c2)c(C)n(C)c1C(=O)C(=O)N[C@H](C)C(F)(F)F] | (R)-N-(2,6-dichloropyridin-4- yl)-1,2,4-trimethyl-5-(2-oxo-2- ((1,1,1-trifluoropropan-2- yl)amino)acetyl)-1H-pyrrole-3- carboxamide | 466 |
| 52 | 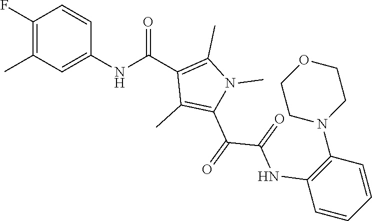 [SMILES: Cc1cc(NC(=O)c2c(C)c(C(=O)C(=O)Nc3ccccc3N3CCOCC3)n(C)c2C)ccc1F] | N-(4-fluoro-3-methylphenyl)- 1,2,4-trimethyl-5-(2-((2- morpholinophenyl)amino)-2- oxoacetyl)-1H-pyrrole-3- carboxamide | 493 |
| 53 | 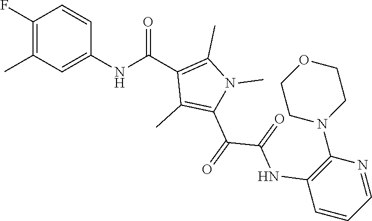 [SMILES: Cc1cc(NC(=O)c2c(C)c(C(=O)C(=O)Nc3cccnc3N3CCOCC3)n(C)c2C)ccc1F] | N-(4-fluoro-3-methylphenyl)- 1,2,4-trimethyl-5-(2-((2- morpholinopyridin-3-yl)amino)- 2-oxoacetyl)-1H-pyrrole-3- carboxamide | 494 |
| 54 | 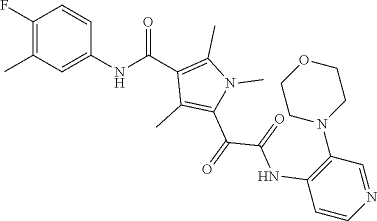 [SMILES: Cc1cc(NC(=O)c2c(C)c(C(=O)C(=O)Nc3ccncc3N3CCOCC3)n(C)c2C)ccc1F] | N-(4-fluoro-3-methylphenyl)- 1,2,4-trimethyl-5-(2-((3- morpholinopyridin-4-yl)amino)- 2-oxoacetyl)-1H-pyrrole-3- carboxamide | 494 |
| 55 | 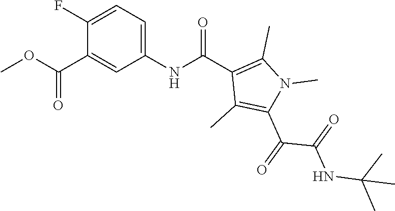 [SMILES: COC(=O)c1cc(NC(=O)c2c(C)c(C(=O)C(=O)NC(C)(C)C)n(C)c2C)ccc1F] | methyl 5-(5-(2-(tert- butylamino)-2-oxoacetyl)-1,2,4- trimethyl-1H-pyrrole-3- carboxamido)-2-fluorobenzoate | 432 |
| 56 | 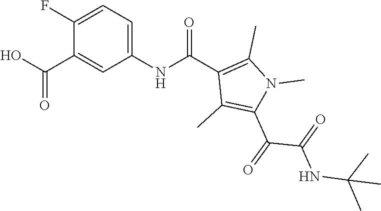 [SMILES: Cc1c(C(=O)Nc2ccc(F)c(C(=O)O)c2)c(C)n(C)c1C(=O)C(=O)NC(C)(C)C] | 5-(5-(2-(tert-butylamino)-2- oxoacetyl)-1,2,4-trimethyl-1H- pyrrole-3-carboxamido)-2- fluorobenzoic acid | 418 |
| 57 | 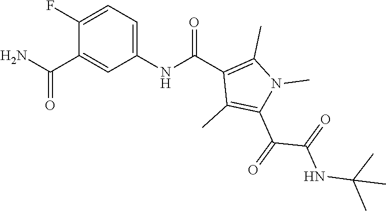 [SMILES: Cc1c(C(=O)Nc2ccc(F)c(C(N)=O)c2)c(C)n(C)c1C(=O)C(=O)NC(C)(C)C] | 5-(2-(tert-butylamino)-2- oxoacetyl)-N-(3-carbamoyl-4- fluorophenyl)-1,2,4-trimethyl- 1H-pyrrole-3-carboxamide | 417 |
| 58 | 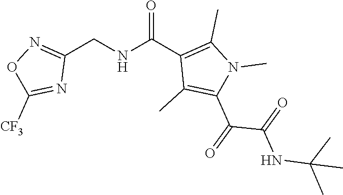 [SMILES: Cc1c(C(=O)NCc2noc(C(F)(F)F)n2)c(C)n(C)c1C(=O)C(=O)NC(C)(C)C] | 5-(2-(tert-butylamino)-2- oxoacetyl)-1,2,4-trimethyl-N- ((5-(trifluoromethyl)-1,2,4- oxadiazol-3-yl)methyl)-1H- pyrrole-3-carboxamide | 430 |
| 59 | 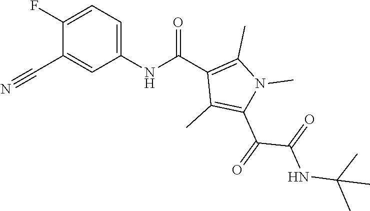 [SMILES: Cc1c(C(=O)Nc2ccc(F)c(C#N)c2)c(C)n(C)c1C(=O)C(=O)NC(C)(C)C] | 5-(2-(tert-butylamino)-2- oxoacetyl)-N-(3-cyano-4- fluorophenyl)-1,2,4-trimethyl- 1H-pyrrole-3-carboxamide | 399 |
| 60 | 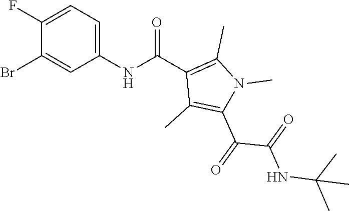 [SMILES: Cc1c(C(=O)Nc2ccc(F)c(Br)c2)c(C)n(C)c1C(=O)C(=O)NC(C)(C)C] | N-(3-bromo-4-fluorophenyl)-5- (2-(tert-butylamino)-2- oxoacetyl)-1,2,4-trimethyl-1H- pyrrole-3-carboxamide | 423 |
| 61 | 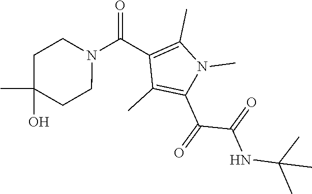 [SMILES: Cc1c(C(=O)N2CCC(C)(O)CC2)c(C)n(C)c1C(=O)C(=O)NC(C)(C)C] | N-(tert-butyl)-2-(4-(4-hydroxy- 4-methylpiperidine-1-carbonyl)- 1,3,5-trimethyl-1H-pyrrol-2-yl)- 2-oxoacetamide | 378 |
| 62 | 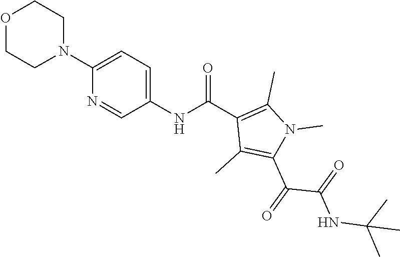 [SMILES: Cc1c(C(=O)Nc2ccc(N3CCOCC3)nc2)c(C)n(C)c1C(=O)C(=O)NC(C)(C)C] | 5-(2-(tert-butylamino)-2- oxoacetyl)-1,2,4-trimethyl-N-(6- morpholinopyridin-3-yl)-1H- pyrrole-3-carboxamide | 442 |
| 63 | 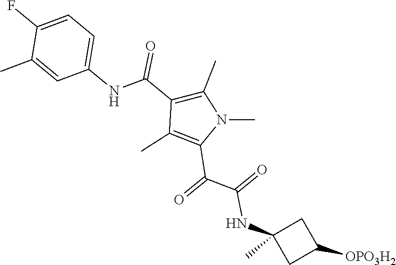 | (1s,3s)-3-(2-(4-((4-fluoro-3- methylphenyl)carbamoyl)-1,3,5- trimethyl-1H-pyrrol-2-yl)-2- oxoacetamido)-3- methylcyclobutyl dihydrogen phosphate | 496 |
| 64 | 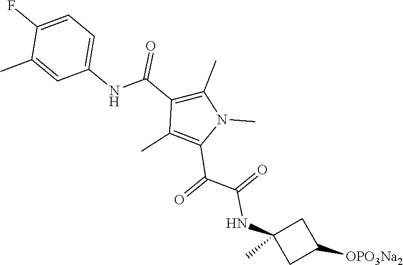 | sodium (1s,3s)-3-(2-(4-((4- fluoro-3- methylphenyl)carbamoyl)-1,3,5- trimethyl-1H-pyrrol-2-yl)-2- oxoacetamido)-3- methylcyclobutyl phosphate | 540 |
| 65 | 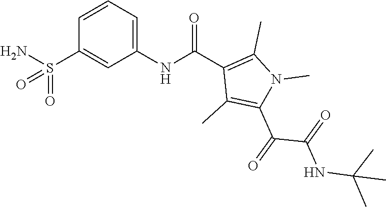 [SMILES: Cc1c(C(=O)Nc2cccc(S(N)(=O)=O)c2)c(C)n(C)c1C(=O)C(=O)NC(C)(C)C] | 5-(2-(tert-butylamino)-2- oxoacetyl)-1,2,4-trimethyl-N-(3- sulfamoylphenyl)-1H-pyrrole-3- carboxamide | 435 |
| 66 | 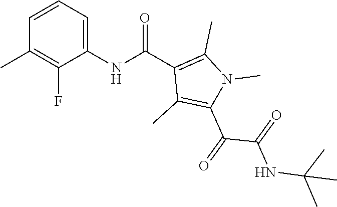 [SMILES: Cc1cccc(NC(=O)c2c(C)c(C(=O)C(=O)NC(C)(C)C)n(C)c2C)c1F] | 5-(2-(tert-butylamino)-2- oxoacetyl)-N-(2-fluoro-3- methylphenyl)-1,2,4-trimethyl- 1H-pyrrole-3-carboxamide | 388 |
| 67 | 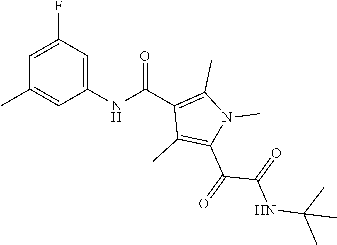 [SMILES: Cc1cc(F)cc(NC(=O)c2c(C)c(C(=O)C(=O)NC(C)(C)C)n(C)c2C)c1] | 5-(2-(tert-butylamino)-2- oxoacetyl)-N-(3-fluoro-5- methylphenyl)-1,2,4-trimethyl- 1H-pyrrole-3-carboxamide | 388 |
| 68 | 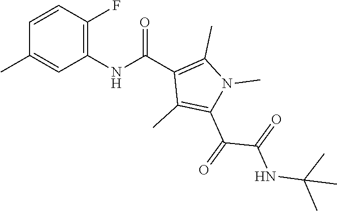 [SMILES: Cc1ccc(F)c(NC(=O)c2c(C)c(C(=O)C(=O)NC(C)(C)C)n(C)c2C)c1] | 5-(2-(tert-butylamino)-2- oxoacetyl)-N-(2-fluoro-5- methylphenyl)-1,2,4-trimethyl- 1H-pyrrole-3-carboxamide | 388 |
| 69 | 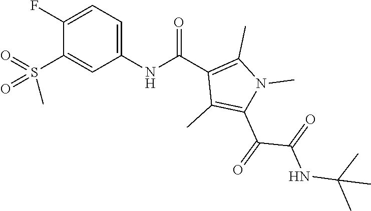 [SMILES: Cc1c(C(=O)Nc2ccc(F)c(S(C)(=O)=O)c2)c(C)n(C)c1C(=O)C(=O)NC(C)(C)C] | 5-(2-(tert-butylamino)-2- oxoacetyl)-N-(4-fluoro-3- (methylsulfonyl)phenyl)-1,2,4- trimethyl-1H-pyrrole-3- carboxamide | 452 |
| 72 | 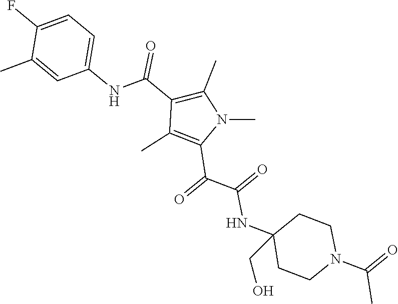 [SMILES: CC(=O)N1CCC(CO)(NC(=O)C(=O)c2c(C)c(C(=O)Nc3ccc(F)c(C)c3)c(C)n2C)CC1] | 5-(2-((1-acetyl-4- (hydroxymethyl)piperidin-4- yl)amino)-2-oxoacetyl)-N-(4- fluoro-3-methylphenyl)-1,2,4- trimethyl-1H-pyrrole-3- carboxamide | 487 |
| 73 | 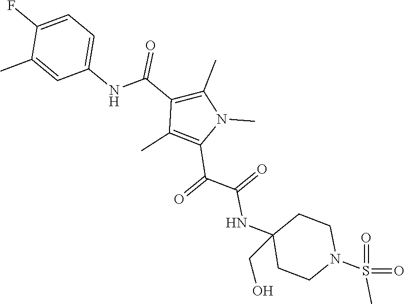 [SMILES: Cc1cc(NC(=O)c2c(C)c(C(=O)C(=O)NC3(CO)CCN(S(C)(=O)=O)CC3)n(C)c2C)ccc1F] | N-(4-fluoro-3-methylphenyl)-5- (2-((4-(hydroxymethyl)-1- (methylsulfonyl)piperidin-4- yl)amino)-2-oxoacetyl)-1,2,4- trimethyl-1H-pyrrole-3- carboxamide | 823 |
| 74 | 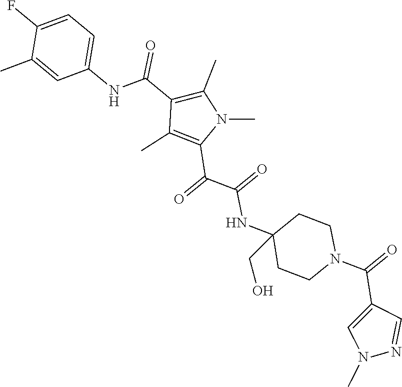 [SMILES: Cc1cc(NC(=O)c2c(C)c(C(=O)C(=O)NC3(CO)CCN(C(=O)c4cnn(C)c4)CC3)n(C)c2C)ccc1F] | N-(4-fluoro-3-methylphenyl)-5- (2-((4-(hydroxymethyl)-1-(1- methyl-1H-pyrazole-4- carbonyl)piperidin-4-yl)amino)- 2-oxoacetyl)-1,2,4-trimethyl- 1H-pyrrole-3-carboxamide | 553 |
| 75 | 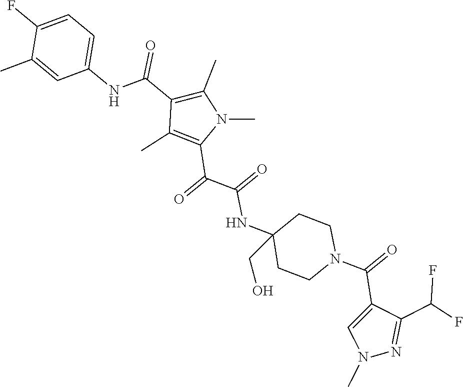 [SMILES: Cc1cc(NC(=O)c2c(C)c(C(=O)C(=O)NC3(CO)CCN(C(=O)c4cn(C)nc4C(F)F)CC3)n(C)c2C)ccc1F] | 5-(2-((1-(3-(difluoromethyl)-1- methyl-1H-pyrazole-4- carbonyl)-4- (hydroxymethyl)piperidin-4- yl)amino)-2-oxoacetyl)-N-(4- fluoro-3-methylphenyl)-1,2,4- trimethyl-1H-pyrrole-3- carboxamide | 603 |
| 76 | 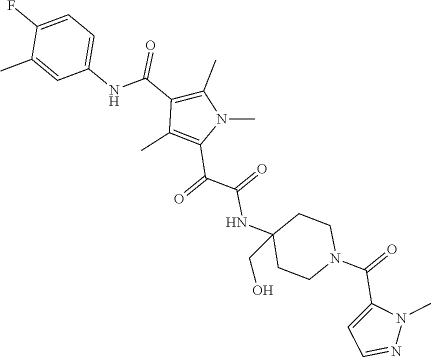 | N-(4-fluoro-3-methylphenyl)-5- (2-((4-(hydroxymethyl)-1-(1- methyl-1H-pyrazole-5- carbonyl)piperidin-4-yl)amino)- 2-oxoacetyl)-1,2,4-trimethyl- 1H-pyrrole-3-carboxamide | 553 |
| 77 | 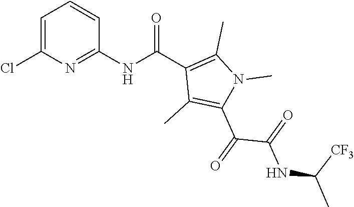 [SMILES: Cc1c(C(=O)Nc2cccc(Cl)n2)c(C)n(C)c1C(=O)C(=O)N[C@H](C)C(F)(F)F] | (R)-N-(6-chloropyridin-2-yl)- 1,2,4-trimethyl-5-(2-oxo-2- ((1,1,1-trifluoropropan-2- yl)amino)acetyl)-1H-pyrrole-3- carboxamide | 431 |
| 78 | 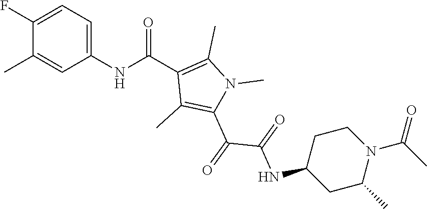 [SMILES: CC(=O)N1CC[C@H](NC(=O)C(=O)c2c(C)c(C(=O)Nc3ccc(F)c(C)c3)c(C)n2C)C[C@H]1C] | 5-(2-(((2R,4S)-1-acetyl-2- methylpiperidin-4-yl)amino)-2- oxoacetyl)-N-(4-fluoro-3- methylphenyl)-1,2,4-trimethyl- 1H-pyrrole-3-carboxamide | 471 |
| 79 | 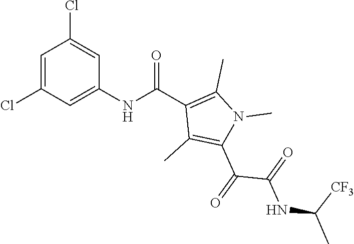 [SMILES: Cc1c(C(=O)Nc2cc(Cl)cc(Cl)c2)c(C)n(C)c1C(=O)C(=O)N[C@H](C)C(F)(F)F] | (R)-N-(3,5-dichlorophenyl)- 1,2,4-trimethyl-5-(2-oxo-2- ((1,1,1-trifluoropropan-2- yl)amino)acetyl)-1H-pyrrole-3- carboxamide | 465 |
| 80 | 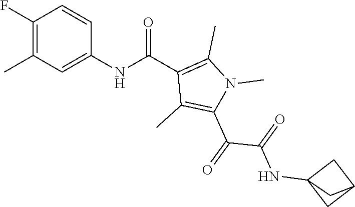 [SMILES: Cc1cc(NC(=O)c2c(C)c(C(=O)C(=O)NC34CC(C3)C4)n(C)c2C)ccc1F] | 5-(2-(bicyclo[1.1.1]pentan-1- ylamino)-2-oxoacetyl)-N-(4- fluoro-3-methylphenyl)-1,2,4- trimethyl-1H-pyrrole-3- carboxamide | 398 |
| 81 | 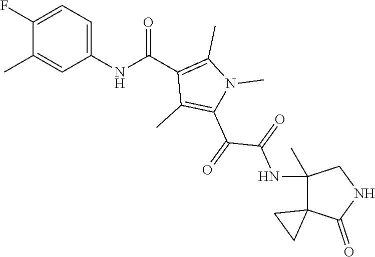 [SMILES: Cc1cc(NC(=O)c2c(C)c(C(=O)C(=O)NC3(C)CNC(=O)C34CC4)n(C)c2C)ccc1F] | N-(4-fluoro-3-methylphenyl)- 1,2,4-trimethyl-5-(2-((7-methyl- 4-oxo-5-azaspiro[2.4]heptan-7- yl)amino)-2-oxoacetyl)-1H- pyrrole-3-carboxamide | 455 |
| 82 | 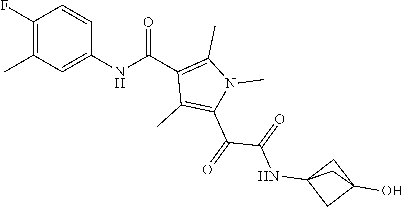 [SMILES: Cc1cc(NC(=O)c2c(C)c(C(=O)C(=O)NC34CC(O)(C3)C4)n(C)c2C)ccc1F] | N-(4-fluoro-3-methylphenyl)-5- (2-((3- hydroxybicyclo[1.1.1]pentan-1- yl)amino)-2-oxoacetyl)-1,2,4- trimethyl-1H-pyrrole-3- carboxamide | 414 |
| 83 | 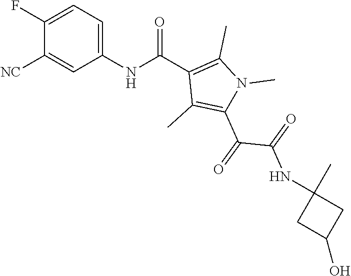 [SMILES: Cc1c(C(=O)Nc2ccc(F)c(C#N)c2)c(C)n(C)c1C(=O)C(=O)NC1(C)CC(O)C1] | N-(3-cyano-4-fluorophenyl)-5- (2-((3-hydroxy-1- methylcyclobutyl)amino)-2- oxoacetyl)-1,2,4-trimethyl-1H- pyrrole-3-carboxamide | 427 |
| 85 | 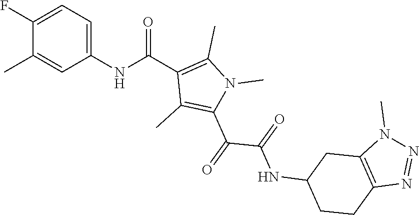 [SMILES: Cc1cc(NC(=O)c2c(C)c(C(=O)C(=O)NC3CCc4nnn(C)c4C3)n(C)c2C)ccc1F] | N-(4-fluoro-3-methylphenyl)- 1,2,4-trimethyl-5-(2-((1-methyl- 4,5,6,7-tetrahydro-1H- benzo[d][1,2,3]triazol-6- yl)amino)-2-oxoacetyl)-1H- pyrrole-3-carboxamide | 467 |
| 86 | 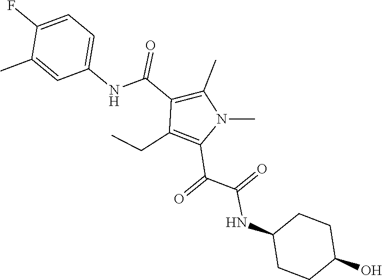 [SMILES: CCc1c(C(=O)Nc2ccc(F)c(C)c2)c(C)n(C)c1C(=O)C(=O)N[C@H]1CC[C@@H](O)CC1] | 4-ethyl-N-(4-fluoro-3- methylphenyl)-5-(2-(((1s,4s)-4- hydroxycyclohexyl)amino)-2- oxoacetyl)-1,2-dimethyl-1H- pyrrole-3-carboxamide | 444 |
| 87 | 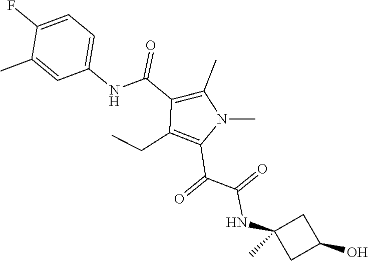 [SMILES: CCc1c(C(=O)Nc2ccc(F)c(C)c2)c(C)n(C)c1C(=O)C(=O)N[C@]1(C)C[C@H](O)C1] | 4-ethyl-N-(4-fluoro-3- methylphenyl)-5-(2-(((1s,3s)-3- hydroxy-1- methylcyclobutyl)amino)-2- oxoacetyl)-1,2-dimethyl-1H- pyrrole-3-carboxamide | 430 |
| 88 | 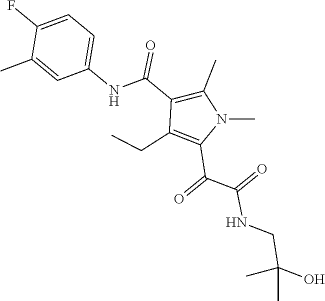 [SMILES: CCc1c(C(=O)Nc2ccc(F)c(C)c2)c(C)n(C)c1C(=O)C(=O)NCC(C)(C)O] | 4-ethyl-N-(4-fluoro-3- methylphenyl)-5-(2-((2- hydroxy-2- methylpropyl)amino)-2- oxoacetyl)-1,2-dimethyl-1H- pyrrole-3-carboxamide | 418 |
| 89 | 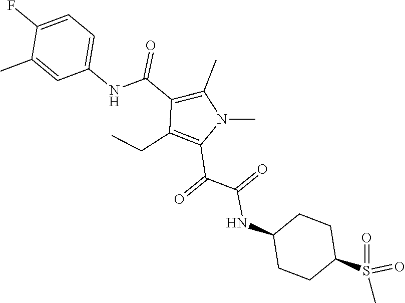 [SMILES: CCc1c(C(=O)Nc2ccc(F)c(C)c2)c(C)n(C)c1C(=O)C(=O)N[C@H]1CC[C@@H](S(C)(=O)=O)CC1] | 4-ethyl-N-(4-fluoro-3- methylphenyl)-1,2-dimethyl-5- (2-(((1s,4s)-4- (methylsulfonyl)cyclohexyl)amino)- 2-oxoacetyl)-1H-pyrrole-3- carboxamide | 506 |
| 90 | 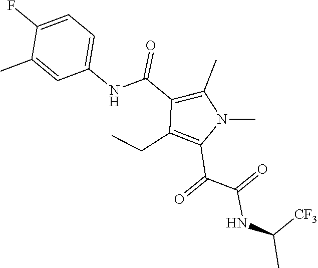 [SMILES: CCc1c(C(=O)Nc2ccc(F)c(C)c2)c(C)n(C)c1C(=O)C(=O)N[C@H](C)C(F)(F)F] | (R)-4-ethyl-N-(4-fluoro-3- methylphenyl)-1,2-dimethyl-5- (2-oxo-2-((1,1,1- trifluoropropan-2- yl)amino)acetyl)-1H-pyrrole-3- carboxamide | 442 |
| 91 | 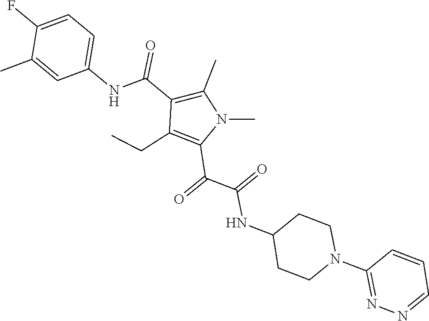 [SMILES: CCc1c(C(=O)Nc2ccc(F)c(C)c2)c(C)n(C)c1C(=O)C(=O)NC1CCN(c2cccnn2)CC1] | 4-ethyl-N-(4-fluoro-3- methylphenyl)-1,2-dimethyl-5- (2-oxo-2-((1-(pyridazin-3- yl)piperidin-4-yl)amino)acetyl)- 1H-pyrrole-3-carboxamide | 507 |
| 92 | 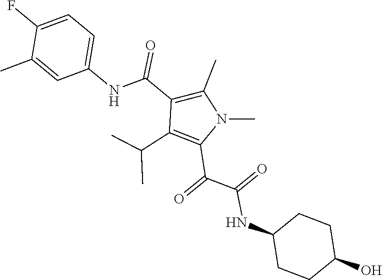 [SMILES: Cc1cc(NC(=O)c2c(C(C)C)c(C(=O)C(=O)N[C@H]3CC[C@@H](O)CC3)n(C)c2C)ccc1F] | N-(4-fluoro-3-methylphenyl)-5- (2-(((1s,4s)-4- hydroxycyclohexyl)amino)-2- oxoacetyl)-4-isopropyl-1,2- dimethyl-1H-pyrrole-3- carboxamide | 458 |
| 93 | 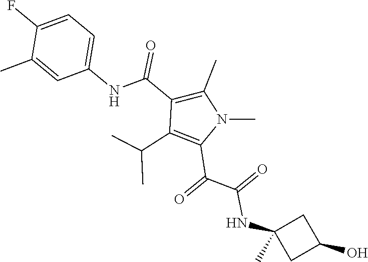 [SMILES: Cc1cc(NC(=O)c2c(C(C)C)c(C(=O)C(=O)N[C@]3(C)C[C@H](O)C3)n(C)c2C)ccc1F] | N-(4-fluoro-3-methylphenyl)-5- (2-(((1s,3s)-3-hydroxy-1- methylcyclobutyl)amino)-2- oxoacetyl)-4-isopropyl-1,2- dimethyl-1H-pyrrole-3- carboxamide | 444 |
| 94 | 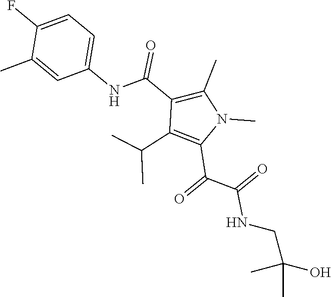 [SMILES: Cc1cc(NC(=O)c2c(C(C)C)c(C(=O)C(=O)NCC(C)(C)O)n(C)c2C)ccc1F] | N-(4-fluoro-3-methylphenyl)-5- (2-((2-hydroxy-2- methylpropyl)amino)-2- oxoacetyl)-4-isopropyl-1,2- dimethyl-1H-pyrrole-3- carboxamide | 432 |
| 95 | 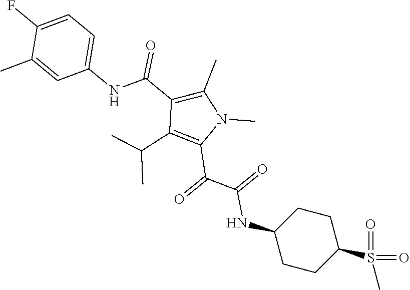 [SMILES: Cc1cc(NC(=O)c2c(C(C)C)c(C(=O)C(=O)N[C@H]3CC[C@@H](S(C)(=O)=O)CC3)n(C)c2C)ccc1F] | N-(4-fluoro-3-methylphenyl)-4- isopropyl-1,2-dimethyl-5-(2- (((1s,4s)-4- (methylsulfonyl)cyclohexyl)amino)- 2-oxoacetyl)-1H-pyrrole-3- carboxamide | 520 |
| 96 | 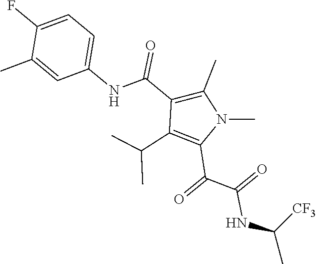 [SMILES: Cc1cc(NC(=O)c2c(C(C)C)c(C(=O)C(=O)N[C@H](C)C(F)(F)F)n(C)c2C)ccc1F] | (R)-N-(4-fluoro-3- methylphenyl)-4-isopropyl-1,2- dimethyl-5-(2-oxo-2-((1,1,1- trifluoropropan-2- yl)amino)acetyl)-1H-pyrrole-3- carboxamide | 456 |
| 97 | 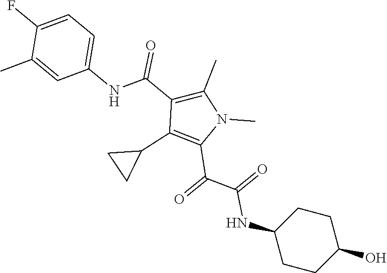 [SMILES: Cc1cc(NC(=O)c2c(C3CC3)c(C(=O)C(=O)N[C@H]3CC[C@@H](O)CC3)n(C)c2C)ccc1F] | 4-cyclopropyl-N-(4-fluoro-3- methylphenyl)-5-(2-(((1s,4s)-4- hydroxycyclohexyl)amino)-2- oxoacetyl)-1,2-dimethyl-1H- pyrrole-3-carboxamide | 456 |
| 98 | 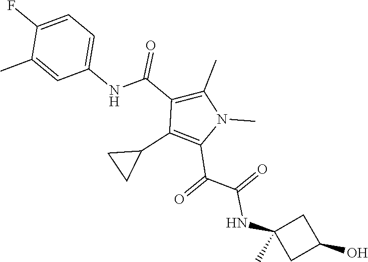 [SMILES: Cc1cc(NC(=O)c2c(C3CC3)c(C(=O)C(=O)N[C@]3(C)C[C@H](O)C3)n(C)c2C)ccc1F] | 4-cyclopropyl-N-(4-fluoro-3- methylphenyl)-5-(2-(((1s,3s)-3- hydroxy-1- methylcyclobutyl)amino)-2- oxoacetyl)-1,2-dimethyl-1H- pyrrole-3-carboxamide | 442 |
| 99 | 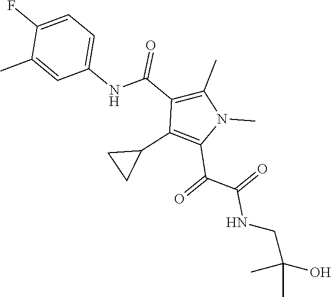 [SMILES: Cc1cc(NC(=O)c2c(C3CC3)c(C(=O)C(=O)NCC(C)(C)O)n(C)c2C)ccc1F] | 4-cyclopropyl-N-(4-fluoro-3- methylphenyl)-5-(2-((2- hydroxy-2- methylpropyl)amino)-2- oxoacetyl)-1,2-dimethyl-1H- pyrrole-3-carboxamide | 430 |
| 100 | 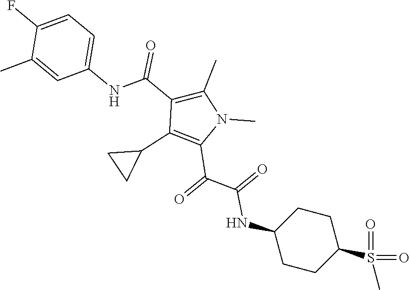 [SMILES: Cc1cc(NC(=O)c2c(C3CC3)c(C(=O)C(=O)N[C@H]3CC[C@@H](S(C)(=O)=O)CC3)n(C)c2C)ccc1F] | 4-cyclopropyl-N-(4-fluoro-3- methylphenyl)-1,2-dimethyl-5- (2-(((1s,4s)-4- (methylsulfonyl)cyclohexyl)amino)- 2-oxoacetyl)-1H-pyrrole-3- carboxamide | 518 |
| 101 | 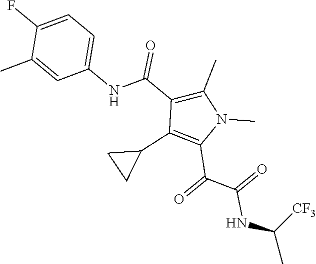 [SMILES: Cc1cc(NC(=O)c2c(C3CC3)c(C(=O)C(=O)N[C@H](C)C(F)(F)F)n(C)c2C)ccc1F] | (R)-4-cyclopropyl-N-(4-fluoro- 3-methylphenyl)-1,2-dimethyl- 5-(2-oxo-2-((1,1,1- trifluoropropan-2- yl)amino)acetyl)-1H-pyrrole-3- carboxamide | 454 |
| 102 | 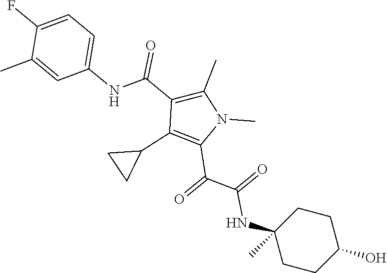 [SMILES: Cc1cc(NC(=O)c2c(C3CC3)c(C(=O)C(=O)N[C@]3(C)CC[C@@H](O)CC3)n(C)c2C)ccc1F] | 4-cyclopropyl-N-(4-fluoro-3- methylphenyl)-5-(2-(((1r,4r)-4- hydroxy-1- methylcyclohexyl)amino)-2- oxoacetyl)-1,2-dimethyl-1H- pyrrole-3-carboxamide | 470 |
| 103 | 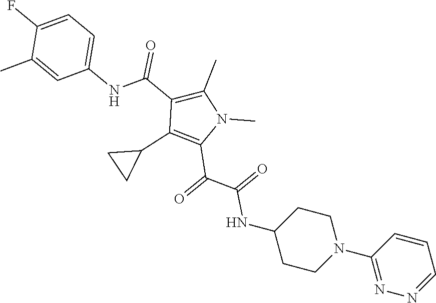 [SMILES: Cc1cc(NC(=O)c2c(C3CC3)c(C(=O)C(=O)NC3CCN(c4cccnn4)CC3)n(C)c2C)ccc1F] | 4-cyclopropyl-N-(4-fluoro-3- methylphenyl)-1,2-dimethyl-5- (2-oxo-2-((1-(pyridazin-3- yl)piperidin-4-yl)amino)acetyl)- 1H-pyrrole-3-carboxamide | 519 |
| 104 | 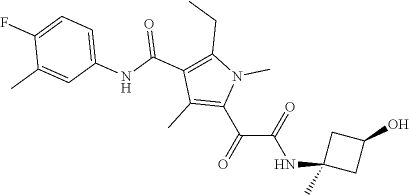 [SMILES: CCc1c(C(=O)Nc2ccc(F)c(C)c2)c(C)c(C(=O)C(=O)N[C@]2(C)C[C@H](O)C2)n1C] | 2-ethyl-N-(4-fluoro-3- methylphenyl)-5-(2-(((1s,3s)-3- hydroxy-1- methylcyclobutyl)amino)-2- oxoacetyl)-1,4-dimethyl-1H- pyrrole-3-carboxamide | 430 |
| 105 | 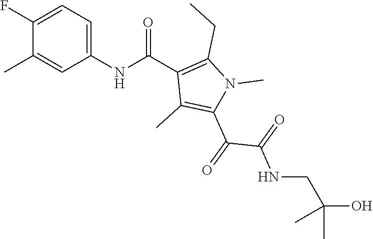 [SMILES: CCc1c(C(=O)Nc2ccc(F)c(C)c2)c(C)c(C(=O)C(=O)NCC(C)(C)O)n1C] | 2-ethyl-N-(4-fluoro-3- methylphenyl)-5-(2-((2- hydroxy-2- methylpropyl)amino)-2- oxoacetyl)-1,4-dimethyl-1H- pyrrole-3-carboxamide | 418 |
| 106 | 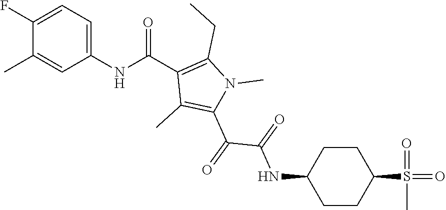 [SMILES: CCc1c(C(=O)Nc2ccc(F)c(C)c2)c(C)c(C(=O)C(=O)N[C@H]2CC[C@@H](S(C)(=O)=O)CC2)n1C] | 2-ethyl-N-(4-fluoro-3- methylphenyl)-1,4-dimethyl-5- (2-(((1s,4s)-4- (methylsulfonyl)cyclohexyl)amino)- 2-oxoacetyl)-1H-pyrrole-3- carboxamide | 506 |
| 107 | 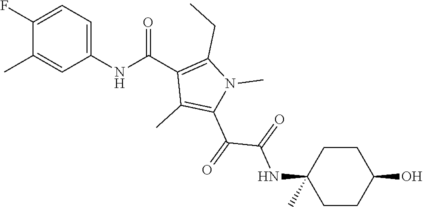 [SMILES: CCc1c(C(=O)Nc2ccc(F)c(C)c2)c(C)c(C(=O)C(=O)N[C@]2(C)CC[C@H](O)CC2)n1C] | 2-ethyl-N-(4-fluoro-3- methylphenyl)-5-(2-(((1s,4s)-4- hydroxy-1- methylcyclohexyl)amino)-2- oxoacetyl)-1,4-dimethyl-1H- pyrrole-3-carboxamide | 458 |
| 108 | 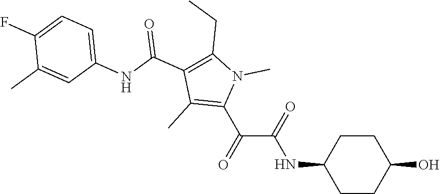 [SMILES: CCc1c(C(=O)Nc2ccc(F)c(C)c2)c(C)c(C(=O)C(=O)N[C@H]2CC[C@@H](O)CC2)n1C] | 2-ethyl-N-(4-fluoro-3- methylphenyl)-5-(2-(((1s,4s)-4- hydroxycyclohexyl)amino)-2- oxoacetyl)-1,4-dimethyl-1H- pyrrole-3-carboxamide | 444 |
| 109 | 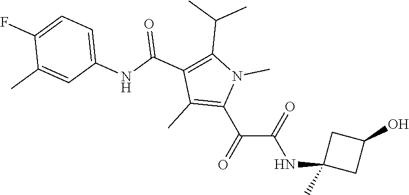 [SMILES: Cc1cc(NC(=O)c2c(C)c(C(=O)C(=O)N[C@]3(C)C[C@H](O)C3)n(C)c2C(C)C)ccc1F] | N-(4-fluoro-3-methylphenyl)-5- (2-(((1s,3s)-3-hydroxy-1- methylcyclobutyl)amino)-2- oxoacetyl)-2-isopropyl-1,4- dimethyl-1H-pyrrole-3- carboxamide | 444 |
| 110 | 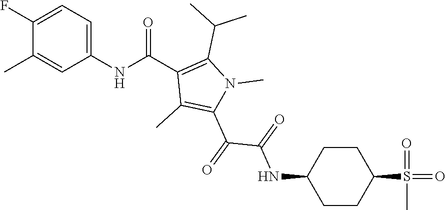 [SMILES: Cc1cc(NC(=O)c2c(C)c(C(=O)C(=O)N[C@H]3CC[C@@H](S(C)(=O)=O)CC3)n(C)c2C(C)C)ccc1F] | N-(4-fluoro-3-methylphenyl)-2- isopropyl-1,4-dimethyl-5-(2- (((1s,4s)-4- (methylsulfonyl)cyclohexyl)amino)- 2-oxoacetyl)-1H-pyrrole-3- carboxamide | 520 |
| 111 | 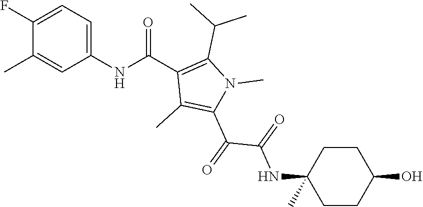 [SMILES: Cc1cc(NC(=O)c2c(C)c(C(=O)C(=O)N[C@]3(C)CC[C@H](O)CC3)n(C)c2C(C)C)ccc1F] | N-(4-fluoro-3-methylphenyl)-5- (2-(((1s,4s)-4-hydroxy-1- methylcyclohexyl)amino)-2- oxoacetyl)-2-isopropyl-1,4- dimethyl-1H-pyrrole-3- carboxamide | 472 |
| 112 | 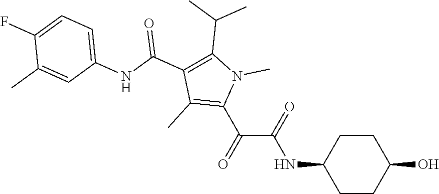 [SMILES: Cc1cc(NC(=O)c2c(C)c(C(=O)C(=O)N[C@H]3CC[C@@H](O)CC3)n(C)c2C(C)C)ccc1F] | N-(4-fluoro-3-methylphenyl)-5- (2-(((1s,4s)-4- hydroxycyclohexyl)amino)-2- oxoacetyl)-2-isopropyl-1,4- dimethyl-1H-pyrrole-3- carboxamide | 458 |
| 113 | 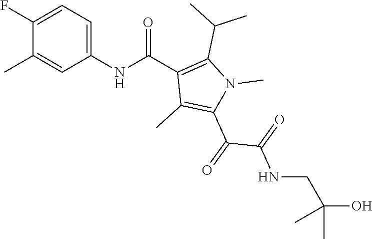 [SMILES: Cc1cc(NC(=O)c2c(C)c(C(=O)C(=O)NCC(C)(C)O)n(C)c2C(C)C)ccc1F] | N-(4-fluoro-3-methylphenyl)-5- (2-((2-hydroxy-2- methylpropyl)amino)-2- oxoacetyl)-2-isopropyl-1,4- dimethyl-1H-pyrrole-3- carboxamide | 432 |
| 114 | 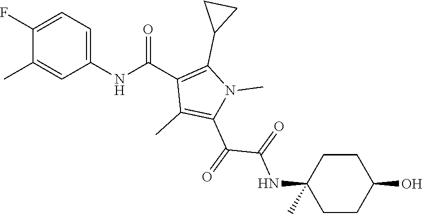 [SMILES: Cc1cc(NC(=O)c2c(C)c(C(=O)C(=O)N[C@]3(C)CC[C@H](O)CC3)n(C)c2C2CC2)ccc1F] | 2-cyclopropyl-N-(4-fluoro-3- methylphenyl)-5-(2-(((1s,4s)-4- hydroxy-1- methylcyclohexyl)amino)-2- oxoacetyl)-1,4-dimethyl-1H- pyrrole-3-carboxamide | 470 |
| 115 | 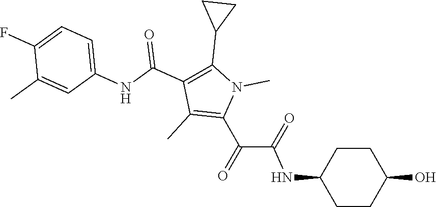 [SMILES: Cc1cc(NC(=O)c2c(C)c(C(=O)C(=O)N[C@H]3CC[C@@H](O)CC3)n(C)c2C2CC2)ccc1F] | 2-cyclopropyl-N-(4-fluoro-3- methylphenyl)-5-(2-(((1s,4s)-4- hydroxycyclohexyl)amino)-2- oxoacetyl)-1,4-dimethyl-1H- pyrrole-3-carboxamide | 456 |
| 116 | 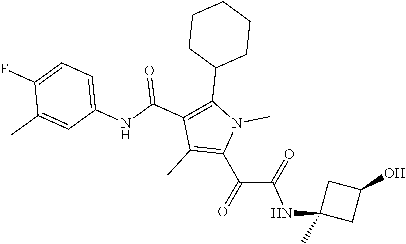 [SMILES: Cc1cc(NC(=O)c2c(C)c(C(=O)C(=O)N[C@]3(C)C[C@H](O)C3)n(C)c2C2CCCCC2)ccc1F] | 2-cyclohexyl-N-(4-fluoro-3- methylphenyl)-5-(2-(((1s,3s)-3- hydroxy-1- methylcyclobutyl)amino)-2- oxoacetyl)-1,4-dimethyl-1H- pyrrole-3-carboxamide | 484 |
| 117 | 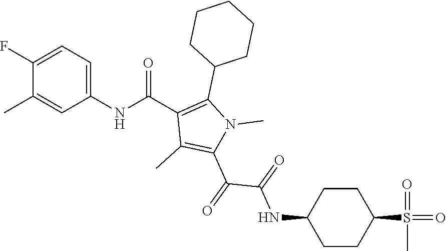 [SMILES: Cc1cc(NC(=O)c2c(C)c(C(=O)C(=O)N[C@H]3CC[C@@H](S(C)(=O)=O)CC3)n(C)c2C2CCCCC2)ccc1F] | 2-cyclohexyl-N-(4-fluoro-3- methylphenyl)-1,4-dimethyl-5- (2-(((1s,4s)-4- (methylsulfonyl)cyclohexyl)amino)- 2-oxoacetyl)-1H-pyrrole-3- carboxamide | 560 |
| 118 | 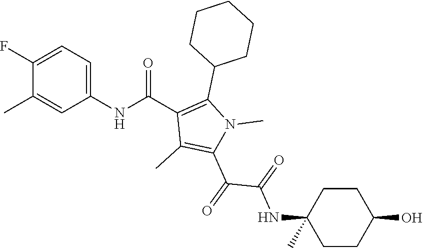 [SMILES: Cc1cc(NC(=O)c2c(C)c(C(=O)C(=O)N[C@]3(C)CC[C@H](O)CC3)n(C)c2C2CCCCC2)ccc1F] | 2-cyclohexyl-N-(4-fluoro-3- methylphenyl)-5-(2-(((1s,4s)-4- hydroxy-1- methylcyclohexyl)amino)-2- oxoacetyl)-1,4-dimethyl-1H- pyrrole-3-carboxamide | 512 |
| 119 | 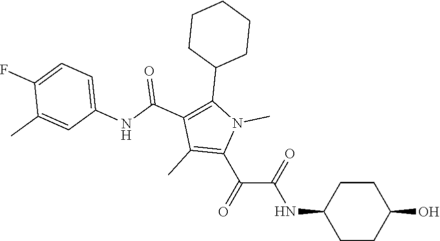 [SMILES: Cc1cc(NC(=O)c2c(C)c(C(=O)C(=O)N[C@H]3CC[C@@H](O)CC3)n(C)c2C2CCCCC2)ccc1F] | 2-cyclohexyl-N-(4-fluoro-3- methylphenyl)-5-(2-(((1s,4s)-4- hydroxycyclohexyl)amino)-2- oxoacetyl)-1,4-dimethyl-1H- pyrrole-3-carboxamide | 498 |
| 120 | 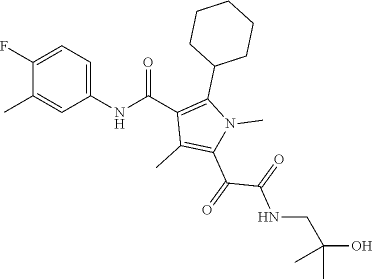 [SMILES: Cc1cc(NC(=O)c2c(C)c(C(=O)C(=O)NCC(C)(C)O)n(C)c2C2CCCCC2)ccc1F] | 2-cyclohexyl-N-(4-fluoro-3- methylphenyl)-5-(2-((2- hydroxy-2- methylpropyl)amino)-2- oxoacetyl)-1,4-dimethyl-1H- pyrrole-3-carboxamide | 472 |
| 121 | 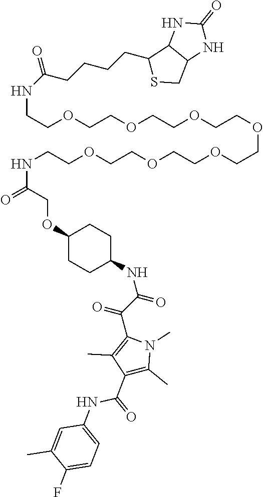 [SMILES: Cc1cc(NC(=O)c2c(C)c(C(=O)C(=O)N[C@H]3CC[C@@H](OCC(=O)NCCOCCOCCOCCOCCOCCOCCOCCNC(=O)CCCCC4SCC5NC(=O)NC54)CC3)n(C)c2C)ccc1F] | 5-(2-(((1s,4s)-4-((2,28-dioxo- 32-(2-oxohexahydro-1H- thieno[3,4-d]imidazol-4-yl)- 6,9,12,15,18,21,24-heptaoxa- 3,27- diazadotriacontyl)oxy)cyclohexyl) amino)-2-oxoacetyl)-N-(4- fluoro-3-methylphenyl)-1,2,4- trimethyl-1H-pyrrole-3- carboxamide | 1063 |
| 122 | 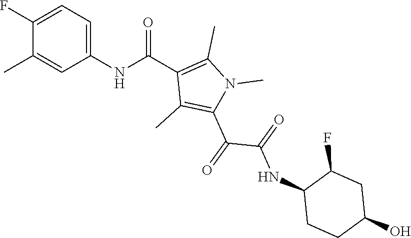 [SMILES: Cc1cc(NC(=O)c2c(C)c(C(=O)C(=O)N[C@@H]3CC[C@H](O)C[C@@H]3F)n(C)c2C)ccc1F] | N-(4-fluoro-3-methylphenyl)-5- (2-(((1R,2S,4S)-2-fluoro-4- hydroxycyclohexyl)amino)-2- oxoacetyl)-1,2,4-trimethyl-1H- pyrrole-3-carboxamide | 448 |
| 123 | 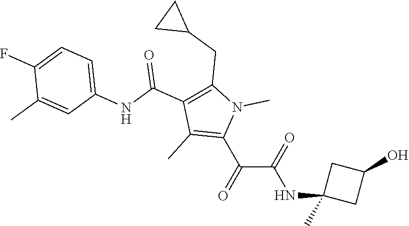 [SMILES: Cc1cc(NC(=O)c2c(C)c(C(=O)C(=O)N[C@]3(C)C[C@H](O)C3)n(C)c2CC2CC2)ccc1F] | 2-(cyclopropylmethyl)-N-(4- fluoro-3-methylphenyl)-5-(2- (((1s,3s)-3-hydroxy-1- methylcyclobutyl)amino)-2- oxoacetyl)-1,4-dimethyl-1H- pyrrole-3-carboxamide | 456 |
| 124 | 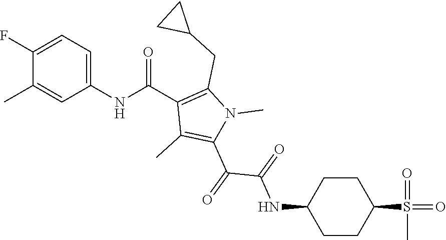 [SMILES: Cc1cc(NC(=O)c2c(C)c(C(=O)C(=O)N[C@H]3CC[C@@H](S(C)(=O)=O)CC3)n(C)c2CC2CC2)ccc1F] | 2-(cyclopropylmethyl)-N-(4- fluoro-3-methylphenyl)-1,4- dimethyl-5-(2-(((1s,4s)-4- (methylsulfonyl)cyclohexyl)amino)- 2-oxoacetyl)-1H-pyrrole-3- carboxamide | 532 |
| 125 | 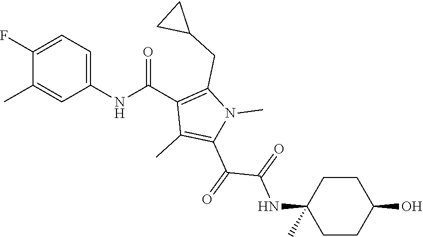 [SMILES: Cc1cc(NC(=O)c2c(C)c(C(=O)C(=O)N[C@]3(C)CC[C@H](O)CC3)n(C)c2CC2CC2)ccc1F] | 2-(cyclopropylmethyl)-N-(4- fluoro-3-methylphenyl)-5-(2- (((1s,4s)-4-hydroxy-1- methylcyclohexyl)amino)-2- oxoacetyl)-1,4-dimethyl-1H- pyrrole-3-carboxamide | 484 |
| 126 | 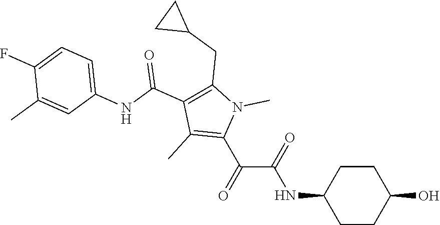 [SMILES: Cc1cc(NC(=O)c2c(C)c(C(=O)C(=O)N[C@H]3CC[C@@H](O)CC3)n(C)c2CC2CC2)ccc1F] | 2-(cyclopropylmethyl)-N-(4- fluoro-3-methylphenyl)-5-(2- (((1s,4s)-4- hydroxycyclohexyl)amino)-2- oxoacetyl)-1,4-dimethyl-1H- pyrrole-3-carboxamide | 470 |
| 127 | 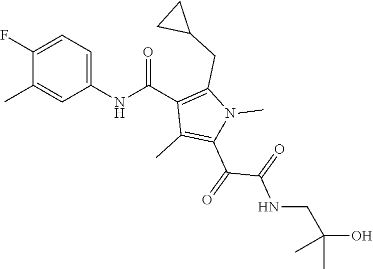 [SMILES: Cc1cc(NC(=O)c2c(C)c(C(=O)C(=O)NCC(C)(C)O)n(C)c2CC2CC2)ccc1F] | 2-(cyclopropylmethyl)-N-(4- fluoro-3-methylphenyl)-5-(2- ((2-hydroxy-2- methylpropyl)amino)-2- oxoacetyl)-1,4-dimethyl-1H- pyrrole-3-carboxamide | 444 |
| 128 | 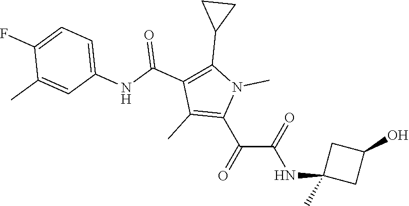 [SMILES: Cc1cc(NC(=O)c2c(C)c(C(=O)C(=O)N[C@]3(C)C[C@H](O)C3)n(C)c2C2CC2)ccc1F] | 2-cyclopropyl-N-(4-fluoro-3- methylphenyl)-5-(2-(((1s,3s)-3- hydroxy-1- methylcyclobutyl)amino)-2- oxoacetyl)-1,4-dimethyl-1H- pyrrole-3-carboxamide | 442 |
| 129 | 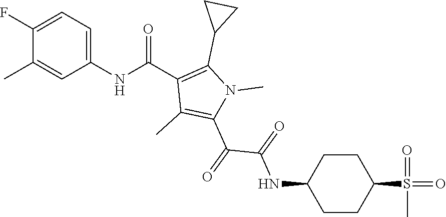 [SMILES: Cc1cc(NC(=O)c2c(C)c(C(=O)C(=O)N[C@H]3CC[C@@H](S(C)(=O)=O)CC3)n(C)c2C2CC2)ccc1F] | 2-cyclopropyl-N-(4-fluoro-3- methylphenyl)-1,4-dimethyl-5- (2-(((1s,4s)-4- (methylsulfonyl)cyclohexyl)amino)- 2-oxoacetyl)-1H-pyrrole-3- carboxamide | 518 |
| 130 | 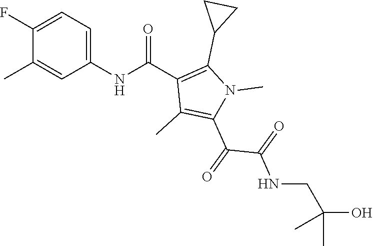 [SMILES: Cc1cc(NC(=O)c2c(C)c(C(=O)C(=O)NCC(C)(C)O)n(C)c2C2CC2)ccc1F] | 2-cyclopropyl-N-(4-fluoro-3- methylphenyl)-5-(2-((2- hydroxy-2- methylpropyl)amino)-2- oxoacetyl)-1,4-dimethyl-1H- pyrrole-3-carboxamide | 430 |
| 131 | 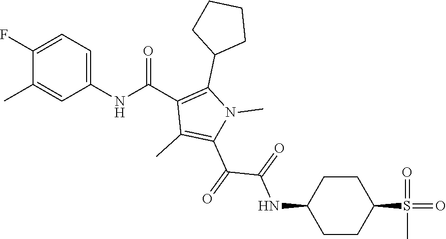 [SMILES: Cc1cc(NC(=O)c2c(C)c(C(=O)C(=O)N[C@H]3CC[C@@H](S(C)(=O)=O)CC3)n(C)c2C2CCCC2)ccc1F] | 2-cyclopentyl-N-(4-fluoro-3- methylphenyl)-1,4-dimethyl-5- (2-(((1s,4s)-4- (methylsulfonyl)cyclohexyl)amino)- 2-oxoacetyl)-1H-pyrrole-3- carboxamide | 546 |
| 132 | 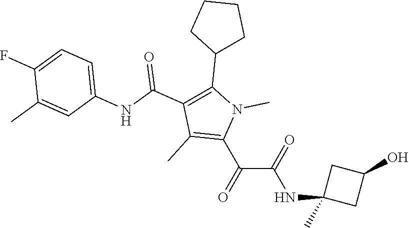 [SMILES: Cc1cc(NC(=O)c2c(C)c(C(=O)C(=O)N[C@]3(C)C[C@H](O)C3)n(C)c2C2CCCC2)ccc1F] | 2-cyclopentyl-N-(4-fluoro-3- methylphenyl)-5-(2-(((1s,3s)-3- hydroxy-1- methylcyclobutyl)amino)-2- oxoacetyl)-1,4-dimethyl-1H- pyrrole-3-carboxamide | 470 |
| 133 | 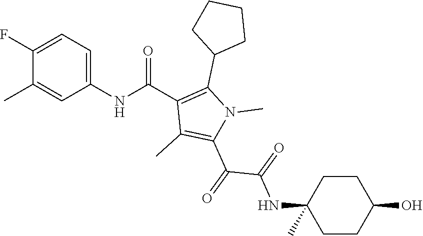 [SMILES: Cc1cc(NC(=O)c2c(C)c(C(=O)C(=O)N[C@]3(C)CC[C@H](O)CC3)n(C)c2C2CCCC2)ccc1F] | 2-cyclopentyl-N-(4-fluoro-3- methylphenyl)-5-(2-(((1s,4s)-4- hydroxy-1- methylcyclohexyl)amino)-2- oxoacetyl)-1,4-dimethyl-1H- pyrrole-3-carboxamide | 498 |
| 134 | 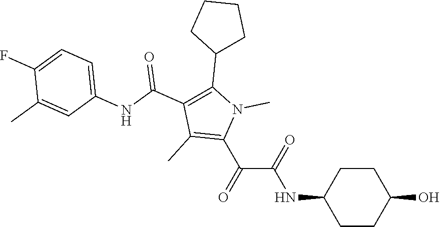 [SMILES: Cc1cc(NC(=O)c2c(C)c(C(=O)C(=O)N[C@H]3CC[C@@H](O)CC3)n(C)c2C2CCCC2)ccc1F] | 2-cyclopentyl-N-(4-fluoro-3- methylphenyl)-5-(2-(((1s,4s)-4- hydroxycyclohexyl)amino)-2- oxoacetyl)-1,4-dimethyl-1H- pyrrole-3-carboxamide | 484 |
| 135 | 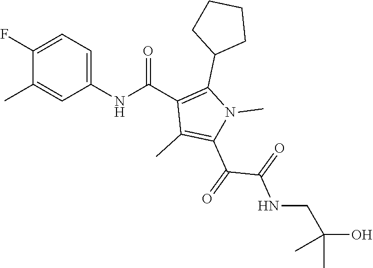 [SMILES: Cc1cc(NC(=O)c2c(C)c(C(=O)C(=O)NCC(C)(C)O)n(C)c2C2CCCC2)ccc1F] | 2-cyclopentyl-N-(4-fluoro-3- methylphenyl)-5-(2-((2- hydroxy-2- methylpropyl)amino)-2- oxoacetyl)-1,4-dimethyl-1H- pyrrole-3-carboxamide | 458 |
| 136 | 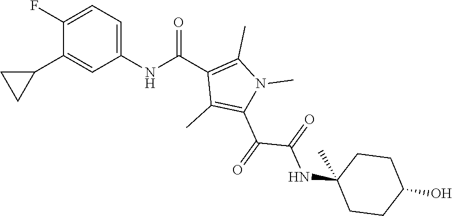 [SMILES: Cc1c(C(=O)Nc2ccc(F)c(C3CC3)c2)c(C)n(C)c1C(=O)C(=O)N[C@]1(C)CC[C@@H](O)CC1] | N-(3-cyclopropyl-4- fluorophenyl)-5-(2-(((1r,4r)-4- hydroxy-1- methylcyclohexyl)amino)-2- oxoacetyl)-1,2,4-trimethyl-1H- pyrrole-3-carboxamide | 470.2 |
| 137 | 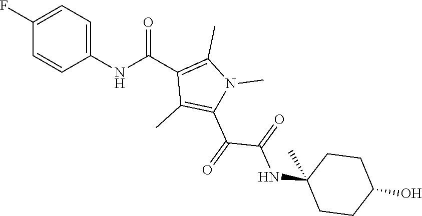 [SMILES: Cc1c(C(=O)Nc2ccc(F)cc2)c(C)n(C)c1C(=O)C(=O)N[C@]1(C)CC[C@@H](O)CC1] | N-(4-fluorophenyl)-5-(2- (((1r,4r)-4-hydroxy-1- methylcyclohexyl)amino)-2- oxoacetyl)-1,2,4-trimethyl-1H- pyrrole-3-carboxamide | 430.2 |
| 138 | 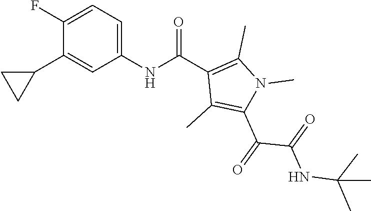 [SMILES: Cc1c(C(=O)Nc2ccc(F)c(C3CC3)c2)c(C)n(C)c1C(=O)C(=O)NC(C)(C)C] | 5-(2-(tert-butylamino)-2- oxoacetyl)-N-(3-cyclopropyl-4- fluorophenyl)-1,2,4-trimethyl- 1H-pyrrole-3-carboxamide | 414.2 |
| 139 | 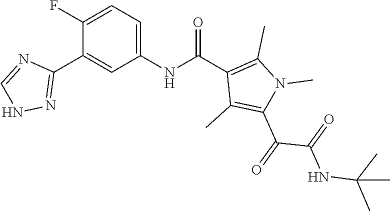 [SMILES: Cc1c(C(=O)Nc2ccc(F)c(-c3nc[nH]n3)c2)c(C)n(C)c1C(=O)C(=O)NC(C)(C)C] | 5-(2-(tert-butylamino)-2- oxoacetyl)-N-(4-fluoro-3-(1H- 1,2,4-triazol-3-yl)phenyl)-1,2,4- trimethyl-1H-pyrrole-3- carboxamide | 441.2 |
| 140 | 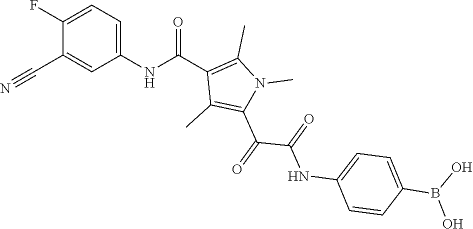 [SMILES: Cc1c(C(=O)Nc2ccc(F)c(C#N)c2)c(C)n(C)c1C(=O)C(=O)Nc1ccc(B(O)O)cc1] | (4-(2-(4-((3-cyano-4- fluorophenyl)carbamoyl)-1,3,5- trimethyl-1H-pyrrol-2-yl)-2- oxoacetamido)phenyl)boronic acid | 463.1 |
| 141 | 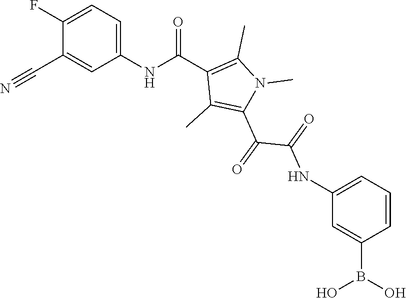 [SMILES: Cc1c(C(=O)Nc2ccc(F)c(C#N)c2)c(C)n(C)c1C(=O)C(=O)Nc1cccc(B(O)O)c1] | (3-(2-(4-((3-cyano-4- fluorophenyl)carbamoyl)-1,3,5- trimethyl-1H-pyrrol-2-yl)-2- oxoacetamido)phenyl)boronic acid | 463.1 |
| 142 | 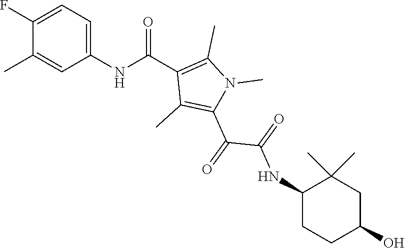 [SMILES: Cc1cc(NC(=O)c2c(C)c(C(=O)C(=O)N[C@@H]3CC[C@H](O)CC3(C)C)n(C)c2C)ccc1F] | N-(4-fluoro-3-methylphenyl)-5- (2-(((1R,4S)-4-hydroxy-2,2- dimethylcyclohexyl)amino)-2- oxoacetyl)-1,2,4-trimethyl-1H- pyrrole-3-carboxamide | 458 |
| 143 | 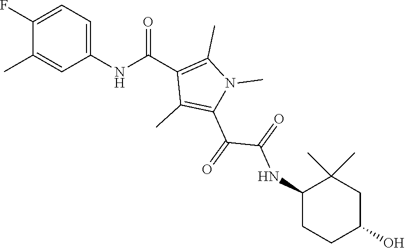 [SMILES: Cc1cc(NC(=O)c2c(C)c(C(=O)C(=O)N[C@@H]3CC[C@@H](O)CC3(C)C)n(C)c2C)ccc1F] | N-(4-fluoro-3-methylphenyl)-5- (2-(((1R,4R)-4-hydroxy-2,2- dimethylcyclohexyl)amino)-2- oxoacetyl)-1,2,4-trimethyl-1H- pyrrole-3-carboxamide | 458 |
| 144 | 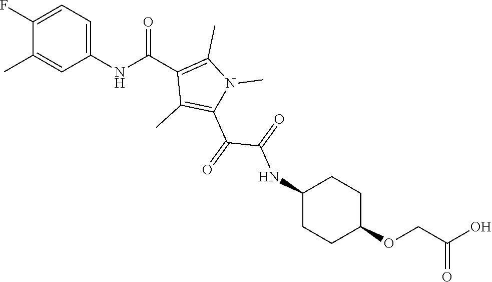 [SMILES: Cc1cc(NC(=O)c2c(C)c(C(=O)C(=O)N[C@H]3CC[C@@H](OCC(=O)O)CC3)n(C)c2C)ccc1F] | 2-(((1s,4s)-4-(2-(4-((4-fluoro-3- methylphenyl)carbamoyl)-1,3,5- trimethyl-1H-pyrrol-2-yl)-2- oxoacetamido)cyclohexyl)oxy) acetic acid | 488 |
| 145 | 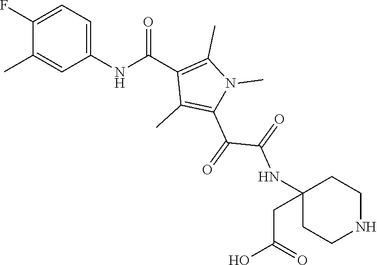 [SMILES: Cc1cc(NC(=O)c2c(C)c(C(=O)C(=O)NC3(CC(=O)O)CCNCC3)n(C)c2C)ccc1F] | 2-(4-(2-(4-((4-fluoro-3- methylphenyl)carbamoyl)-1,3,5- trimethyl-1H-pyrrol-2-yl)-2- oxoacetamido)piperidin-4- yl)acetic acid | 473 |
| 146 | 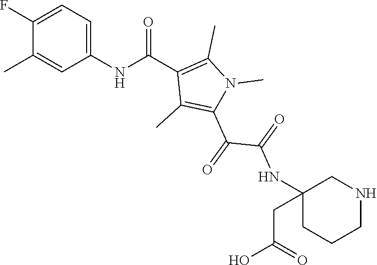 [SMILES: Cc1cc(NC(=O)c2c(C)c(C(=O)C(=O)NC3(CC(=O)O)CCCNC3)n(C)c2C)ccc1F] | 2-(3-(2-(4-((4-fluoro-3- methylphenyl)carbamoyl)-1,3,5- trimethyl-1H-pyrrol-2-yl)-2- oxoacetamido)piperidin-3- yl)acetic acid | 473 |
| 147 | 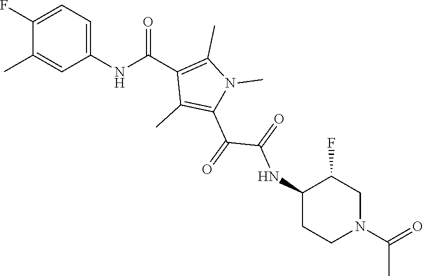 [SMILES: CC(=O)N1CC[C@@H](NC(=O)C(=O)c2c(C)c(C(=O)Nc3ccc(F)c(C)c3)c(C)n2C)[C@H](F)C1] | 5-(2-(((3R,4R)-1-acetyl-3- fluoropiperidin-4-yl)amino)-2- oxoacetyl)-N-(4-fluoro-3- methylphenyl)-1,2,4-trimethyl- 1H-pyrrole-3-carboxamide | 475 |
| 148 | 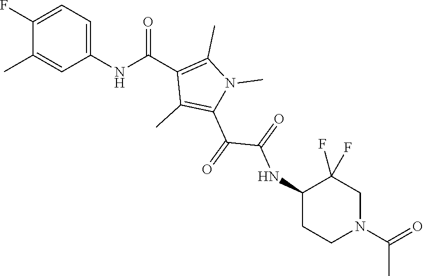 [SMILES: CC(=O)N1CC[C@@H](NC(=O)C(=O)c2c(C)c(C(=O)Nc3ccc(F)c(C)c3)c(C)n2C)C(F)(F)C1] | (R)-5-(2-((1-acetyl-3,3- difluoropiperidin-4-yl)amino)-2- oxoacetyl)-N-(4-fluoro-3- methylphenyl)-1,2,4-trimethyl- 1H-pyrrole-3-carboxamide | 493 |
| 149 | 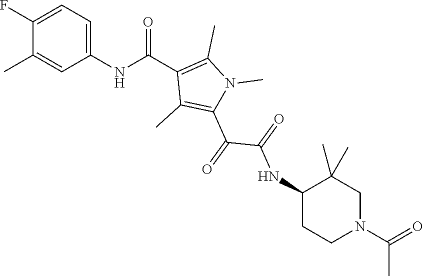 [SMILES: CC(=O)N1CC[C@@H](NC(=O)C(=O)c2c(C)c(C(=O)Nc3ccc(F)c(C)c3)c(C)n2C)C(C)(C)C1] | (R)-5-(2-((1-acetyl-3,3- dimethylpiperidin-4-yl)amino)- 2-oxoacetyl)-N-(4-fluoro-3- methylphenyl)-1,2,4-trimethyl- 1H-pyrrole-3-carboxamide | 485 |
| 150 | 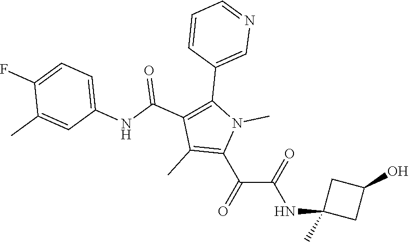 [SMILES: Cc1cc(NC(=O)c2c(C)c(C(=O)C(=O)N[C@]3(C)C[C@H](O)C3)n(C)c2-c2cccnc2)ccc1F] | N-(4-fluoro-3-methylphenyl)-5- (2-(((1s,3s)-3-hydroxy-1- methylcyclobutyl)amino)-2- oxoacetyl)-1,4-dimethyl-2- (pyridin-3-yl)-1H-pyrrole-3- carboxamide N-(4-fluoro-3-methylphenyl)- | 479 |
| 151 | 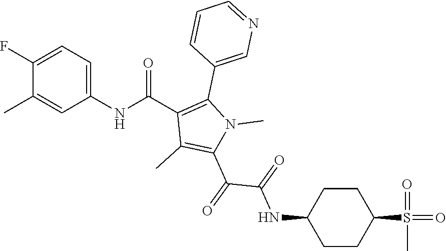 [SMILES: Cc1cc(NC(=O)c2c(C)c(C(=O)C(=O)N[C@H]3CC[C@@H](S(C)(=O)=O)CC3)n(C)c2-c2cccnc2)ccc1F] | N-(4-fluoro-3-methylphenyl)- 1,4-dimethyl-5-(2-(((1s,4s)-4- (methylsulfonyl)cyclohexyl)amino)- 2-oxoacetyl)-2-(pyridin-3- yl)-1H-pyrrole-3-carboxamide | 555 |
| 152 | 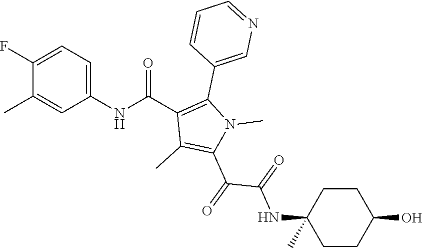 [SMILES: Cc1cc(NC(=O)c2c(C)c(C(=O)C(=O)N[C@]3(C)CC[C@H](O)CC3)n(C)c2-c2cccnc2)ccc1F] | N-(4-fluoro-3-methylphenyl)-5- (2-(((1s,4s)-4-hydroxy-1- methylcyclohexyl)amino)-2- oxoacetyl)-1,4-dimethyl-2- (pyridin-3-yl)-1H-pyrrole-3- carboxamide | 507 |
| 153 | 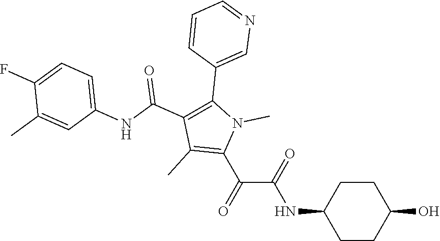 [SMILES: Cc1cc(NC(=O)c2c(C)c(C(=O)C(=O)N[C@H]3CC[C@@H](O)CC3)n(C)c2-c2cccnc2)ccc1F] | N-(4-fluoro-3-methylphenyl)-5- (2-(((1s,4s)-4- hydroxycyclohexyl)amino)-2- oxoacetyl)-1,4-dimethyl-2- (pyridin-3-yl)-1H-pyrrole-3- carboxamide | 493 |
| 154 | 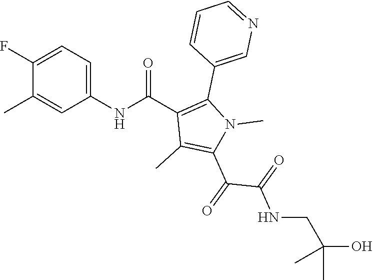 [SMILES: Cc1cc(NC(=O)c2c(C)c(C(=O)C(=O)NCC(C)(C)O)n(C)c2-c2cccnc2)ccc1F] | N-(4-fluoro-3-methylphenyl)-5- (2-((2-hydroxy-2- methylpropyl)amino)-2- oxoacetyl)-1,4-dimethyl-2- (pyridin-3-yl)-1H-pyrrole-3- carboxamide | 467 |
| 155 | 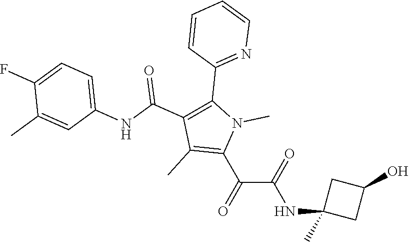 [SMILES: Cc1cc(NC(=O)c2c(C)c(C(=O)C(=O)N[C@]3(C)C[C@H](O)C3)n(C)c2-c2ccccn2)ccc1F] | N-(4-fluoro-3-methylphenyl)-5- (2-(((1s,3s)-3-hydroxy-1- methylcyclobutyl)amino)-2- oxoacetyl)-1,4-dimethyl-2- (pyridin-2-yl)-1H-pyrrole-3- carboxamide | 479 |
| 156 | 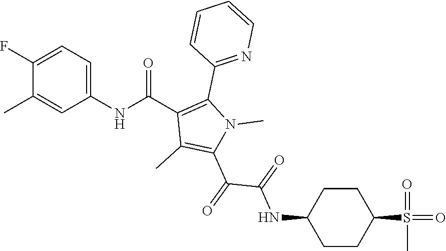 [SMILES: Cc1cc(NC(=O)c2c(C)c(C(=O)C(=O)N[C@H]3CC[C@@H](S(C)(=O)=O)CC3)n(C)c2-c2ccccn2)ccc1F] | N-(4-fluoro-3-methylphenyl)- 1,4-dimethyl-5-(2-(((1s,4s)-4- (methylsulfonyl)cyclohexyl)amino)- 2-oxoacetyl)-2-(pyridin-2- yl)-1H-pyrrole-3-carboxamide | 555 |
| 157 | 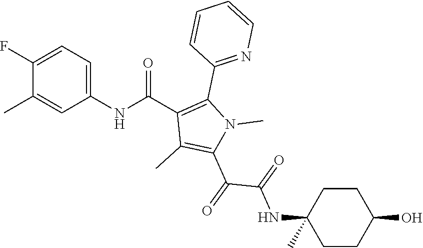 [SMILES: Cc1cc(NC(=O)c2c(C)c(C(=O)C(=O)N[C@]3(C)CC[C@H](O)CC3)n(C)c2-c2ccccn2)ccc1F] | N-(4-fluoro-3-methylphenyl)-5- (2-(((1s,4s)-4-hydroxy-1- methylcyclohexyl)amino)-2- oxoacetyl)-1,4-dimethyl-2- (pyridin-2-yl)-1H-pyrrole-3- carboxamide | 507 |
| 158 | 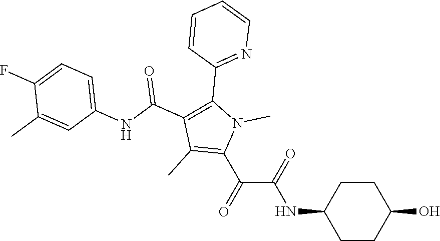 [SMILES: Cc1cc(NC(=O)c2c(C)c(C(=O)C(=O)N[C@H]3CC[C@@H](O)CC3)n(C)c2-c2ccccn2)ccc1F] | N-(4-fluoro-3-methylphenyl)-5- (2-(((1s,4s)-4- hydroxycyclohexyl)amino)-2- oxoacetyl)-1,4-dimethyl-2- (pyridin-2-yl)-1H-pyrrole-3- carboxamide | 493 |
| 159 | 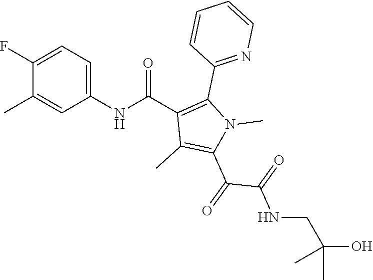 [SMILES: Cc1cc(NC(=O)c2c(C)c(C(=O)C(=O)NCC(C)(C)O)n(C)c2-c2ccccn2)ccc1F] | N-(4-fluoro-3-methylphenyl)-5- (2-((2-hydroxy-2- methylpropyl)amino)-2- oxoacetyl)-1,4-dimethyl-2- (pyridin-2-yl)-1H-pyrrole-3- carboxamide | 467 |
| 160 | 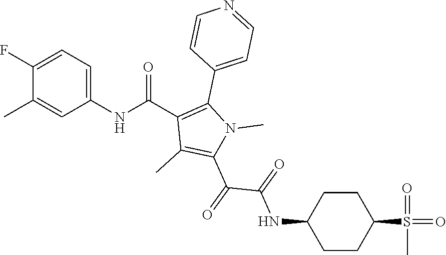 [SMILES: Cc1cc(NC(=O)c2c(C)c(C(=O)C(=O)N[C@H]3CC[C@@H](S(C)(=O)=O)CC3)n(C)c2-c2ccncc2)ccc1F] | N-(4-fluoro-3-methylphenyl)- 1,4-dimethyl-5-(2-(((1s,4s)-4- (methylsulfonyl)cyclohexyl)amino)- 2-oxoacetyl)-2-(pyridin-4- yl)-1H-pyrrole-3-carboxamide | 555 |
| 161 | 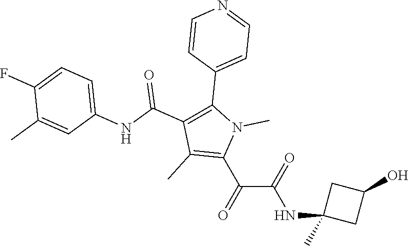 [SMILES: Cc1cc(NC(=O)c2c(C)c(C(=O)C(=O)N[C@]3(C)C[C@H](O)C3)n(C)c2-c2ccncc2)ccc1F] | N-(4-fluoro-3-methylphenyl)-5- (2-(((1s,3s)-3-hydroxy-1- methylcyclobutyl)amino)-2- oxoacetyl)-1,4-dimethyl-2- (pyridin-4-yl)-1H-pyrrole-3- carboxamide | 479 |
| 162 | 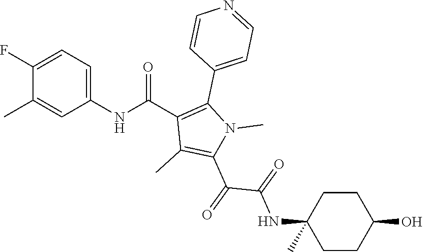 [SMILES: Cc1cc(NC(=O)c2c(C)c(C(=O)C(=O)N[C@]3(C)CC[C@H](O)CC3)n(C)c2-c2ccncc2)ccc1F] | N-(4-fluoro-3-methylphenyl)-5- (2-(((1s,4s)-4-hydroxy-1- methylcyclohexyl)amino)-2- oxoacetyl)-1,4-dimethyl-2- (pyridin-4-yl)-1H-pyrrole-3- carboxamide | 507 |
| 163 | 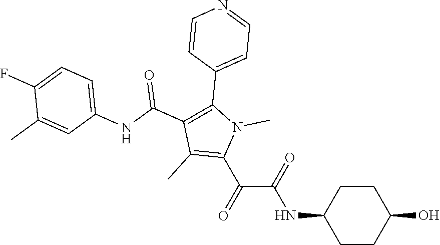 [SMILES: Cc1cc(NC(=O)c2c(C)c(C(=O)C(=O)N[C@H]3CC[C@@H](O)CC3)n(C)c2-c2ccncc2)ccc1F] | N-(4-fluoro-3-methylphenyl)-5- (2-(((1s,4s)-4- hydroxycyclohexyl)amino)-2- oxoacetyl)-1,4-dimethyl-2- (pyridin-4-yl)-1H-pyrrole-3- carboxamide | 493 |
| 164 | 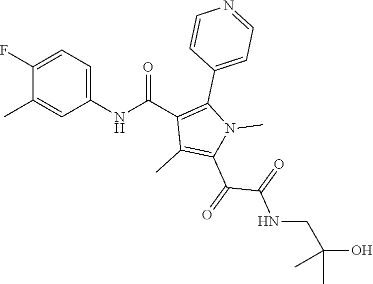 [SMILES: Cc1cc(NC(=O)c2c(C)c(C(=O)C(=O)NCC(C)(C)O)n(C)c2-c2ccncc2)ccc1F] | N-(4-fluoro-3-methylphenyl)-5- (2-((2-hydroxy-2- methylpropyl)amino)-2- oxoacetyl)-1,4-dimethyl-2- (pyridin-4-yl)-1H-pyrrole-3- carboxamide | 467 |
| 165 | 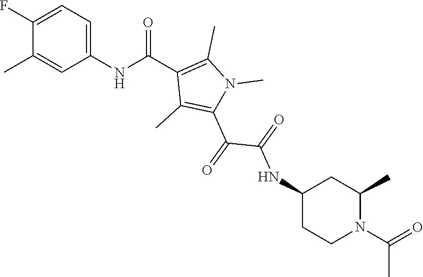 [SMILES: CC(=O)N1CC[C@@H](NC(=O)C(=O)c2c(C)c(C(=O)Nc3ccc(F)c(C)c3)c(C)n2C)C[C@H]1C] | 5-(2-(((2R,4R)-1-acetyl-2- methylpiperidin-4-yl)amino)-2- oxoacetyl)-N-(4-fluoro-3- methylphenyl)-1,2,4-trimethyl- 1H-pyrrole-3-carboxamide | 471 |
| 166 | 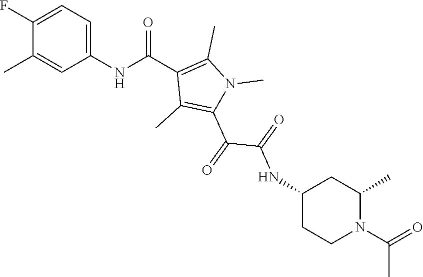 [SMILES: CC(=O)N1CC[C@H](NC(=O)C(=O)c2c(C)c(C(=O)Nc3ccc(F)c(C)c3)c(C)n2C)C[C@@H]1C] | 5-(2-(((2S,4S)-1-acetyl-2- methylpiperidin-4-yl)amino)-2- oxoacetyl)-N-(4-fluoro-3- methylphenyl)-1,2,4-trimethyl- 1H-pyrrole-3-carboxamide | 471 |
| 167 | 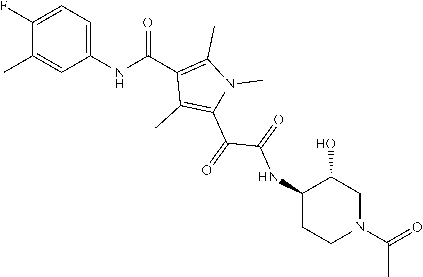 [SMILES: CC(=O)N1CC[C@@H](NC(=O)C(=O)c2c(C)c(C(=O)Nc3ccc(F)c(C)c3)c(C)n2C)[C@H](O)C1] | 5-(2-(((3R,4R)-1-acetyl-3- hydroxypiperidin-4-yl)amino)- 2-oxoacetyl)-N-(4-fluoro-3- methylphenyl)-1,2,4-trimethyl- 1H-pyrrole-3-carboxamide | 473 |
| 168 | 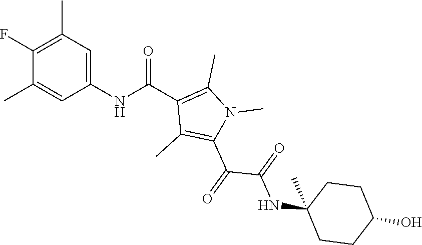 [SMILES: Cc1cc(NC(=O)c2c(C)c(C(=O)C(=O)N[C@]3(C)CC[C@@H](O)CC3)n(C)c2C)cc(C)c1F] | N-(4-fluoro-3,5- dimethylphenyl)-5-(2-(((1r,4r)- 4-hydroxy-1- methylcyclohexyl)amino)-2- oxoacetyl)-1,2,4-trimethyl-1H- pyrrole-3-carboxamide | 458.2 |
| 169 | 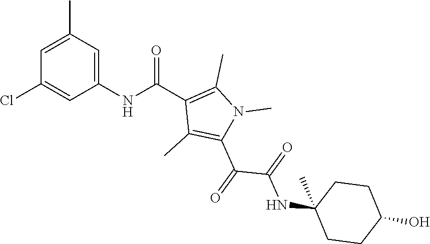 [SMILES: Cc1cc(Cl)cc(NC(=O)c2c(C)c(C(=O)C(=O)N[C@]3(C)CC[C@@H](O)CC3)n(C)c2C)c1] | N-(3-chloro-5-methylphenyl)-5- (2-(((1r,4r)-4-hydroxy-1- methylcyclohexyl)amino)-2- oxoacetyl)-1,2,4-trimethyl-1H- pyrrole-3-carboxamide | 460.9 |
| 170 | 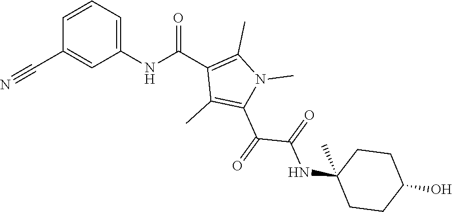 [SMILES: Cc1c(C(=O)Nc2cccc(C#N)c2)c(C)n(C)c1C(=O)C(=O)N[C@]1(C)CC[C@@H](O)CC1] | N-(3-cyanophenyl)-5-(2- (((1r,4r)-4-hydroxy-1- methylcyclohexyl)amino)-2- oxoacetyl)-1,2,4-trimethyl-1H- pyrrole-3-carboxamide | 437.2 |
| 171 | 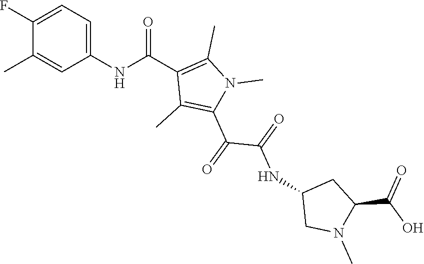 [SMILES: Cc1cc(NC(=O)c2c(C)c(C(=O)C(=O)N[C@@H]3C[C@@H](C(=O)O)N(C)C3)n(C)c2C)ccc1F] | (2S,4R)-4-(2-(4-((4-fluoro-3- methylphenyl)carbamoyl)-1,3,5- trimethyl-1H-pyrrol-2-yl)-2- oxoacetamido)-1- methylpyrrolidine-2-carboxylic acid | 459 |
| 172 | 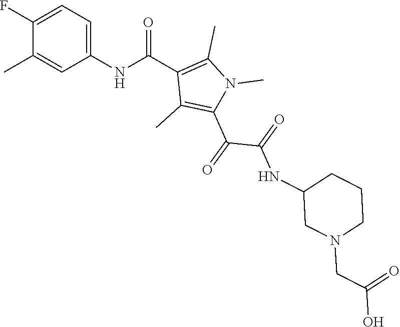 [SMILES: Cc1cc(NC(=O)c2c(C)c(C(=O)C(=O)NC3CCCN(CC(=O)O)C3)n(C)c2C)ccc1F] | 2-(3-(2-(4-((4-fluoro-3- methylphenyl)carbamoyl)-1,3,5- trimethyl-1H-pyrrol-2-yl)-2- oxoacetamido)piperidin-1- yl)acetic acid | 473 |
| 173 | 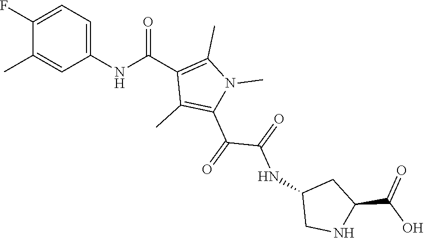 [SMILES: Cc1cc(NC(=O)c2c(C)c(C(=O)C(=O)N[C@H]3CN[C@H](C(=O)O)C3)n(C)c2C)ccc1F] | (2S,4R)-4-(2-(4-((4-fluoro-3- methylphenyl)carbamoyl)-1,3,5- trimethyl-1H-pyrrol-2-yl)-2- oxoacetamido)pyrrolidine-2- carboxylic acid | 445 |
| 174 | 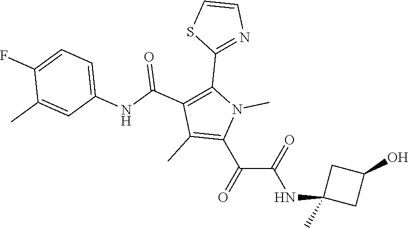 [SMILES: Cc1cc(NC(=O)c2c(C)c(C(=O)C(=O)N[C@]3(C)C[C@H](O)C3)n(C)c2-c2nccs2)ccc1F] | N-(4-fluoro-3-methylphenyl)-5- (2-(((1s,3s)-3-hydroxy-1- methylcyclobutyl)amino)-2- oxoacetyl)-1,4-dimethyl-2- (thiazol-2-yl)-1H-pyrrole-3- carboxamide | 485 |
| 175 | 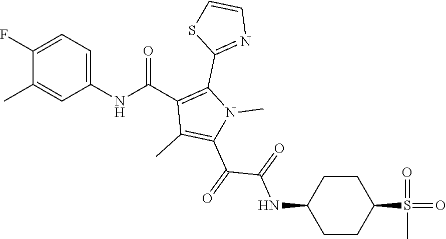 [SMILES: Cc1cc(NC(=O)c2c(C)c(C(=O)C(=O)N[C@H]3CC[C@@H](S(C)(=O)=O)CC3)n(C)c2-c2nccs2)ccc1F] | N-(4-fluoro-3-methylphenyl)- 1,4-dimethyl-5-(2-(((1s,4s)-4- (methylsulfonyl)cyclohexyl)amino)- 2-oxoacetyl)-2-(thiazol-2- yl)-1H-pyrrole-3-carboxamide | 561 |
| 176 | 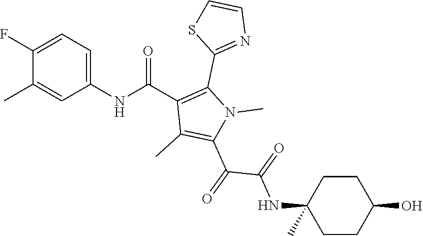 [SMILES: Cc1cc(NC(=O)c2c(C)c(C(=O)C(=O)N[C@]3(C)CC[C@H](O)CC3)n(C)c2-c2nccs2)ccc1F] | N-(4-fluoro-3-methylphenyl)-5- (2-(((1s,4s)-4-hydroxy-1- methylcyclohexyl)amino)-2- oxoacetyl)-1,4-dimethyl-2- (thiazol-2-yl)-1H-pyrrole-3- carboxamide | 513 |
| 177 | 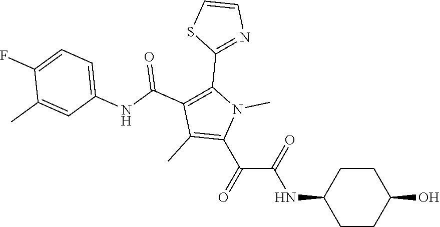 [SMILES: Cc1cc(NC(=O)c2c(C)c(C(=O)C(=O)N[C@H]3CC[C@@H](O)CC3)n(C)c2-c2nccs2)ccc1F] | N-(4-fluoro-3-methylphenyl)-5- (2-(((1s,4s)-4- hydroxycyclohexyl)amino)-2- oxoacetyl)-1,4-dimethyl-2- (thiazol-2-yl)-1H-pyrrole-3- carboxamide | 499 |
| 178 | 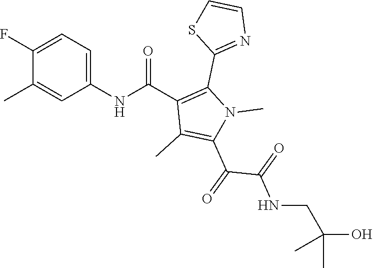 [SMILES: Cc1cc(NC(=O)c2c(C)c(C(=O)C(=O)NCC(C)(C)O)n(C)c2-c2nccs2)ccc1F] | N-(4-fluoro-3-methylphenyl)-5- (2-((2-hydroxy-2- methylpropyl)amino)-2- oxoacetyl)-1,4-dimethyl-2- (thiazol-2-yl)-1H-pyrrole-3- carboxamide | 473 |
| 179 | 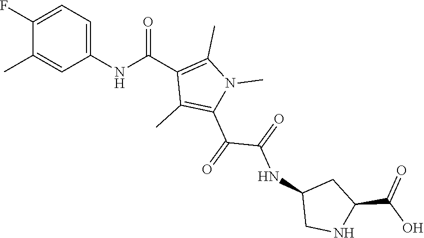 [SMILES: Cc1cc(NC(=O)c2c(C)c(C(=O)C(=O)N[C@@H]3CN[C@H](C(=O)O)C3)n(C)c2C)ccc1F] | (2S,4S)-4-(2-(4-((4-fluoro-3- methylphenyl)carbamoyl)-1,3,5- trimethyl-1H-pyrrol-2-yl)-2- oxoacetamido)pyrrolidine-2- carboxylic acid | 459 |
| 180 | 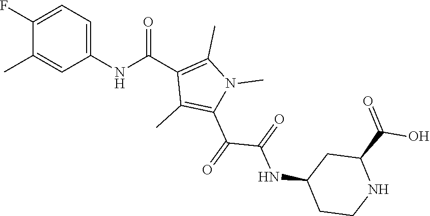 [SMILES: Cc1cc(NC(=O)c2c(C)c(C(=O)C(=O)N[C@@H]3CCN[C@H](C(=O)O)C3)n(C)c2C)ccc1F] | (2S,4R)-4-(2-(4-((4-fluoro-3- methylphenyl)carbamoyl)-1,3,5- trimethyl-1H-pyrrol-2-yl)-2- oxoacetamido)piperidine-2- carboxylic acid | 473 |
| 181 | 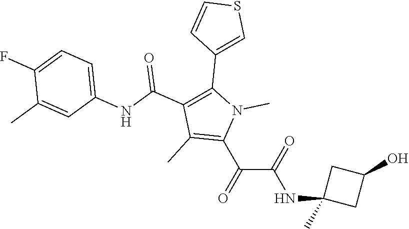 [SMILES: Cc1cc(NC(=O)c2c(C)c(C(=O)C(=O)N[C@]3(C)C[C@H](O)C3)n(C)c2-c2ccsc2)ccc1F] | N-(4-fluoro-3-methylphenyl)-5- (2-(((1s,3s)-3-hydroxy-1- methylcyclobutyl)amino)-2- oxoacetyl)-1,4-dimethyl-2- (thiophen-3-yl)-1H-pyrrole-3- carboxamide | 484 |
| 182 | 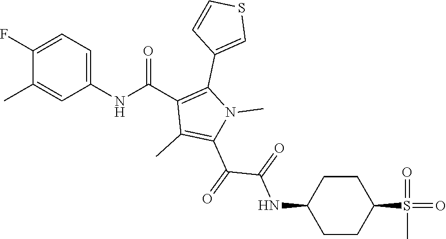 [SMILES: Cc1cc(NC(=O)c2c(C)c(C(=O)C(=O)N[C@H]3CC[C@@H](S(C)(=O)=O)CC3)n(C)c2-c2ccsc2)ccc1F] | N-(4-fluoro-3-methylphenyl)- 1,4-dimethyl-5-(2-(((1s,4s)-4- (methylsulfonyl)cyclohexyl)amino)- 2-oxoacetyl)-2-(thiophen-3- yl)-1H-pyrrole-3-carboxamide | 560 |
| 183 | 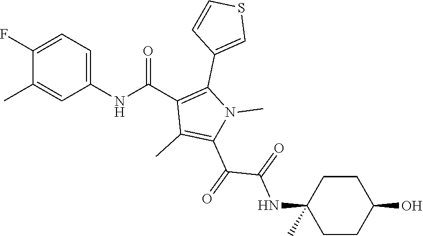 [SMILES: Cc1cc(NC(=O)c2c(C)c(C(=O)C(=O)N[C@]3(C)CC[C@H](O)CC3)n(C)c2-c2ccsc2)ccc1F] | N-(4-fluoro-3-methylphenyl)-5- (2-(((1s,4s)-4-hydroxy-1- methylcyclohexyl)amino)-2- oxoacetyl)-1,4-dimethyl-2- (thiophen-3-yl)-1H-pyrrole-3- carboxamide | 512 |
| 184 | 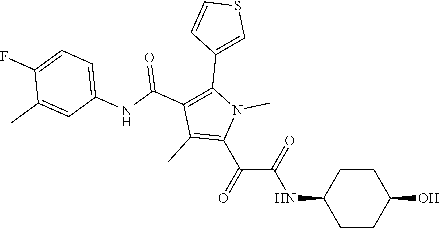 [SMILES: Cc1cc(NC(=O)c2c(C)c(C(=O)C(=O)N[C@H]3CC[C@@H](O)CC3)n(C)c2-c2ccsc2)ccc1F] | N-(4-fluoro-3-methylphenyl)-5- (2-(((1s,4s)-4- hydroxycyclohexyl)amino)-2- oxoacetyl)-1,4-dimethyl-2- (thiophen-3-yl)-1H-pyrrole-3- carboxamide | 498 |
| 185 | 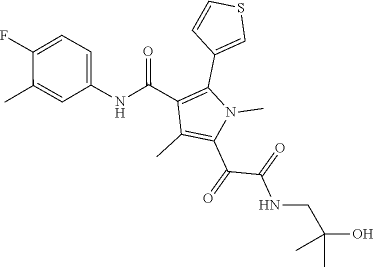 [SMILES: Cc1cc(NC(=O)c2c(C)c(C(=O)C(=O)NCC(C)(C)O)n(C)c2-c2ccsc2)ccc1F] | N-(4-fluoro-3-methylphenyl)-5- (2-((2-hydroxy-2- methylpropyl)amino)-2- oxoacetyl)-1,4-dimethyl-2- (thiophen-3-yl)-1H-pyrrole-3- carboxamide | 472 |
| 186 | 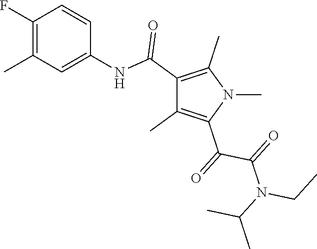 [SMILES: CCN(C(=O)C(=O)c1c(C)c(C(=O)Nc2ccc(F)c(C)c2)c(C)n1C)C(C)C] | 5-(2-(ethyl(isopropyl)amino)-2- oxoacetyl)-N-(4-fluoro-3- methylphenyl)-1,2,4-trimethyl- 1H-pyrrole-3-carboxamide | 402 |
| 187 | 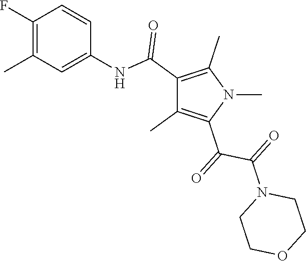 [SMILES: Cc1cc(NC(=O)c2c(C)c(C(=O)C(=O)N3CCOCC3)n(C)c2C)ccc1F] | N-(4-fluoro-3-methylphenyl)- 1,2,4-trimethyl-5-(2- morpholino-2-oxoacetyl)-1H- pyrrole-3-carboxamide | 402 |
| 188 | 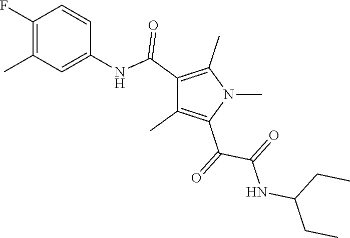 [SMILES: CCC(CC)NC(=O)C(=O)c1c(C)c(C(=O)Nc2ccc(F)c(C)c2)c(C)n1C] | N-(4-fluoro-3-methylphenyl)- 1,2,4-trimethyl-5-(2-oxo-2- (pentan-3-ylamino)acetyl)-1H- pyrrole-3-carboxamide | 402 |

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography, or preferably, by separation/resolution techniques based upon differences in solubility. In some embodiments, the optically pure enantiomer is then recovered, along with the resolving agent, by any practical means that would not result in racemization.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability. Further, substitution with heavy isotopes such as deuterium, i.e., ²H, produces certain therapeutic advantages resulting from greater metabolic stability, for example increased in vivo half-life or reduced dosage requirements.

In some embodiments, the compounds described herein are labeled by other means, including, but not limited to, the use of chromophores or fluorescent moieties, bioluminescent labels, or chemiluminescent labels.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Examples of pharmaceutically acceptable salts include those salts prepared by reaction of the compounds described herein with a mineral, organic acid or inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate metaphosphate, methoxybenzoate, methylbenzoate, monohydrogenphosphate, 1-napthalenesulfonate, 2-napthalenesulfonate, nicotinate, nitrate, pamoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, suberate, sebacate, sulfonate, tartrate, thiocyanate, tosylate undecanoate and xylenesulfonate.

Further, the compounds described herein can be prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, p-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid and muconic acid. In some embodiments, other acids, such as oxalic, while not in themselves pharmaceutically acceptable, are employed in the preparation of salts useful as intermediates in obtaining the compounds disclosed herein, solvate, or stereoisomer thereof and their pharmaceutically acceptable acid addition salts.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine. Representative salts include the alkali or alkaline earth salts, like lithium, sodium, potassium, calcium, and magnesium, and aluminum salts and the like. Illustrative examples of bases include sodium hydroxide, potassium hydroxide, choline hydroxide, sodium carbonate, N⁺(C₁₋₄ alkyl)₄, and the like.

Representative organic amines useful for the formation of base addition salts include ethylamine, diethylamine, ethylenediamine, ethanolamine, diethanolamine, piperazine and the like. It should be understood that the compounds described herein also include the quaternization of any basic nitrogen-containing groups they contain. In some embodiments, water or oil-soluble or dispersible products are obtained by such quaternization.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents including, but not limited to, dioxane, tetrahydrofuran or methanol. In addition, the compounds provided herein can exist in unsolvated as well as solvated forms. In general, the solvated forms are considered equivalent to the unsolvated forms for the purposes of the compounds and methods provided herein.

### Tautomers

In some situations, compounds exist as tautomers. The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Method of Treatment

The compounds described herein find use in a variety of applications for human and animal health. In some embodiments, the compounds described herein are inhibitors of hepatitis B virus (HBV).

In some embodiments, the compounds described herein are capsid inhibitor. In some embodiments, the compounds described herein are used in treating HBV infection and related conditions, including chronic hepatitis B, HBV/HDV co-infection, HBV/HCV co-infection, HBV/HIV co-infection, inflammation, necrosis, cirrhosis, hepatocellular carcinoma, hepatic decompensation and hepatic injury from an HBV infection.

In some embodiments, the efficacy of treatment is determined using quantification of viral load or other evidence of infection, such as through measurement of HBeAg (hepatitis B e-antigen), HBsAg, HBV DNA levels, ALT (Alanine Transaminase) activity levels, serum HBV levels, and the like, thereby allowing adjustment of treatment dose, treatment frequency, and treatment length.

In some embodiments, the compounds described herein reduce viral load in an individual suffering from an HBV infection.

### Pharmaceutical Compositions/Formulations

The compounds described herein are administered to a subject in need thereof, either alone or in combination with pharmaceutically acceptable carriers, excipients or diluents, in a pharmaceutical composition, according to standard pharmaceutical practice. In one embodiment, the compounds of this invention may be administered to animals. The compounds can be administered orally or parenterally, including the intravenous, intramuscular, intraperitoneal, subcutaneous, rectal and topical routes of administration.

In another aspect, provided herein are pharmaceutical compositions comprising a compound describe herein, or a pharmaceutically acceptable salt, solvate, or stereoisomer thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compounds described herein, or a pharmaceutically acceptable salt, solvate, or stereoisomer thereof are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

### Combination

Disclosed herein are methods of treating hepatitis B using a compound disclosed herein in combination with additional therapeutic agents useful for treating chronic HBV infection.

In some embodiments, the compound disclosed herein in combination with additional therapeutic agents useful for treating an HBV infection are administered simultaneously. In some embodiments, the compound disclosed herein in combination with additional therapeutic agents useful for treating an HBV infection are administered sequentially.

Non-limiting examples of additional therapeutic agents useful for treating HBV infections include: reverse transcriptase inhibitors; HBV polymerase inhibitors, capsid inhibitors; cccDNA formation inhibitors; RNA destabilizers; checkpoint inhibitors (e.g., PD-1/PD-L1 inhibitors); therapeutic vaccines; RNA interference (RNAi) therapeutics; antisense-based therapeutics, HBV entry inhibitors; TLR agonists; RIG-I agonists; or interferons.

### Reverse Transcriptase Inhibitors

In some embodiments, the compound described herein is used in combination with a reverse transcriptase inhibitor. In certain embodiments, the reverse transcriptase inhibitor is a reverse transcriptase inhibitor (NARTI or NRTI). In other embodiments, the reverse transcriptase inhibitor is a nucleotide analog reverse transcriptase inhibitor (NtARTI or NtRTI). Reverse transcriptase inhibitors include, but are not limited to, entecavir, clevudine, telbivudine, lamivudine, adefovir, and tenofovir, tenofovir disoproxil, tenofovir alafenamide, adefovir dipovoxil, or any combination thereof. In some embodiments, the compound described herein is used in combination with tenofovir. In some embodiments, the compound described herein is used in combination with entecavir.

### HBV Polymerase Inhibitors

In some embodiments, the compound described herein is used in combination with an HBV polymerase inhibitor. In some embodiments, the HBV polymerase inhibitor is entecavir, lamivudine, telbivudine, adefovir, tenofovir disoproxil fumarate, tenofovir alafenamide fumarate (TAF), tenofovir disoproxil orotate, or tenofovir disopropxil aspartate.

### Capsid Inhibitors

In some embodiments, the compound described herein is used in combination with a capsid inhibitor. For example, a capsid assembly inhibitor includes, but is not limited to, any compound that inhibits capsid assembly, induces formation of non-capsid polymers, promotes excess capsid assembly or misdirected capsid assembly, affects capsid stabilization, and/or inhibits encapsidation of RNA (pgRNA). Capsid inhibitors also include any compound that inhibits capsid function in a downstream event(s) within the replication process (e.g., viral DNA synthesis, transport of relaxed circular DNA (rcDNA) into the nucleus, covalently closed circular DNA (cccDNA) formation, virus maturation, budding and/or release, and the like). For example, in certain embodiments, the inhibitor detectably inhibits the expression level or biological activity of the capsid protein as measured, e.g., using an assay described herein. In some embodiments, the capsid inhibitor is NVR 3-778, GLS-4, AB-423, AB-506, JNJ-56136379, JNJ-64530440, ABI-H0731, ABI-H2158, ABI-H3733, EDP-514, GLP-26, ALG-000184, ALG-001024, ALG-001075, QL-007, QL-OA6a, CB-HBV-001, and RO7049389.

cccDNA Formation Inhibitors (cccDNA)

In some embodiments, the compound described herein is used in combination with a covalently closed circular DNA (cccDNA). Covalently closed circular DNA (cccDNA) is generated in the cell nucleus from viral rcDNA and serves as the transcription template for viral mRNAs. In some embodiments, the cccDNA formation inhibitor includes compounds that are capable of inhibiting the formation and/or stability of cccDNA either directly or indirectly. In some embodiments, a cccDNA formation inhibitor includes, but is not limited to, any compound that inhibits capsid disassembly, rcDNA entry into the nucleus, and/or the conversion of rcDNA into cccDNA. In certain embodiments, the inhibitor detectably inhibits the formation and/or stability of the cccDNA as measured, e.g., using an assay described herein.

### RNA Destabilizer

In some embodiments, the compound described herein is used in combination with an RNA destabilizer. In some embodiments, an RNA destabilizer refers to a molecule, or a salt or solvate thereof, that reduces the total amount of HBV RNA in mammalian cell culture or in a live human subject. In a non-limiting example, an RNA destabilizer reduces the amount of the RNA transcript(s) encoding one or more of the following HBV proteins: surface antigen, core protein, RNA polymerase, and e antigen. In some embodiments, the RNA destabilizer is RG7834 or AB-452.

### Checkpoint Inhibitors

In some embodiments, the compound described herein is used in combination with a checkpoint inhibitor. In some embodiments, checkpoint inhibitors include any compound that is capable of inhibiting immune checkpoint molecules that are regulators of the immune system (e.g., stimulate or inhibit immune system activity). For example, some checkpoint inhibitors block inhibitory checkpoint molecules, thereby stimulating immune system function, such as stimulation of T cell activity against cancer cells. A non-limiting example of a checkpoint inhibitor is a PD-L1 inhibitor or a PD-1 inhibitor. In some embodiments, the PD-1 inhibitor is pembrolizumab, nivolumab, cemiplimab, spartalizumab, camrelizumab, sintilimab, tislelizumab, toripalimab, AMP-224, or AMP-514. In some embodiments, the PD-L1 inhibitor is atezolizumab, avelumab, durvalumab, KN035, CK-301, AUNP12, CA-170, or BMS-986189.

### Therapeutic Vaccines

In some embodiments, the compound described herein is used in combination with a therapeutic vaccine. In some embodiments, the therapeutic vaccine is HBsAG-HBIG, HB-Vac, ABX203, NASVAC, GS-4774, GX-110 (also known as HB-110E), CVI-HBV-002, RG7944 (also known as INO-1800), TG-1050, FP-02 (Hepsyn-B), AIC649, VGX-6200, KW-2, TomegaVax-HBV, ISA-204, NU-500, INX-102-00557 HBV MVA, or PepTcell.

### RNA Interference (RNAi) Therapeutics

In some embodiments, the compound described herein is used in combination with an RNA interference (RNAi) therapeutic. In some embodiments, the RNA interference therapeutic is TKM-HBV (also known as ARB-1467), ARB-1740, ARC-520, ARC-521, BB-HB-331, REP-2139, ALN-HBV, ALN-PDL, LUNAR-HBV, GS3228836, or GS3389404.

### HBV Entry Inhibitors

In some embodiments, the compound described herein is used in combination with an HBV entry inhibitor. In some embodiments, the HBV entry inhibitor is bulevirtide, IVIG-Tonrol, or GC-1102.

### TLR Agonists

In some embodiments, the compound described herein is used in combination with a TLR agonist (TLR7, 8 and/or 9). In some embodiments, the TLR agonist is RG7795, GS-9620, SM360320, or AZD 8848.

### RIG-I Agonists

In some embodiments, the compound described herein is used in combination with a RIG-I agonist. In some embodiments, the RIG-I agonist is inarigivir.

### Interferons

In some embodiments, the compound described herein is used in combination with an interferon. In some embodiments, the interferon is interferon alpha (IFN-a), interferon alpha-2a, recombinant interferon alpha-2a, peginterferon alpha-2a, interferon alpha-2b, recombinant interferon alpha-2b, interferon alpha-2b XL, peginterferon alpha-2b, glycosylated interferon alpha-2b, interferon alpha-2c, recombinant interferon alpha-2c, interferon beta, interferon beta-1a, peginterferon beta-1a, interferon delta, interferon lambda (IFN-1), peginterferon lambda-1, interferon omega, interferon tau, interferon gamma (IFN-g), interferon alfacon-1, interferon alpha-n1, interferon alpha-n3, albinterferon alpha-2b, BLX-883, DA-3021, PI 101 (also known as AOP2014), PEG-infergen, belerofon, INTEFEN-IFN, albumin/interferon alpha 2a fusion protein, rHSA-IFN alpha 2a, rHSA-IFN alpha 2b, PEG-IFN-SA, interferon alpha biobetter; in particular, peginterferon alpha-2a, peginterferon alpha-2b, glycosylated interferon alpha-2b, peginterferon beta-1a, or peginterferon lambda-1.

### Preparation of Compounds

### Example 1: Synthesis of N-(4-fluoro-3-methylphenyl)-1,2,4-trimethyl-5-(2-(((1s,4s)-4-(methylsulfonyl)cyclohexyl)amino)-2-oxoacetyl)-1H-pyrrole-3-carboxamide

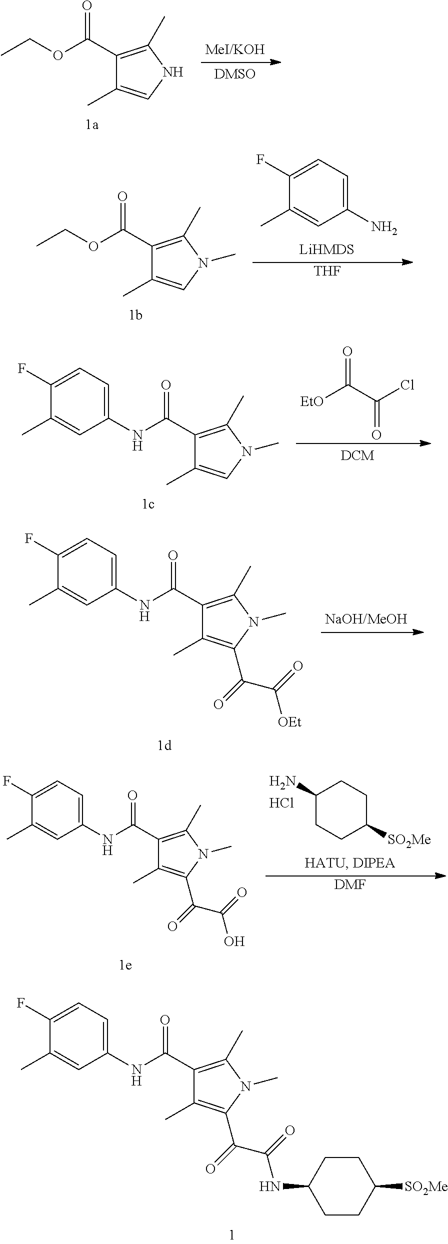

### Step 1: Synthesis of ethyl 1,2,4-trimethyl-1H-pyrrole-3-carboxylate (1b)

Iodomethane (31.8 g, 224.3 mmol) was added to a mixture of commercially available 1a (25 g, 149.5 mmol) and KOH (16.8 g, 299 mmol) in DMSO (250 mL) at 0° C. The reaction mixture was warmed to ambient temperature and maintained for 16 h. The reaction mixture was extracted with diethyl ether (4×). The combined organic extracts were washed with water, brine, dried over anhydrous Na2SO4, filtered and concentrated in vacuo to afford the title compound 1b as brown-colored solid (24.6 g, 91% yield) which was used without further purification. ESI-MS, m/z 182 [M+H]⁺.

### Step 2: Synthesis of N-(4-fluoro-3-methylphenyl)-1,2,4-trimethyl-1H-pyrrole-3-carboxamide (1c)

Lithium bis(trimethylsilyl)amide solution (1 N/THF, 272 mL, 272 mmol) was added dropwise over 45 min to a solution containing 2 (24.6 g, 136 mmol) and 3-methyl-4-fluoroaniline (18.8 g, 149 mmol) in THF (270 mL) at 0° C. The reaction mixture was allowed to warm slowly to ambient temperature. After 16 h, the reaction mixture was quenched with NH₄Cl (sat'd) and water. The layers were separated and the aqueous phase was extracted with EtOAc (3×). The combined organic extracts were washed successively with NH₄Cl (sat'd) and brine, dried over anhydrous Na₂SO₄, filtered and concentrated in vacuo. The crude residue was suspended in 1:1 EtOAc/hexanes and stirred for 1 h at 40° C., then cooled to ambient temperature and filtered. The filter cake was washed with hexanes and dried to afford the title compound 1c as tan-colored solid (31.8 g, 90% yield). ¹H NMR (300 MHz, DMSO-d₆): δ 9.25 (s, 1H), 7.58 (d, J=7.2 Hz, 1H), 7.45 (m, 1H), 7.02 (t, J=9.6 Hz, 1H), 6.44 (s, 1H), 3.43 (s, 3H), 2.26 (s, 3H), 2.19 (s, 3H), 2.06 (s, 3H) ppm. ESI-MS, m/z 261 [M+H]⁺.

### Step 3: Synthesis of ethyl 2-(4-((4-fluoro-3-methylphenyl)carbamoyl)-1,3,5-trimethyl-1H-pyrrol-2-yl)-2-oxoacetate (1d)

To a solution of 1c (31.8 g, 121.9 mmol) in DCM (500 mL) at 0° C. was added ethyl chlorooxoacetate (24.9 g, 182.8 mmol) dropwise over 30 min. The reaction mixture was allowed to warm slowly to ambient temperature. After 16 h, the reaction mixture was washed successively with water and NaHCO₃ (sat'd) and then concentrated in vacuo to afford 1d (44 g, quant.) as a brown-colored solid which was used without any further purification. ESI-MS, m/z 361 [M+H]⁺.

### Step 4: Synthesis of 2-(4-((4-fluoro-3-methylphenyl)carbamoyl)-1,3,5-trimethyl-1H-pyrrol-2-yl)-2-oxoacetic acid (1e)

To a solution of 1d (43.9 g, 121.9 mmol) in THF (200 mL) and MeOH (200 mL) was added 1 N NaOH (300 mL). After 15 min, the reaction mixture was partially concentrated to remove THF/MeOH, diluted with EtOAc and partially concentrated again. The heterogeneous mixture was diluted with water and washed with EtOAc (4×). The heterogeneous aqueous layer was acidified (pH 1) with conc. HCl and extracted with EtOAc (4×). The heterogeneous organic layer was washed with brine and the solids were collected by filtration. The filter cake was washed with hexanes. The crude solids were suspended in EtOAc (200 mL) and hexanes (200 mL) and stirred for 1 h at 45° C., then cooled to ambient temperature, collected by filtration, and washed with hexanes. The solids were further dried in vacuo to provide 1e (34.21 g, 84% yield) as an off-white solid. ¹H NMR (300 MHz, DMSO-d₆): δ 9.96 (s, 1H), 7.60 (d, J=6.9 Hz, 1H), 7.46 (m, 1H), 7.07 (t, J=9.6 Hz, 1H), 3.78 (s, 3H), 2.32 (s, 3H), 2.23 (s, 3H), 2.20 (s, 3H) ppm. ESI-MS, m/z 333 [M+H]⁺.

### Step 5: Synthesis of N-(4-fluoro-3-methylphenyl)-1,2,4-trimethyl-5-(2-(((1s,4s)-4-(methylsulfonyl)cyclohexyl)amino)-2-oxoacetyl)-1H-pyrrole-3-carboxamide (1)

HATU (68 mg, 0.18 mmol) was added to a solution of 1e (50 mg, 0.15 mmol) in DMF (1 mL) at 0° C. After 20 min, (1S,4S)-4-(methylsulfonyl)cyclohexan-1-amine hydrochloride (36 mg, 0.17) and DIPEA (78 mg, 0.60 mmol) were added. After 20 h the reaction mixture was quenched with aqueous TFA (4%, 0.4 mL), then, extracted with EtOAc (10 mL). The organic layer was washed with water and brine, concentrated in vacuo, then, purified by reverse phase chromatography eluted with ACN and water, and dried using lyophilization to afford the title product as white solid. ESI-MS, m/z 360 (M+H)⁺.

### Example 2: N-(2,6-dichloropyridin-4-yl)-5-(2-(((1s,4s)-4-hydroxycyclohexyl)amino)-2-oxoacetyl)-1,2,4-trimethyl-1H-pyrrole-3-carboxamide

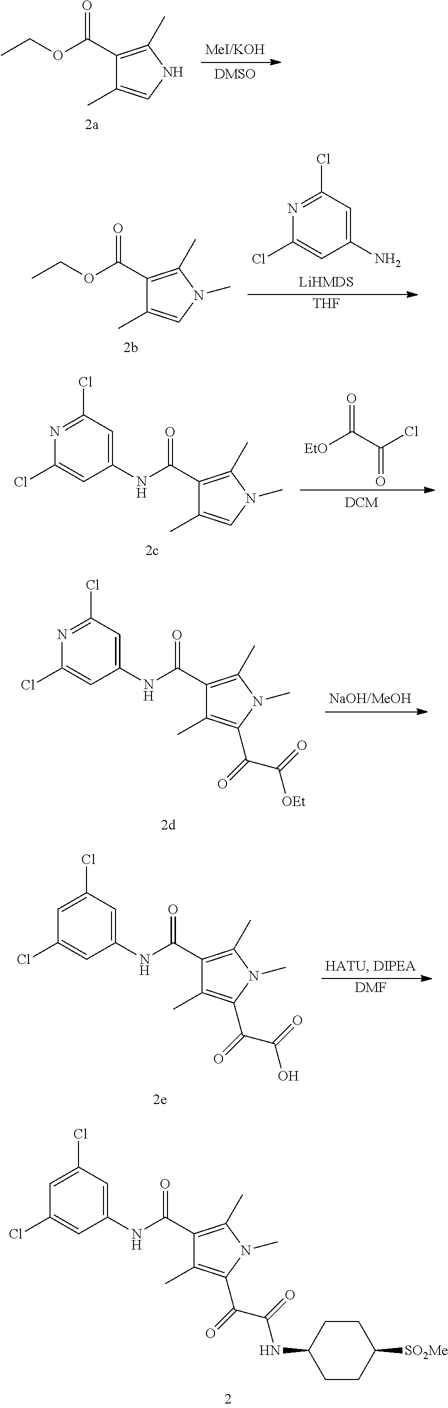

### Step 1: Synthesis of ethyl 1,2,4-trimethyl-1H-pyrrole-3-carboxylate (2b)

MeI (3.2 g, 22 mmol) was added to a mixture of 2a (2 g, 12 mmol) and KOH (2 g, 35 mmol) in DMSO (40 mL) at 0° C. The reaction mixture was warmed to rt for 16 hrs. The reaction mixture was extracted with 4×Et₂O. The combined extracts were washed with water and brine, dried over Na₂SO₄, filtered and concentrated in vacuo to afford the title compound 2b as brown solid (2 g) which was used without further purification. ESI-MS, m/z 182 (M+H)⁺.

### Step 2: Synthesis of N-(2,6-dichloropyridin-4-yl)-1,2,4-trimethyl-1H-pyrrole-3-carboxamide (2c)

LiHMDS (40 mL, 1 N in THF) was added dropwise to a solution of 2b (2 g, 11 mmol) and 2,6-dichloropyridin-4-amine (3.5 g, 22 mmol) in THF (25 mL) at 0° C. The reaction mixture was allowed to warm slowly to rt for 2 hrs, then, warmed to 80° C. for 16 hrs. The reaction mixture was cooled to rt, quenched with NH₄Cl (sat) and water. The layers were separated and the aqueous was extracted 3×EtOAc. The combined organics were washed with NH₄Cl (sat) and brine, dried over Na₂SO₄, filtered and concentrated. The crude residue was purified by flash chromatography on silica gel (EtOAc/Hexanes 0˜100%) to afford 2c as yellow solid (2.2 g). ESI-MS, m/z 298.1 (M+H)⁺.

### Step 3: Synthesis of ethyl 2-(4-((2,6-dichloropyridin-4-yl)carbamoyl)-1,3,5-trimethyl-1H-pyrrol-2-yl)-2-oxoacetate (2d)

To a solution of 2c (1 g, 3.4 mmol) in DCM (10 mL) at 0° C. was added ethyl chlorooxoacetate (2 g, 15 mmol) dropwise over 30 mins and the reaction mixture was allowed to warm slowly to ambient temperature. After 16 h the reaction mixture was washed with H₂O and NaHCO₃ (sat) and then concentrated in vacuo to afford the title compound 2d (1.1 g) as a brown solid which was used without any further purification. ESI-MS, m/z 398/400 (M+H)⁺.

### Step 4: Synthesis of 2-(4-((2,6-dichloropyridin-4-yl)carbamoyl)-1,3,5-trimethyl-1H-pyrrol-2-yl)-2-oxoacetic acid (2e)

To a solution of 1d (1 g, 2.5 mmol) in MeOH (3 mL) was added 2N NaOH (2 mL). After 15 min the reaction mixture was partially concentrated to remove organics, diluted with EtOAc and partially concentrated again. The heterogeneous mixture was diluted with water and washed EtOAc. The heterogeneous aqueous was acidified with 2N HCl to pH ˜2 and extracted with 4×EtOAc. The organic layer was washed with brine, and concentrated to afford the title compound 2e as white solid. ESI-MS, m/z 370/372 (M+H)⁺.

### Step 5: Synthesis of N-(2,6-dichloropyridin-4-yl)-5-(2-(((1s,4s)-4-hydroxycyclohexyl)amino)-2-oxoacetyl)-1,2,4-trimethyl-1H-pyrrole-3-carboxamide (2)

To a solution of 2e (20 mg), cis-4-aminocyclohexan-1-ol (15 mg) and HATU (50 mg) in DMF (0.5 mL) was added DIPEA (20 mg) at 0° C. The reaction mixture was warmed to rt overnight. The reaction mixture was quenched with aqueous HCl (0.2 N), and extracted with EtOAc. The organic layer was washed with water and brine, concentrated in vacuo, then, purified by reverse phase chromatography eluted with ACN and water, and dried using lyophilization to afford the title product as white solid. ESI-MS, m/z 467.1/469.1 (M+H)⁺.

### Example 3 and 6: Synthesis of 5-(2-((trans-1-cyclopropyl-4-hydroxycyclohexyl)amino)-2-oxoacetyl)-N-(4-fluoro-3-methylphenyl)-1,2,4-trimethyl-1H-pyrrole-3-carboxamide and 5-(2-((cis-1-cyclopropyl-4-hydroxycyclohexyl)amino)-2-oxoacetyl)-N-(4-fluoro-3-methylphenyl)-1,2,4-trimethyl-1H-pyrrole-3-carboxamide

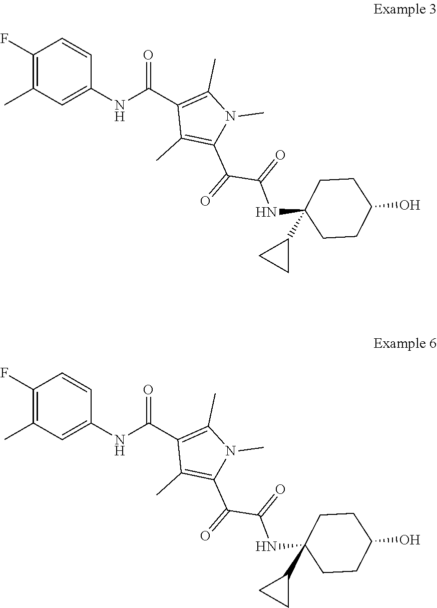 [SMILES: Cc1cc(NC(=O)c2c(C)c(C(=O)C(=O)N[C@]3(C4CC4)CC[C@@H](O)CC3)n(C)c2C)ccc1F ; Cc1cc(NC(=O)c2c(C)c(C(=O)C(=O)N[C@]3(C4CC4)CC[C@H](O)CC3)n(C)c2C)ccc1F]

The title compounds were prepared following the procedure described in Example 1, using 4-amino-4-cyclopropylcyclohexan-1-ol. The trans- and cis-isomer were separated and purified by reverse phase chromatography eluted with ACN and water and dried using lyophilization to afford the title products as white solids. ESI-MS m/z 470.2 (M+H)⁺.

### Example 4 and 5: Synthesis of 5-(2-((trans-1-ethyl-4-hydroxycyclohexyl)amino)-2-oxoacetyl)-N-(4-fluoro-3-methylphenyl)-1,2,4-trimethyl-1H-pyrrole-3-carboxamide and 5-(2-((cis-1-ethyl-4-hydroxycyclohexyl)amino)-2-oxoacetyl)-N-(4-fluoro-3-methylphenyl)-1,2,4-trimethyl-1H-pyrrole-3-carboxamide

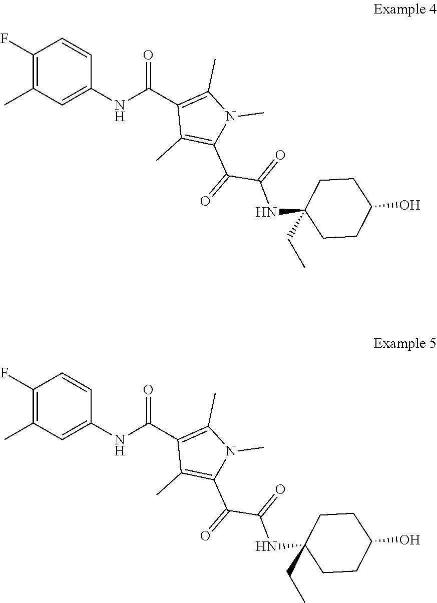 [SMILES: CC[C@]1(NC(=O)C(=O)c2c(C)c(C(=O)Nc3ccc(F)c(C)c3)c(C)n2C)CC[C@H](O)CC1 ; CC[C@]1(NC(=O)C(=O)c2c(C)c(C(=O)Nc3ccc(F)c(C)c3)c(C)n2C)CC[C@@H](O)CC1]

The title compounds were prepared following the procedure described in Example 1, using 4-amino-4-ethylcyclohexan-1-ol. The trans- and cis-isomer were separated and purified by reverse phase chromatography eluted with ACN and water and dried using lyophilization to afford the title products as white solids. ESI-MS m/z 458.2 (M+H)⁺.

### Example 7: 4-ethyl-N-(4-fluoro-3-methylphenyl)-5-(2-(((1s,4s)-4-hydroxy-1-methylcyclohexyl)amino)-2-oxoacetyl)-1,2-dimethyl-1H-pyrrole-3-carboxamide

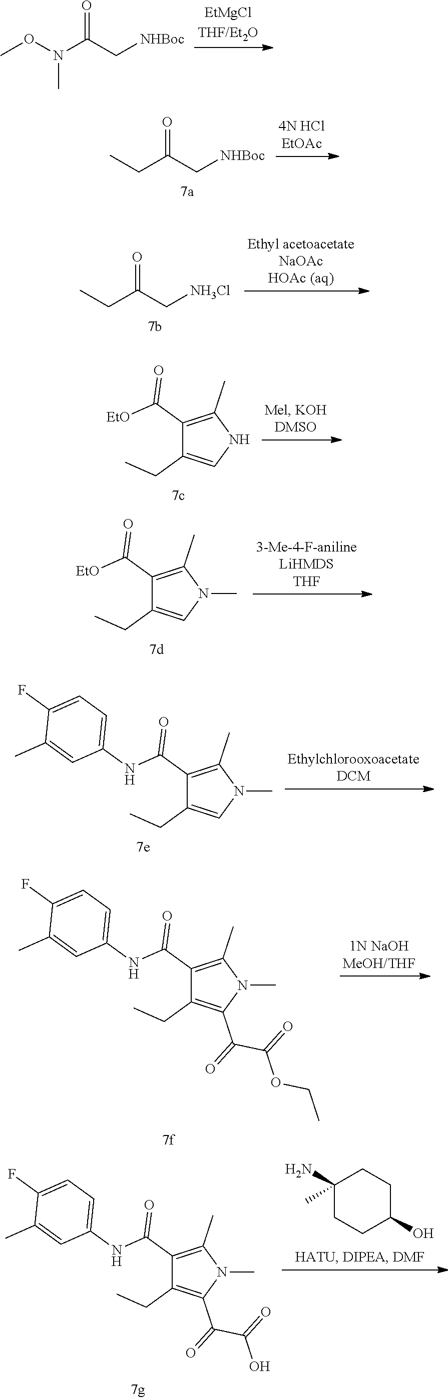

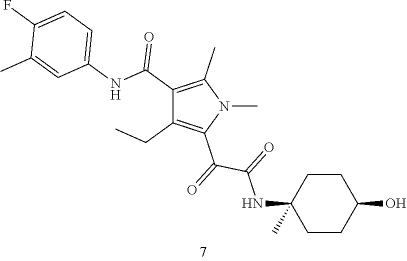 [SMILES: CCc1c(C(=O)Nc2ccc(F)c(C)c2)c(C)n(C)c1C(=O)C(=O)N[C@]1(C)CC[C@H](O)CC1]

### Step 1: Synthesis of tert-butyl (2-oxobutyl)carbamate (7)

To a solution of commercially available tert-butyl (2-(methoxy(methyl)amino)-2-oxoethyl)carbamate (8 g, 36.6 mmol) in THF (240 mL) and Et₂O (80 mL) at 0° C. was added EtMgCl (73 mL, 146.4 mmol, 2 M/THF) dropwise via addition funnel over a period of 30 min. The reaction mixture was allowed to warm gradually to ambient temperature. After 16 h the reaction mixture was poured into 1 N HCl and separated. The aqueous layer was extracted 3×Et₂O and the combined organics were washed with brine, dried over Na₂SO₄, filtered and concentrated to afford the title compound 7a (5.85 g, 86%) as a pale yellow oil which was taken forward without any further purification. ESI-MS, m/z 188 (M+H)⁺.

### Step 2: Synthesis of 1-aminobutan-2-one (7b)

To a solution of 7a (5.85 g, 31.24 mmol) in EtOAc (50 mL) at 0° C. was added HCl (23.4 mL, 93.73 mmol, 4N/dioxane) and the mixture was allowed to warm gradually to ambient temperature. After 16 h the reaction mixture was concentrated and the resultant solids were triturated with Et₂O, filtered and washed with Et₂O to afford the title compound 7b (3.46 g, 90%) as a pale yellow solid which was taken forward without further purification. ESI-MS, m/z 88 (M+H)⁺.

### Step 3: Synthesis of ethyl 4-ethyl-2-methyl-1H-pyrrole-3-carboxylate (7c)

A mixture of 7b (3.46 g, 25.14 mmol), ethyl acetoacetate (3.60 g, 27.66 mmol) and NaOAc (3.13 g, 38.1 mmol) in 75% Aqueous HOAc (20 mL) was heating to 100° C. After 3 h the reaction mixture was cooled to ambient temperature, poured into water, and extracted 4×Et₂O. The combined organics were washed successively with 2×H₂O, 2×NaHCO₃ (sat), and brine, dried over Na₂SO₄, filtered and concentrated. The crude residue was purified by flash chromatography on silica gel eluting with EtOAc and hexanes to afford the title compound 7c (3.1 g, 68%) as an off-white solid. ESI-MS, m/z 182 [M+H]⁺.

### Step 4: Synthesis of ethyl 4-ethyl-1,2-dimethyl-1H-pyrrole-3-carboxylate (7d)

The title compound was prepared following the procedure described in Example 1, Step 1, using 7c as the starting material. ESI-MS, m/z 196 (M+H)⁺.

### Step 5: Synthesis of 4-ethyl-N-(4-fluoro-3-methylphenyl)-1,2-dimethyl-1H-pyrrole-3-carboxamide (7e)

The title compound was prepared following the procedure described in Example 1, Step 2, using 7d as the starting material. ESI-MS, m/z 275 (M+H)⁺.

### Step 6: Synthesis of ethyl 2-(3-ethyl-4-((4-fluoro-3-methylphenyl)carbamoyl)-1,5-dimethyl-1H-pyrrol-2-yl)-2-oxoacetate (7f)

The title compound was prepared following the procedure described in Example 1, Step 3, using 7e as the starting material. ESI-MS, m/z 375 (M+H)⁺.

### Step 7: Synthesis of 2-(3-ethyl-4-((4-fluoro-3-methylphenyl)carbamoyl)-1,5-dimethyl-1H-pyrrol-2-yl)-2-oxoacetic acid (7g)

The title compound was prepared following the procedure described in Example 1, Step 4, using 7f as the starting material. ESI-MS, m/z 347 (M+H)⁺.

### Step 7: Synthesis of 4-ethyl-N-(4-fluoro-3-methylphenyl)-5-(2-(((1s,4s)-4-hydroxy-1-methylcyclohexyl)amino)-2-oxoacetyl)-1,2-dimethyl-1H-pyrrole-3-carboxamide (7)

The title compound was prepared following the procedure described in Example 1, Step 5, using 7g as the starting material and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS, m/z 458 (M+H)⁺.

### Example 8: Synthesis of N-(4-fluoro-3-methylphenyl)-1,2,4-trimethyl-5-(2-(((1r,4r)-4-(methylsulfonyl)cyclohexyl)amino)-2-oxoacetyl)-1H-pyrrole-3-carboxamide

The title compound was prepared following the procedure described in Example 1, Step 5, using (1R,4R)-4-(methylsulfonyl)cyclohexan-1-amine hydrochloride. The final product was purified by reverse phase chromatography eluted with ACN and water and dried using lyophilization to afford the title products as white solids. ESI-MS, m/z 492 (M+H)⁺.

### Example 9: N-(2,6-dichloropyridin-4-yl)-5-(2-(((1s,4s)-4-hydroxycyclohexyl)amino)-2-oxoacetyl)-1,2,4-trimethyl-1H-pyrrole-3-carboxamide

The title compounds were prepared following the procedure described in Example 2, Step 5 using trans-4-amino-4-methylcyclohexan-1-ol instead of cis-4-aminocyclohexan-1-ol. ESI-MS, m/z 481.1/483.1 (M+H)⁺.

### Example 10: N-(3,5-dichlorophenyl)-5-(2-(((1s,4s)-4-hydroxycyclohexyl)amino)-2-oxoacetyl)-1,2,4-trimethyl-1H-pyrrole-3-carboxamide

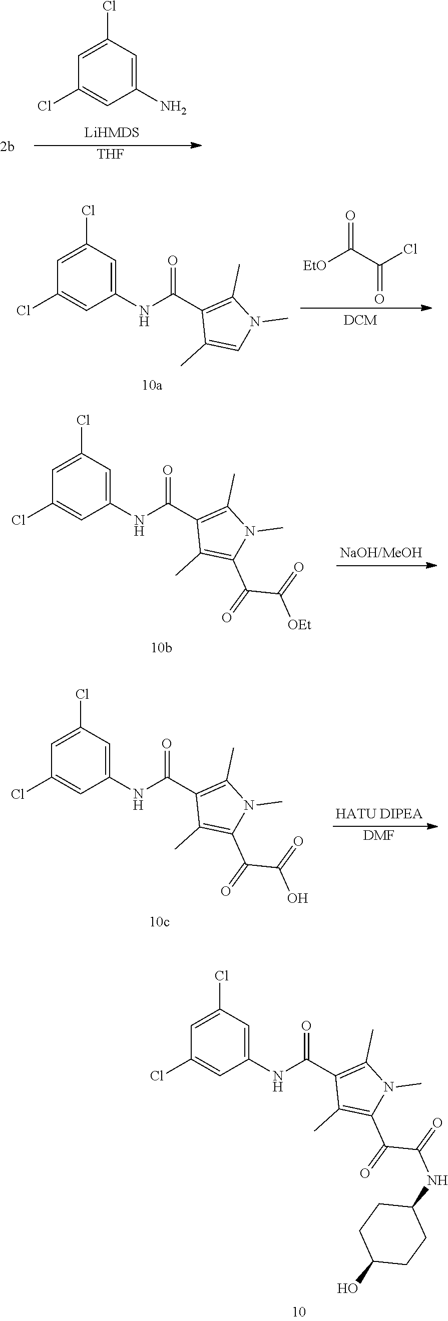

### Step 1: Synthesis of N-(2,6-dichloropyridin-4-yl)-1,2,4-trimethyl-1H-pyrrole-3-carboxamide (10a)

LiHMDS (20 mL, 1 N in THF) was added dropwise to a solution of 2b (1 g, 5.5 mmol) and 3,5-dichloroaniline (1.5 g, 9 mmol) in THF (20 mL) at 0° C. The reaction mixture was allowed to warm to rt for 20 hrs. The reaction mixture was quenched with NH₄Cl (sat) and water. The layers were separated and the aqueous was extracted 3×EtOAc. The combined organics were washed with NH₄Cl (sat) and brine, dried over Na₂SO₄, filtered and concentrated. The crude residue was purified by flash chromatography on silica gel (EtOAc/Hexanes 0˜100%) to afford 10a as yellow solid (1 g). ESI-MS, m/z 296/298 (M+H)⁺.

### Step 2: Synthesis of ethyl 2-(4-((3,5-dichlorophenyl)carbamoyl)-1,3,5-trimethyl-1H-pyrrol-2-yl)-2-oxoacetate (10b)

The title compounds were prepared following the procedure described in Example 2, Step 3 using 10a instead of 2b. ESI-MS, m/z 397/399 (M+H)⁺.

### Step 2: Synthesis of 2-(4-((3,5-dichlorophenyl)carbamoyl)-1,3,5-trimethyl-1H-pyrrol-2-yl)-2-oxoacetic acid (10c)

The title compounds were prepared following the procedure described in Example 2, Step 4 using 10b instead of 2c. ESI-MS, m/z 369/371 (M+H)⁺.

### Step 3: Synthesis of N-(3,5-dichlorophenyl)-5-(2-(((1s,4s)-4-hydroxycyclohexyl)amino)-2-oxoacetyl)-1,2,4-trimethyl-1H-pyrrole-3-carboxamide (10)

The title compounds were prepared following the procedure described in Example 2, Step 5 using 10c instead of 2d. ESI-MS, m/z 465/467 (M+H)⁺.

### Example 11: N-(2,6-dichloropyridin-4-yl)-5-(2-(((1s,4s)-4-hydroxy-1-methylcyclohexyl)amino)-2-oxoacetyl)-1,2,4-trimethyl-1H-pyrrole-3-carboxamide (11)

The title compounds were prepared following the procedure described in Example 2, Step 5 using cis-4-amino-4-methylcyclohexan-1-ol instead of cis-4-aminocyclohexan-1-ol. ESI-MS, m/z 481.1/483.1 (M+H)⁺.

### Example 12: N-(6-chloropyridin-2-yl)-5-(2-(((1s,4s)-4-hydroxycyclohexyl)amino)-2-oxoacetyl)-1,2,4-trimethyl-1H-pyrrole-3-carboxamide

The title compounds were prepared following the procedure described in Example 2, Step 1 to step 5 using 6-chloropyridin-2-amine instead of 2,6-dichloropyridin-4-amine. ESI-MS, m/z 433.1 (M+H)⁺.

### Example 13: Synthesis of 5-(2-(((1S,2R)-3,3-difluoro-2-hydroxycyclohexyl)amino)-2-oxoacetyl)-N-(4-fluoro-3-methylphenyl)-1,2,4-trimethyl-1H-pyrrole-3-carboxamide

The title compound was prepared following the procedure described in Example 1, Step 5, using (1R,6S)-6-amino-2,2-difluorocyclohexan-1-ol. The final product was purified by reverse phase chromatography eluted with ACN and water and dried using lyophilization to afford the title products as white solids. ESI-MS, m/z 466 (M+H)⁺.

### Example 14: Synthesis of N-(4-fluoro-3-methylphenyl)-5-(2-(((1R,2S,4R)-2-fluoro-4-hydroxycyclohexyl)amino)-2-oxoacetyl)-1,2,4-trimethyl-1H-pyrrole-3-carboxamide

The title compound was prepared following the procedure described in Example 1, Step 5, using (1R,3S,4R)-4-amino-3-fluorocyclohexan-1-ol. The final product was purified by reverse phase chromatography eluted with ACN and water and dried using lyophilization to afford the title products as white solids. ESI-MS, m/z 448 (M+H)⁺.

### Example 15: Synthesis of N-(4-fluoro-3-methylphenyl)-5-(2-(((1R,2R,4S)-2-fluoro-4-hydroxycyclohexyl)amino)-2-oxoacetyl)-1,2,4-trimethyl-1H-pyrrole-3-carboxamide

The title compound was prepared following the procedure described in Example 1, Step 5, using (1S,3R,4R)-4-amino-3-fluorocyclohexan-1-ol. The final product was purified by reverse phase chromatography eluted with ACN and water and dried using lyophilization to afford the title products as white solids. ESI-MS, m/z 448 (M+H)⁺.

### Example 16: Synthesis of N-(3-cyano-4-fluorophenyl)-5-(2-((trans-4-hydroxy-1-methylcyclohexyl)amino)-2-oxoacetyl)-1,2,4-trimethyl-1H-pyrrole-3-carboxamide

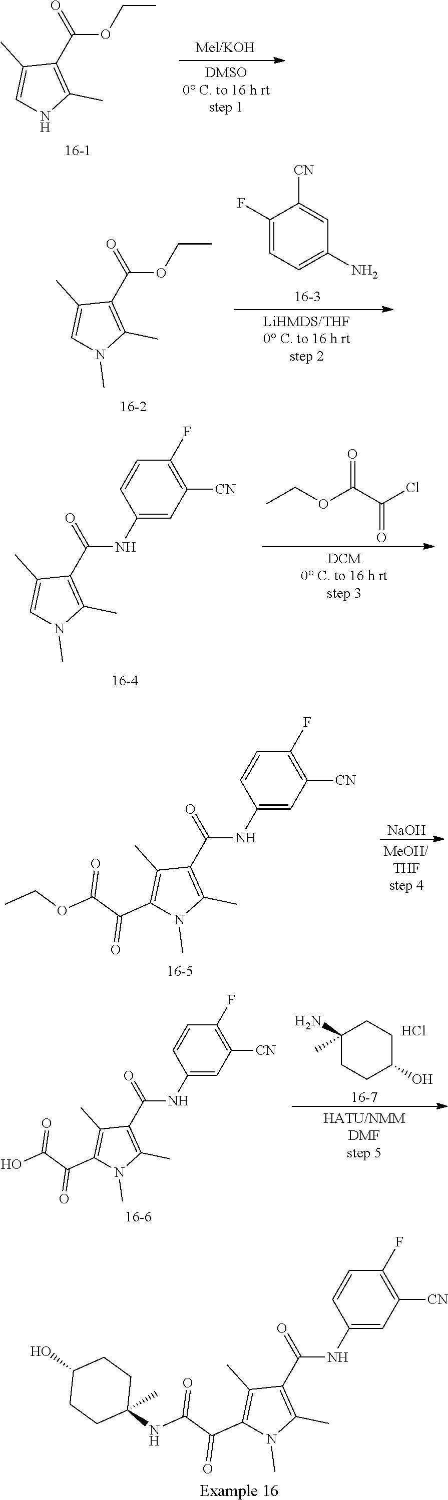

### Step 1: Synthesis of ethyl 1,2,4-trimethyl-1H-pyrrole-3-carboxylate (16-2)

MeI (31.8 g, 224.3 mmol) was added to a mixture of 16-1 (25 g, 149.5 mmol) and KOH (16.8 g, 299 mmol) in DMSO (250 mL) at 0° C. The reaction mixture was warmed to room temperature for 16 h. The reaction mixture was extracted with 4×Et₂O. The combined extracts were washed with water and brine, dried over Na₂SO₄, filtered and concentrated in vacuo to afford the title compound 16-2 as brown solid (24.6 g, 91%) which was used without further purification. ESI-MS, m/z 182 (M+H)⁺.

### Step 2: Synthesis of N-(3-cyano-4-fluorophenyl)-1,2,4-trimethyl-1H-pyrrole-3-carboxamide (16-4)

Lithium bis(trimethylsilyl)amide solution (1 M in THF, 10 mL, 10 mmol) was added dropwise over 5 min to a solution containing 16-2 (0.9 g, 5.0 mmol) and 5-amino-2-fluorobenzonitrile 16-3 (0.82 g, 6 mmol) in THF (20 mL) at 0° C. The reaction mixture was allowed to warm slowly to ambient temperature. The reaction mixture was warmed slowly to ambient temperature. After 16 h the reaction mixture was quenched with NH₄Cl (sat) and water. The layers were separated and the aqueous was extracted with 3×EtOAc. The combined organics were washed with water and brine, dried over Na₂SO₄, filtered and concentrated. The crude residue was suspended in 1:1 EtOAc/hexanes, stirred for 1 h at room temperature, and filtered. The filter cake was washed with hexanes and dried to afford the title compound 16-4 as tan solid. ESI-MS, m/z 272.2 (M+H)⁺.

### Step 3: Synthesis of ethyl 2-(4-((3-cyano-4-fluorophenyl)carbamoyl)-1,3,5-trimethyl-1H-pyrrol-2-yl)-2-oxoacetate (16-5)

To a solution of 16-4 (1.1 g, 4 mmol) in DCM (20 mL) at 0° C. was added ethyl chlorooxoacetate (1.1 g, 8 mmol) dropwise over 10 min and the reaction mixture was allowed to warm slowly to ambient temperature. After 16 h the reaction mixture was washed with H₂O and NaHCO₃ (sat) and then concentrated in vacuo to afford the title compound 16-5 as a brown solid which was used without any further purification. ESI-MS, m/z 455.2 (M+H)⁺.

### Step 4: Synthesis of 2-(4-((3-cyano-4-fluorophenyl)carbamoyl)-1,3,5-trimethyl-1H-pyrrol-2-yl)-2-oxoacetic acid (16-6)

To a solution of crude 16-5 (1.5 g, 3.8 mmol) in THF (10 mL) and MeOH (10 mL) was added 1N NaOH (10 mL). After 15 min the reaction mixture was concentrated to removed THF/methanol. The aqueous was acidified with conc. HCl to pH=1. The resultant solid was collected by filtration and washed with water. The solids were further dried in vacuo to provide 16-6 as pink solid. ESI-MS, m/z 344.1 (M+H)⁺.

### Step 5: Synthesis of N-(3-cyano-4-fluorophenyl)-5-(2-((trans-4-hydroxy-1-methylcyclohexyl)amino)-2-oxoacetyl)-1,2,4-trimethyl-1H-pyrrole-3-carboxamide

The title compounds were prepared following the procedure described in Example 1, using 16-6 and trans-4-amino-4-methylcyclohexan-1-ol. The final product was purified by reverse phase chromatography eluted with ACN and water and dried using lyophilization to afford the title products as white solids. ESI-MS m/z 455.2 (M+H)⁺.

### Example 17: Synthesis of N-(3-cyano-4-fluorophenyl)-5-(2-((cis-4-hydroxycyclohexyl)amino)-2-oxoacetyl)-1,2,4-trimethyl-1H-pyrrole-3-carboxamide

The title compounds were prepared following the procedure described in Example 16, using 16-6 and cis-4-aminocyclohexan-1-ol. The final product was purified by reverse phase chromatography eluted with ACN and water and dried using lyophilization to afford the title products as white solids. ESI-MS m/z 441.2 (M+H)⁺.

### Example 18: Synthesis of N-(3-bromo-4-fluorophenyl)-5-(2-((trans-4-hydroxy-1-methylcyclohexyl)amino)-2-oxoacetyl)-1,2,4-trimethyl-1H-pyrrole-3-carboxamide

The title compounds were prepared following the procedure described in Example 16 step 1 to 5, replacing 16-3 with 3-bromo-4-fluoroaniline. The final product was purified by reverse phase chromatography eluted with ACN and water and dried using lyophilization to afford the title products as white solids. ESI-MS m/z 508.2 (M+H)⁺.

### Example 19: Synthesis of N-(3-bromo-4-fluorophenyl)-5-(2-((cis-4-hydroxycyclohexyl)amino)-2-oxoacetyl)-1,2,4-trimethyl-1H-pyrrole-3-carboxamide

The title compounds were prepared following the procedure described in Example 16 step 1 to 5, replacing 16-3 with 3-bromo-4-fluoroaniline. The final product was purified by reverse phase chromatography eluted with ACN and water and dried using lyophilization to afford the title products as white solids. ESI-MS m/z 494.2 (M+H)⁺.

### Example 20: Synthesis of N-(4-fluoro-3-methylphenyl)-5-(2-(((1R,2S,3R,4S)-3-(hydroxymethyl)bicyclo[2.2.1]heptan-2-yl)amino)-2-oxoacetyl)-1,2,4-trimethyl-1H-pyrrole-3-carboxamide

The title compound was prepared following the procedure described in Example 1, Step 5, using ((1S,2R,3S,4R)-3-aminobicyclo[2.2.1]heptan-2-yl)methanol. The final product was purified by reverse phase chromatography eluted with ACN and water and dried using lyophilization to afford the title products as white solids. ESI-MS, m/z 456 (M+H)⁺.

### Example 21: Synthesis of N-(4-fluoro-3-methylphenyl)-5-(2-(((1S,2S,3R,4R)-3-(hydroxymethyl)bicyclo[2.2.1]heptan-2-yl)amino)-2-oxoacetyl)-1,2,4-trimethyl-1H-pyrrole-3-carboxamide

The title compound was prepared following the procedure described in Example 1, Step 5, using ((1R,2R,3S,4S)-3-aminobicyclo[2.2.1]heptan-2-yl)methanol. The final product was purified by reverse phase chromatography eluted with ACN and water and dried using lyophilization to afford the title products as white solids. ESI-MS, m/z 456 (M+H)⁺.

### Example 22: Synthesis of 5-(2-(((1S,2S,4R)-bicyclo[2.2.1]heptan-2-yl)amino)-2-oxoacetyl)-N-(4-fluoro-3-methylphenyl)-1,2,4-trimethyl-1H-pyrrole-3-carboxamide

The title compound was prepared following the procedure described in Example 1, Step 5, using (1S,2S,4R)-bicyclo[2.2.1]heptan-2-amine. The final product was purified by reverse phase chromatography eluted with ACN and water and dried using lyophilization to afford the title products as white solids. ESI-MS, m/z 426 (M+H)⁺.

### Example 23: Synthesis of N-(4-fluoro-3-methylphenyl)-5-(2-(((1R,4R)-5-hydroxybicyclo[2.2.1]heptan-2-yl)amino)-2-oxoacetyl)-1,2,4-trimethyl-1H-pyrrole-3-carboxamide

The title compound was prepared following the procedure described in Example 1, Step 5, using (1R,4R)-5-aminobicyclo[2.2.1]heptan-2-ol. The final product was purified by reverse phase chromatography eluted with ACN and water and dried using lyophilization to afford the title products as white solids. ESI-MS, m/z 442 (M+H)⁺.

### Example 24: Synthesis of N-(4-fluoro-3-methylphenyl)-5-(2-(((1S,4R)-6-hydroxybicyclo[2.2.1]heptan-2-yl)amino)-2-oxoacetyl)-1,2,4-trimethyl-1H-pyrrole-3-carboxamide

The title compound was prepared following the procedure described in Example 1, Step 5, using (1S,4R)-6-aminobicyclo[2.2.1]heptan-2-ol. The final product was purified by reverse phase chromatography eluted with ACN and water and dried using lyophilization to afford the title products as white solids. ESI-MS, m/z 442 (M+H)⁺.

### Example 25: Synthesis of 5-(2-((trans-4-hydroxy-1-methylcyclohexyl)amino)-2-oxoacetyl)-1,2,4-trimethyl-N-(3,4,5-trifluorophenyl)-1H-pyrrole-3-carboxamide

The title compounds were prepared following the procedure described in Example 16 step 1 to 5, replacing 16-3 with 3,4,5-trifluoroaniline. The final product was purified by reverse phase chromatography eluted with ACN and water and dried using lyophilization to afford the title products as white solids. ESI-MS m/z 466.2 (M+H)⁺.

### Example 26: Synthesis of Ethyl 2-(((1s,4s)-4-(2-(4-((4-fluoro-3-methylphenyl)carbamoyl)-1,3,5-trimethyl-1H-pyrrol-2-yl)-2-oxoacetamido)cyclohexyl)oxy)acetate

The title compound was prepared following the procedure described in Example 1, Step 5, using ethyl 2-(((1s,4s)-4-aminocyclohexyl)oxy)acetate. The final product was purified by reverse phase chromatography eluted with ACN and water and dried using lyophilization to afford the title products as white solids. ESI-MS, m/z 516 (M+H)⁺.

### Example 27: Synthesis of 2-(4-(2-(4-((4-fluoro-3-methylphenyl)carbamoyl)-1,3,5-trimethyl-1H-pyrrol-2-yl)-2-oxoacetamido)-4-methylpiperidin-1-yl)acetic acid

The title compounds were prepared following the procedure described in Example 1. The final product was purified by reverse phase chromatography eluted with ACN and water and dried using lyophilization to afford the title products as white solids. ESI-MS m/z 487.2 (M+H)⁺.

### Example 28: Synthesis of (S)-2-(3-(2-(4-((4-fluoro-3-methylphenyl)carbamoyl)-1,3,5-trimethyl-1H-pyrrol-2-yl)-2-oxoacetamido)pyrrolidin-1-yl)acetic acid

The title compounds were prepared following the procedure described in Example 1. The final product was purified by reverse phase chromatography eluted with ACN and water and dried using lyophilization to afford the title products as white solids. ESI-MS m/z 459.2 (M+H)⁺.

### Example 29: Synthesis of N-(4-hydroxybicyclo[2.2.2]octan-1-yl)-1,2,4-trimethyl-5-(2-oxo-2-(pyridin-3-ylamino)acetyl)-1H-pyrrole-3-carboxamide

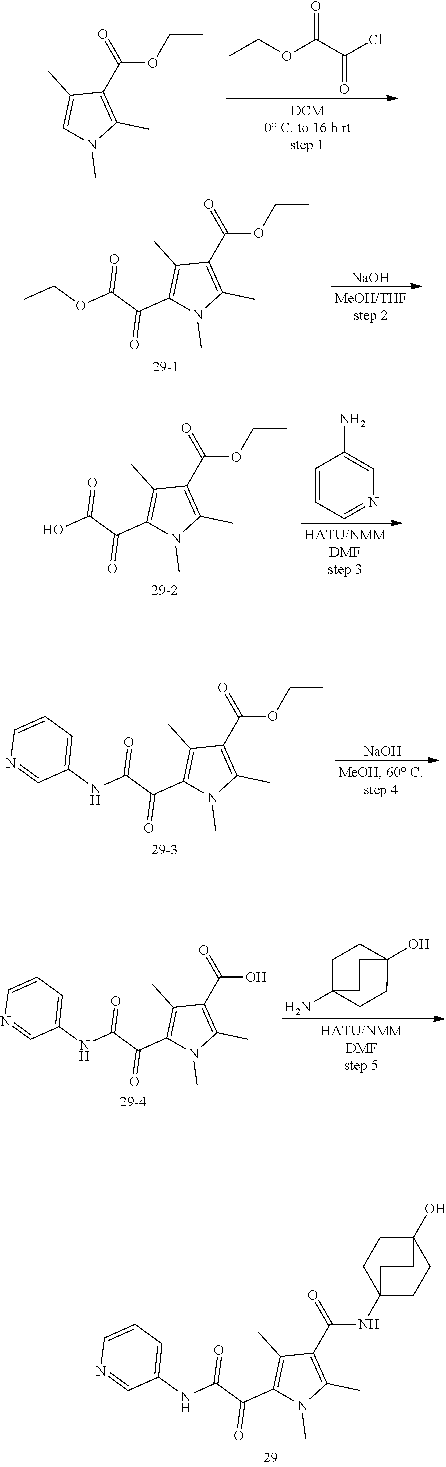

### Step 1: Synthesis of ethyl 5-(2-ethoxy-2-oxoacetyl)-1,2,4-trimethyl-1H-pyrrole-3-carboxylate (29-1)

Compound 29-1 was synthesized following procedure described in Example 16, step 3. The final product was purified by flash chromatography. ESI-MS, m/z 282.2 (M+H)⁺.

### Step 2: Synthesis of 2-(4-(ethoxycarbonyl)-1,3,5-trimethyl-1H-pyrrol-2-yl)-2-oxoacetic acid (29-2)

Compound 29-2 was synthesized following procedure described in Example 16, step 4. ESI-MS, m/z 254.2 (M+H)⁺.

### Step 3: Synthesis of ethyl 1,2,4-trimethyl-5-(2-oxo-2-(pyridin-3-ylamino)acetyl)-1H-pyrrole-3-carboxylate (29-3)

Compound 29-3 was synthesized following procedure described in Example 16, step 5. ESI-MS, m/z 330.2 (M+H)⁺.

### Step 4: Synthesis of 1,2,4-trimethyl-5-(2-oxo-2-(pyridin-3-ylamino)acetyl)-1H-pyrrole-3-carboxylic acid (29-4)

Compound 29-4 was synthesized following procedure described in Example 16, step 4 using MeOH as solvent at 60° C. ESI-MS, m/z 302.2 (M+H)⁺.

### Step 5: Synthesis of N-(4-hydroxybicyclo[2.2.2]octan-1-yl)-1,2,4-trimethyl-5-(2-oxo-2-(pyridin-3-ylamino)acetyl)-1H-pyrrole-3-carboxamide

The title compound were prepared following the procedure described in Example 1. The final product was purified by reverse phase chromatography eluted with ACN and water and dried using lyophilization to afford the title products as white solids. ESI-MS m/z 425.2 (M+H)⁺.

### Example 30: Synthesis of 1,2,4-trimethyl-N-(1-(3-methyl-1,2,4-oxadiazol-5-yl)cyclopropyl)-5-(2-oxo-2-(pyridin-3-ylamino)acetyl)-1H-pyrrole-3-carboxamide

The title compound was prepared following the procedure described in Example 29. The final product was purified by reverse phase chromatography eluted with ACN and water and dried using lyophilization to afford the title products as white solids. ESI-MS m/z 423.2 (M+H)⁺.

### Example 31: Synthesis of N-(4-fluoro-3-methylphenyl)-1,2,4-trimethyl-5-(2-(((1s,3s)-3-(methylsulfonyl)cyclobutyl)amino)-2-oxoacetyl)-1H-pyrrole-3-carboxamide

The title compound was prepared following the procedure described in Example 1, Step 5, using (1S,3S)-3-(methylsulfonyl)cyclobutan-1-amine. The final product was purified by reverse phase chromatography eluted with ACN and water and dried using lyophilization to afford the title products as white solids. ESI-MS, m/z 464 (M+H)⁺.

### Example 32: N-(4-fluoro-3-methylphenyl)-1,2,4-trimethyl-5-(2-(((1r,3r)-3-(methylsulfonyl)cyclobutyl)amino)-2-oxoacetyl)-1H-pyrrole-3-carboxamide

The title compound was prepared following the procedure described in Example 1, Step 5, using (1R,3R)-3-(methylsulfonyl)cyclobutan-1-amine. The final product was purified by reverse phase chromatography eluted with ACN and water and dried using lyophilization to afford the title products as white solids. ESI-MS, m/z 464 (M+H)⁺.

### Example 33: N-(3,4-difluorophenyl)-3-(2-((1-((3,4-difluorophenyl)carbamoyl)-4-methylpiperidin-4-yl)amino)-2-oxoacetyl)-2-methyl-5,6,7,8-tetrahydroindolizine-1-carboxamide

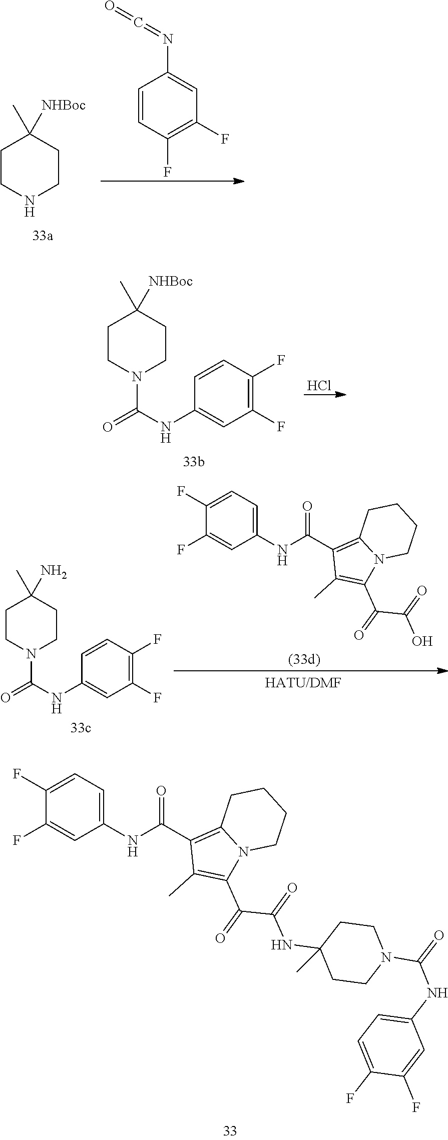

### Step 1: Synthesis of tert-butyl (1-((3,4-difluorophenyl)carbamoyl)-4-methylpiperidin-4-yl)carbamate (33b)

1,2-difluoro-4-isocyanatobenzene (0.8 g, 5.2 mmol) was added dropwise to a solution of 33a (1 g, 4.8 mmol) in DCM (15 mL) at 0° C. The reaction mixture was warmed to 30° C. for 16 hrs. The reaction mixture was filtered, and dried to afford 33b as white solid (1.8 g). ESI-MS, m/z 392.1 (M+23)⁺.

### Step 2: Synthesis of 4-amino-N-(3,4-difluorophenyl)-4-methylpiperidine-1-carboxamide (33c)

TFA (6 mL) was added dropwise to a solution of 33b (1.8 g) in DCM (5 mL) at 0° C. The reaction mixture was warmed to rt for 2 hrs, then, concentrated in vacuo. The residue was dissolved in 1N HCl/CH₃CN, and lyophilized to give 33c as white solid (1.5 g): ¹HNMR (300 MHz, DMSO-d6) 8.8 (s, 1H), 8.1 (br, 3H), 7.6-7.7 (m, 1H), 7.2-7.3 (m, 2H), 3.8-3.84 (m, 2H), 3.1-3.2 (m, 2H), 1.5-1.7 (m, 4H), 1.32 (s, 3H).

### Step 3: Synthesis of N-(3,4-difluorophenyl)-3-(2-((1-((3,4-difluorophenyl)carbamoyl)-4-methylpiperidin-4-yl)amino)-2-oxoacetyl)-2-methyl-5,6,7,8-tetrahydroindolizine-1-carboxamide

To a solution of 33c (20 mg), 33d (30 mg) and HATU (50 mg) in DMF (0.5 mL) at rt was added DIPEA (30 mg) at 0° C. The reaction mixture was warmed to rt overnight. The reaction mixture was quenched with aqueous HCl (0.2 N), and extracted with EtOAc. The organic layer was washed with water and brine, concentrated in vacuo, then, purified by reverse phase chromatography eluted with ACN and water, and dried using lyophilization to afford the title product as white solid. ESI-MS, m/z 614 (M+H)⁺.

### Example 35: (R)—N-(3,4-difluorophenyl)-3-(2-(4-((4-fluoro-3-methylphenyl)carbamoyl)-1,3,5-trimethyl-1H-pyrrol-2-yl)-2-oxoacetamido)piperidine-1-carboxamide

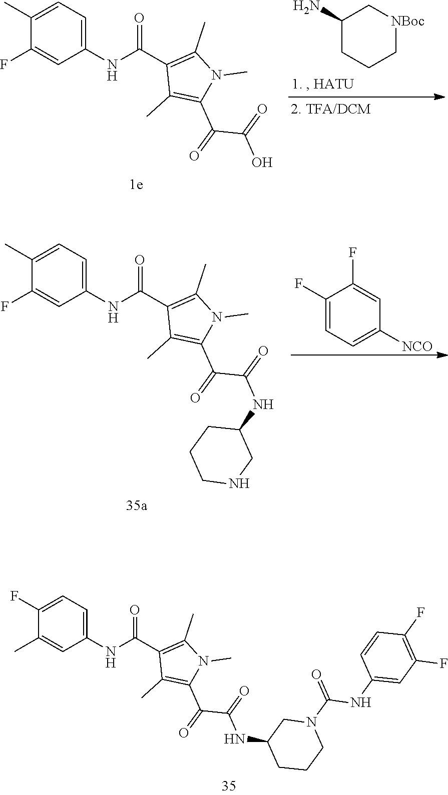

### Step 1: Synthesis of (R)—N-(3-fluoro-4-methylphenyl)-1,2,4-trimethyl-5-(2-oxo-2-(piperidin-3-ylamino)acetyl)-1H-pyrrole-3-carboxamide (35a)

To a solution of 1e (0.5 g, 1.5 mmol), tert-butyl (R)-3-aminopiperidine-1-carboxylate (0.3 g, 1.5 mmol) and HATU (0.65 g, 1.7 mmol) in DMF (4 mL) was added DIPEA (0.25 g) at 0° C. The reaction mixture was warmed to rt overnight. The reaction mixture was quenched with aqueous HCl (0.2 N), and extracted with EtOAc. The organic layer was washed with water and brine, concentrated in vacuo. TFA (4 mL) was added dropwise to a solution of the residue in DCM (4 mL) at 0° C. The mixture was warmed to rt for 3 hrs, concentrated in vacuo, and purified by reverse phase chromatography eluted with ACN and water, and dried using lyophilization to afford the title product as white solid. ESI-MS, m/z 415.2 (M+H)⁺.

### Step 2: Synthesis of (R)—N-(3,4-difluorophenyl)-3-(2-(4-((4-fluoro-3-methylphenyl)carbamoyl)-1,3,5-trimethyl-1H-pyrrol-2-yl)-2-oxoacetamido)piperidine-1-carboxamide

Triethylamine (0.1 mL) was added dropwise to a mixture of 1,2-difluoro-4-isocyanatobenzene (20 mg, 0.13 mmol) and 35a (20 mg) in DCM (2 mL) at 0° C. After 30 min., the reaction mixture was warmed to 30° C. for 16 hrs. The reaction mixture was concentrated and purified by reverse phase chromatography eluted with ACN and water, and dried using lyophilization to afford the title product as white solid. ESI-MS, m/z 570.3 (M+H)⁺.

### Example 36: N-(4-fluoro-3-methylphenyl)-1,2,4-trimethyl-5-(2-((4-methyl-1-(morpholine-4-carbonyl)piperidin-4-yl)amino)-2-oxoacetyl)-1H-pyrrole-3-carboxamide

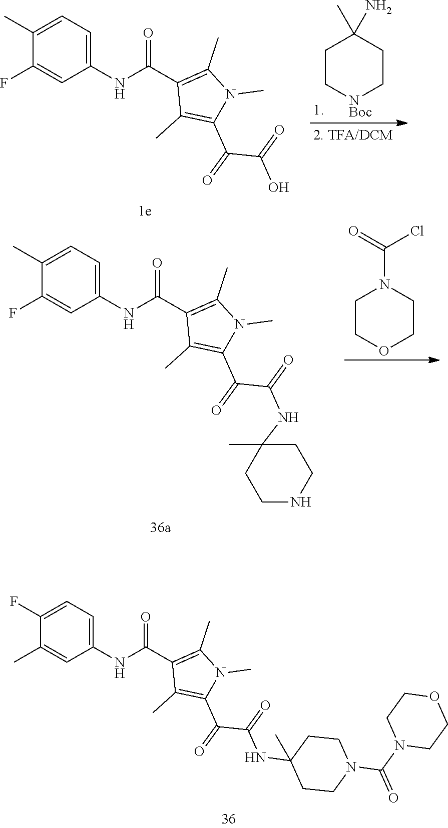 [SMILES: Cc1ccc(NC(=O)c2c(C)c(C(=O)C(=O)O)n(C)c2C)cc1F ; CC1(N)CCN(C(=O)OC(C)(C)C)CC1 ; Cc1ccc(NC(=O)c2c(C)c(C(=O)C(=O)NC3(C)CCNCC3)n(C)c2C)cc1F ; O=C(Cl)N1CCOCC1 ; Cc1cc(NC(=O)c2c(C)c(C(=O)C(=O)NC3(C)CCN(C(=O)N4CCOCC4)CC3)n(C)c2C)ccc1F]

### Step 1: Synthesis of N-(3-fluoro-4-methylphenyl)-1,2,4-trimethyl-5-(2-((4-methylpiperidin-4-yl)amino)-2-oxoacetyl)-1H-pyrrole-3-carboxamide (36a)

The title compounds were prepared following the procedure described in Example 35, step 1, using tert-butyl 4-amino-4-methylpiperidine-1-carboxylate instead of tert-butyl (R)-3-aminopiperidine-1-carboxylate. ESI-MS, m/z 429.3 (M+H)⁺.

### Step 2: Synthesis of N-(4-fluoro-3-methylphenyl)-1,2,4-trimethyl-5-(2-((4-methyl-1-(morpholine-4-carbonyl)piperidin-4-yl)amino)-2-oxoacetyl)-1H-pyrrole-3-carboxamide (36)

Triethylamine (0.1 g) was added to a solution of 36a (20 mg) and morpholine-4-carbonyl chloride (30 mg, 0.2 mmol) in DMA (1 mL) at 0° C. The mixture was warmed to rt for 2 hrs and 50° C. for 6 hrs. After cooled to rt, the reaction was quenched with 0.5 N HCl, and purified by reverse phase chromatography eluted with ACN and water, and dried using lyophilization to afford the title product as white solid. ESI-MS, m/z 542.3 (M+H)⁺.

### Example 37: N-(3,4-difluorophenyl)-2-methyl-3-(2-(((3S,4S)-4-morpholinotetrahydrofuran-3-yl)amino)-2-oxoacetyl)-5,6,7,8-tetrahydroindolizine-1-carboxamide

The title compounds were prepared following the procedure described in Example 2, step 5, using (3S,4S)-4-morpholinotetrahydrofuran-3-amine and 33c. The final product was purified by reverse phase chromatography eluted with ACN and water and dried using lyophilization to afford the title products as white solids. ESI-MS, m/z 517.2 (M+H)⁺.

### Example 38: 5-(2-((4-ethyl-1-(1-methyl-1H-pyrazole-4-carbonyl)piperidin-4-yl)amino)-2-oxoacetyl)-N-(4-fluoro-3-methylphenyl)-1,2,4-trimethyl-1H-pyrrole-3-carboxamide

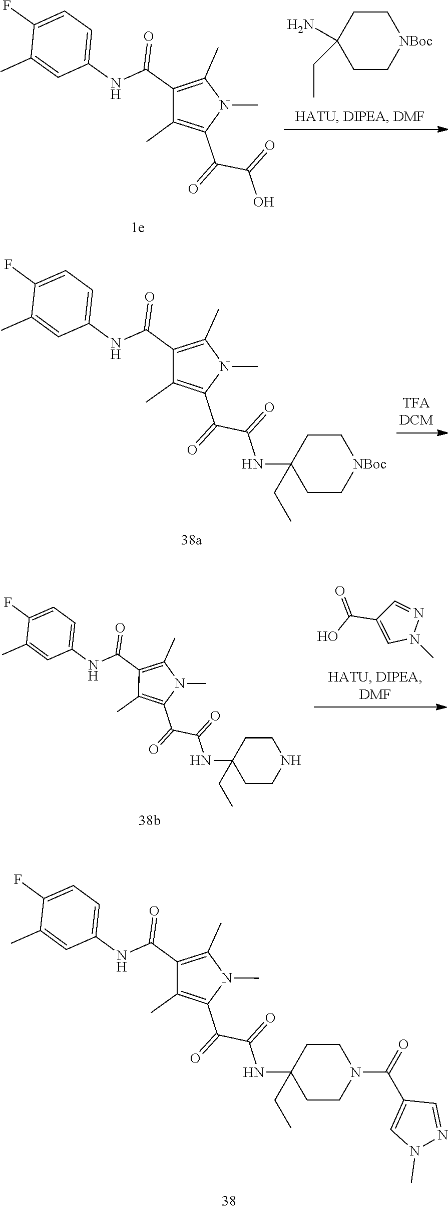

### Step 1: Synthesis of tert-butyl 4-ethyl-4-(2-(4-((4-fluoro-3-methylphenyl)carbamoyl)-1,3,5-trimethyl-1H-pyrrol-2-yl)-2-oxoacetamido)piperidine-1-carboxylate (38a)

The title compound was prepared following the procedure described in Example 1, Step 5, using tert-butyl 4-amino-4-ethylpiperidine-1-carboxylate. The final product was purified by precipitation from EtOAc/hexanes to afford the title products as a white solid. ESI-MS, m/z 543 (M+H)⁺.

### Step 2: Synthesis of 5-(2-((4-ethylpiperidin-4-yl)amino)-2-oxoacetyl)-N-(4-fluoro-3-methylphenyl)-1,2,4-trimethyl-1H-pyrrole-3-carboxamide (38b)

To a suspension of 38a (970 mg, 1.79 mmol) in DCM (5 mL) was added TFA (5 mL). After 1 h the reaction mixture was concentrated 3× from DCM to afford the title compound 38b (1.05 g) as a white solid which was taken forward without any further purification. ESI-MS, m/z 443 (M+H)⁺.

### Step 3: Synthesis of N-(4-fluoro-3-methylphenyl)-1,2,4-trimethyl-5-(2-(((1r,3r)-3-(methylsulfonyl)cyclobutyl)amino)-2-oxoacetyl)-1H-pyrrole-3-carboxamide (38)

To a solution of 38b (60 mg, 0.136 mmol), 1-methyl-1H-pyrazole-4-carboxylic acid (19 mg, 0.15 mmol) and HATU (62 mg, 0.163 mmol) in DMF (1 mL) was added DIPEA (53 mg, 0.41 mmol). After 16 h the reaction mixture was purified directly via reverse phase chromatography eluted with ACN and water and dried using lyophilization to afford the title products as white solids. ESI-MS, m/z 551 (M+H)⁺.

### Example 39: 5-(2-((4-ethyl-1-(1-methyl-1H-pyrazole-5-carbonyl)piperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 38, Step 3, using 1-methyl-1H-pyrazole-5-carboxylic acid. The final product was purified by reverse phase chromatography eluted with ACN and water and dried using lyophilization to afford the title products as white solids. ESI-MS, m/z 551 (M+H)⁺.

### Example 40: 5-(2-((1-(3-(difluoromethyl)-1-methyl-1H-pyrazole-4-carbonyl)-4-ethylpiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 38, Step 3, using 3-(difluoromethyl)-1-methyl-1H-pyrazole-4-carboxylic acid. The final product was purified by reverse phase chromatography eluted with ACN and water and dried using lyophilization to afford the title products as white solids. ESI-MS, m/z 601 (M+H)⁺.

### Example 41: 5-(2-((1-acetyl-4-ethylpiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 38, Step 3, using acetic acid. The final product was purified by reverse phase chromatography eluted with ACN and water and dried using lyophilization to afford the title products as white solids. ESI-MS, m/z 485 (M+H)⁺.

### Example 42: 5-(2-((4-ethyl-1-(methylsulfonyl)piperidin-4-yl)amino)-2-oxoacetyl)-N-(4-fluoro-3-methylphenyl)-1,2,4-trimethyl-1H-pyrrole-3-carboxamide

To a suspension of 38b (60 mg, 0.136 mmol) in DCM (1 mL) were added MsCl (17 mg, 0.15 mmol) and DIPEA (53 mg, 0.41 mmol). After 1 h, DMF (0.5 mL) was added. After 16 h DCM was removed and the residue was purified directly by reverse phase chromatography eluted with ACN and water and dried using lyophilization to afford the title product 42 (27 mg, 38%) as a white solid. ESI-MS, m/z 521 (M+H)⁺.

### Example 43: 5-(2-((4-ethyl-1-(thiazol-2-yl)piperidin-4-yl)amino)-2-oxoacetyl)-N-(4-fluoro-3-methylphenyl)-1,2,4-trimethyl-1H-pyrrole-3-carboxamide

To a solution of 38b (60 mg, 0.136 mmol) in DMF (1 mL) were added 2-Bromo-thiazole (67 mg, 0.41 mmol) and K₂CO₃ (23 mg, 0.163 mmol) and the mixture was heated to 100° C. After 16 h, the reaction mixture was cooled to ambient temperature and purified directly by reverse phase chromatography, eluted with ACN and water, and dried using lyophilization to afford the title product 43 (33 mg, 46%) as a white solid. ESI-MS, m/z 526 (M+H)⁺.

### Example 44: 5-(2-((4-ethyl-1-(pyridazin-3-yl)piperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 43 using 3-bromopyridazine. The final product was purified by precipitation from EtOAc/hexanes to afford the title product as an off-white solid. ESI-MS, m/z 521 (M+H)⁺.

### Example 45: Synthesis of (4-(5-(2-(tert-butylamino)-2-oxoacetyl)-1,2,4-trimethyl-1H-pyrrole-3-carboxamido)phenyl)boronic acid

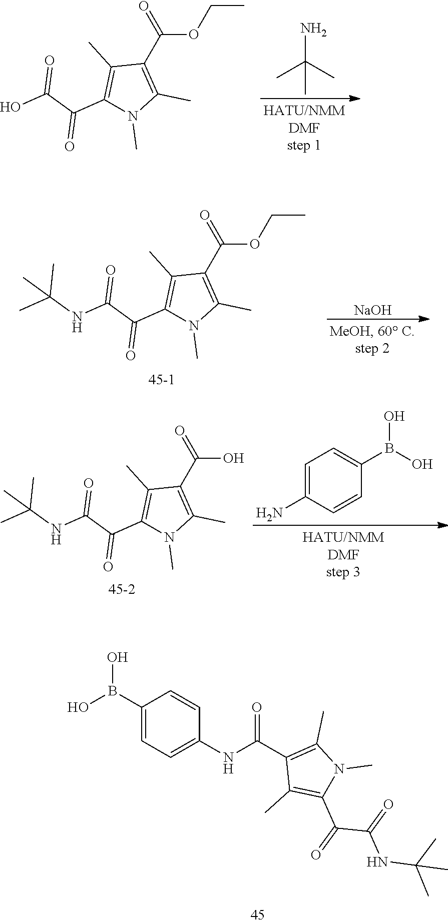

Compound 45-2 was synthesized following procedure described in Example 29 step 3 using t-butyl amine. The title compound 46 was prepared following the procedure described in Example 29 step 5. The final product was purified by reverse phase chromatography eluted with ACN and water and dried using lyophilization to afford the title products as white solids. ESI-MS m/z 400.2 (M+H)⁺.

### Example 46: (3-(5-(2-(tert-butylamino)-2-oxoacetyl)-1,2,4-trimethyl-1H-pyrrole-3-carboxamido)phenyl)boronic acid

The title compound was prepared following the procedure described in Example 45. The final product was purified by reverse phase chromatography eluted with ACN and water and dried using lyophilization to afford the title products as off-white solids. ESI-MS, m/z 400.2 (M+H)⁺.

### Example 47: 5-(2-(tert-butylamino)-2-oxoacetyl)-1,2,4-trimethyl-N-(1-(4-methylthiazol-2-yl)cyclopentyl)-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 445.2 (M+H)⁺.

### Example 48: 5-(2-(tert-butylamino)-2-oxoacetyl)-N-(1,4-dioxepan-6-yl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 380.2 (M+H)⁺.

### Example 49: N-(3,4-difluorophenyl)-3-(2-(4-((4-fluoro-3-methylphenyl)carbamoyl)-1,3,5-trimethyl-1H-pyrrol-2-yl)-2-oxoacetamido)-3-methylpiperidine-1-carboxamide

The title compounds were prepared following the procedure described in Example 33, using tert-butyl 3-amino-3-methylpiperidine-1-carboxylate instead of 33a. The final product was purified by reverse phase chromatography eluted with ACN and water and dried using lyophilization to afford the title products as white solids. ESI-MS, m/z 584 (M+H)⁺.

### Example 50: 5-(2-((4-ethyl-1-(pyridazin-3-yl)piperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 43 using 2-chloropyrazine. The final product was purified by precipitation from EtOAc/hexanes to afford the title product as an off-white solid. ESI-MS, m/z 521 (M+H)⁺.

### Example 51: (R)—N-(2,6-dichloropyridin-4-yl)-1,2,4-trimethyl-5-(2-oxo-2-((1,1,1-trifluoropropan-2-yl)amino)acetyl)-1H-pyrrole-3-carboxamide

The title compounds were prepared following the procedure described in Example 2, step 5 using (R)-1,1,1-trifluoropropan-2-amine instead of cis-4-aminocyclohexan-1-ol. The final product was purified by reverse phase chromatography eluted with ACN and water and dried using lyophilization to afford the title products as white solids. ESI-MS, m/z 465/467 (M+H)⁺.

### Example 52: N-(4-fluoro-3-methylphenyl)-1,2,4-trimethyl-5-(2-((2-morpholinophenyl)amino)-2-oxoacetyl)-1H-pyrrole-3-carboxamide

The title compounds were prepared following the procedure described in Example 2, step 5, using 2-morpholinoaniline and 1e. The final product was purified by reverse phase chromatography eluted with ACN and water and dried using lyophilization to afford the title products as white solids. ESI-MS, m/z 493.2 (M+H)⁺.

### Example 53: N-(4-fluoro-3-methylphenyl)-1,2,4-trimethyl-5-(2-((2-morpholinopyridin-3-yl)amino)-2-oxoacetyl)-1H-pyrrole-3-carboxamide

The title compounds were prepared following the procedure described in Example 2, step 5, using 2-morpholinopyridin-3-amine and 1e. The final product was purified by reverse phase chromatography eluted with ACN and water and dried using lyophilization to afford the title products as white solids. ESI-MS, m/z 494.2 (M+H)⁺.

### Example 54: N-(4-fluoro-3-methylphenyl)-1,2,4-trimethyl-5-(2-((2-morpholinopyridin-3-yl)amino)-2-oxoacetyl)-1H-pyrrole-3-carboxamide

The title compounds were prepared following the procedure described in Example 2, step 5, using 3-morpholinopyridin-4-amine and 1e. The final product was purified by reverse phase chromatography eluted with ACN and water and dried using lyophilization to afford the title products as white solids. ESI-MS, m/z 494.2 (M+H)⁺.

### Example 55: methyl 5-(5-(2-(tert-butylamino)-2-oxoacetyl)-1,2,4-trimethyl-1H-pyrrole-3-carboxamido)-2-fluorobenzoate

The title compound was prepared following the procedure described in Example 45. The final product was purified by reverse phase chromatography eluted with ACN and water and dried using lyophilization to afford the title products as off-white solids. ESI-MS, m/z 432.1 (M+H)⁺.

### Example 56: 5-(5-(2-(tert-butylamino)-2-oxoacetyl)-1,2,4-trimethyl-1H-pyrrole-3-carboxamido)-2-fluorobenzoic acid

The title compound was prepared by hydrolysis of the compound in Example 55. The final product was purified by reverse phase chromatography eluted with ACN and water and dried using lyophilization to afford the title products as off-white solids. ESI-MS, m/z 418.2 (M+H)⁺.

### Example 57: 5-(2-(tert-butylamino)-2-oxoacetyl)-N-(3-carbamoyl-4-fluorophenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 417.2 (M+H)⁺.

### Example 58: 5-(2-(tert-butylamino)-2-oxoacetyl)-1,2,4-trimethyl-N-((5-(trifluoromethyl)-1,2,4-oxadiazol-3-yl)methyl)-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 430.2 (M+H)⁺.

### Example 59: 5-(2-(tert-butylamino)-2-oxoacetyl)-N-(3-cyano-4-fluorophenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 399.2 (M+H)⁺.

### Example 60: N-(3-bromo-4-fluorophenyl)-5-(2-(tert-butylamino)-2-oxoacetyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 452.0 (M+H)⁺.

### Example 61: N-(tert-butyl)-2-(4-(4-hydroxy-4-methylpiperidine-1-carbonyl)-1,3,5-trimethyl-1H-pyrrol-2-yl)-2-oxoacetamide

The title compound was prepared following the procedure described in Example 45. The final product was purified by reverse phase chromatography eluted with ACN and water and dried using lyophilization to afford the title products as off-white solids. ESI-MS, m/z 378.2 (M+H)⁺.

### Example 62: 5-(2-(tert-butylamino)-2-oxoacetyl)-1,2,4-trimethyl-N-(6-morpholinopyridin-3-yl)-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 442.2 (M+H)⁺.

### Example 63: (1s,3s)-3-(2-(4-((4-fluoro-3-methylphenyl)carbamoyl)-1,3,5-trimethyl-1H-pyrrol-2-yl)-2-oxoacetamido)-3-methylcyclobutyl dihydrogen phosphate

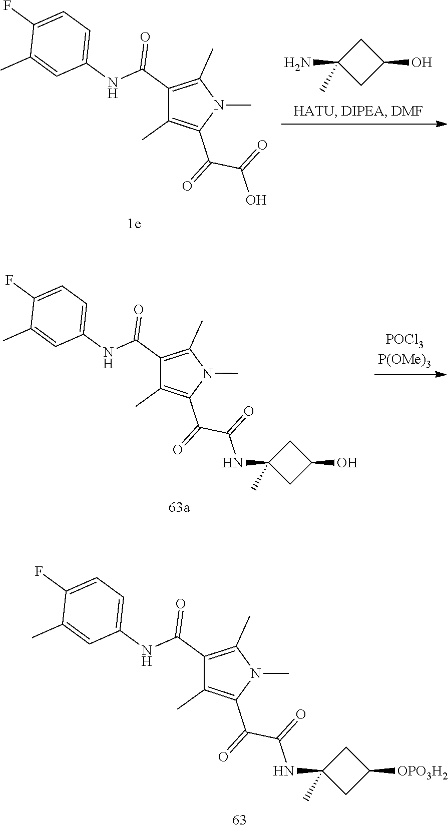

### Step 1: Synthesis of N-(4-fluoro-3-methylphenyl)-5-(2-(((1s,3s)-3-hydroxy-1-methylcyclobutyl)amino)-2-oxoacetyl)-1,2,4-trimethyl-1H-pyrrole-3-carboxamide (63a)

The title compound was prepared following the procedure described in Example 1, Step 5, using (1s,3s)-3-amino-3-methylcyclobutan-1-ol. The final product was purified by precipitation from EtOAc/hexanes to afford the title products as an off-white solid. ESI-MS, m/z 416 (M+H)⁺.

### Step 1: Synthesis of (1s,3s)-3-(2-(4-((4-fluoro-3-methylphenyl)carbamoyl)-1,3,5-trimethyl-1H-pyrrol-2-yl)-2-oxoacetamido)-3-methylcyclobutyl dihydrogen phosphate (63)

To a solution of 63a (2.8 g, 6.6 mmol) in P(OMe)₃ (40 mL) at 0° C. was added POCl₃ (1.89 mL). After 3 h the reaction mixture was quenched slowly with NaHCO₃ (sat) (100 mL). After 16 h the mixture was filtered and extracted 3×EtOAc whereupon a crystallization occurred. The resultant solids were filtered and washed with EtOAc to afford the title compound 63 (2.59 g) as a white crystalline solid. ESI-MS, m/z 496 (M+H)⁺.

### Example 64: (1s,3s)-3-(2-(4-((4-fluoro-3-methylphenyl)carbamoyl)-1,3,5-trimethyl-1H-pyrrol-2-yl)-2-oxoacetamido)-3-methylcyclobutyl dihydrogen phosphate

To a suspension of 63 (2.59 g, 5.22 mmol) in MeOH (25 mL) was added NaOMe (578 mg, 10.70 mmol). After 4 h the mixture was filtered and washed with MeOH to afford the title compound 64 (2.48 g. 88%) as a white solid. ESI-MS, m/z 496 (M+H)⁺.

### Example 65: 5-(2-(tert-butylamino)-2-oxoacetyl)-1,2,4-trimethyl-N-(3-sulfamoylphenyl)-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 435.2 (M+H)⁺.

### Example 66: 5-(2-(tert-butylamino)-2-oxoacetyl)-N-(2-fluoro-3-methylphenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 388.2 (M+H)⁺.

### Example 67: 5-(2-(tert-butylamino)-2-oxoacetyl)-N-(3-fluoro-5-methylphenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 388.2 (M+H)⁺.

### Example 68: 5-(2-(tert-butylamino)-2-oxoacetyl)-N-(2-fluoro-5-methylphenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 388.2 (M+H)⁺.

### Example 69: 5-(2-(tert-butylamino)-2-oxoacetyl)-N-(4-fluoro-3-(methylsulfonyl)phenyl)-1,2,4-trimethyl-1H-pyrrole-3-carboxamide

The title compound was prepared following the procedure described in Example 45. The final product was purified by reverse phase chromatography eluted with ACN and water and dried using lyophilization to afford the title products as off-white solids. ESI-MS, m/z 452.2 (M+H)⁺.

### Example 72: 5-(2-((1-acetyl-4-(hydroxymethyl)piperidin-4-yl)amino)-2-oxoacetyl)-N-(4-fluoro-3-methylphenyl)-1,2,4-trimethyl-1H-pyrrole-3-carboxamide

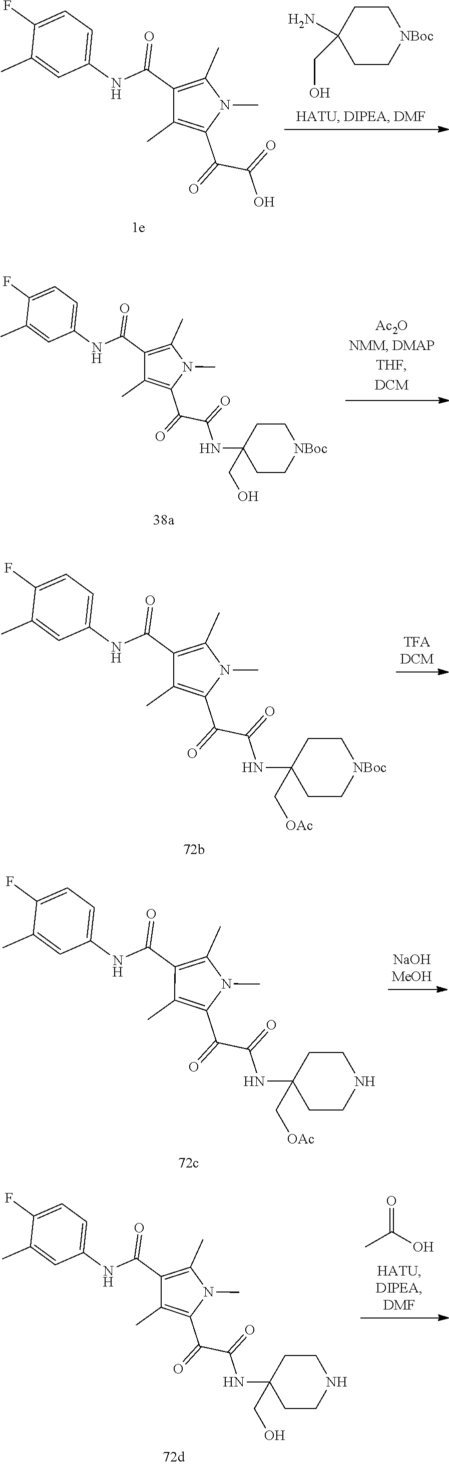

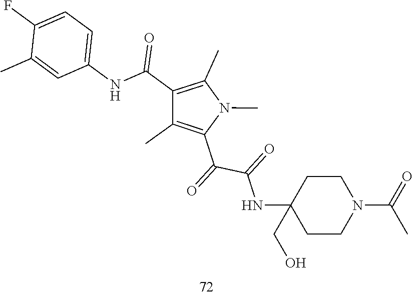 [SMILES: CC(=O)N1CCC(CO)(NC(=O)C(=O)c2c(C)c(C(=O)Nc3ccc(F)c(C)c3)c(C)n2C)CC1]

### Step 1: Synthesis of tert-butyl 4-(2-(4-((4-fluoro-3-methylphenyl)carbamoyl)-1,3,5-trimethyl-1H-pyrrol-2-yl)-2-oxoacetamido)-4-(hydroxymethyl)piperidine-1-carboxylate (72a)

The title compound was prepared following the procedure described in Example 1, Step 5, using tert-butyl 4-amino-4-(hydroxymethyl)piperidine-1-carboxylate. The crude reaction mixture was diluted into 1N HCl and extracted 3×EtOAc. The combined organics were washed with 1N HCl, NaHCO₃ (sat) and brine, dried over Na₂SO₄, filtered and concentrated to afford the title compound 72a which was brought forward without further purification. ESI-MS, m/z 545 (M+H)⁺.

### Step 2: Synthesis of tert-butyl 4-(acetoxymethyl)-4-(2-(4-((4-fluoro-3-methylphenyl)carbamoyl)-1,3,5-trimethyl-1H-pyrrol-2-yl)-2-oxoacetamido)piperidine-1-carboxylate (72b)

To a suspension of 72a (279 mg, 0.513 mmol) in DCM (3 mL) and THF (3 mL) at 0° C. were added NMM (104 mg, 1.03 mmol), DMAP (cat) and Ac₂O (63 mg, 0.616 mmol). After 1 h, the reaction mixture was warmed to ambient temperature. After 3 h the mixture was diluted with EtOAc, washed 2×1N HCl, NaHCO₃ (sat), and brine, dried over Na₂SO₄, filtered and concentrated to afford the title compound 72b (293 mg, 97%) which was taken forward without further purification. ESI-MS, m/z 587 (M+H)⁺.

### Step 3: Synthesis of (4-(2-(4-((4-fluoro-3-methylphenyl)carbamoyl)-1,3,5-trimethyl-1H-pyrrol-2-yl)-2-oxoacetamido)piperidin-4-yl)methyl acetate (72c)

To a suspension of 72b (293 mg, 0.50 mmol) in DCM (5 mL) at ambient temperature was added TFA (1 mL) and a brown solution was formed. After 30 min the reaction mixture was concentrated. The crude residue was dissolved in EtOAc, washed 2×NaHCO₃ (sat) and brine, dried over Na₂SO₄, filtered and concentrated to afford the title compound 72c (320 mg, quant) as an off-white solid which was taken forward without further purification. ESI-MS, m/z 487 (M+H)⁺.

### Step 4: Synthesis of N-(4-fluoro-3-methylphenyl)-5-(2-((4-(hydroxymethyl)piperidin-4-yl)amino)-2-oxoacetyl)-1,2,4-trimethyl-1H-pyrrole-3-carboxamide (72d)

To a solution of 72c (243 mg, 0.5 mmol) in MeOH (5 mL) was added 1N NaOH (1 mL). After 1 h the reaction mixture was concentrated to a crude residue which was taken forward without further purification. ESI-MS, m/z 445 (M+H)⁺.

### Step 5: Synthesis of 5-(2-((1-acetyl-4-(hydroxymethyl)piperidin-4-yl)amino)-2-oxoacetyl)-N-(4-fluoro-3-methylphenyl)-1,2,4-trimethyl-1H-pyrrole-3-carboxamide (72)

The title compound was prepared following the procedure described in Example 38, Step 3, using 72d as the starting amine and acetic acid as the carboxylic acid. The final product was purified by reverse phase chromatography eluted with ACN and water and dried using lyophilization to afford the title products as white solids. ESI-MS, m/z 487 (M+H)⁺.

### Example 73: N-(4-fluoro-3-methylphenyl)-5-(2-((4-(hydroxymethyl)-1-(methylsulfonyl)piperidin-4-yl)amino)-2-oxoacetyl)-1,2,4-trimethyl-1H-pyrrole-3-carboxamide

The title compound was prepared following the procedure described in Example 42, using 72d as the starting amine. The final product was purified by reverse phase chromatography eluted with ACN and water and dried using lyophilization to afford the title products as white solids. ESI-MS, m/z 523 (M+H)⁺.

### Example 74: N-(4-fluoro-3-methylphenyl)-5-(2-((4-(hydroxymethyl)-1-(1-methyl-1H-pyrazole-4-carbonyl)piperidin-4-yl)amino)-2-oxoacetyl)-1,2,4-trimethyl-1H-pyrrole-3-carboxamide

The title compound was prepared following the procedure described in Example 38, step 3, using 72d as the starting amine. The final product was purified by reverse phase chromatography eluted with ACN and water and dried using lyophilization to afford the title products as white solids. ESI-MS, m/z 553 (M+H)⁺.

### Example 75: 5-(2-((1-(3-(difluoromethyl)-1-methyl-1H-pyrazole-4-carbonyl)-4-(hydroxymethyl)piperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 38, step 3, using 72d as the starting amine and 3-(difluoromethyl)-1-methyl-1H-pyrazole-4-carboxylic acid as the starting acid. The final product was purified by reverse phase chromatography eluted with ACN and water and dried using lyophilization to afford the title products as white solids. ESI-MS, m/z 603 (M+H)⁺.

### Example 76: N-(4-fluoro-3-methylphenyl)-5-(2-((4-(hydroxymethyl)-1-(1-methyl-1H-pyrazole-5-carbonyl)piperidin-4-yl)amino)-2-oxoacetyl)-1,2,4-trimethyl-1H-pyrrole-3-carboxamide

The title compound was prepared following the procedure described in Example 38, step 3, using 72d as the starting amine and 1-methyl-1H-pyrazole-5-carboxylic acid as the starting acid. The final product was purified by reverse phase chromatography eluted with ACN and water and dried using lyophilization to afford the title products as white solids. ESI-MS, m/z 553 (M+H)⁺.

### Example 77: (R)—N-(6-chloropyridin-2-yl)-1,2,4-trimethyl-5-(2-oxo-2-((1,1,1-trifluoropropan-2-yl)amino)acetyl)-1H-pyrrole-3-carboxamide

The title compounds were prepared following the procedure described in Example 12 using (R)-1,1,1-trifluoropropan-2-amine and instead of cis-4-aminocyclohexan-1-ol. The final product was purified by reverse phase chromatography eluted with ACN and water and dried using lyophilization to afford the title products as white solids. ESI-MS, m/z 431.1 (M+H)⁺.

### Example 78: 5-(2-(((2R,4S)-1-acetyl-2-methylpiperidin-4-yl)amino)-2-oxoacetyl)-N-(4-fluoro-3-methylphenyl)-1,2,4-trimethyl-1H-pyrrole-3-carboxamide

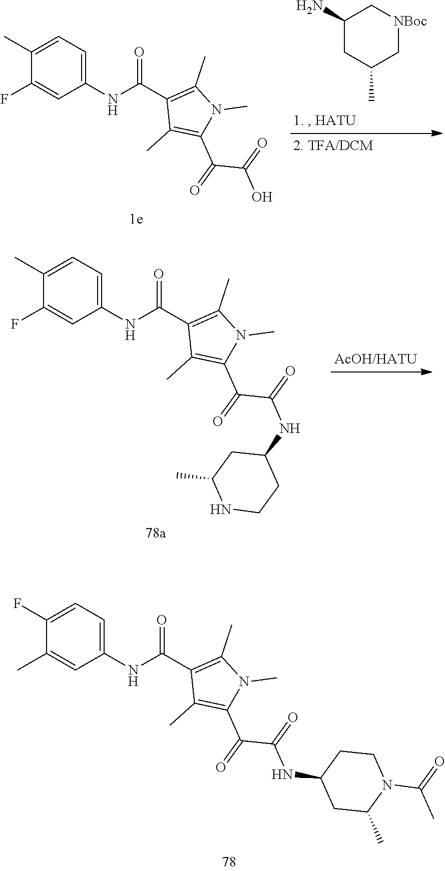 [SMILES: Cc1ccc(NC(=O)c2c(C)c(C(=O)C(=O)O)n(C)c2C)cc1F ; C[C@@H]1C[C@@H](N)CN(C(=O)OC(C)(C)C)C1 ; Cc1ccc(NC(=O)c2c(C)c(C(=O)C(=O)N[C@H]3CCN[C@H](C)C3)n(C)c2C)cc1F ; CC(=O)N1CC[C@H](NC(=O)C(=O)c2c(C)c(C(=O)Nc3ccc(F)c(C)c3)c(C)n2C)C[C@H]1C]

### Step 1: Synthesis of N-(3-fluoro-4-methylphenyl)-1,2,4-trimethyl-5-(2-(((2R,4S)-2-methylpiperidin-4-yl)amino)-2-oxoacetyl)-1H-pyrrole-3-carboxamide (78a)

The title compounds were prepared following the procedure described in Example 35, step 1, using tert-butyl (2R,4S)-4-amino-2-methylpiperidine-1-carboxylate instead of tert-butyl (R)-3-aminopiperidine-1-carboxylate. ESI-MS, m/z 429.2 (M+H)⁺.

### Step 2: Synthesis of 5-(2-(((2R,4S)-1-acetyl-2-methylpiperidin-4-yl)amino)-2-oxoacetyl)-N-(4-fluoro-3-methylphenyl)-1,2,4-trimethyl-1H-pyrrole-3-carboxamide

The title compounds were prepared following the procedure described in Example 2, step 5, using 78a and acetic acid. The final product was purified by reverse phase chromatography eluted with ACN and water and dried using lyophilization to afford the title products as white solids. ESI-MS, m/z 470.5 (M+H)⁺.

### Example 79: (R)—N-(3,5-dichlorophenyl)-1,2,4-trimethyl-5-(2-oxo-2-((1,1,1-trifluoropropan-2-yl)amino)acetyl)-1H-pyrrole-3-carboxamide

The title compounds were prepared following the procedure described in Example 10 using (R)-1,1,1-trifluoropropan-2-amine and instead of cis-4-aminocyclohexan-1-ol. The final product was purified by reverse phase chromatography eluted with ACN and water and dried using lyophilization to afford the title products as white solids. ESI-MS, m/z 463/465 (M+H)⁺.

### Example 80: 5-(2-(bicyclo[1.1.1]pentan-1-ylamino)-2-oxoacetyl)-N-(4-fluoro-3-methylphenyl)-1,2,4-trimethyl-1H-pyrrole-3-carboxamide

The title compounds were prepared following the procedure described in Example 2, step 5, using 1e and bicyclo[1.1.1]pentan-1-amine. The final product was purified by reverse phase chromatography eluted with ACN and water and dried using lyophilization to afford the title products as white solids. ESI-MS, m/z 398.2 (M+H)⁺.

### Example 81: N-(4-fluoro-3-methylphenyl)-1,2,4-trimethyl-5-(2-((7-methyl-4-oxo-5-azaspiro[2.4]heptan-7-yl)amino)-2-oxoacetyl)-1H-pyrrole-3-carboxamide

The title compounds were prepared following the procedure described in Example 2, step 5, using 1e and 7-amino-7-methyl-5-azaspiro[2.4]heptan-4-one. The final product was purified by reverse phase chromatography eluted with ACN and water and dried using lyophilization to afford the title products as white solids. ESI-MS, m/z 455.2 (M+H)⁺.

### Example 82: N-(4-fluoro-3-methylphenyl)-5-(2-((3-hydroxybicyclo[1.1.1]pentan-1-yl)amino)-2-oxoacetyl)-1,2,4-trimethyl-1H-pyrrole-3-carboxamide

The title compounds were prepared following the procedure described in Example 2, step 5, using 1e and 3-aminobicyclo[1.1.1]pentan-1-ol. The final product was purified by reverse phase chromatography eluted with ACN and water and dried using lyophilization to afford the title products as white solids. ESI-MS, m/z 414.2 (M+H)⁺.

### Example 83: Synthesis of N-(3-cyano-4-fluorophenyl)-5-(2-((3-hydroxy-1-methylcyclobutyl)amino)-2-oxoacetyl)-1,2,4-trimethyl-1H-pyrrole-3-carboxamide

The title compounds were prepared following the procedure described in Example 16, using 16-6 and 3-amino-3-methylcyclobutan-1-ol. The final product was purified by reverse phase chromatography eluted with ACN and water and dried using lyophilization to afford the title products as white solids. ESI-MS m/z 427.2 (M+H)⁺.

### Example 85: N-(4-fluoro-3-methylphenyl)-1,2,4-trimethyl-5-(2-((1-methyl-4,5,6,7-tetrahydro-1H-benzo[d][1,2,3]triazol-6-yl)amino)-2-oxoacetyl)-1H-pyrrole-3-carboxamide

The title compounds were prepared following the procedure described in Example 2, step 5, using 1e and 1-methyl-4,5,6,7-tetrahydro-1H-benzo[d][1,2,3]triazol-6-amine. The final product was purified by reverse phase chromatography eluted with ACN and water and dried using lyophilization to afford the title products as white solids. ESI-MS, m/z 467.2 (M+H)⁺.

### Example 86: 2-(3-ethyl-4-((4-fluoro-3-methylphenyl)carbamoyl)-1,5-dimethyl-1H-pyrrol-2-yl)-2-oxoacetic acid

The title compound was prepared following the procedure described in Example 1, Step 5, using 7g as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS, m/z 444 (M+H)⁺.

### Example 87: 4-ethyl-N-(4-fluoro-3-methylphenyl)-5-(2-(((1s,3s)-3-hydroxy-1-methylcyclobutyl)amino)-2-oxoacetyl)-1,2-dimethyl-1H-pyrrole-3-carboxamide

The title compound was prepared following the procedure described in Example 1, Step 5, using 7g as the starting carboxylic acid and (1s,3s)-3-amino-3-methylcyclobutan-1-ol as the starting amine. The final product was purified by reverse phase chromatography eluted with ACN and water and dried using lyophilization to afford the title products as white solids. ESI-MS, m/z 430 (M+H)⁺.

### Example 88: 4-ethyl-N-(4-fluoro-3-methylphenyl)-5-(2-((2-hydroxy-2-methylpropyl)amino)-2-oxoacetyl)-1,2-dimethyl-1H-pyrrole-3-carboxamide

The title compound was prepared following the procedure described in Example 1, Step 5, using 7g as the starting carboxylic acid and 1-amino-2-methylpropan-2-ol as the starting amine. The final product was purified by reverse phase chromatography eluted with ACN and water and dried using lyophilization to afford the title products as white solids. ESI-MS, m/z 418 (M+H)⁺.

### Example 89: 4-ethyl-N-(4-fluoro-3-methylphenyl)-1,2-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 1, Step 5, using 7g as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS, m/z 506 (M+H)⁺.

### Example 90: (R)-4-ethyl-N-(4-fluoro-3-methylphenyl)-1,2-dimethyl-5-(2-oxo-2-((1,1,1-trifluoropropan-2-yl)amino)acetyl)-1H-pyrrole-3-carboxamide

The title compound was prepared following the procedure described in Example 1, Step 5, using 7g as the starting carboxylic acid and (R)-1,1,1-trifluoropropan-2-amine as the starting amine. The final product was purified by reverse phase chromatography eluted with ACN and water and dried using lyophilization to afford the title products as white solids. ESI-MS, m/z 442 (M+H)⁺.

### Example 91: 4-ethyl-N-(4-fluoro-3-methylphenyl)-1,2-dimethyl-5-(2-oxo-2-((1-(pyridazin-3-yl)piperidin-4-yl)amino)acetyl)-1H-pyrrole-3-carboxamide

The title compound was prepared following the procedure described in Example 1, Step 5, using 7g as the starting carboxylic acid and 1-(pyridazin-3-yl)piperidin-4-amine as the starting amine. The final product was purified by reverse phase chromatography eluted with ACN and water and dried using lyophilization to afford the title products as white solids. ESI-MS, m/z 507 (M+H)⁺.

### Example 92: N-(4-fluoro-3-methylphenyl)-5-(2-(((1s,4s)-4-hydroxycyclohexyl)amino)-2-oxoacetyl)-4-isopropyl-1,2-dimethyl-1H-pyrrole-3-carboxamide

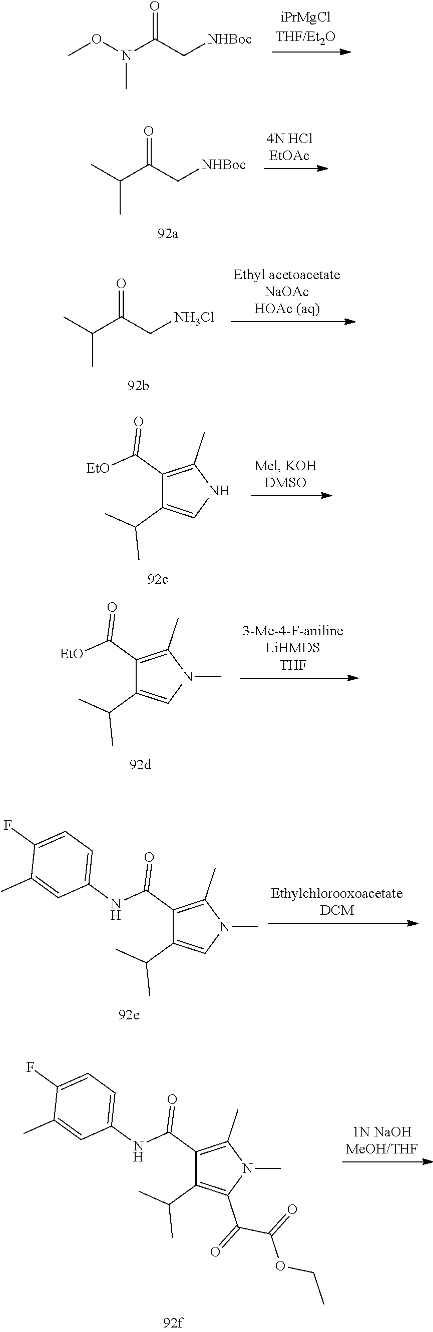

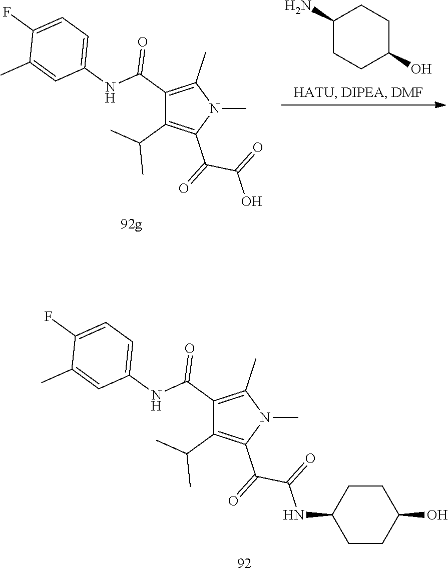 [SMILES: Cc1cc(NC(=O)c2c(C(C)C)c(C(=O)C(=O)O)n(C)c2C)ccc1F ; N[C@H]1CC[C@@H](O)CC1 ; Cc1cc(NC(=O)c2c(C(C)C)c(C(=O)C(=O)N[C@H]3CC[C@@H](O)CC3)n(C)c2C)ccc1F]

### Step 1: Synthesis of tert-butyl (3-methyl-2-oxobutyl)carbamate (92a)

The title compound was prepared following the procedure described in Example 7, Step 1, using isopropyl magnesium chloride as the Grignard reagent. ESI-MS, m/z 202 (M+H)⁺.

### Step 2: Synthesis of 1-amino-3-methylbutan-2-one hydrochloride (92b)

The title compound was prepared following the procedure described in Example 7, Step 2, using 92a as the starting material. ESI-MS, m/z 102 [M+H]⁺.

### Step 3: Synthesis of ethyl 4-isopropyl-2-methyl-1H-pyrrole-3-carboxylate (92c)

The title compound was prepared following the procedure described in Example 7, Step 3, using 92b as the starting material. ESI-MS, m/z 196^{[M+H]+}.

### Step 4: Synthesis of ethyl 4-isopropyl-1,2-dimethyl-1H-pyrrole-3-carboxylate (92d)

The title compound was prepared following the procedure described in Example 1, Step 1, using 92c as the starting material. ESI-MS, m/z 210 (M+H)⁺.

### Step 5: Synthesis of 4-isopropyl-N-(4-fluoro-3-methylphenyl)-1,2-dimethyl-1H-pyrrole-3-carboxamide (92e)

The title compound was prepared following the procedure described in Example 1, Step 2, using 92d as the starting material. ESI-MS, m/z 289 (M+H)⁺.

### Step 6: Synthesis of ethyl 2-(3-isopropyl-4-((4-fluoro-3-methylphenyl)carbamoyl)-1,5-dimethyl-1H-pyrrol-2-yl)-2-oxoacetate (92f)

The title compound was prepared following the procedure described in Example 1, Step 3, using 92e as the starting material. ESI-MS, m/z 389 (M+H)⁺.

### Step 7: Synthesis of 2-(3-isopropyl-4-((4-fluoro-3-methylphenyl)carbamoyl)-1,5-dimethyl-1H-pyrrol-2-yl)-2-oxoacetic acid (92g)

The title compound was prepared following the procedure described in Example 1, Step 4, using 92f as the starting material. ESI-MS, m/z 361 (M+H)⁺.

### Step 8: N-(4-fluoro-3-methylphenyl)-5-(2-(((1s,4s)-4-hydroxycyclohexyl)amino)-2-oxoacetyl)-4-isopropyl-1,2-dimethyl-1H-pyrrole-3-carboxamide (92)

The title compound was prepared following the procedure described in Example 1, Step 5, using 92g as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS, m/z 458 (M+H)⁺.

### Example 93: N-(4-fluoro-3-methylphenyl)-5-(2-(((1s,3s)-3-hydroxy-1-methylcyclobutyl)amino)-2-oxoacetyl)-4-isopropyl-1,2-dimethyl-1H-pyrrole-3-carboxamide

The title compound was prepared following the procedure described in Example 1, Step 5, using 92g as the starting carboxylic acid and (1s,3s)-3-amino-3-methylcyclobutan-1-ol as the starting amine. The final product was purified by reverse phase chromatography eluted with ACN and water and dried using lyophilization to afford the title products as white solids. ESI-MS, m/z 444 (M+H)⁺.

### Example 94: 4-isopropyl-N-(4-fluoro-3-methylphenyl)-5-(2-((2-hydroxy-2-methylpropyl)amino)-2-oxoacetyl)-1,2-dimethyl-1H-pyrrole-3-carboxamide

The title compound was prepared following the procedure described in Example 1, Step 5, using 92g as the starting carboxylic acid and 1-amino-2-methylpropan-2-ol as the starting amine. The final product was purified by reverse phase chromatography eluted with ACN and water and dried using lyophilization to afford the title products as white solids. ESI-MS, m/z 432 (M+H)⁺.

### Example 95: 4-isopropyl-N-(4-fluoro-3-methylphenyl)-1,2-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 1, Step 5, using 92g as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS, m/z 520 (M+H)⁺.

### Example 96: (R)-4-isopropyl-N-(4-fluoro-3-methylphenyl)-1,2-dimethyl-5-(2-oxo-2-((1,1,1-trifluoropropan-2-yl)amino)acetyl)-1H-pyrrole-3-carboxamide

The title compound was prepared following the procedure described in Example 1, Step 5, using 92g as the starting carboxylic acid and (R)-1,1,1-trifluoropropan-2-amine as the starting amine. The final product was purified by reverse phase chromatography eluted with ACN and water and dried using lyophilization to afford the title products as white solids. ESI-MS, m/z 456 (M+H)⁺.

### Example 97: 4-cyclopropyl-N-(4-fluoro-3-methylphenyl)-5-(2-(((1s,4s)-4-hydroxycyclohexyl)amino)-2-oxoacetyl)-1,2-dimethyl-1H-pyrrole-3-carboxamide

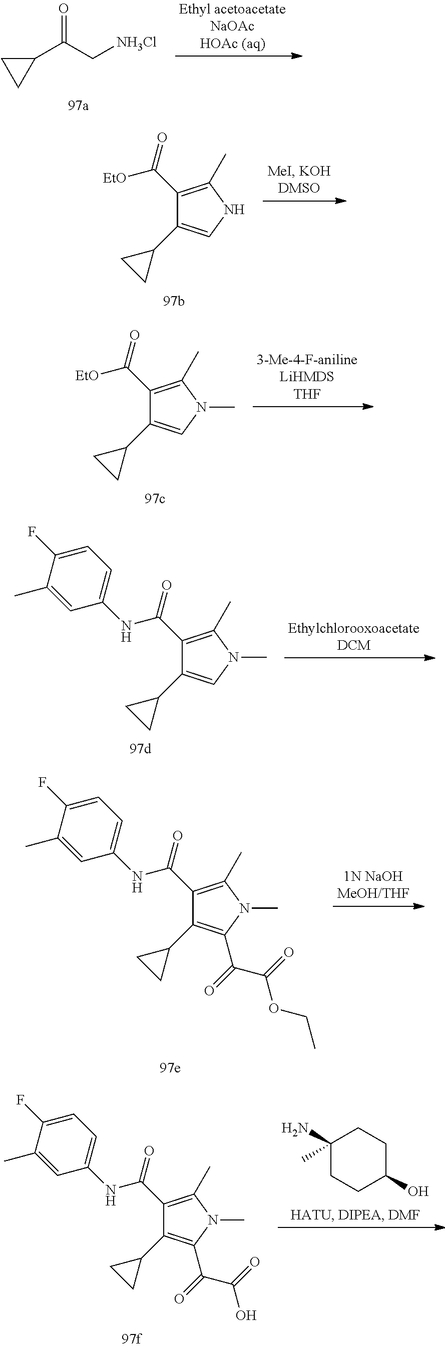

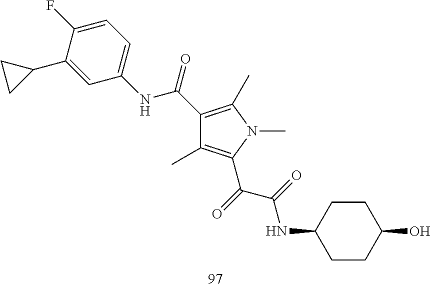 [SMILES: Cc1c(C(=O)Nc2ccc(F)c(C3CC3)c2)c(C)n(C)c1C(=O)C(=O)N[C@H]1CC[C@@H](O)CC1]

### Step 1: Synthesis of ethyl 4-cyclopropyl-2-methyl-1H-pyrrole-3-carboxylate (97b)

The title compound was prepared following the procedure described in Example 7, Step 3, using the commercially available 2-amino-1-cyclopropylethan-1-one (97a) as the starting material. ESI-MS, m/z 194 (M+H)⁺.

### Step 2: Synthesis of ethyl 4-cyclopropyl-1,2-dimethyl-1H-pyrrole-3-carboxylate (97c)

The title compound was prepared following the procedure described in Example 1, Step 1, using 97b as the starting material. ESI-MS, m/z 208 (M+H)⁺.

### Step 3: Synthesis of 4-cyclopropyl-N-(4-fluoro-3-methylphenyl)-1,2-dimethyl-1H-pyrrole-3-carboxamide (97d)

The title compound was prepared following the procedure described in Example 1, Step 2, using 97c as the starting material. ESI-MS, m/z 287Y (M+H)⁺.

### Step 4: Synthesis of ethyl 2-(3-cyclopropyl-4-((4-fluoro-3-methylphenyl)carbamoyl)-1,5-dimethyl-1H-pyrrol-2-yl)-2-oxoacetate (97e)

The title compound was prepared following the procedure described in Example 1, Step 3, using 97d as the starting material. ESI-MS, m/z 387 (M+H)⁺.

### Step 5: Synthesis of 2-(3-cyclopropyl-4-((4-fluoro-3-methylphenyl)carbamoyl)-1,5-dimethyl-1H-pyrrol-2-yl)-2-oxoacetic acid (97f)

The title compound was prepared following the procedure described in Example 1, Step 4, using 97e as the starting material. ESI-MS, m/z 359 (M+H)⁺.

### Step 6: 4-cyclopropyl-N-(4-fluoro-3-methylphenyl)-5-(2-(((1s,4s)-4-hydroxycyclohexyl)amino)-2-oxoacetyl)-1,2-dimethyl-1H-pyrrole-3-carboxamide (97)

The title compound was prepared following the procedure described in Example 1, Step 5, using 97f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS, m/z 456 (M+H)⁺.

### Example 98: 4-cyclopropyl-N-(4-fluoro-3-methylphenyl)-5-(2-(((1s,3s)-3-hydroxy-1-methylcyclobutyl)amino)-2-oxoacetyl)-1,2-dimethyl-1H-pyrrole-3-carboxamide

The title compound was prepared following the procedure described in Example 1, Step 5, using 97f as the starting carboxylic acid and (1s,3s)-3-amino-3-methylcyclobutan-1-ol as the starting amine. The final product was purified by reverse phase chromatography eluted with ACN and water and dried using lyophilization to afford the title products as white solids. ESI-MS, m/z 442 (M+H)⁺.

### Example 99: 4-cyclopropyl-N-(4-fluoro-3-methylphenyl)-5-(2-((2-hydroxy-2-methylpropyl)amino)-2-oxoacetyl)-1,2-dimethyl-1H-pyrrole-3-carboxamide

The title compound was prepared following the procedure described in Example 1, Step 5, using 97f as the starting carboxylic acid and 1-amino-2-methylpropan-2-ol as the starting amine. The final product was purified by reverse phase chromatography eluted with ACN and water and dried using lyophilization to afford the title products as white solids. ESI-MS, m/z 430 (M+H)⁺.

### Example 100: 4-cyclopropyl-N-(4-fluoro-3-methylphenyl)-1,2-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 1, Step 5, using 97f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS, m/z 518 (M+H)⁺.

### Example 101: (R)-4-cyclopropyl-N-(4-fluoro-3-methylphenyl)-1,2-dimethyl-5-(2-oxo-2-((1,1,1-trifluoropropan-2-yl)amino)acetyl)-1H-pyrrole-3-carboxamide

The title compound was prepared following the procedure described in Example 1, Step 5, using 97f as the starting carboxylic acid and (R)-1,1,1-trifluoropropan-2-amine as the starting amine. The final product was purified by reverse phase chromatography eluted with ACN and water and dried using lyophilization to afford the title products as white solids. ESI-MS, m/z 454 (M+H)⁺.

### Example 102: 4-cyclopropyl-N-(4-fluoro-3-methylphenyl)-5-(2-(((1r,4r)-4-hydroxy-1-methylcyclohexyl)amino)-2-oxoacetyl)-1,2-dimethyl-1H-pyrrole-3-carboxamide

The title compound was prepared following the procedure described in Example 1, Step 5, using 97f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS, m/z 470 (M+H)⁺.

### Example 103: 4-cyclopropyl-N-(4-fluoro-3-methylphenyl)-1,2-dimethyl-5-(2-oxo-2-((1-(pyridazin-3-yl)piperidin-4-yl)amino)acetyl)-1H-pyrrole-3-carboxamide

The title compound was prepared following the procedure described in Example 1, Step 5, using 97f as the starting carboxylic acid and 1-(pyridazin-3-yl)piperidin-4-amine as the starting amine. The final product was purified by reverse phase chromatography eluted with ACN and water and dried using lyophilization to afford the title products as white solids. ESI-MS, m/z 519 (M+H)⁺.

### Example 104: 2-ethyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide

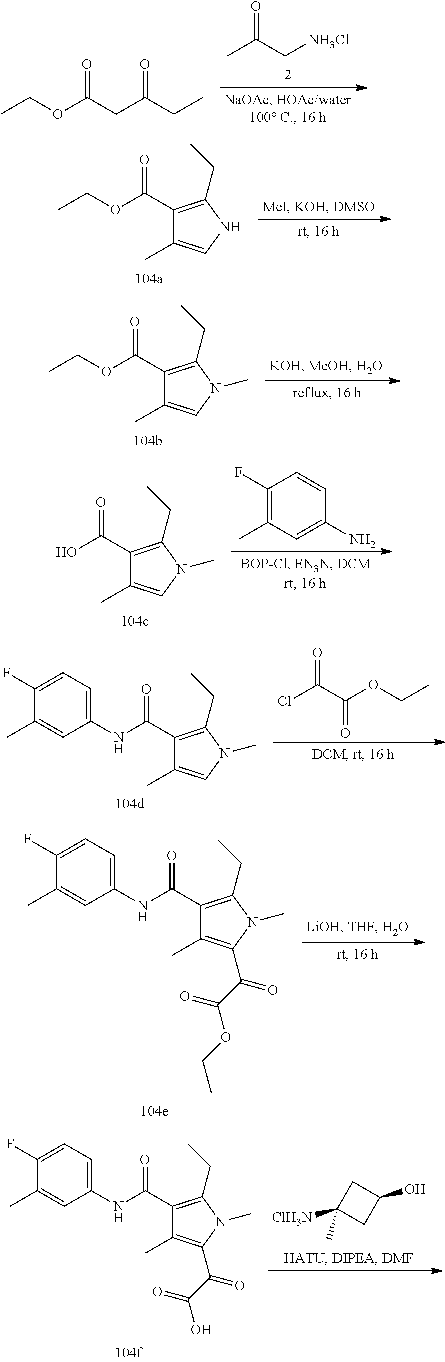

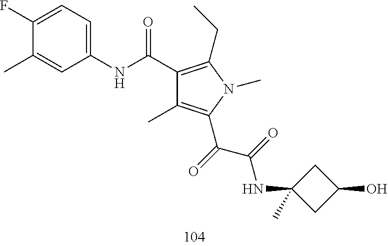 [SMILES: CCc1c(C(=O)Nc2ccc(F)c(C)c2)c(C)c(C(=O)C(=O)N[C@]2(C)C[C@H](O)C2)n1C]

### Step 1: Synthesis of ethyl 2-ethyl-4-methyl-1H-pyrrole-3-carboxylate (104a)

To a solution of ethyl 3-oxopentanoate (3 g, 20.8 mmol), NaOAc (3.42 g, 41.7 mmol) in HOAc (20 mL) and H₂O (20 mL) was added 1-aminopropan-2-one hydrochloride (2.27 g, 20.8 mmol) at ambient temperature. The reaction mixture was stirred 100° C. for 16 h. The resulting mixture was concentrated under vacuum. The residue was extracted with EA (100 mL×3). The organic layer was washed with brine, dried over Na₂SO₄, concentrated under vacuum to give the title compound 104a as a brown oil, 3.37 g, yield: 89.4%. ESI-MS m/z 182 (M+H)⁺.

### Step 2: Synthesis of ethyl 2-ethyl-1,4-dimethyl-1H-pyrrole-3-carboxylate (104b)

To a solution of 104a (3.37 g, 18.6 mmol) in DMSO (40 mL) was added KOH (2.09 g, 37.2 mmol). The resulting solution was cooled in an ice-bath, and MeI (5.29 g, 37.2 mmol) was added dropwise over 15 min. The reaction was stirred at 0° C. for 0.5 h, then the ice bath was removed, and the mixture was allowed to warm to room temperature and stirred for 16 h. The resulting mixture was diluted with EtOAc (100 mL), washed with NH₄Cl (aq) (200 mL) and brine (200 mL), dried over Na₂SO₄, concentrated under vacuum to give the title compound 104b a brown oil, 3.2 g, yield: 88.1%. ESI-MS m/z 196 (M+H)⁺.

### Step 3: Synthesis of 2-ethyl-1,4-dimethyl-1H-pyrrole-3-carboxylic acid (104c)

To a solution of 104b (3.2 g, 16.4 mmol) in MeOH (30 mL) and H₂O (30 mL) was added KOH (4.68 g, 82.0 mmol) and the reaction mixture was warmed to 90° C. for 16 h. The resulting mixture was concentrated under vacuum then acidified with 1 N HCl (aq) to pH=2-3. The mixture was extracted with EtOAc (100 mL×3). The organic layer was washed with brine, dried over Na₂SO₄, concentrated under vacuum to give the title compound 104c as a yellow solid, 2.4 g, yield: 87.6%. ESI-MS m/z 168 (M+H)⁺.

### Step 4: Synthesis of 2-ethyl-N-(4-fluoro-3-methylphenyl)-1,4-dimethyl-1H-pyrrole-3-carboxamide (104d)

To a solution of 2-ethyl-1,4-dimethyl-1H-pyrrole-3-carboxylic acid (2.3 g, 13.8 mmol), TEA (4.17 g, 41.3 mmol) and BOP-Cl (5.25 g, 20.6 mmol) in DCM (45 mL) at ambient temperature was added 4-fluoro-3-methylaniline (2.58 g, 20.6 mmol). The reaction mixture was stirred at room temperature for 16 h. The reaction mixture was diluted with water (100 mL) and extracted with EtOAc (50 mL×3). The organic extracts were combined, washed with water, brine, dried over Na₂SO₄, and concentrated in vacuo to give the crude product. The crude product was purified by flash chromatography on silica gel (PE/EA=5:1) to give the title compound 104d as a yellow oil, 1.9 g, 48.3%. ESI-MS m/z 275 (M+H)⁺.

### Step 5: Synthesis of ethyl 2-(5-ethyl-4-((4-fluoro-3-methylphenyl)carbamoyl)-1,3-dimethyl-1H-pyrrol-2-yl)-2-oxoacetate (104e)

To a solution of 104d (1.9 g, 6.9 mmol) in DCM (40 mL) at 0° C. was added ethyl 2-chloro-2-oxoacetate (1.4 g, 10.4 mmol). After 1 h the reaction mixture was warmed to room temperature and stirred for an additional 16 h. The reaction mixture was diluted with water (100 mL) and extracted with EtOAc (50 mL×3). The organic extracts were combined, washed with water, brine, dried over Na₂SO₄, and concentrated in vacuo to give the crude product. The crude product was purified by flash chromatography on silica gel (PE/EA=5:1) to give the title compound 104e as a yellow oil, 1.5 g, 57.8%. ESI-MS m/z 375 (M+H)⁺.

### Step 6: Synthesis of 2-(5-ethyl-4-((4-fluoro-3-methylphenyl)carbamoyl)-1,3-dimethyl-1H-pyrrol-2-yl)-2-oxoacetic acid (104f)

To a solution of 104e (1.5 g, 4.0 mmol) in THF (10 mL) and H₂O (10 mL) was added LiOH (505 mg, 12.0 mmol). The reaction mixture was stirred at ambient temperature for 16 h then acidified with 1 N HCl (aq) to pH=2-3. The mixture was extracted with EtOAc (50 mL×3). The organic layer was washed with brine, dried over Na₂SO₄, concentrated under vacuum to give the title compound 104f as a light yellow solid, 1.3 g, yield: 93.7%. ESI-MS m/z 347 (M+H)⁺.

### Step 7: Synthesis of 2-ethyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide (104)

To a solution of 104f (120 mg, 0.35 mmol), DIPEA (134 mg, 1.05 mmol) and HATU (198 mg, 0.52 mmol) in DMF (2 mL) was added (1s,3s)-3-amino-3-methylcyclobutan-1-ol hydrochloride (111 mg, 0.52 mmol) at room temperature. The reaction mixture was stirred at room temperature for 16 h. The reaction mixture was diluted with water (20 mL) and extracted with EtOAc (10 mL×3). The organic extracts were combined, washed with water, brine, dried over Na2SO4, and concentrated in vacuo to give the crude product. The crude product was purified by prep-HPLC to give the title compound 104 as a white solid, 60 mg, 34.3%. ESI-MS m/z 430 (M+H)⁺.

### Example 105: 2-ethyl-N-(4-fluoro-3-methylphenyl)-5-(2-((2-hydroxy-2-methylpropyl)amino)-2-oxoacetyl)-1,4-dimethyl-1H-pyrrole-3-carboxamide

The title compound was prepared following the procedure described in Example 104, Step 7, using 1-amino-2-methylpropan-2-ol as the starting amine. The final product was purified by reverse phase chromatography eluted with ACN and water and dried using lyophilization to afford the title products as white solids. ESI-MS m/z 418 (M+H)⁺.

### Example 106: 2-ethyl-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 104, Step 7, using (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 506 (M+H)⁺.

### Example 107: 2-ethyl-N-(4-fluoro-3-methylphenyl)-5-(2-(((1s,4s)-4-hydroxy-1-methylcyclohexyl)amino)-2-oxoacetyl)-1,4-dimethyl-1H-pyrrole-3-carboxamide

The title compound was prepared following the procedure described in Example 104, Step 7, using (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 458 (M+H)⁺.

### Example 108: 2-ethyl-N-(4-fluoro-3-methylphenyl)-5-(2-(((1s,4s)-4-hydroxycyclohexyl)amino)-2-oxoacetyl)-1,4-dimethyl-1H-pyrrole-3-carboxamide

The title compound was prepared following the procedure described in Example 104, Step 7, using (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 444 (M+H)⁺.

### Example 109: N-(4-fluoro-3-methylphenyl)-5-(2-(((1s,3s)-3-hydroxy-1-methylcyclobutyl)amino)-2-oxoacetyl)-2-isopropyl-1,4-dimethyl-1H-pyrrole-3-carboxamide

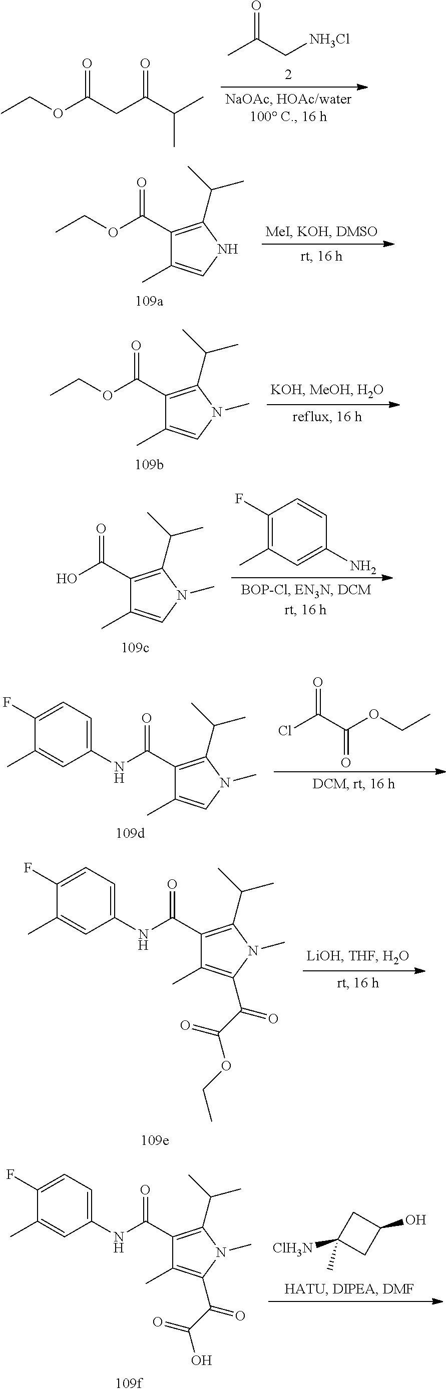

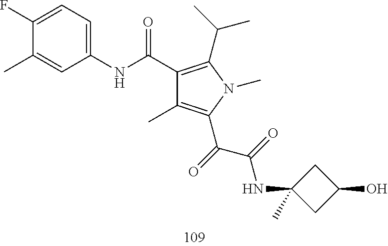 [SMILES: Cc1cc(NC(=O)c2c(C)c(C(=O)C(=O)N[C@]3(C)C[C@H](O)C3)n(C)c2C(C)C)ccc1F]

### Step 1: Synthesis of ethyl 2-isopropyl-4-methyl-1H-pyrrole-3-carboxylate (109a)

The title compound was prepared following the procedure described in Example 104, Step 1, using the commercially available ethyl 4-methyl-3-oxopentanoate as the starting material. ESI-MS, m/z 196 (M+H)⁺.

### Step 2: Synthesis of ethyl 2-isopropyl-1,4-dimethyl-1H-pyrrole-3-carboxylate (109b)

The title compound was prepared following the procedure described in Example 104, Step 2, using 109a as the starting material. ESI-MS, m/z 210 (M+H)⁺.

### Step 3: Synthesis of 2-isopropyl-1,4-dimethyl-1H-pyrrole-3-carboxylic acid (109c)

The title compound was prepared following the procedure described in Example 104, Step 3, using 109b as the starting material. ESI-MS, m/z 181 (M+H)⁺.

### Step 4: Synthesis of 2-isopropyl-N-(4-fluoro-3-methylphenyl)-1,4-dimethyl-1H-pyrrole-3-carboxamide (109d)

The title compound was prepared following the procedure described in Example 104, Step 4, using 109c as the starting material. ESI-MS, m/z 289 (M+H)⁺.

### Step 5: Synthesis of ethyl 2-(5-isopropyl-4-((4-fluoro-3-methylphenyl)carbamoyl)-1,3-dimethyl-1H-pyrrol-2-yl)-2-oxoacetate (109e)

The title compound was prepared following the procedure described in Example 104, Step 5, using 109d as the starting material. ESI-MS, m/z 389 (M+H)⁺.

### Step 6: Synthesis of 2-(5-isopropyl-4-((4-fluoro-3-methylphenyl)carbamoyl)-1,3-dimethyl-1H-pyrrol-2-yl)-2-oxoacetic acid (109f)

The title compound was prepared following the procedure described in Example 104, Step 6, using 109e as the starting material. ESI-MS, m/z 361 (M+H)⁺.

### Step 7: Synthesis of 2-isopropyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide (109)

The title compound was prepared following the procedure described in Example 104, Step 7, using 109f as the starting carboxylic acid. The final product was purified by reverse phase chromatography eluted with ACN and water and dried using lyophilization to afford the title products as white solids. ESI-MS, m/z 444 (M+H)⁺.

### Example 110: N-(4-fluoro-3-methylphenyl)-2-isopropyl-1,4-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 104, Step 7, using 109f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 520 (M+H)⁺.

### Example 111: N-(4-fluoro-3-methylphenyl)-5-(2-(((1s,4s)-4-hydroxy-1-methylcyclohexyl)amino)-2-oxoacetyl)-2-isopropyl-1,4-dimethyl-1H-pyrrole-3-carboxamide

The title compound was prepared following the procedure described in Example 104, Step 7, using 109f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 472 (M+H)⁺.

### Example 112: N-(4-fluoro-3-methylphenyl)-5-(2-(((1s,4s)-4-hydroxycyclohexyl)amino)-2-oxoacetyl)-2-isopropyl-1,4-dimethyl-1H-pyrrole-3-carboxamide

The title compound was prepared following the procedure described in Example 104, Step 7, using 109f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 458 (M+H)⁺.

### Example 113: N-(4-fluoro-3-methylphenyl)-5-(2-((2-hydroxy-2-methylpropyl)amino)-2-oxoacetyl)-2-isopropyl-1,4-dimethyl-1H-pyrrole-3-carboxamide

The title compound was prepared following the procedure described in Example 104, Step 7, using 109f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 432 (M+H)⁺.

### Example 114: 2-cyclopropyl-N-(4-fluoro-3-methylphenyl)-5-(2-(((1s,4s)-4-hydroxy-1-methylcyclohexyl)amino)-2-oxoacetyl)-1,4-dimethyl-1H-pyrrole-3-carboxamide

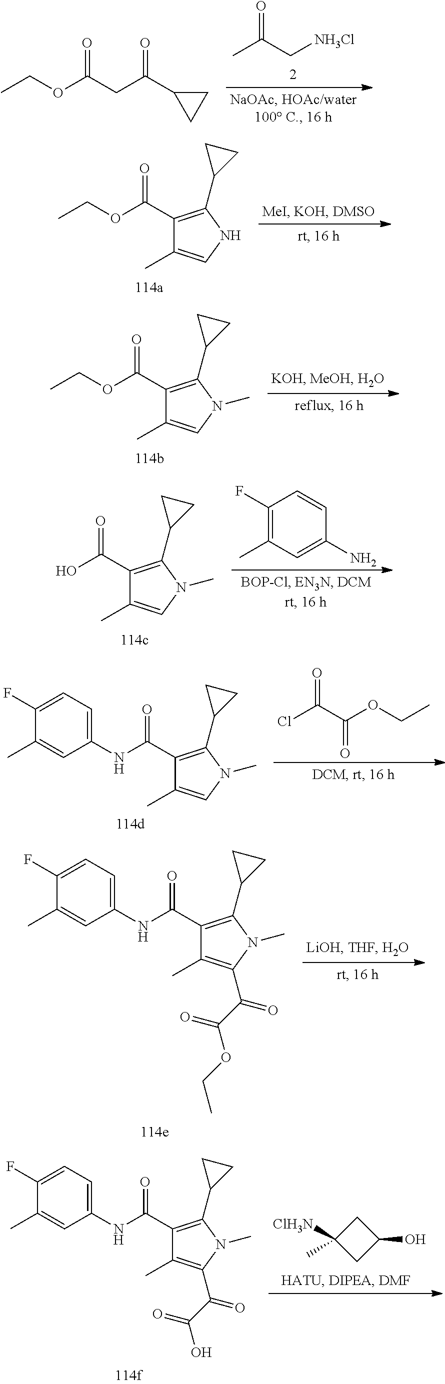

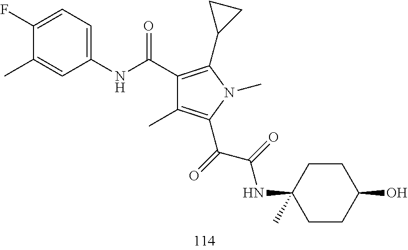 [SMILES: Cc1cc(NC(=O)c2c(C)c(C(=O)C(=O)N[C@]3(C)CC[C@H](O)CC3)n(C)c2C2CC2)ccc1F]

### Step 1: Synthesis of ethyl 2-cyclopropyl-4-methyl-1H-pyrrole-3-carboxylate (114a)

The title compound was prepared following the procedure described in Example 104, Step 1, using the commercially available ethyl 3-cyclopropyl-3-oxopropanoate as the starting material. ESI-MS, m/z 194 (M+H)⁺.

### Step 2: Synthesis of ethyl 2-cyclopropyl-1,4-dimethyl-1H-pyrrole-3-carboxylate (114b)

The title compound was prepared following the procedure described in Example 104, Step 2, using 114a as the starting material. ESI-MS, m/z 208 (M+H)⁺.

### Step 3: Synthesis of 2-cyclopropyl-1,4-dimethyl-1H-pyrrole-3-carboxylic acid (114c)

The title compound was prepared following the procedure described in Example 104, Step 3, using 114b as the starting material. ESI-MS, m/z 180 (M+H)⁺.

### Step 4: Synthesis of 2-cyclopropyl-N-(4-fluoro-3-methylphenyl)-1,4-dimethyl-1H-pyrrole-3-carboxamide (114d)

The title compound was prepared following the procedure described in Example 104, Step 4, using 114c as the starting material. ESI-MS, m/z 287 (M+H)⁺.

### Step 5: Synthesis of ethyl 2-(5-cyclopropyl-4-((4-fluoro-3-methylphenyl)carbamoyl)-1,3-dimethyl-1H-pyrrol-2-yl)-2-oxoacetate (114e)

The title compound was prepared following the procedure described in Example 104, Step 5, using 114d as the starting material. ESI-MS, m/z 387 (M+H)⁺.

### Step 6: Synthesis of 2-(5-cyclopropyl-4-((4-fluoro-3-methylphenyl)carbamoyl)-1,3-dimethyl-1H-pyrrol-2-yl)-2-oxoacetic acid (114f)

The title compound was prepared following the procedure described in Example 104, Step 6, using 114e as the starting material. ESI-MS, m/z 359 (M+H)⁺.

### Step 7: Synthesis of 2-cyclopropyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide (114)

The title compound was prepared following the procedure described in Example 104, Step 7, using 114f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS, m/z 470 (M+H)⁺.

### Example 115: 2-cyclopropyl-N-(4-fluoro-3-methylphenyl)-5-(2-(((1s,4s)-4-hydroxycyclohexyl)amino)-2-oxoacetyl)-1,4-dimethyl-1H-pyrrole-3-carboxamide

The title compound was prepared following the procedure described in Example 104, Step 7, using 114f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 456 (M+H)⁺.

### Example 116: 2-cyclohexyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide

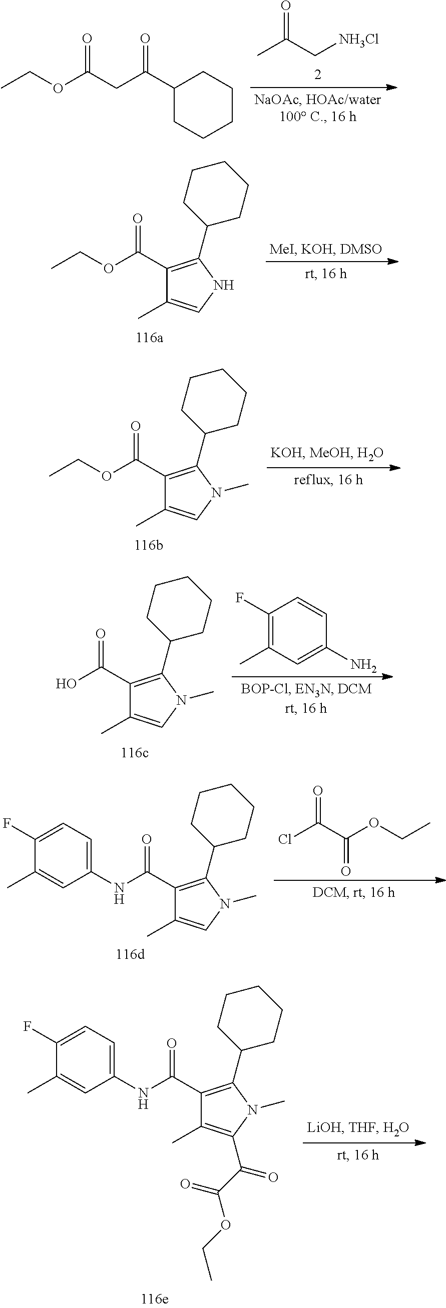

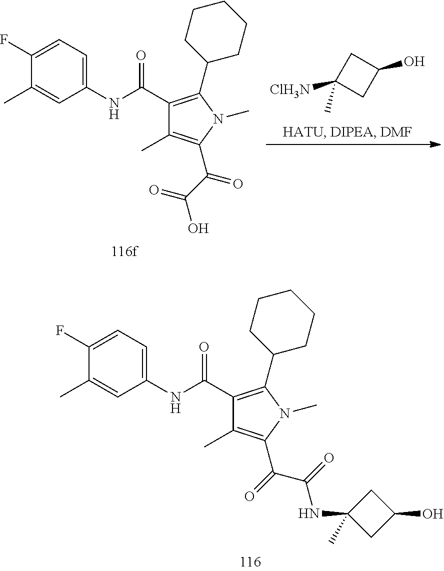

### Step 1: Synthesis of ethyl 2-cyclohexyl-4-methyl-1H-pyrrole-3-carboxylate (116a)

The title compound was prepared following the procedure described in Example 104, Step 1, using the commercially available ethyl 3-cyclohexyl-3-oxopropanoate as the starting material. ESI-MS, m/z 236 (M+H)⁺.

### Step 2: Synthesis of ethyl 2-cyclohexyl-1,4-dimethyl-1H-pyrrole-3-carboxylate (116b)

The title compound was prepared following the procedure described in Example 104, Step 2, using 116a as the starting material. ESI-MS, m/z 250 (M+H)⁺.

### Step 3: Synthesis of 2-cyclohexyl-1,4-dimethyl-1H-pyrrole-3-carboxylic acid (116c)

The title compound was prepared following the procedure described in Example 104, Step 3, using 116b as the starting material. ESI-MS, m/z 222 (M+H)⁺.

### Step 4: Synthesis of 2-cyclohexyl-N-(4-fluoro-3-methylphenyl)-1,4-dimethyl-1H-pyrrole-3-carboxamide (116d)

The title compound was prepared following the procedure described in Example 104, Step 4, using 116c as the starting material. ESI-MS, m/z 329 (M+H)⁺.

### Step 5: Synthesis of ethyl 2-(5-cyclohexyl-4-((4-fluoro-3-methylphenyl)carbamoyl)-1,3-dimethyl-1H-pyrrol-2-yl)-2-oxoacetate (116e)

The title compound was prepared following the procedure described in Example 104, Step 5, using 116d as the starting material. ESI-MS, m/z 429 (M+H)⁺.

### Step 6: Synthesis of 2-(5-cyclohexyl-4-((4-fluoro-3-methylphenyl)carbamoyl)-1,3-dimethyl-1H-pyrrol-2-yl)-2-oxoacetic acid (116f)

The title compound was prepared following the procedure described in Example 104, Step 6, using 116e as the starting material. ESI-MS, m/z 401 (M+H)⁺.

### Step 7: 2-cyclohexyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide (116)

The title compound was prepared following the procedure described in Example 104, Step 7, using 116f as the starting carboxylic acid. The final product was purified by reverse phase chromatography eluted with ACN and water and dried using lyophilization to afford the title products as white solids. ESI-MS, m/z 484 (M+H)⁺.

### Example 117: 2-cyclohexyl-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 104, Step 7, using 116f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 560 (M+H)⁺.

### Example 118: 2-cyclohexyl-N-(4-fluoro-3-methylphenyl)-5-(2-(((1s,4s)-4-hydroxy-1-methylcyclohexyl)amino)-2-oxoacetyl)-1,4-dimethyl-1H-pyrrole-3-carboxamide

The title compound was prepared following the procedure described in Example 104, Step 7, using 116f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 512 (M+H)⁺.

### Example 119: 2-cyclohexyl-N-(4-fluoro-3-methylphenyl)-5-(2-(((1s,4s)-4-hydroxycyclohexyl)amino)-2-oxoacetyl)-1,4-dimethyl-1H-pyrrole-3-carboxamide

The title compound was prepared following the procedure described in Example 104, Step 7, using 116f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 498 (M+H)⁺.

### Example 120: 2-cyclohexyl-N-(4-fluoro-3-methylphenyl)-5-(2-((2-hydroxy-2-methylpropyl)amino)-2-oxoacetyl)-1,4-dimethyl-1H-pyrrole-3-carboxamide

The title compound was prepared following the procedure described in Example 104, Step 7, using 116f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 472 (M+H)⁺.

### Example 121: 5-(2-(((1s,4s)-4-((2,28-dioxo-32-(2-oxohexahydro-1H-thieno[3,4-d]imidazol-4-yl)-6,9,12,15,18,21,24-heptaoxa-3,27-diazadotriacontyl)oxy)cyclohexyl)amino)-2-oxoacetyl)-N-(4-fluoro-3-methylphenyl)-1,2,4-trimethyl-1H-pyrrole-3-carboxamide

To solution of 144 (19 mg, 0.039 mmol), biotin-dPeg₇-NH₂ (26 mg, 0.043 mmol) and HATU (18 mg, 0.047 mmol) in DMF (1 mL) was added DIPEA (15 mg, 0.117 mmol). After 16 h the reaction mixture was purified directly via reverse phase chromatography eluted with ACN and water and dried using lyophilization to afford the title product as a white solid ESI-MS m/z 1063 (M+H)⁺.

### Example 122: N-(4-fluoro-3-methylphenyl)-5-(2-(((1R,2S,4S)-2-fluoro-4-hydroxycyclohexyl)amino)-2-oxoacetyl)-1,2,4-trimethyl-1H-pyrrole-3-carboxamide

The title compound was prepared following the procedure described in Example 1, Step 5, using ((1R,2S, 4S)-2-fluoro-4-hydroxycyclohexylamine hydrochloride as the amine. The final product was purified by reverse phase chromatography eluted with ACN and water and dried using lyophilization to afford the title products as white solids. ESI-MS, m/z 448 (M+H)⁺.

### Example 123: 2-(cyclopropylmethyl)-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide

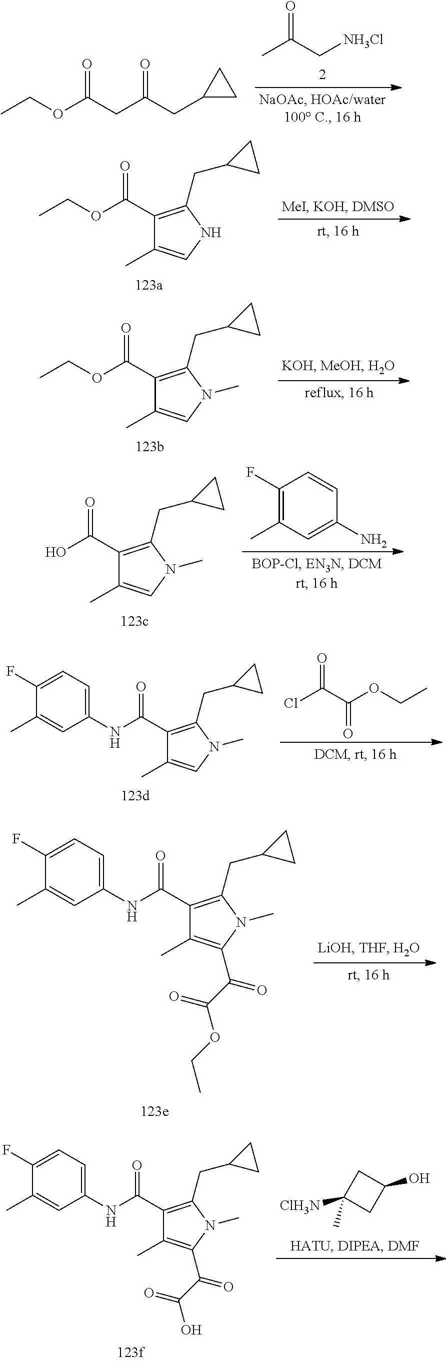

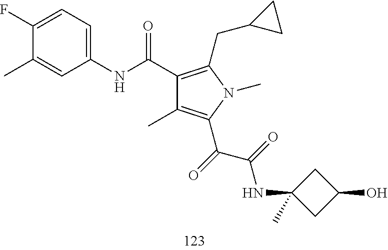 [SMILES: Cc1cc(NC(=O)c2c(C)c(C(=O)C(=O)N[C@]3(C)C[C@H](O)C3)n(C)c2CC2CC2)ccc1F]

### Step 1: Synthesis of ethyl 2-(cyclopropylmethyl)-4-methyl-1H-pyrrole-3-carboxylate (123a)

The title compound was prepared following the procedure described in Example 104, Step 1, using the commercially available ethyl 4-cyclopropyl-3-oxobutanoate as the starting material. ESI-MS, m/z 208 (M+H)⁺.

### Step 2: Synthesis of ethyl 2-(cyclopropylmethyl)-1,4-dimethyl-H-pyrrole-3-carboxylate (123b)

The title compound was prepared following the procedure described in Example 104, Step 2, using 123a as the starting material. ESI-MS, m/z 222 (M+H)⁺.

### Step 3: Synthesis of 2-(cyclopropylmethyl)-1,4-dimethyl-H-pyrrole-3-carboxylic acid (123c)

The title compound was prepared following the procedure described in Example 104, Step 3, using 123b as the starting material. ESI-MS, m/z 194 (M+H)⁺.

### Step 4: Synthesis of 2-(cyclopropylmethyl)-N-(4-fluoro-3-methylphenyl)-1,4-dimethyl-1H-pyrrole-3-carboxamide (123d)

The title compound was prepared following the procedure described in Example 104, Step 4, using 123c as the starting material. ESI-MS, m/z 301 (M+H)⁺.

### Step 5: Synthesis of ethyl 2-(5-(cyclopropylmethyl)-4-((4-fluoro-3-methylphenyl)carbamoyl)-1,3-dimethyl-1H-pyrrol-2-yl)-2-oxoacetate (123e)

The title compound was prepared following the procedure described in Example 104, Step 5, using 123d as the starting material. ESI-MS, m/z 401 (M+H)⁺.

### Step 6: Synthesis of 2-(5-(cyclopropylmethyl)-4-((4-fluoro-3-methylphenyl)carbamoyl)-1,3-dimethyl-1H-pyrrol-2-yl)-2-oxoacetic acid (123f)

The title compound was prepared following the procedure described in Example 104, Step 6, using 123e as the starting material. ESI-MS, m/z 373 (M+H)⁺.

### Step 7: Synthesis of 2-(cyclopropylmethyl)-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide (123)

The title compound was prepared following the procedure described in Example 104, Step 7, using 123f as the starting carboxylic acid. The final product was purified by reverse phase chromatography eluted with ACN and water and dried using lyophilization to afford the title products as white solids. ESI-MS, m/z 456 (M+H)⁺.

### Example 124: 2-(cyclopropylmethyl)-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 104, Step 7, using 123f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 532 (M+H)⁺.

### Example 125: 2-(cyclopropylmethyl)-N-(4-fluoro-3-methylphenyl)-5-(2-(((1s,4s)-4-hydroxy-1-methylcyclohexyl)amino)-2-oxoacetyl)-1,4-dimethyl-1H-pyrrole-3-carboxamide

The title compound was prepared following the procedure described in Example 104, Step 7, using 123f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 484 (M+H)⁺.

### Example 126: 2-(cyclopropylmethyl)-N-(4-fluoro-3-methylphenyl)-5-(2-(((1s,4s)-4-hydroxycyclohexyl)amino)-2-oxoacetyl)-1,4-dimethyl-1H-pyrrole-3-carboxamide

The title compound was prepared following the procedure described in Example 104, Step 7, using 123f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 470 (M+H)⁺.

### Example 127: 2-(cyclopropylmethyl)-N-(4-fluoro-3-methylphenyl)-5-(2-((2-hydroxy-2-methylpropyl)amino)-2-oxoacetyl)-1,4-dimethyl-1H-pyrrole-3-carboxamide

The title compound was prepared following the procedure described in Example 104, Step 7, using 123f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 444 (M+H)⁺.

### Example 128: 2-cyclopropyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide

The title compound was prepared following the procedure described in Example 104, Step 7, using 114f as the starting carboxylic acid and (1s,3s)-3-amino-3-methylcyclobutan-1-ol hydrochloride as the starting amine. The final product was purified by reverse phase chromatography eluted with ACN and water and dried using lyophilization to afford the title products as white solids. ESI-MS m/z 442 (M+H)⁺.

### Example 129: 2-cyclopropyl-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide

The title compound was prepared following the procedure described in Example 104, Step 7, using 114f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 518 (M+H)⁺.

### Example 130: 2-cyclopropyl-N-(4-fluoro-3-methylphenyl)-5-(2-((2-hydroxy-2-methylpropyl)amino)-2-oxoacetyl)-1,4-dimethyl-1H-pyrrole-3-carboxamide

The title compound was prepared following the procedure described in Example 104, Step 7, using 114f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 430 (M+H)⁺.

### Example 131: 2-cyclopentyl-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide

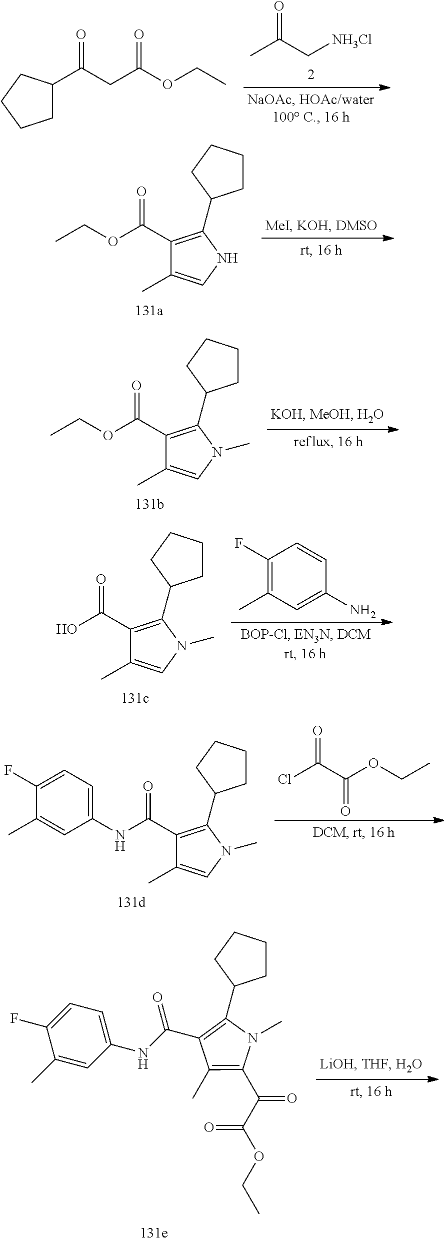

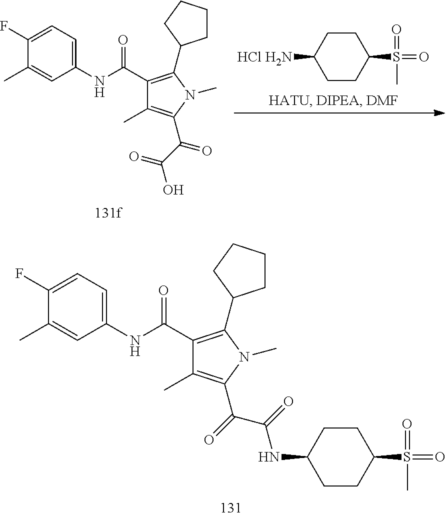

### Step 1: Synthesis of ethyl 2-cyclopentyl-4-methyl-1H-pyrrole-3-carboxylate (131a)

The title compound was prepared following the procedure described in Example 104, Step 1, using the commercially available ethyl 3-cyclopentyl-3-oxopropanoate as the starting material. ESI-MS, m/z 222 (M+H)⁺.

### Step 2: Synthesis of ethyl 2-cyclopentyl-1,4-dimethyl-1H-pyrrole-3-carboxylate (131b)

The title compound was prepared following the procedure described in Example 104, Step 2, using 131a as the starting material. ESI-MS, m/z 236 (M+H)⁺.

### Step 3: Synthesis of 2-cyclopentyl-1,4-dimethyl-1H-pyrrole-3-carboxylic acid (131c)

The title compound was prepared following the procedure described in Example 104, Step 3, using 131b as the starting material. ESI-MS, m/z 208 (M+H)⁺.

### Step 4: Synthesis of 2-cyclopentyl-N-(4-fluoro-3-methylphenyl)-1,4-dimethyl-1H-pyrrole-3-carboxamide (131d)

The title compound was prepared following the procedure described in Example 104, Step 4, using 131c as the starting material. ESI-MS, m/z 315 (M+H)⁺.

### Step 5: Synthesis of ethyl 2-(5-cyclopentyl-4-((4-fluoro-3-methylphenyl)carbamoyl)-1,3-dimethyl-1H-pyrrol-2-yl)-2-oxoacetate (131e)

The title compound was prepared following the procedure described in Example 104, Step 5, using 131d as the starting material. ESI-MS, m/z 415 (M+H)⁺.

### Step 6: Synthesis of 2-(5-cyclopentyl-4-((4-fluoro-3-methylphenyl)carbamoyl)-1,3-dimethyl-1H-pyrrol-2-yl)-2-oxoacetic acid (131f)

The title compound was prepared following the procedure described in Example 104, Step 6, using 131e as the starting material. ESI-MS, m/z 387 (M+H)⁺.

### Step 7: 2-cyclopentyl-N-(4-fluoro-3-methylphenyl)-1,4-dimethyl-5-(2-(((1s,4s)-4-(methylsulfonyl)cyclohexyl)amino)-2-oxoacetyl)-1H-pyrrole-3-carboxamide (131)

The title compound was prepared following the procedure described in Example 104, Step 7, using 131f as the starting carboxylic acid. The final product was purified by reverse phase chromatography eluted with ACN and water and dried using lyophilization to afford the title products as white solids. ESI-MS, m/z 546 (M+H)⁺.

### Example 132: 2-cyclopentyl-N-(4-fluoro-3-methylphenyl)-5-(2-(((1s,3s)-3-hydroxy-1-methylcyclobutyl)amino)-2-oxoacetyl)-1,4-dimethyl-1H-pyrrole-3-carboxamide

The title compound was prepared following the procedure described in Example 104, Step 7, using 131f as the starting carboxylic acid and (1s,3s)-3-amino-3-methylcyclobutan-1-ol hydrochloride as the starting amine. The final product was purified by reverse phase chromatography eluted with ACN and water and dried using lyophilization to afford the title products as white solids. ESI-MS m/z 470 (M+H)⁺.

### Example 133: 2-cyclopentyl-N-(4-fluoro-3-methylphenyl)-5-(2-(((1s,4s)-4-hydroxy-1-methylcyclohexyl)amino)-2-oxoacetyl)-1,4-dimethyl-1H-pyrrole-3-carboxamide

The title compound was prepared following the procedure described in Example 104, Step 7, using 131f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 498 (M+H)⁺.

### Example 134: 2-cyclopentyl-N-(4-fluoro-3-methylphenyl)-5-(2-(((1s,4s)-4-hydroxycyclohexyl)amino)-2-oxoacetyl)-1,4-dimethyl-1H-pyrrole-3-carboxamide

The title compound was prepared following the procedure described in Example 104, Step 7, using 131f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 484 (M+H)⁺.

### Example 135: 2-cyclopentyl-N-(4-fluoro-3-methylphenyl)-5-(2-((2-hydroxy-2-methylpropyl)amino)-2-oxoacetyl)-1,4-dimethyl-1H-pyrrole-3-carboxamide

The title compound was prepared following the procedure described in Example 104, Step 7, using 131f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 458 (M+H)⁺.

### Example 136: Synthesis of N-(3-cyclopropyl-4-fluorophenyl)-5-(2-(((1r,4r)-4-hydroxy-1-methylcyclohexyl)amino)-2-oxoacetyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 470.2 (M+H)⁺.

### Example 137: Synthesis of N-(4-fluorophenyl)-5-(2-(((1r,4r)-4-hydroxy-1-methylcyclohexyl)amino)-2-oxoacetyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 430.2 (M+H)⁺.

### Example 138: Synthesis of 5-(2-(tert-butylamino)-2-oxoacetyl)-N-(3-cyclopropyl-4-fluorophenyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 414.2 (M+H)⁺.

### Example 139: Synthesis of 5-(2-(tert-butylamino)-2-oxoacetyl)-N-(4-fluoro-3-(1H-1,2,4-triazol-3-yl)phenyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 441.2 (M+H)⁺.

### Example 140: Synthesis of (4-(2-(4-((3-cyano-4-fluorophenyl)carbamoyl)-1,3,5-trimethyl-1H-pyrrol-2-yl)-2-oxoacetamido)phenyl)boronic acid

The title compounds were prepared following the procedure described in Example 1. The desired product was separated and purified by reverse phase chromatography eluted with ACN and water and dried using lyophilization to afford the title products as white solids. ESI-MS m/z 463.1 (M+H)⁺.

### Example 141: Synthesis of (3-(2-(4-((3-cyano-4-fluorophenyl)carbamoyl)-1,3,5-trimethyl-1H-pyrrol-2-yl)-2-oxoacetamido)phenyl)boronic acid

The title compounds were prepared following the procedure described in Example 1. The desired product was separated and purified by reverse phase chromatography eluted with ACN and water and dried using lyophilization to afford the title products as white solids. ESI-MS m/z 463.1 (M+H)⁺.

### Example 142: N-(4-fluoro-3-methylphenyl)-5-(2-(((1R,4S)-4-hydroxy-2,2-dimethylcyclohexyl)amino)-2-oxoacetyl)-1,2,4-trimethyl-1H-pyrrole-3-carboxamide

The title compound was prepared following the procedure described in Example 1, Step 5, using 3,3-dimethyl-4-aminocyclohexanol (mixture of diastereomers) as the amine. The final product was purified by reverse phase chromatography eluted with ACN and water where it was the earlier eluting product and dried using lyophilization to afford the title products as white solids. ESI-MS, m/z 458 (M+H)⁺.

### Example 143: N-(4-fluoro-3-methylphenyl)-5-(2-(((1R,4R)-4-hydroxy-2,2-dimethylcyclohexyl)amino)-2-oxoacetyl)-1,2,4-trimethyl-1H-pyrrole-3-carboxamide

The title compound was prepared following the procedure described in Example 1, Step 5, using 3,3-dimethyl-4-aminocyclohexanol (mixture of diastereomers) as the amine. The final product was purified by reverse phase chromatography eluted with ACN and water where it was the later eluting product and dried using lyophilization to afford the title products as white solids. ESI-MS, m/z 458 (M+H)⁺.

### Example 144: 2-(((1s,4s)-4-(2-(4-((4-fluoro-3-methylphenyl)carbamoyl)-1,3,5-trimethyl-1H-pyrrol-2-yl)-2-oxoacetamido)cyclohexyl)oxy)acetic acid

To a solution of 26 (23 mg, 0.045 mmol) in THF (1 mL) and MeOH (1 mL) was added 1 N NaOH (0.14 mL). After 1 h the reaction mixture was concentrated. The final product was purified by reverse phase chromatography eluted with ACN and water and dried using lyophilization to afford the title products as white solids. ESI-MS, m/z 488 (M+H)⁺.

### Example 144: 2-(((1s,4s)-4-(2-(4-((4-fluoro-3-methylphenyl)carbamoyl)-1,3,5-trimethyl-1H-pyrrol-2-yl)-2-oxoacetamido)cyclohexyl)oxy)acetic acid

To a solution of 26 (23 mg, 0.045 mmol) in THF (1 mL) and MeOH (1 mL) was added 1 N NaOH (0.14 mL). After 1 h the reaction mixture was concentrated. The final product was purified by reverse phase chromatography eluted with ACN and water and dried using lyophilization to afford the title products as white solids. ESI-MS, m/z 488 (M+H)⁺.

### Example 145: 2-(4-(2-(4-((4-fluoro-3-methylphenyl)carbamoyl)-1,3,5-trimethyl-1H-pyrrol-2-yl)-2-oxoacetamido)piperidin-4-yl)acetic acid

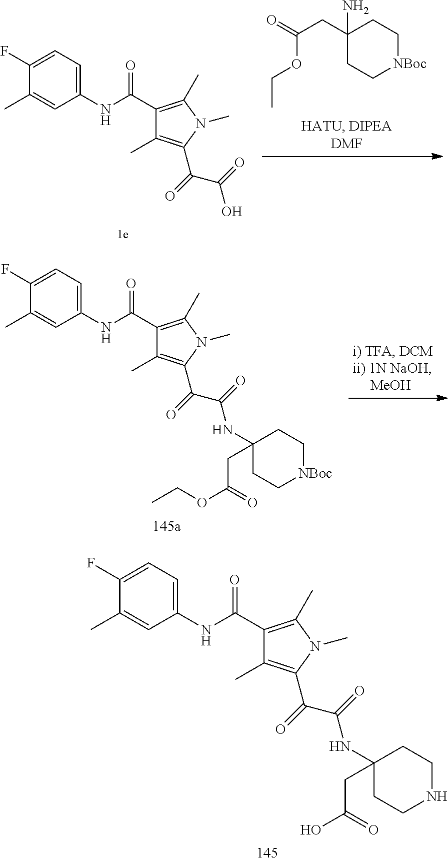 [SMILES: Cc1cc(NC(=O)c2c(C)c(C(=O)C(=O)O)n(C)c2C)ccc1F ; CCOC(=O)CC1(N)CCN(C(=O)OC(C)(C)C)CC1 ; CCOC(=O)CC1(NC(=O)C(=O)c2c(C)c(C(=O)Nc3ccc(F)c(C)c3)c(C)n2C)CCN(C(=O)OC(C)(C)C)CC1 ; Cc1cc(NC(=O)c2c(C)c(C(=O)C(=O)NC3(CC(=O)O)CCNCC3)n(C)c2C)ccc1F]

### Step 1: Synthesis of tert-butyl 4-(2-ethoxy-2-oxoethyl)-4-(2-(4-((4-fluoro-3-methylphenyl)carbamoyl)-1,3,5-trimethyl-1H-pyrrol-2-yl)-2-oxoacetamido)piperidine-1-carboxylate (145a)

The title compound was prepared following the procedure described in Example 1, Step 5, using tert-butyl 4-amino-4-(2-ethoxy-2-oxoethyl)piperidine-1-carboxylate as the amine and was used without further purification. ESI-MS, m/z 587 (M+H)⁺.

### Step 2: Synthesis of 2-(4-(2-(4-((4-fluoro-3-methylphenyl)carbamoyl)-1,3,5-trimethyl-1H-pyrrol-2-yl)-2-oxoacetamido)piperidin-4-yl)acetic acid (145)

To a solution of 145a (88 mg, 0.15 mmol) in DCM (5 mL) was added TFA (0.5 mL). After 2 h the reaction mixture was concentrated. The residue was dissolved in MeOH (5 mL) and 1 N NaOH (1 mL) was added. After 16 h the reaction mixture was concentrated. The final product was purified by reverse phase chromatography eluted with ACN and water and dried using lyophilization to afford the title products as white solids. ESI-MS, m/z 473 (M+H)⁺.

### Example 146: 2-(3-(2-(4-((4-fluoro-3-methylphenyl)carbamoyl)-1,3,5-trimethyl-1H-pyrrol-2-yl)-2-oxoacetamido)piperidin-3-yl)acetic acid

The title compound was prepared following the procedure described in Example 145, Step 1 and Step 2, using tert-butyl 2-amino-2-(2-methoxy-2-oxoethyl)piperidine-1-carboxylate as the amine. The final product was purified by reverse phase chromatography eluted with ACN and water and dried using lyophilization to afford the title products as white solids. ESI-MS, m/z 473 (M+H)⁺.

### Example 147: 5-(2-(((3R,4R)-1-acetyl-3-fluoropiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl (3R,4R)-4-amino-3-fluoropiperidine-1-carboxylate as the amine. The final product was purified by reverse phase chromatography eluted with ACN and water and dried using lyophilization to afford the title products as white solids. ESI-MS, m/z 475 (M+H)⁺.

### Example 148: (R)-5-(2-((1-acetyl-3,3-difluoropiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl 4-amino-3,3-difluoropiperidine-1-carboxylate as the amine. The final product was purified by reverse phase chromatography eluted with ACN and water and dried using lyophilization to afford the title products as white solids. ESI-MS, m/z 493 (M+H)⁺.

### Example 149: (R)-5-(2-((1-acetyl-3,3-dimethylpiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl 4-amino-3,3-dimethylpiperidine-1-carboxylate as the amine. The final product was purified by reverse phase chromatography eluted with ACN and water and dried using lyophilization to afford the title products as white solids. ESI-MS, m/z 485 (M+H)⁺.

### Example 150: N-(4-fluoro-3-methylphenyl)-5-(2-(((1s,3s)-3-hydroxy-1-methylcyclobutyl)amino)-2-oxoacetyl)-1,4-dimethyl-2-(pyridin-3-yl)-1H-pyrrole-3-carboxamide

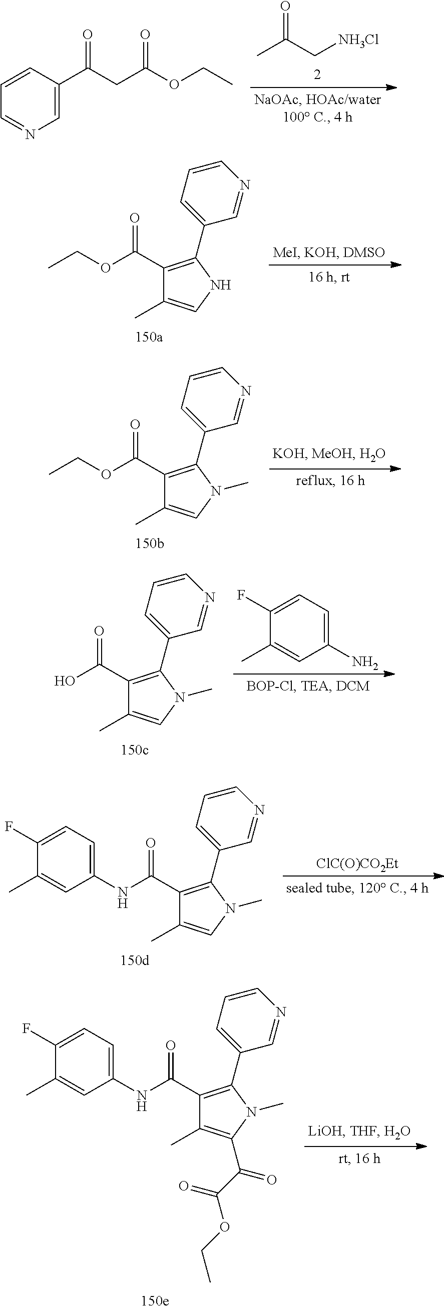

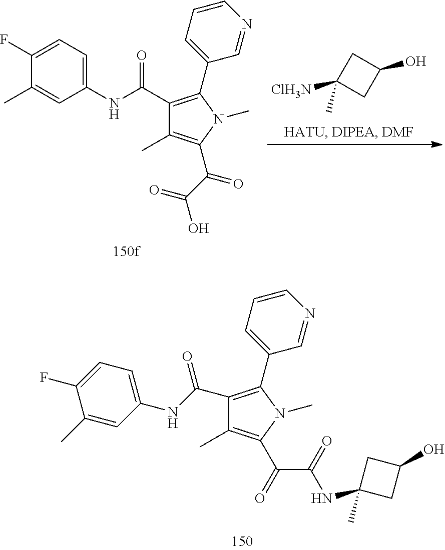

### Step 1: Synthesis of ethyl 4-methyl-2-(pyridin-3-yl)-1H-pyrrole-3-carboxylate (150a)

The title compound was prepared following the procedure described in Example 104, Step 1, using the commercially available ethyl 3-oxo-3-(pyridin-3-yl)propanoate as the starting material. ESI-MS, m/z 231 (M+H)⁺.

### Step 2: Synthesis of ethyl 1,4-dimethyl-2-(pyridin-3-yl)-1H-pyrrole-3-carboxylate (150b)

The title compound was prepared following the procedure described in Example 104, Step 2, using 150a as the starting material. ESI-MS, m/z 245 (M+H)⁺.

### Step 3: Synthesis of 1,4-dimethyl-2-(pyridin-3-yl)-1H-pyrrole-3-carboxylic acid (150c)

The title compound was prepared following the procedure described in Example 104, Step 3, using 150b as the starting material. ESI-MS, m/z 217 (M+H)⁺.

### Step 4: Synthesis of 5-(2-ethoxy-2-oxoacetyl)-1,4-dimethyl-2-(pyridin-3-yl)-1H-pyrrole-3-carboxylic acid (150d)

The title compound was prepared following the procedure described in Example 104, Step 4, using 150c as the starting material. ESI-MS, m/z 324 (M+H)⁺.

### Step 5: Synthesis of ethyl 2-(4-((4-fluoro-3-methylphenyl)carbamoyl)-1,3-dimethyl-5-(pyridin-3-yl)-1H-pyrrol-2-yl)-2-oxoacetate (150e)

A solution of 150d (635 mg, 1.96 mmol) in ethyl 2-chloro-2-oxoacetate (18 mL) was stirred in a sealed tube at 120° C. for 4 h. The reaction was concentrated and the crude product was purified by flash chromatography on silica gel (PE/EA=1:1) to give the title compound 150e as a yellow oil, 0.5 g, 60%. ESI-MS m/z 424 (M+H)⁺.

### Step 6: Synthesis of ethyl 2-(4-((4-fluoro-3-methylphenyl)carbamoyl)-1,3-dimethyl-5-(pyridin-3-yl)-1H-pyrrol-2-yl)-2-oxoacetate (150f)

The title compound was prepared following the procedure described in Example 104, Step 6, using 150e as the starting material. ESI-MS, m/z 396 (M+H)⁺.

### Step 7: Synthesis of N-(4-fluoro-3-methylphenyl)-5-(2-(((1s,3s)-3-hydroxy-1-methylcyclobutyl)amino)-2-oxoacetyl)-1,4-dimethyl-2-(pyridin-3-yl)-1H-pyrrole-3-carboxamide (150)

The title compound was prepared following the procedure described in Example 104, Step 7, using 150f as the starting carboxylic acid. The final product was purified by reverse phase chromatography eluted with ACN and water and dried using lyophilization to afford the title products as white solids. ESI-MS, m/z 479 (M+H)⁺.

### Example 151: N-(4-fluoro-3-methylphenyl)-1,4-dimethyl-5-(2-(((1s,4s)-4-(methylsulfonyl)cyclohexyl)amino)-2-oxoacetyl)-2-(pyridin-3-yl)-1H-pyrrole-3-carboxamide

The title compound was prepared following the procedure described in Example 104, Step 7, using 150f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 555 (M+H)⁺.

### Example 152: N-(4-fluoro-3-methylphenyl)-5-(2-(((1s,4s)-4-hydroxy-1-methylcyclohexyl)amino)-2-oxoacetyl)-1,4-dimethyl-2-(pyridin-3-yl)-1H-pyrrole-3-carboxamide

The title compound was prepared following the procedure described in Example 104, Step 7, using 150f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 507 (M+H)⁺.

### Example 153: N-(4-fluoro-3-methylphenyl)-5-(2-(((1s,4s)-4-hydroxycyclohexyl)amino)-2-oxoacetyl)-1,4-dimethyl-2-(pyridin-3-yl)-1H-pyrrole-3-carboxamide

The title compound was prepared following the procedure described in Example 104, Step 7, using 150f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 493 (M+H)⁺.

### Example 154: N-(4-fluoro-3-methylphenyl)-5-(2-((2-hydroxy-2-methylpropyl)amino)-2-oxoacetyl)-1,4-dimethyl-2-(pyridin-3-yl)-1H-pyrrole-3-carboxamide

The title compound was prepared following the procedure described in Example 104, Step 7, using 150f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 467 (M+H)⁺.

### Example 155: N-(4-fluoro-3-methylphenyl)-5-(2-(((1s,3s)-3-hydroxy-1-methylcyclobutyl)amino)-2-oxoacetyl)-1,4-dimethyl-2-(pyridin-2-yl)-1H-pyrrole-3-carboxamide

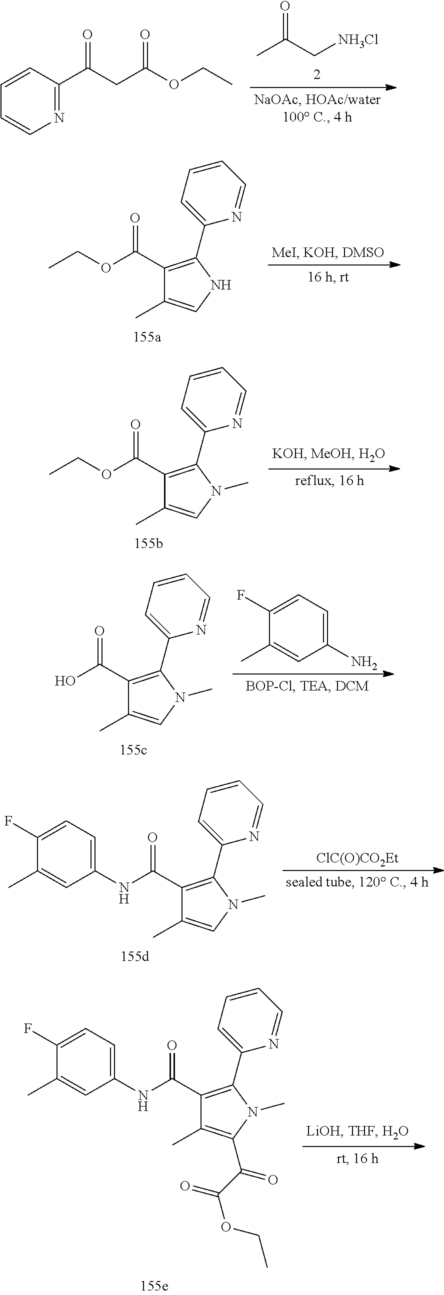

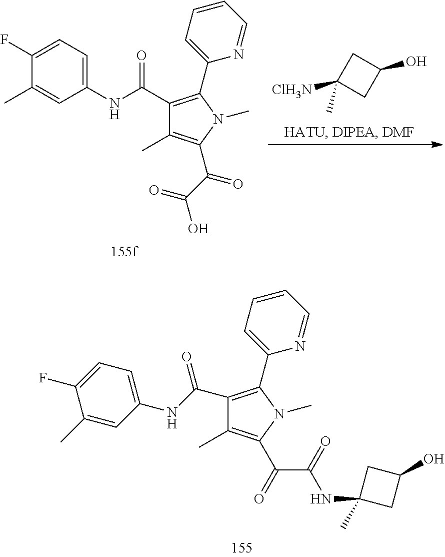

### Step 1: Synthesis of ethyl 4-methyl-2-(pyridin-2-yl)-1H-pyrrole-3-carboxylate (155a)

The title compound was prepared following the procedure described in Example 104, Step 1, using the commercially available ethyl 3-oxo-3-(pyridin-2-yl)propanoate as the starting material. ESI-MS, m/z 231 (M+H)⁺.

### Step 2: Synthesis of ethyl 1,4-dimethyl-2-(pyridin-2-yl)-1H-pyrrole-3-carboxylate (155b)

The title compound was prepared following the procedure described in Example 104, Step 2, using 155a as the starting material. ESI-MS, m/z 245 (M+H)⁺.

### Step 3: Synthesis of 1,4-dimethyl-2-(pyridin-2-yl)-1H-pyrrole-3-carboxylic acid (155c)

The title compound was prepared following the procedure described in Example 104, Step 3, using 155b as the starting material. ESI-MS, m/z 217 (M+H)⁺.

### Step 4: Synthesis of 5-(2-ethoxy-2-oxoacetyl)-1,4-dimethyl-2-(pyridin-2-yl)-1H-pyrrole-3-carboxylic acid (155d)

The title compound was prepared following the procedure described in Example 104, Step 4, using 155c as the starting material. ESI-MS, m/z 324 (M+H)⁺.

### Step 5: Synthesis of ethyl 2-(4-((4-fluoro-3-methylphenyl)carbamoyl)-1,3-dimethyl-5-(pyridin-2-yl)-1H-pyrrol-2-yl)-2-oxoacetate (155e)

The title compound was prepared following the procedure described in Example 150, Step 5, using 155d as the starting material. ESI-MS m/z 424 (M+H)⁺.

### Step 6: Synthesis of ethyl 2-(4-((4-fluoro-3-methylphenyl)carbamoyl)-1,3-dimethyl-5-(pyridin-2-yl)-1H-pyrrol-2-yl)-2-oxoacetate (155f)

The title compound was prepared following the procedure described in Example 104, Step 6, using 155e as the starting material. ESI-MS, m/z 396 (M+H)⁺.

### Step 7: Synthesis of N-(4-fluoro-3-methylphenyl)-5-(2-(((1s,3s)-3-hydroxy-1-methylcyclobutyl)amino)-2-oxoacetyl)-1,4-dimethyl-2-(pyridin-2-yl)-1H-pyrrole-3-carboxamide (150)

The title compound was prepared following the procedure described in Example 104, Step 7, using 155f as the starting carboxylic acid. The final product was purified by reverse phase chromatography eluted with ACN and water and dried using lyophilization to afford the title products as white solids. ESI-MS, m/z 479 (M+H)⁺.

### Example 156: N-(4-fluoro-3-methylphenyl)-1,4-dimethyl-5-(2-(((1s,4s)-4-(methylsulfonyl)cyclohexyl)amino)-2-oxoacetyl)-2-(pyridin-2-yl)-1H-pyrrole-3-carboxamide

The title compound was prepared following the procedure described in Example 104, Step 7, using 155f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 555 (M+H)⁺.

### Example 157: N-(4-fluoro-3-methylphenyl)-5-(2-(((1s,4s)-4-hydroxy-1-methylcyclohexyl)amino)-2-oxoacetyl)-1,4-dimethyl-2-(pyridin-2-yl)-1H-pyrrole-3-carboxamide

The title compound was prepared following the procedure described in Example 104, Step 7, using 155f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 407 (M+H)⁺.

### Example 158: N-(4-fluoro-3-methylphenyl)-5-(2-(((1s,4s)-4-hydroxycyclohexyl)amino)-2-oxoacetyl)-1,4-dimethyl-2-(pyridin-2-yl)-1H-pyrrole-3-carboxamide

The title compound was prepared following the procedure described in Example 104, Step 7, using 155f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 493 (M+H)⁺.

### Example 159: N-(4-fluoro-3-methylphenyl)-5-(2-((2-hydroxy-2-methylpropyl)amino)-2-oxoacetyl)-1,4-dimethyl-2-(pyridin-2-yl)-1H-pyrrole-3-carboxamide

The title compound was prepared following the procedure described in Example 104, Step 7, using 155f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 467 (M+H)⁺.

### Example 160: N-(4-fluoro-3-methylphenyl)-1,4-dimethyl-5-(2-(((1s,4s)-4-(methylsulfonyl)cyclohexyl)amino)-2-oxoacetyl)-2-(pyridin-4-yl)-1H-pyrrole-3-carboxamide

The title compound was prepared following the procedure described in Example 104, Step 7, using 161f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 555 (M+H)⁺.

### Example 161: N-(4-fluoro-3-methylphenyl)-5-(2-(((1s,3s)-3-hydroxy-1-methylcyclobutyl)amino)-2-oxoacetyl)-1,4-dimethyl-2-(pyridin-4-yl)-1H-pyrrole-3-carboxamide

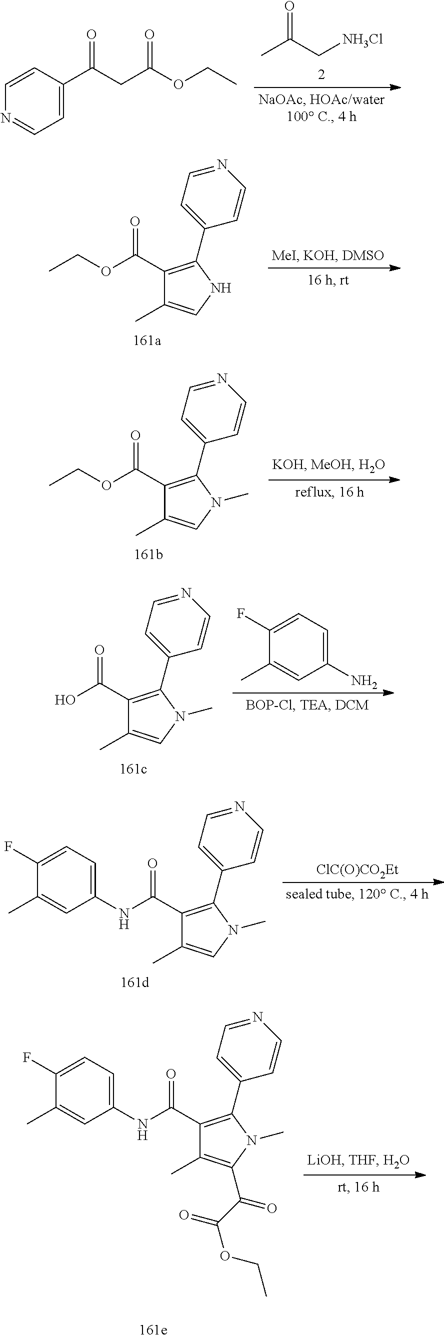

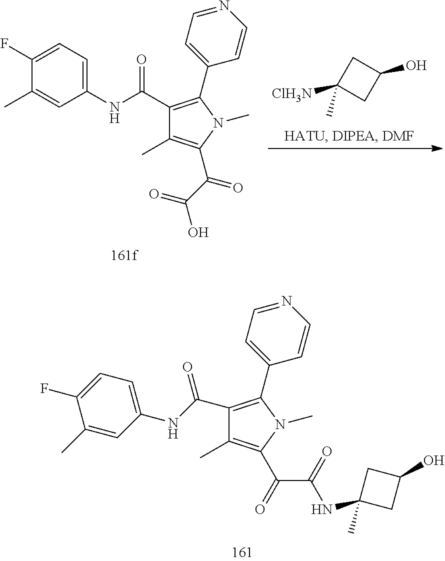

### Step 1: Synthesis of ethyl 4-methyl-2-(pyridin-4-yl)-1H-pyrrole-3-carboxylate (161a)

The title compound was prepared following the procedure described in Example 104, Step 1, using the commercially available ethyl 3-oxo-3-(pyridin-4-yl)propanoate as the starting material. ESI-MS, m/z 231 (M+H)⁺.

### Step 2: Synthesis of ethyl 1,4-dimethyl-2-(pyridin-4-yl)-1H-pyrrole-3-carboxylate (161b)

The title compound was prepared following the procedure described in Example 104, Step 2, using 161a as the starting material. ESI-MS, m/z 245 (M+H)⁺.

### Step 3: Synthesis of 1,4-dimethyl-2-(pyridin-4-yl)-1H-pyrrole-3-carboxylic acid (161c)

The title compound was prepared following the procedure described in Example 104, Step 3, using 161b as the starting material. ESI-MS, m/z 217 (M+H)⁺.

### Step 4: Synthesis of 5-(2-ethoxy-2-oxoacetyl)-1,4-dimethyl-2-(pyridin-4-yl)-1H-pyrrole-3-carboxylic acid (161d)

The title compound was prepared following the procedure described in Example 104, Step 4, using 161c as the starting material. ESI-MS, m/z 324 (M+H)⁺.

### Step 5: Synthesis of ethyl 2-(4-((4-fluoro-3-methylphenyl)carbamoyl)-1,3-dimethyl-5-(pyridin-4-yl)-1H-pyrrol-2-yl)-2-oxoacetate (161e)

The title compound was prepared following the procedure described in Example 150, Step 5, using 161d as the starting material. ESI-MS m/z 424 (M+H)⁺.

### Step 6: Synthesis of ethyl 2-(4-((4-fluoro-3-methylphenyl)carbamoyl)-1,3-dimethyl-5-(pyridin-4-yl)-1H-pyrrol-2-yl)-2-oxoacetate (161f)

The title compound was prepared following the procedure described in Example 104, Step 6, using 161e as the starting material. ESI-MS, m/z 396 (M+H)⁺.

### Step 7: Synthesis of N-(4-fluoro-3-methylphenyl)-5-(2-(((1s,3s)-3-hydroxy-1-methylcyclobutyl)amino)-2-oxoacetyl)-1,4-dimethyl-2-(pyridin-4-yl)-1H-pyrrole-3-carboxamide (161)

The title compound was prepared following the procedure described in Example 104, Step 7, using 161f as the starting carboxylic acid. The final product was purified by reverse phase chromatography eluted with ACN and water and dried using lyophilization to afford the title products as white solids. ESI-MS, m/z 479 (M+H)⁺.

### Example 162: N-(4-fluoro-3-methylphenyl)-5-(2-(((1s,4s)-4-hydroxy-1-methylcyclohexyl)amino)-2-oxoacetyl)-1,4-dimethyl-2-(pyridin-4-yl)-1H-pyrrole-3-carboxamide

The title compound was prepared following the procedure described in Example 104, Step 7, using 161f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 507 (M+H)⁺.

### Example 163: N-(4-fluoro-3-methylphenyl)-5-(2-(((1s,4s)-4-hydroxycyclohexyl)amino)-2-oxoacetyl)-1,4-dimethyl-2-(pyridin-4-yl)-1H-pyrrole-3-carboxamide

The title compound was prepared following the procedure described in Example 104, Step 7, using 161f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 493 (M+H)⁺.

### Example 164: N-(4-fluoro-3-methylphenyl)-5-(2-((2-hydroxy-2-methylpropyl)amino)-2-oxoacetyl)-1,4-dimethyl-2-(pyridin-4-yl)-1H-pyrrole-3-carboxamide

The title compound was prepared following the procedure described in Example 104, Step 7, using 161f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 467 (M+H)⁺.

### Example 165: 5-(2-(((2R,4R)-1-acetyl-2-methylpiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl (2R,4R)-4-amino-2-methylpiperidine-1-carboxylate as the amine. The final product was purified by reverse phase chromatography eluted with ACN and water and dried using lyophilization to afford the title products as white solids. ESI-MS, m/z 471 (M+H)⁺.

### Example 166: 5-(2-(((2S,4S)-1-acetyl-2-methylpiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl (2S,4S)-4-amino-2-methylpiperidine-1-carboxylate as the amine. The final product was purified by reverse phase chromatography eluted with ACN and water and dried using lyophilization to afford the title products as white solids. ESI-MS, m/z 471 (M+H)⁺.

### Example 167: 5-(2-(((3R,4R)-1-acetyl-3-hydroxypiperidin-4-yl)amino)-2-oxoacetyl)-N-(4-fluoro-3-methylphenyl)-1,2,4-trimethyl-1H-pyrrole-3-carboxamide

The title compound was prepared following the procedure described in Example 78, Step 1 and Step 2, using tert-butyl (3R,4R)-4-amino-3-hydroxypiperidine-1-carboxylate as the amine. The final product was purified by reverse phase chromatography eluted with ACN and water and dried using lyophilization to afford the title products as white solids. ESI-MS, m/z 473 (M+H)⁺.

### Example 168: Synthesis of N-(4-fluoro-3,5-dimethylphenyl)-5-(2-(((1r,4r)-4-hydroxy-1-methylcyclohexyl)amino)-2-oxoacetyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 458.2 (M+H)⁺.

### Example 169: Synthesis of N-(3-chloro-5-methylphenyl)-5-(2-(((1r,4r)-4-hydroxy-1-methylcyclohexyl)amino)-2-oxoacetyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 460.9 (M+H)⁺.

### Example 170: N-(3-cyanophenyl)-5-(2-(((1r,4r)-4-hydroxy-1-methylcyclohexyl)amino)-2-oxoacetyl)-1,2,4-trimethyl-1H-pyrrole-3-carboxamide

The title compounds were prepared following the procedure described in Example 1. The desired product was separated and purified by reverse phase chromatography eluted with ACN and water and dried using lyophilization to afford the title products as white solids. ESI-MS m/z 437.2 (M+H)⁺.

### Example 171: (2S,4R)-4-(2-(4-((4-fluoro-3-methylphenyl)carbamoyl)-1,3,5-trimethyl-1H-pyrrol-2-yl)-2-oxoacetamido)-1-methylpyrrolidine-2-carboxylic acid

To a solution of 1e (50 mg, 0.15 mmol), methyl (2S,4R)-4-amino-1-methylpyrrolidine-2-carboxylate bishydrochloride (39 mg, 0.17 mmol) and HATU (68 mg, 0.18 mmol) in DMF (1 mL) was added DIPEA (78 mg, 0.6 mmol). After 2 h the reaction mixture was diluted with NaHCO₃ (sat) and extracted with EtOAc. The combined organic layers were washed with NaHCO₃ (sat), and brine, then concentrated. The crude residue was dissolved in MeOH (3 mL) and 1 N NaOH (0.5 mL) was added. After 1 h the reaction mixture was concentrated. The desired product was separated and purified by reverse phase chromatography eluted with ACN and water and dried using lyophilization to afford the title products as white solids. ESI-MS m/z 459 (M+H)⁺.

### Example 172: 2-(3-(2-(4-((4-fluoro-3-methylphenyl)carbamoyl)-1,3,5-trimethyl-1H-pyrrol-2-yl)-2-oxoacetamido)piperidin-1-yl)acetic acid

The title compounds were prepared following the procedure described in Example 171. The desired product was separated and purified by reverse phase chromatography eluted with ACN and water and dried using lyophilization to afford the title products as white solids. ESI-MS m/z 473 (M+H)⁺.

### Example 173: (2S,4R)-4-(2-(4-((4-fluoro-3-methylphenyl)carbamoyl)-1,3,5-trimethyl-1H-pyrrol-2-yl)-2-oxoacetamido)pyrrolidine-2-carboxylic acid

To a solution of 1e (50 mg, 0.15 mmol), 1-(tert-butyl) 2-methyl (2S,4R)-4-aminopyrrolidine-1,2-dicarboxylate (42 mg, 0.17 mmol) and HATU (68 mg, 0.18 mmol) in DMF (1 mL) was added DIPEA (78 mg, 0.6 mmol). After 2 h the reaction mixture was diluted with 1 N HCl and extracted with EtOAc. The combined organic layers were washed with 1 N HCl, NaHCO₃ (sat), and brine, then concentrated. The crude residue was dissolved in DCM (5 mL) and TFA (1 mL) was added. After 1 h the reaction mixture was concentrated. The crude residue was dissolved in MeOH (5 mL) and 1 N NaOH (1 mL) was added. After 2 h the reaction mixture was concentrated. The desired product was separated and purified by reverse phase chromatography eluted with ACN and water and dried using lyophilization to afford the title products as white solids. ESI-MS m/z 445 (M+H)⁺.

### Example 174: N-(4-fluoro-3-methylphenyl)-5-(2-(((1s,3s)-3-hydroxy-1-methylcyclobutyl)amino)-2-oxoacetyl)-1,4-dimethyl-2-(thiazol-2-yl)-1H-pyrrole-3-carboxamide

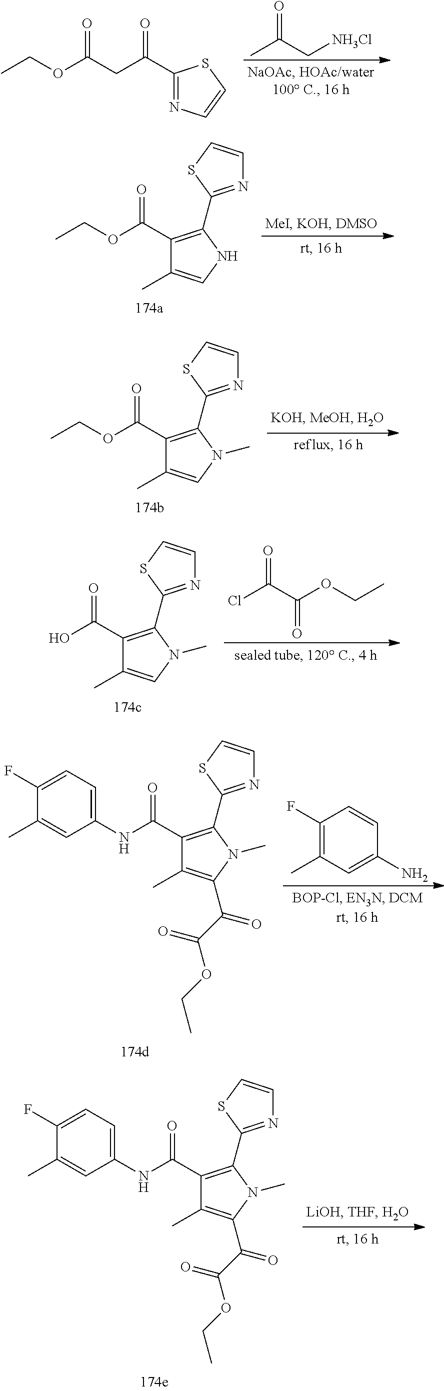

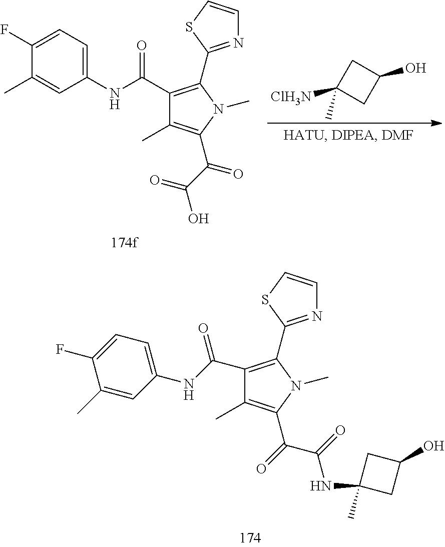

### Step 1: Synthesis of ethyl 4-methyl-2-(thiazol-2-yl)-1H-pyrrole-3-carboxylate (174a)

The title compound was prepared following the procedure described in Example 104, Step 1, using the commercially available ethyl 3-oxo-3-(thiazol-2-yl)propanoate as the starting material. ESI-MS, m/z 237 (M+H)⁺.

### Step 2: Synthesis of ethyl 1,4-dimethyl-2-(thiazol-2-yl)-1H-pyrrole-3-carboxylate (174b)

The title compound was prepared following the procedure described in Example 104, Step 2, using 174a as the starting material. ESI-MS, m/z 251 (M+H)⁺.

### Step 3: Synthesis of 1,4-dimethyl-2-(thiazol-2-yl)-1H-pyrrole-3-carboxylic acid (174c)

The title compound was prepared following the procedure described in Example 104, Step 3, using 174b as the starting material. ESI-MS, m/z 223 (M+H)⁺.

### Step 4: Synthesis of 5-(2-ethoxy-2-oxoacetyl)-1,4-dimethyl-2-(thiazol-2-yl)-1H-pyrrole-3-carboxylic acid (174d)

The title compound was prepared following the procedure described in Example 150, Step 5, using 174c as the starting material. ESI-MS, m/z 323 (M+H)⁺.

### Step 5: Synthesis of ethyl 2-(4-((4-fluoro-3-methylphenyl)carbamoyl)-1,3-dimethyl-5-(thiazol-2-yl)-1H-pyrrol-2-yl)-2-oxoacetate (174e)

The title compound was prepared following the procedure described in Example 104, Step 4, using 174d as the starting material. ESI-MS, m/z 430 (M+H)⁺.

### Step 6: Synthesis of 2-(4-((4-fluoro-3-methylphenyl)carbamoyl)-1,3-dimethyl-5-(thiazol-2-yl)-1H-pyrrol-2-yl)-2-oxoacetic acid (174f)

The title compound was prepared following the procedure described in Example 104, Step 6, using 174e as the starting material. ESI-MS, m/z 402 (M+H)⁺.

### Step 7: Synthesis of N-(4-fluoro-3-methylphenyl)-5-(2-(((1s,3s)-3-hydroxy-1-methylcyclobutyl)amino)-2-oxoacetyl)-1,4-dimethyl-2-(thiazol-2-yl)-1H-pyrrole-3-carboxamide (174)

The title compound was prepared following the procedure described in Example 104, Step 7, using 174f as the starting carboxylic acid. The final product was purified by reverse phase chromatography eluted with ACN and water and dried using lyophilization to afford the title products as white solids. ESI-MS, m/z 485 (M+H)⁺.

### Example 175: N-(4-fluoro-3-methylphenyl)-1,4-dimethyl-5-(2-(((1s,4s)-4-(methylsulfonyl)cyclohexyl)amino)-2-oxoacetyl)-2-(thiazol-2-yl)-1H-pyrrole-3-carboxamide

The title compound was prepared following the procedure described in Example 104, Step 7, using 174f as the starting carboxylic acid and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 561 (M+H)⁺.

### Example 176: N-(4-fluoro-3-methylphenyl)-5-(2-(((1s,4s)-4-hydroxy-1-methylcyclohexyl)amino)-2-oxoacetyl)-1,4-dimethyl-2-(thiazol-2-yl)-1H-pyrrole-3-carboxamide

The title compound was prepared following the procedure described in Example 104, Step 7, using 174f as the starting carboxylic acid and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 513 (M+H)⁺.

### Example 177: N-(4-fluoro-3-methylphenyl)-5-(2-(((1s,4s)-4-hydroxycyclohexyl)amino)-2-oxoacetyl)-1,4-dimethyl-2-(thiazol-2-yl)-1H-pyrrole-3-carboxamide

The title compound was prepared following the procedure described in Example 104, Step 7, using 174f as the starting carboxylic acid and (1s,4s)-4-aminocyclohexan-1-ol hydrochloride as the starting amine. The final product was purified by reverse phase chromatography eluted with ACN and water and dried using lyophilization to afford the title products as white solids. ESI-MS m/z 499 (M+H)⁺.

### Example 178: N-(4-fluoro-3-methylphenyl)-5-(2-((2-hydroxy-2-methylpropyl)amino)-2-oxoacetyl)-1,4-dimethyl-2-(thiazol-2-yl)-1H-pyrrole-3-carboxamide

The title compound was prepared following the procedure described in Example 104, Step 7, using 174f as the starting carboxylic acid and 1-amino-2-methylpropan-2-ol as the starting amine. The final product was purified by reverse phase chromatography eluted with ACN and water and dried using lyophilization to afford the title products as white solids. ESI-MS m/z 473 (M+H)⁺.

### Example 179: (2S,4S)-4-(2-(4-((4-fluoro-3-methylphenyl)carbamoyl)-1,3,5-trimethyl-1H-pyrrol-2-yl)-2-oxoacetamido)pyrrolidine-2-carboxylic acid

The title compound was prepared following the procedure described in Example 173 using 1-(tert-butyl) 2-methyl (2S,4S)-4-aminopyrrolidine-1,2-dicarboxylate as the starting amine. The final product was purified by reverse phase chromatography eluted with ACN and water and dried using lyophilization to afford the title products as white solids. ESI-MS m/z 459 (M+H)⁺.

### Example 180: (2S,4R)-4-(2-(4-((4-fluoro-3-methylphenyl)carbamoyl)-1,3,5-trimethyl-1H-pyrrol-2-yl)-2-oxoacetamido)piperidine-2-carboxylic acid

The title compound was prepared following the procedure described in Example 173 using 1-(tert-butyl) 2-methyl (2S,4R)-4-aminopiperidine-1,2-dicarboxylates the starting amine. The final product was purified by reverse phase chromatography eluted with ACN and water and dried using lyophilization to afford the title products as white solids. ESI-MS m/z 473 (M+H)⁺.

### Example 181: N-(4-fluoro-3-methylphenyl)-5-(2-(((1s,3s)-3-hydroxy-1-methylcyclobutyl)amino)-2-oxoacetyl)-1,4-dimethyl-2-(thiophen-3-yl)-1H-pyrrole-3-carboxamide

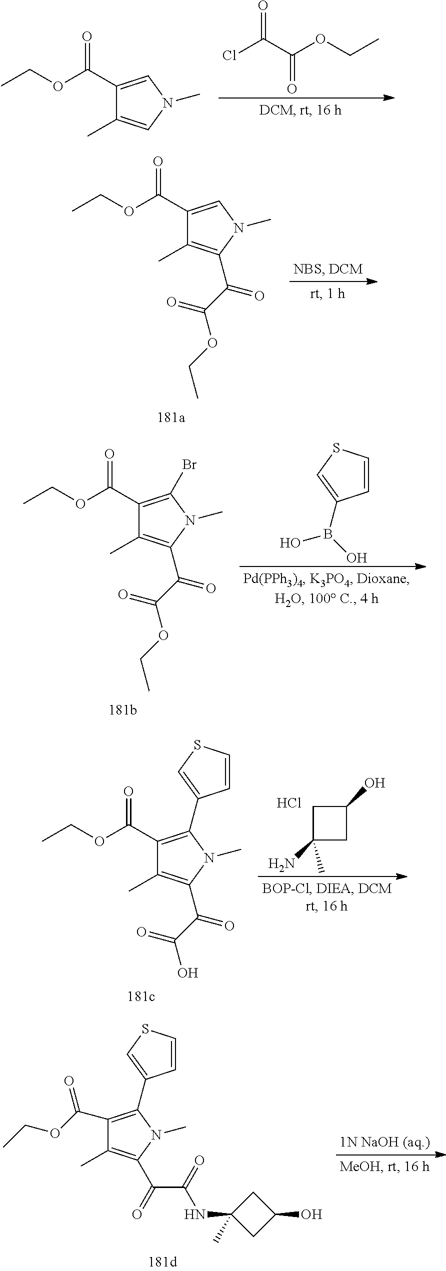

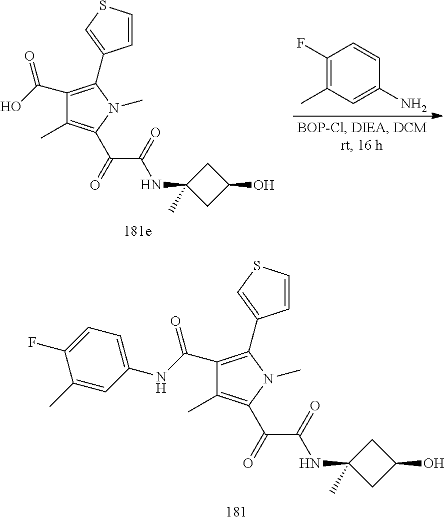

### Step 1: Synthesis of ethyl 5-(2-ethoxy-2-oxoacetyl)-1,4-dimethyl-1H-pyrrole-3-carboxylate (181a)

To a solution of ethyl 1,4-dimethyl-1H-pyrrole-3-carboxylate (6 g, 36 mmol) in DCM (100 mL) at 0° C. was added ethyl 2-chloro-2-oxoacetate (7.37 g, 54 mmol). After 1 h the reaction mixture was warmed to room temperature and stirred for an additional 16 h. The reaction mixture was diluted with water (100 mL) and extracted with DCM (50 mL×3). The organic extracts were combined, washed with water, brine, dried over Na₂SO₄, and concentrated in vacuo to give the crude product. The crude product was purified by flash chromatography on silica gel (PE/EA=3:1) to give the title compound 181a as a yellow oil, 9.3 g, 96.9%. ESI-MS m/z 268 (M+H)⁺.

### Step 2: Synthesis of ethyl 2-bromo-5-(2-ethoxy-2-oxoacetyl)-1,4-dimethyl-1H-pyrrole-3-carboxylate (181b)

To a solution of 181a (9.3 g, 34.8 mmol) in DCM (100 mL) was added NBS (12.4 g, 69.6 mmol). The resulting solution was stirred at rt for 1 h and diluted with H₂O (100 mL), extracted with DCM (50 mL×3). The organic extracts were combined, washed with water, brine, dried over Na₂SO₄, and concentrated in vacuo to give the crude product. The crude product was purified by flash chromatography on silica gel (PE/EA=3:1) to give the title compound 181b as a yellow oil, 10.4 g, 86.5%. ESI-MS m/z 346 (M+H)⁺.

### Step 3: Synthesis of 2-(4-(ethoxycarbonyl)-1,3-dimethyl-5-(thiophen-3-yl)-1H-pyrrol-2-yl)-2-oxoacetic acid (181c)

To a solution of 181b (1.2 g, 3.47 mmol), thiophen-3-ylboronic acid (890 mg, 6.94 mmol) in dioxane (12 mL) and H₂O (6 mL) was added Pd(PPh₃)₄ (803 mg, 0.694 mmol), K₃PO₄ (2.95 g, 13.91 mmol). The reaction solution was stirred at 100° C. for 4 h under N₂. The resulting mixture was concentrated under vacuum. The residue was purified by flash chromatography on silica gel (MeOH/DCM=1:20) to give the title compound 181c (950 mg, 85%) as a yellow oil. ESI-MS m/z 322 (M+H)⁺.

### Step 4: Synthesis of ethyl 5-(2-(((1s,3s)-3-hydroxy-1-methylcyclobutyl)amino)-2-oxoacetyl)-1,4-dimethyl-2-(thiophen-3-yl)-1H-pyrrole-3-carboxylate (181d)

To a solution of 181c (450 mg, 1.4 mmol), DIPEA (542 mg, 4.2 mmol) and BOP-Cl (534 mg, 2.1 mmol) in DCM (10 mL) at ambient temperature was added (1s,3s)-3-amino-3-methylcyclobutan-1-ol hydrochloride (288 mg, 2.1 mmol). The reaction mixture was stirred at room temperature for 16 h. The reaction mixture was diluted with water (50 mL) and extracted with EtOAc (30 mL×3). The organic extracts were combined, washed with water, brine, dried over Na₂SO₄, and concentrated in vacuo to give the crude product. The crude product was purified by flash chromatography on silica gel (PE/EA=5:1) to give the title compound 181d as a yellow solid, 300 mg, 53%. ESI-MS m/z 405 (M+H)⁺.

### Step 5: Synthesis of 5-(2-(((1s,3s)-3-hydroxy-1-methylcyclobutyl)amino)-2-oxoacetyl)-1,4-dimethyl-2-(thiophen-3-yl)-1H-pyrrole-3-carboxylic acid (181e)

To a solution of 181d (300 mg, 0.74 mmol) in MeOH (10 mL) and 1N NaOH (15 mL, 15 mmol) and the reaction mixture was stirred at rt for 16 h. The resulting mixture was concentrated under vacuum then acidified with 1 N HCl (aq) to pH=2-3. The mixture was extracted with DCM (50 mL×3). The organic layer was washed with brine, dried over Na₂SO₄, concentrated under vacuum to give the title compound 181e as a yellow solid, 0.2 g, yield: 71.6%. ESI-MS m/z 377 (M+H)⁺.

### Step 7: Synthesis of N-(4-fluoro-3-methylphenyl)-5-(2-(((1s,3s)-3-hydroxy-1-methylcyclobutyl)amino)-2-oxoacetyl)-1,4-dimethyl-2-(thiophen-3-yl)-1H-pyrrole-3-carboxamide (181)

To a solution of 181e (200 mg, 0.532 mmol), DIPEA (206 mg, 1.6 mmol) and BOP-Cl (203 mg, 0.8 mmol) in DCM (5 mL) at ambient temperature was added 4-fluoro-3-methylaniline (100 mg, 0.8 mmol). The reaction mixture was stirred at room temperature for 16 h. The reaction mixture was diluted with water (50 mL) and extracted with DCM (50 mL×3). The organic extracts were combined, washed with water, brine, dried over Na₂SO₄, and concentrated in vacuo to give the crude product. The crude product was purified by prep-HPLC to give the title compound 181 as a white solid, 160 mg, 62.3%. ESI-MS m/z 484 (M+H)⁺

### Example 182: N-(4-fluoro-3-methylphenyl)-1,4-dimethyl-5-(2-(((1s,4s)-4-(methylsulfonyl)cyclohexyl)amino)-2-oxoacetyl)-2-(thiophen-3-yl)-1H-pyrrole-3-carboxamide

The title compound was prepared following the procedure described in Example 181, Step 4,5,6, using 181c and (1S,4S)-4-(methylsulfonyl)cyclohexan-1-amine hydrochloride as the starting amine. The final product was purified by reverse phase chromatography eluted with ACN and water and dried using lyophilization to afford the title products as white solids. ESI-MS m/z 560 (M+H)⁺.

### Example 183: N-(4-fluoro-3-methylphenyl)-5-(2-(((1s,4s)-4-hydroxy-1-methylcyclohexyl)amino)-2-oxoacetyl)-1,4-dimethyl-2-(thiophen-3-yl)-1H-pyrrole-3-carboxamide

The title compound was prepared following the procedure described in Example 104, Step 4,5,6, using 181c and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 512 (M+H)⁺.

### Example 184: N-(4-fluoro-3-methylphenyl)-5-(2-(((1s,4s)-4-hydroxycyclohexyl)amino)-2-oxoacetyl)-1,4-dimethyl-2-(thiophen-3-yl)-1H-pyrrole-3-carboxamide

The title compound was prepared following the procedure described in Example 104, Step 4,5,6, using 181c and (1s,4s)-4-amino-4-methylcyclohexan-1-ol hydrochloride as the starting amine. The final product was purified by reverse phase chromatography eluted with ACN and water and dried using lyophilization to afford the title products as white solids. ESI-MS m/z 498 (M+H)⁺.

### Example 185: N-(4-fluoro-3-methylphenyl)-5-(2-((2-hydroxy-2-methylpropyl)amino)-2-oxoacetyl)-1,4-dimethyl-2-(thiophen-3-yl)-1H-pyrrole-3-carboxamide

The title compound was prepared following the procedure described in Example 104, step 4,5,6, using 181c and 1-amino-2-methylpropan-2-ol as the starting amine. The final product was purified by reverse phase chromatography eluted with ACN and water and dried using lyophilization to afford the title products as white solids. ESI-MS m/z 472 (M+H)⁺.

### Example 186: 5-(2-(ethyl(isopropyl)amino)-2-oxoacetyl)-N-(4-fluoro-3-methylphenyl)-1,2,4-trimethyl-1H-pyrrole-3-carboxamide

The title compound was prepared following the procedure described in Example 1, Step 5, using N-ethylpropan-2-amine as the starting amine. The final product was purified by reverse phase chromatography eluted with ACN and water and dried using lyophilization to afford the title products as white solids. ESI-MS m/z 402 (M+H)⁺.

### Example 187: N-(4-fluoro-3-methylphenyl)-1,2,4-trimethyl-5-(2-morpholino-2-oxoacetyl)-1H-pyrrole-3-carboxamide

The title compound was prepared following the procedure described in Example 1, Step 5, using morpholine as the starting amine. The final product was purified by reverse phase chromatography eluted with ACN and water and dried using lyophilization to afford the title products as white solids. ESI-MS m/z 402 (M+H)⁺.

### Example 188: N-(4-fluoro-3-methylphenyl)-1,2,4-trimethyl-5-(2-oxo-2-(pentan-3-ylamino)acetyl)-1H-pyrrole-3-carboxamide

The title compound was prepared following the procedure described in Example 1, Step 5, using 3-pentylamine as the starting amine. The final product was purified by reverse phase chromatography eluted with ACN and water and dried using lyophilization to afford the title products as white solids. ESI-MS m/z 402 (M+H)⁺.

### Example I: Oral Composition of a Compounds of Formula (I), or a Pharmaceutically Acceptable Salt, Solvate, or Stereoisomer Thereof

To prepare a pharmaceutical composition for oral delivery, 400 mg of compound described herein, or a pharmaceutically acceptable salt, solvate, or stereoisomer thereof and the following ingredients are mixed intimately and pressed into single scored tablets.

Tablet Formulation

| Ingredient | Quantity per tablet (mg) |
|---|---|
| compound | 400 |
| cornstarch | 50 |
| croscarmellose sodium | 25 |
| lactose | 120 |
| magnesium stearate | 5 |

The following ingredients are mixed intimately and loaded into a hard-shell gelatin capsule.

Capsule Formulation

| Ingredient | Quantity per capsule (mg) |
|---|---|
| compound | 200 |
| lactose spray dried | 148 |
| magnesium stearate | 2 |

### Example II: In Vitro Antiviral Assays

The anti-HBV activity of the Capsid Assembly Modulators (CAMs) was evaluated in a cell based assay utilizing the human hepatoma cell line HepAD38 (Ladner, S K., et al., 1998). HepAD38 cells were derived from the parental line, HepG2, that were stably transfected with a construct containing an HBV genome (genotype D, serotype ayw) under the control of a tetracycline repressible CMV promoter. Upon removal of tetracycline, viral pre-genomic RNA (pgRNA) and mRNAs are expressed and infectious viral particles are assembled and secreted into the culture medium providing a reliable, robust system to measure multiple steps of the HBV life cycle. Disruption of capsid formation results in reduced levels of DNA-containing virus particles that are released into the culture supernatant. To quantify the effect of CAMs on HBV replication, we developed a sensitive QPCR-based assay that measures extracellular HBV DNA levels upon treatment of HepAD38 cells with various concentrations of test compounds.

HepAD38 cells were maintained in DMEM/F12 medium containing 10% FBS, 400 μg/mL G418 and 0.3 μg/mL tetracycline (tet+ media) to maintain repression of HBV replication. To evaluate each compound, HepAD38 cells were seeded into 24-well collagen coated culture plates (Corning BioCoat) at a density of 200,000 cells per well in 1 mL of medium without tetracycline (tet-media) and allowed to adhere overnight at 37° C., 5% CO₂ in a humidified incubator. The following day, media was refreshed and a dose range of each compound was prepared by performing 1 log₁₀ serial dilutions in 100% DMSO at 200× the desired assay concentration. Dilutions were then added to the cells resulting in a final dose range of 1 μM to 10 pM and the plates were returned to the incubator. Following 7 days of incubation, culture supernatants were harvested and HBV DNA levels were evaluated by QPCR and compared to the vehicle treated control wells (i.e. DMSO alone).

To quantify HBV DNA levels, cell culture supernatants were diluted 1:10 in sterile, nuclease-free water (Gibco). The diluted supernatants were subsequently added to a PCR master mix containing 1× Roche Light Cycler Master Mix, 0.5 μM forward primer, 0.5 μM reverse primer (Fwd: 5′-TTGGTGTCTTTCGGAGTGTG (SEQ ID NO 1); Rev: 5′-AGGGGCATTTGGTGGTCTAT (SEQ ID NO 2)), 0.2 μM Roche Universal Probe Library Probe 25. The volume was brought to 20 μL with nuclease-free water and amplification of the HBV target sequence was performed using a Roche LightCycler 480 QPCR instrument. PCR extended out to 45 cycles with each cycle consisting of a denaturation step at 95° C. for 10 sec., followed by an annealing step at 60° C. for 10 sec. and a brief extension step at 72° C. for 1 sec.

Extracellular HBV DNA levels, expressed in copies/mL, were determined by comparison to a standard curve (10²-10⁹ copies/mL) using the Roche LightCycler analysis software. These values were subsequently converted to percent inhibition of HBV replication by dividing the HBV DNA levels in the experimental samples with those obtained from the vehicle control (˜1-2×105 copies/mL). Potency, expressed as an EC₅₀ (the effective concentration required to inhibit 50% of HBV replication), was calculated from the dose-response curve using a 4-parameter non-linear regression analysis (GraphPad Prism). The nucleoside analog inhibitor entecavir was used as a positive control to validate each assay run. The EC₅₀ value of entecavir in the HepAD38 assay was 0.5 nM, as previously reported in the literature.

Table 2 summarizes the antiviral activity of the exemplary compounds. A: EC₅₀>1 μM; B: EC₅₀ values between 0.5 μM and 1 μM, inclusive; C: EC₅₀ values between 0.05 μM and 0.499 μM, inclusive; D: EC₅₀ values<0.05 μM. NT=not tested. NA=not applicable.

TABLE 2
Summary of anti-HBV replication in HepAD38 cells.

| | Anti- | | Anti- | | Anti- | | Anti- | | Anti- |
|---|---|---|---|---|---|---|---|---|---|
| | HBV | | HBV | | HBV | | HBV | | HBV |
| Ex. | EC₅₀ | Ex. | EC₅₀ | Ex. | EC₅₀ | Ex. | EC₅₀ | Ex. | EC₅₀ |
| 1 | D | 2 | D | 3 | D | 4 | D | 5 | D |
| 6 | D | 7 | D | 8 | D | 9 | D | 10 | D |
| 11 | D | 12 | C | 13 | D | 14 | D | 15 | D |
| 16 | D | 17 | D | 18 | D | 19 | D | 20 | D |
| 21 | D | 22 | D | 23 | D | 24 | D | 25 | D |
| 26 | A | 27 | B | 28 | A | 29 | A | 30 | A |
| 31 | D | 32 | C | 33 | D | 39 | D | 35 | B |
| 36 | D | 37 | C | 38 | D | 44 | D | 40 | D |
| 41 | D | 42 | D | 43 | D | 49 | C | 45 | B |
| 46 | B | 47 | A | 48 | A | 54 | B | 50 | D |
| 51 | D | 52 | C | 53 | D | 59 | D | 55 | A |
| 56 | B | 57 | A | 58 | A | 64 | A | 60 | D |
| 61 | B | 62 | A | 63 | A | 69 | A | 65 | A |
| 66 | D | 67 | D | 68 | D | 74 | D | 75 | D |
| 76 | D | 72 | D | 73 | D | 79 | D | 80 | D |
| 81 | D | 77 | C | 78 | D | 85 | D | 86 | D |
| 87 | D | 82 | D | 83 | D | 90 | D | 91 | C |
| 92 | C | 88 | B | 89 | D | 95 | A | 96 | A |
| 97 | B | 93 | A | 94 | B | 100 | D | 101 | D |
| 102 | D | 98 | C | 99 | A | 105 | D | 106 | D |
| 107 | D | 103 | B | 104 | D | 110 | B | 111 | D |
| 112 | C | 108 | D | 109 | C | 115 | D | 116 | B |
| 117 | A | 113 | C | 114 | D | 118 | B | 119 | B |
| 120 | A | 121 | A | 122 | D | 123 | D | 124 | C |
| 125 | D | 126 | D | 127 | D | 128 | D | 129 | D |
| 130 | D | 131 | A | 132 | C | 133 | D | 134 | C |
| 135 | C | 136 | D | 137 | D | 138 | D | 139 | A |
| 140 | D | 141 | C | 142 | D | 143 | D | 144 | C |
| 145 | C | 146 | A | 147 | D | 148 | D | 149 | D |
| 150 | A | 151 | A | 152 | A | 153 | A | 154 | C |
| 155 | A | 156 | A | 157 | C | 158 | A | 159 | A |
| 160 | A | 161 | A | 162 | A | 163 | A | 164 | A |
| 165 | D | 166 | C | 167 | B | 168 | D | 169 | D |
| 170 | D | 171 | A | 172 | A | 173 | B | 174 | B |
| 175 | A | 176 | C | 177 | A | 178 | A | 179 | A |
| 180 | A | 181 | B | 182 | A | 183 | C | 184 | A |
| 185 | B | 186 | B | 187 | B | 188 | D | | |

### Example III: In Vitro Cytotoxicity Assays

To evaluate antiviral selectivity, the cytotoxic activity of each compound was determined using a standard cell viability assay performed on the parental HepG2 cell line. Cell viability was determined by measuring the conversion of 3-(4,5-dimethylthiazol-2-yl)-2,5-diphenyltetrazolium bromide (MTT) to the insoluble formazan salt crystal that occurs in live cells. Briefly, HepG2 cells were seeded in 96-well plates at a density of 20,000 cells per well in EMEM+1000 FBS (complete growth medium) and allowed to adhere overnight in a 37° C., 500 CO₂ humidified incubator. The next day, test agents were prepared by performing 8 half-log₁₀ serial dilutions in 100% DMSO at 200× the final desired concentration in the assay. Compounds were tested over a range of concentrations from 30 μM to 1.0 nM in the assay. HepG2 cells were incubated in the presence of various concentrations of CAMs for 7 days in a 37° C., 500 CO₂ humidified incubator. At the completion of the 7-day incubation period, MTT reagent was added to each well and the mixture was incubated for an additional 3-4 hours. At the completion of the incubation period, all wells were aspirated to remove the culture medium. The formazan crystals were solubilized from the cell monolayers with 100% DMSO. Plates were briefly mixed on an orbital shaker and absorbance was measured at 492 nm using a Perkin-Elmer EnVision multi-label plate reader. All absorbance values were converted to a percentage of the signal obtained from the vehicle treated controls. Absorbance values at 492 nm are directly proportional to the number of viable cells present in the sample. A CC₅₀ value (cytotoxic concentration that results in loss of 50% cell viability) was calculated from the dose-response curve by 4-parameter, non-linear regression analysis using the GraphPad Prism software. The positive control compound, staurosporine, reduced the viability of HepG2 cells in a dose-dependent manner (CC₅₀=100 nM).

Table 3 summarizes the cytotoxicity assay data in the hepatocyte cell line HepG2 for the example compounds. A: CC₅₀>30 μM; B: CC₅₀ values between 5 μM and 30 μM, inclusive; C: CC₅₀ values between 0.5 μM and 4.99 μM, inclusive; D: CC₅₀ values<0.5 μM. NT=not tested. NA=not applicable.

TABLE 3
Summary of cytotoxicity results in HepG2 cells for example compounds.

| | HepG2 | | HepG2 | | HepG2 | | HepG2 | | HepG2 |
|---|---|---|---|---|---|---|---|---|---|
| Ex. | CC₅₀ | Ex. | CC₅₀ | Ex. | CC₅₀ | Ex. | CC₅₀ | Ex. | CC₅₀ |
| 1 | A | 2 | A | 3 | A | 4 | A | 5 | A |
| 6 | A | 7 | A | 8 | B | 9 | A | 10 | B |
| 11 | A | 12 | A | 13 | A | 14 | A | 15 | A |
| 16 | A | 17 | A | 18 | A | 19 | A | 20 | A |
| 21 | A | 22 | B | 23 | A | 24 | A | 25 | A |
| 26 | A | 27 | A | 28 | A | 29 | A | 30 | A |
| 31 | A | 32 | A | 33 | C | 39 | A | 35 | B |
| 36 | A | 37 | A | 38 | A | 44 | A | 40 | A |
| 41 | A | 42 | A | 43 | B | 49 | A | 45 | A |
| 46 | A | 47 | A | 48 | A | 54 | A | 50 | A |
| 51 | A | 52 | B | 53 | A | 59 | A | 55 | A |
| 56 | A | 57 | A | 58 | A | 64 | A | 60 | A |
| 61 | A | 62 | A | 63 | A | 69 | A | 65 | A |
| 66 | A | 67 | A | 68 | A | 74 | A | 75 | A |
| 76 | A | 72 | A | 73 | A | 79 | B | 80 | B |
| 81 | A | 77 | A | 78 | A | 85 | A | 86 | A |
| 87 | A | 82 | A | 83 | A | 90 | B | 91 | A |
| 92 | A | 88 | A | 89 | A | 95 | A | 96 | A |
| 97 | A | 93 | A | 94 | A | 100 | A | 101 | A |
| 102 | A | 98 | A | 99 | A | 105 | A | 106 | A |
| 107 | A | 103 | A | 104 | A | 110 | A | 111 | A |
| 112 | A | 108 | B | 109 | A | 115 | A | 116 | A |
| 117 | A | 113 | A | 114 | A | 118 | A | 119 | B |
| 120 | A | 121 | A | 122 | B | 123 | A | 124 | A |
| 125 | A | 126 | A | 127 | B | 128 | A | 129 | A |
| 130 | B | 131 | B | 132 | A | 133 | A | 134 | A |
| 135 | A | 136 | A | 137 | A | 138 | A | 139 | A |
| 140 | A | 141 | A | 142 | A | 143 | A | 144 | A |
| 145 | A | 146 | A | 147 | A | 148 | A | 149 | A |
| 150 | A | 151 | A | 152 | A | 153 | A | 154 | A |
| 155 | A | 156 | A | 157 | A | 158 | A | 159 | A |
| 160 | A | 161 | A | 162 | A | 163 | A | 164 | A |
| 165 | A | 166 | A | 167 | A | 168 | A | 169 | B |
| 170 | A | 171 | A | 172 | A | 173 | A | 174 | A |
| 175 | A | 176 | A | 177 | A | 178 | A | 179 | A |
| 180 | A | 181 | B | 182 | A | 183 | A | 184 | B |
| 185 | B | 186 | A | 187 | A | 188 | B | | |

What is claimed is:

1. A compound of Formula (I), or a pharmaceutically acceptable salt, solvate, or stereoisomer thereof: 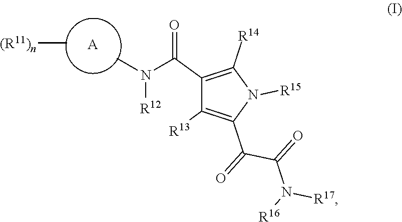
wherein:
Ring A is aryl, heteroaryl, cycloalkyl, or heterocycloalkyl;
each R¹¹ is independently halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R¹;
or two R¹¹ on adjacent atoms are taken together with the atoms to which they are attached to form a cycloalkyl, heterocycloalkyl, aryl, or heteroaryl; each optionally substituted with one, two, or three R²;
R¹² is hydrogen or C₁-C₆alkyl;
R¹³ is —CN, —OH, —SH, —SW, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³;
R¹⁴ is —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
provided that one of R¹³ or R¹⁴ is not —CH₃;
R¹⁵ is hydrogen, —S(═O)R^{a}, —S(═O)₂R^{a}, —S(═O)₂NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁵;
R¹⁶ and R¹⁷ are each independently hydrogen, —CN, —OR²⁰, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, cycloalkenyl, heterocycloalkyl, heterocycloalkenyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R⁶;
or R¹⁶ and R¹⁷ are taken together with the nitrogen atom to which they are attached to form a heterocycloalkyl or a heterocycloalkenyl; each optionally substituted with one, two, or three R⁷;
each R²⁰ is independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is optionally substituted with one, two, or three R⁶;
n is 0-4;
each R¹, R², R³, R⁴, R⁵, and R⁷ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R⁶ is independently oxo, halogen, —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —B(OR^{b})(OR^{c}), —C(═O)R^{a}, —OC(═O)R^{a}, —C(═O)OR^{b}, —OC(═O)OR, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is independently optionally substituted with one, two, or three R^{6a};
each R^{6a} is independently oxo, halogen, —CN, —OH, —OR^{a}, —NR^{b}R^{c}, —C(═O)R^{a}, —C(═O)OR^{b}, —C(═O)NR^{b}R^{c}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
each R^{a} is independently C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl; and
each R^{b} and R^{c} are independently hydrogen, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, or heteroaryl; wherein each alkyl, alkenyl, alkynyl, cycloalkyl, heterocycloalkyl, aryl, and heteroaryl is independently optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl;
or R^{b} and R^{c} are taken together with the atom to which they are attached to form a heterocycloalkyl optionally substituted with one, two, or three oxo, halogen, —CN, —OH, —OMe, —S(═O)Me, —S(═O)₂Me, —NH₂, —S(═O)₂NH₂, —C(═O)Me, —C(═O)OH, —C(═O)OMe, C₁-C₆alkyl, C₁-C₆haloalkyl, C₁-C₆hydroxyalkyl, or C₁-C₆aminoalkyl.

2. The compound of claim 1, or a pharmaceutically acceptable salt, solvate, or stereoisomer thereof, wherein:
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is —CN, —OH, —OR^{a}, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴;
or
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴.

3. The compound of claim 1 or 2, or a pharmaceutically acceptable salt, solvate, or stereoisomer thereof, wherein:
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴.

4. The compound of claim 1 or 2, or a pharmaceutically acceptable salt, solvate, or stereoisomer thereof, wherein:
R¹³ is —CN, —OH, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₁-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R³; and
R¹⁴ is —CN, —OH, —OW, —SH, —SR^{a}, —S(═O)R^{a}, —S(═O)₂R^{a}, —NO₂, —NR^{b}R^{c}, —NHS(═O)₂R^{a}, —S(═O)₂NR^{b}R^{c}, —C(═O)R^{a}, —OC(═O)R^{a}, —C(═O)OR^{b}, —OC(═O)OR^{b}, —C(═O)NR^{b}R^{c}, —OC(═O)NR^{b}R^{c}, —NR^{b}C(═O)NR^{b}R^{c}, —NR^{b}C(═O)R^{a}, —NR^{b}C(═O)OR^{b}, C₂-C₆alkyl, C₁-C₆haloalkyl, C₁-C₆hydroxyalkyl, C₁-C₆aminoalkyl, C₂-C₆alkenyl, C₂-C₆alkynyl, cycloalkyl, heterocycloalkyl, aryl, heteroaryl, —C₁-C₆alkyl(aryl), —C₁-C₆alkyl(heteroaryl), —C₁-C₆alkyl(cycloalkyl), or —C₁-C₆alkyl(heterocycloalkyl); wherein each alkyl, alkenyl, alkynyl, cycloalkyl, heterocycloalkyl, aryl, and heteroaryl is optionally substituted with one, two, or three R⁴.

5. The compound of any one of claims 1-3, or a pharmaceutically acceptable salt, solvate, or stereoisomer thereof, wherein:
R¹³ is C₂-C₆alkyl, C₁-C₆haloalkyl, or cycloalkyl; and
R¹⁴ is C₁-C₆alkyl, C₁-C₆haloalkyl, cycloalkyl, —C₁-C₆alkyl(cycloalkyl), or heteroaryl.

6. The compound of any one of claims 1-3 or 5, or a pharmaceutically acceptable salt, solvate, or stereoisomer thereof, wherein:
R¹³ is C₂-C₆alkyl or cycloalkyl; and
R¹⁴ is C₁-C₆alkyl.

7. The compound of any one of claims 1, 2, or 4, or a pharmaceutically acceptable salt, solvate, or stereoisomer thereof, wherein:
R¹³ is C₁-C₆alkyl, C₁-C₆haloalkyl, or cycloalkyl; and
R¹⁴ is C₂-C₆alkyl, C₁-C₆haloalkyl, cycloalkyl, —C₁-C₆alkyl(cycloalkyl), or heteroaryl.

8. The compound of any one of claims 1, 2, 4, or 7, or a pharmaceutically acceptable salt, solvate, or stereoisomer thereof, wherein:
R¹³ is C₁-C₆alkyl; and
R¹⁴ is C₂-C₆alkyl, cycloalkyl, —C₁-C₆alkyl(cycloalkyl), or heteroaryl.

9. The compound of any one of claims 1-8, or a pharmaceutically acceptable salt, solvate, or stereoisomer thereof, wherein:
R¹⁵ is hydrogen, C₁-C₆alkyl, C₁-C₆haloalkyl, or C₁-C₆hydroxyalkyl.

10. The compound of any one of claims 1-9, or a pharmaceutically acceptable salt, solvate, or stereoisomer thereof, wherein:
R¹⁵ is C₁-C₆alkyl.

11. The compound of any one of claims 1-10, or a pharmaceutically acceptable salt, solvate, or stereoisomer thereof, wherein:
R¹² is hydrogen.

12. The compound of any one of claims 1-11, or a pharmaceutically acceptable salt, solvate, or stereoisomer thereof, wherein:
Ring A is aryl or heteroaryl.

13. The compound of any one of claims 1-12, or a pharmaceutically acceptable salt, solvate, or stereoisomer thereof, wherein:
Ring A is phenyl or pyridyl.

14. The compound of any one of claims 1-13, or a pharmaceutically acceptable salt, solvate, or stereoisomer thereof, wherein:
Ring A is phenyl.

15. The compound of any one of claims 1-14, or a pharmaceutically acceptable salt, solvate, or stereoisomer thereof, wherein:
each R¹¹ is independently halogen, —CN, —OH, —OR^{a}, —NR^{b}R^{c}, —C(═O)OR^{b}, —C(═O)NR^{b}R^{c}, C₁-C₆alkyl, C₁-C₆haloalkyl, or cycloalkyl.

16. The compound of any one of claims 1-15, or a pharmaceutically acceptable salt, solvate, or stereoisomer thereof, wherein:
each R¹¹ is independently halogen, —CN, C₁-C₆alkyl, or C₁-C₆haloalkyl.

17. The compound of any one of claims 1-16, or a pharmaceutically acceptable salt, solvate, or stereoisomer thereof, wherein:
each R¹¹ is independently halogen or C₁-C₆alkyl.

18. The compound of any one of claims 1-17, or a pharmaceutically acceptable salt, solvate, or stereoisomer thereof, wherein:
n is 0-3.

19. The compound of any one of claims 1-18, or a pharmaceutically acceptable salt, solvate, or stereoisomer thereof, wherein:
n is 1.

20. The compound of any one of claims 1-18, or a pharmaceutically acceptable salt, solvate, or stereoisomer thereof, wherein:
n is 2.

21. The compound of any one of claims 1-18, or a pharmaceutically acceptable salt, solvate, or stereoisomer thereof, wherein:
n is 3.

22. The compound of any one of claims 1-21, or a pharmaceutically acceptable salt, solvate, or stereoisomer thereof, wherein:
R¹⁶ is hydrogen or C₁-C₆alkyl.

23. The compound of any one of claims 1-22, or a pharmaceutically acceptable salt, solvate, or stereoisomer thereof, wherein:
R¹⁶ is hydrogen.

24. The compound of any one of claims 1-23, or a pharmaceutically acceptable salt, solvate, or stereoisomer thereof, wherein:
R¹⁷ is —OR²⁰, C₁-C₆alkyl, C₁-C₆haloalkyl, C₁-C₆hydroxyalkyl, C₁-C₆aminoalkyl, C₂-C₆alkynyl, cycloalkyl, cycloalkenyl, heterocycloalkyl, aryl, heteroaryl, —C₁-C₆alkyl(heteroaryl), —C₁-C₆alkyl(cycloalkyl), or —C₁-C₆alkyl(heterocycloalkyl); wherein each alkyl, alkynyl, cycloalkyl, cycloalkenyl, heterocycloalkyl, aryl, and heteroaryl is optionally substituted with one, two, or three R⁶.

25. The compound of any one of claims 1-24, or a pharmaceutically acceptable salt, solvate, or stereoisomer thereof, wherein:
R¹⁷ is C₁-C₆alkyl, C₁-C₆haloalkyl, C₁-C₆hydroxyalkyl, cycloalkyl, heterocycloalkyl, —C₁-C₆alkyl(heteroaryl), or —C₁-C₆alkyl(cycloalkyl); wherein each alkyl, cycloalkyl, heterocycloalkyl, and heteroaryl is optionally substituted with one, two, or three R⁶.

26. The compound of any one of claims 1-25, or a pharmaceutically acceptable salt, solvate, or stereoisomer thereof, wherein:
R¹⁷ is C₁-C₆alkyl, C₁-C₆haloalkyl, C₁-C₆hydroxyalkyl, cycloalkyl, or heterocycloalkyl;
wherein each alkyl, cycloalkyl, heterocycloalkyl, and heteroaryl is optionally substituted with one, two, or three R⁶.

27. The compound of any one of claims 1-26, or a pharmaceutically acceptable salt, solvate, or stereoisomer thereof, wherein:
R¹⁷ is C₁-C₆hydroxyalkyl, cycloalkyl, or heterocycloalkyl; each optionally substituted with one, two, or three R⁶.

28. The compound of any one of claims 1-27, or a pharmaceutically acceptable salt, solvate, or stereoisomer thereof, wherein:
R¹⁷ is C₁-C₆alkyl or cycloalkyl; each optionally substituted with one, two, or three R⁶.

29. The compound of any one of claims 1-28, or a pharmaceutically acceptable salt, solvate, or stereoisomer thereof, wherein:
R¹⁷ is C₁-C₆alkyl optionally substituted with one, two, or three R⁶.

30. The compound of any one of claims 1-29, or a pharmaceutically acceptable salt, solvate, or stereoisomer thereof, wherein:
R¹⁷ is cycloalkyl optionally substituted with one, two, or three R⁶.

31. The compound of any one of claims 1-30, or a pharmaceutically acceptable salt, solvate, or stereoisomer thereof, wherein:
each R⁶ is independently oxo, halogen, —CN, —OH, —OR^{a}, —S(═O)₂R^{a}, —NR^{b}R^{c}, —NHS(═O)₂R^{a}, —S(═O)₂NR^{b}R^{c}, —B(OR^{b})(OR^{c}), —C(═O)R^{a}, —C(═O)OR^{b}, —C(═O)NR^{b}R^{c}, —NR^{b}C(═O)R^{a}, C₁-C₆alkyl, C₁-C₆haloalkyl, C₁-C₆hydroxyalkyl, C₁-C₆aminoalkyl, cycloalkyl, heterocycloalkyl, aryl, or heteroaryl; wherein each alkyl, cycloalkyl, heterocycloalkyl, aryl, and heteroaryl is independently optionally substituted with one, two, or three R^{6a}.

32. The compound of any one of claims 1-31, or a pharmaceutically acceptable salt, solvate, or stereoisomer thereof, wherein:
each R⁶ is independently halogen, —CN, —OH, —OR^{a}, —NR^{b}R^{c}, —C(═O)OR^{b}, —C(═O)NR^{b}R^{c}, C₁-C₆alkyl, C₁-C₆haloalkyl, C₁-C₆hydroxyalkyl, cycloalkyl, or heteroaryl; wherein each alkyl, cycloalkyl, heterocycloalkyl, and heteroaryl is independently optionally substituted with one, two, or three R^{6a}.

33. The compound of any one of claims 1-32, or a pharmaceutically acceptable salt, solvate, or stereoisomer thereof, wherein:
each R^{6a} is independently halogen, —CN, —OH, —OR^{a}, —NR^{b}R^{c}, C₁-C₆alkyl, or C₁-C₆haloalkyl.

34. The compound of any one of claims 1-21, or a pharmaceutically acceptable salt, solvate, or stereoisomer thereof, wherein:
R¹⁶ and R¹⁷ are taken together with the nitrogen atom to which they are attached to form a heterocycloalkyl optionally substituted with one, two, or three R⁷.

35. The compound of any one of claims 1-21 or 34, or a pharmaceutically acceptable salt, solvate, or stereoisomer thereof, wherein:
each R⁷ is independently oxo, halogen, —CN, —OH, —OR^{a}, —NR^{b}R^{c}, C₁-C₆alkyl, C₁-C₆haloalkyl, or C₁-C₆hydroxyalkyl.

36. The compound of any one of claims 1-21, 34 or 35, or a pharmaceutically acceptable salt, solvate, or stereoisomer thereof, wherein:
each R⁷ is independently —OH or C₁-C₆alkyl.

37. A compound, or a pharmaceutically acceptable salt, solvate, or stereoisomer thereof, selected from the group consisting of:

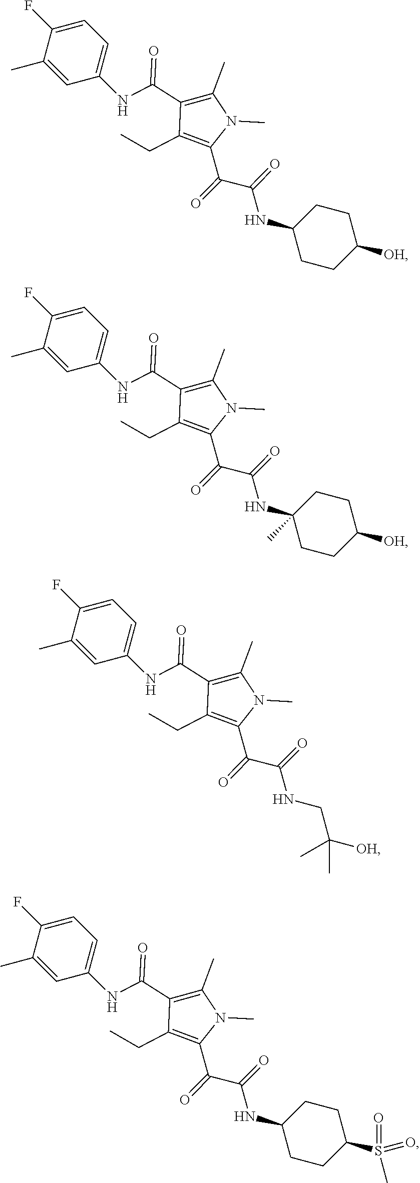 [SMILES: CCc1c(C(=O)Nc2ccc(F)c(C)c2)c(C)n(C)c1C(=O)C(=O)N[C@H]1CC[C@@H](O)CC1 ; CCc1c(C(=O)Nc2ccc(F)c(C)c2)c(C)n(C)c1C(=O)C(=O)N[C@]1(C)CC[C@H](O)CC1 ; CCc1c(C(=O)Nc2ccc(F)c(C)c2)c(C)n(C)c1C(=O)C(=O)NCC(C)(C)O ; CCc1c(C(=O)Nc2ccc(F)c(C)c2)c(C)n(C)c1C(=O)C(=O)N[C@H]1CC[C@@H](S(C)(=O)=O)CC1]

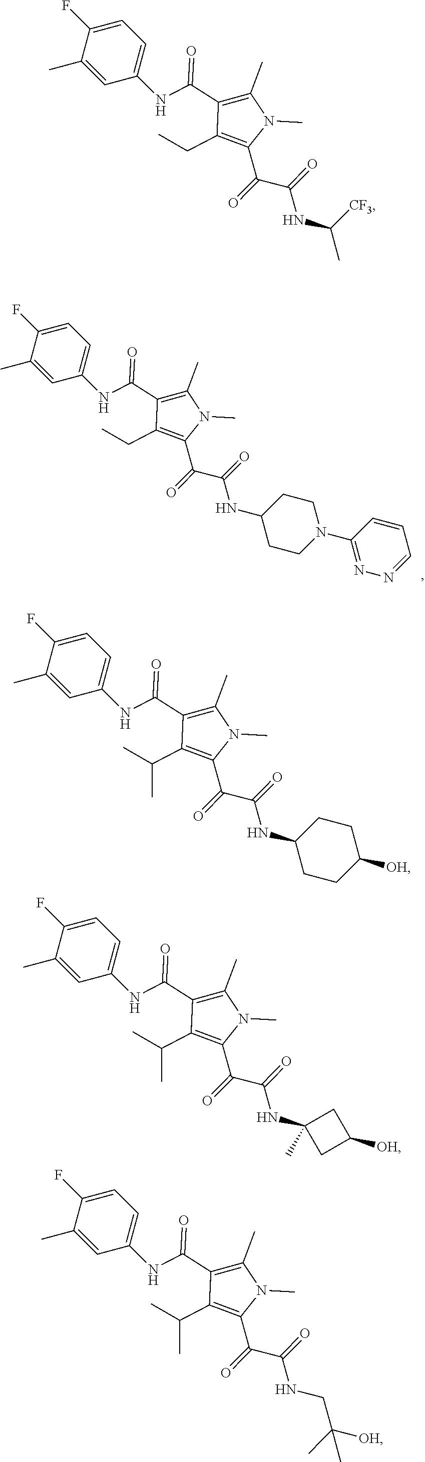 [SMILES: CCc1c(C(=O)Nc2ccc(F)c(C)c2)c(C)n(C)c1C(=O)C(=O)N[C@H](C)C(F)(F)F ; CCc1c(C(=O)Nc2ccc(F)c(C)c2)c(C)n(C)c1C(=O)C(=O)NC1CCN(c2cccnn2)CC1 ; Cc1cc(NC(=O)c2c(C(C)C)c(C(=O)C(=O)N[C@H]3CC[C@@H](O)CC3)n(C)c2C)ccc1F ; Cc1cc(NC(=O)c2c(C(C)C)c(C(=O)C(=O)N[C@]3(C)C[C@H](O)C3)n(C)c2C)ccc1F ; Cc1cc(NC(=O)c2c(C(C)C)c(C(=O)C(=O)NCC(C)(C)O)n(C)c2C)ccc1F]

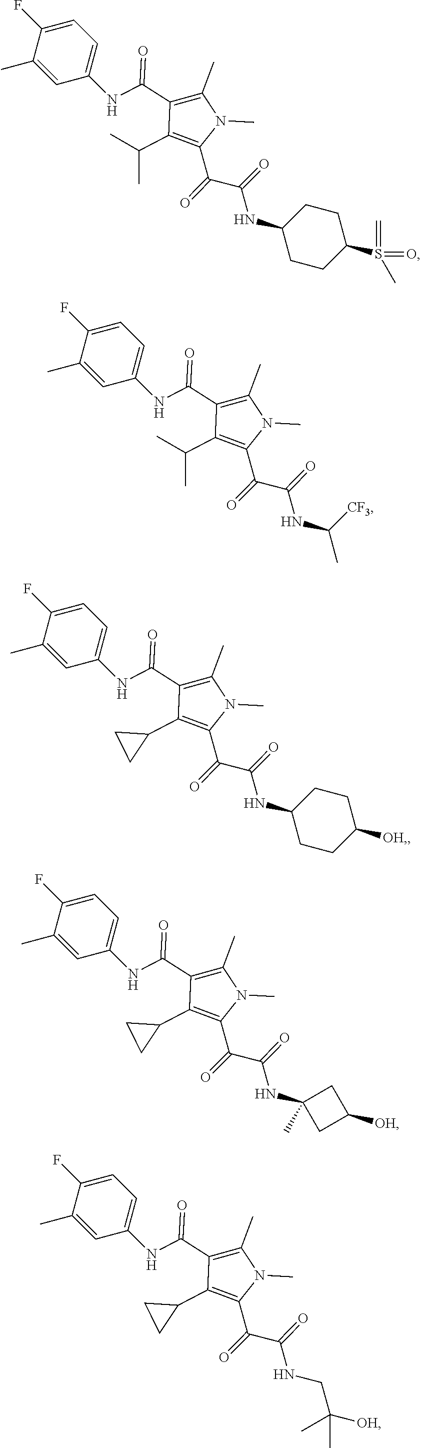 [SMILES: C=S(C)(=O)[C@H]1CC[C@@H](NC(=O)C(=O)c2c(C(C)C)c(C(=O)Nc3ccc(F)c(C)c3)c(C)n2C)CC1 ; Cc1cc(NC(=O)c2c(C(C)C)c(C(=O)C(=O)N[C@H](C)C(F)(F)F)n(C)c2C)ccc1F ; Cc1cc(NC(=O)c2c(C3CC3)c(C(=O)C(=O)N[C@H]3CC[C@@H](O)CC3)n(C)c2C)ccc1F ; Cc1cc(NC(=O)c2c(C3CC3)c(C(=O)C(=O)N[C@]3(C)C[C@H](O)C3)n(C)c2C)ccc1F ; Cc1cc(NC(=O)c2c(C3CC3)c(C(=O)C(=O)NCC(C)(C)O)n(C)c2C)ccc1F]

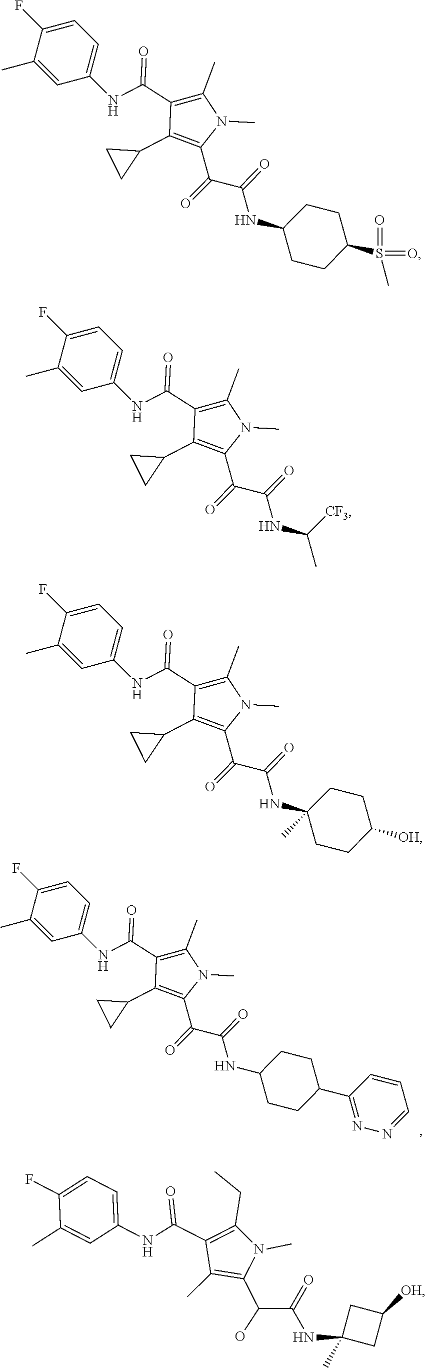 [SMILES: Cc1cc(NC(=O)c2c(C3CC3)c(C(=O)C(=O)N[C@H]3CC[C@@H](S(C)(=O)=O)CC3)n(C)c2C)ccc1F ; Cc1cc(NC(=O)c2c(C3CC3)c(C(=O)C(=O)N[C@H](C)C(F)(F)F)n(C)c2C)ccc1F ; Cc1cc(NC(=O)c2c(C3CC3)c(C(=O)C(=O)N[C@]3(C)CC[C@@H](O)CC3)n(C)c2C)ccc1F ; Cc1cc(NC(=O)c2c(C3CC3)c(C(=O)C(=O)NC3CCC(c4cccnn4)CC3)n(C)c2C)ccc1F ; CCc1c(C(=O)Nc2ccc(F)c(C)c2)c(C)c(C(O)C(=O)N[C@]2(C)C[C@H](O)C2)n1C]

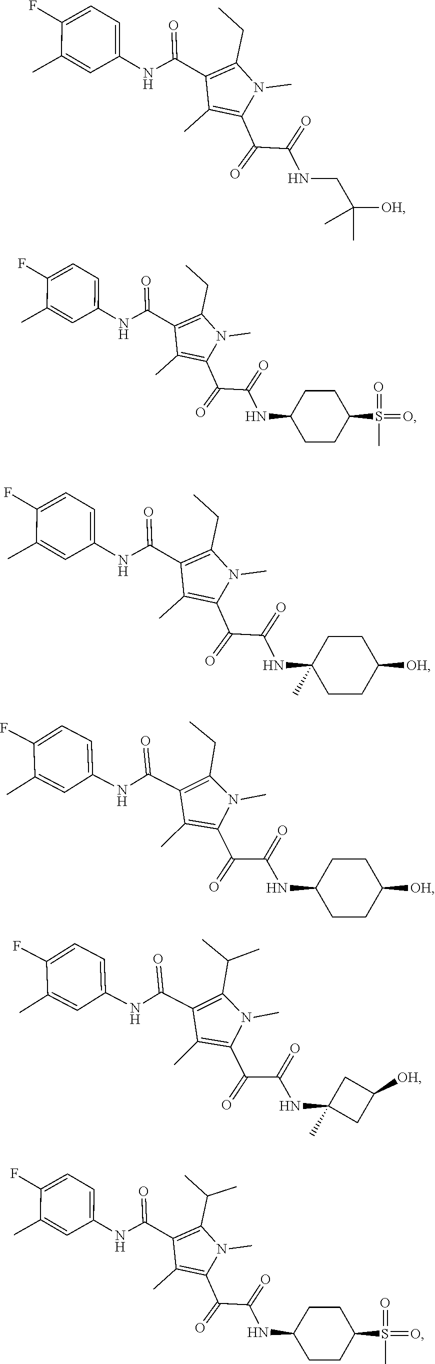 [SMILES: CCc1c(C(=O)Nc2ccc(F)c(C)c2)c(C)c(C(=O)C(=O)NCC(C)(C)O)n1C ; CCc1c(C(=O)Nc2ccc(F)c(C)c2)c(C)c(C(=O)C(=O)N[C@H]2CC[C@@H](S(C)(=O)=O)CC2)n1C ; CCc1c(C(=O)Nc2ccc(F)c(C)c2)c(C)c(C(=O)C(=O)N[C@]2(C)CC[C@H](O)CC2)n1C ; CCc1c(C(=O)Nc2ccc(F)c(C)c2)c(C)c(C(=O)C(=O)N[C@H]2CC[C@@H](O)CC2)n1C ; Cc1cc(NC(=O)c2c(C)c(C(=O)C(=O)N[C@]3(C)C[C@H](O)C3)n(C)c2C(C)C)ccc1F ; Cc1cc(NC(=O)c2c(C)c(C(=O)C(=O)N[C@H]3CC[C@@H](S(C)(=O)=O)CC3)n(C)c2C(C)C)ccc1F]

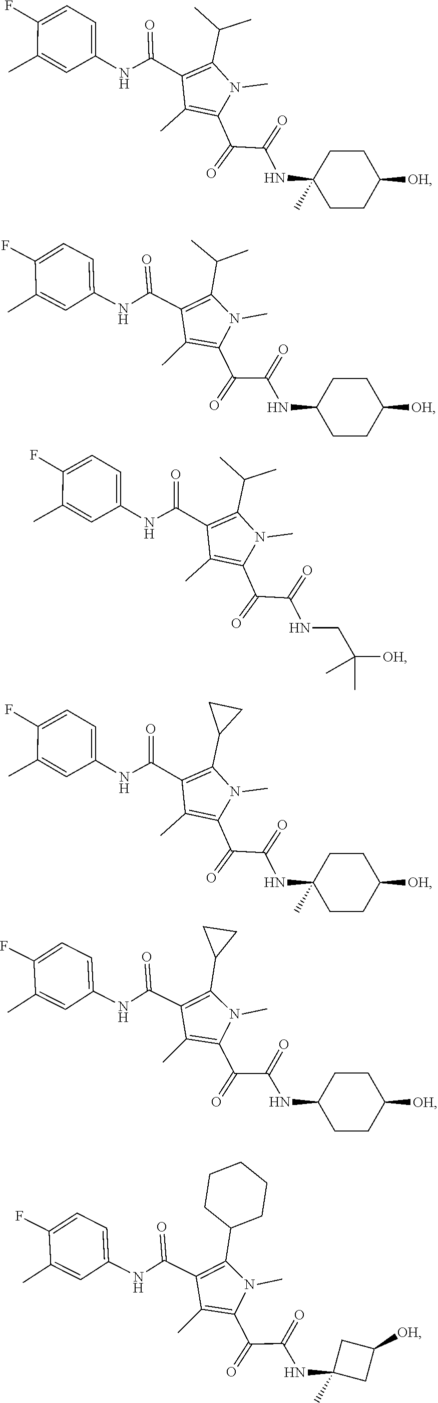 [SMILES: Cc1cc(NC(=O)c2c(C)c(C(=O)C(=O)N[C@]3(C)CC[C@H](O)CC3)n(C)c2C(C)C)ccc1F ; Cc1cc(NC(=O)c2c(C)c(C(=O)C(=O)N[C@H]3CC[C@@H](O)CC3)n(C)c2C(C)C)ccc1F ; Cc1cc(NC(=O)c2c(C)c(C(=O)C(=O)NCC(C)(C)O)n(C)c2C(C)C)ccc1F ; Cc1cc(NC(=O)c2c(C)c(C(=O)C(=O)N[C@]3(C)CC[C@H](O)CC3)n(C)c2C2CC2)ccc1F ; Cc1cc(NC(=O)c2c(C)c(C(=O)C(=O)N[C@H]3CC[C@@H](O)CC3)n(C)c2C2CC2)ccc1F ; Cc1cc(NC(=O)c2c(C)c(C(=O)C(=O)N[C@]3(C)C[C@H](O)C3)n(C)c2C2CCCCC2)ccc1F]

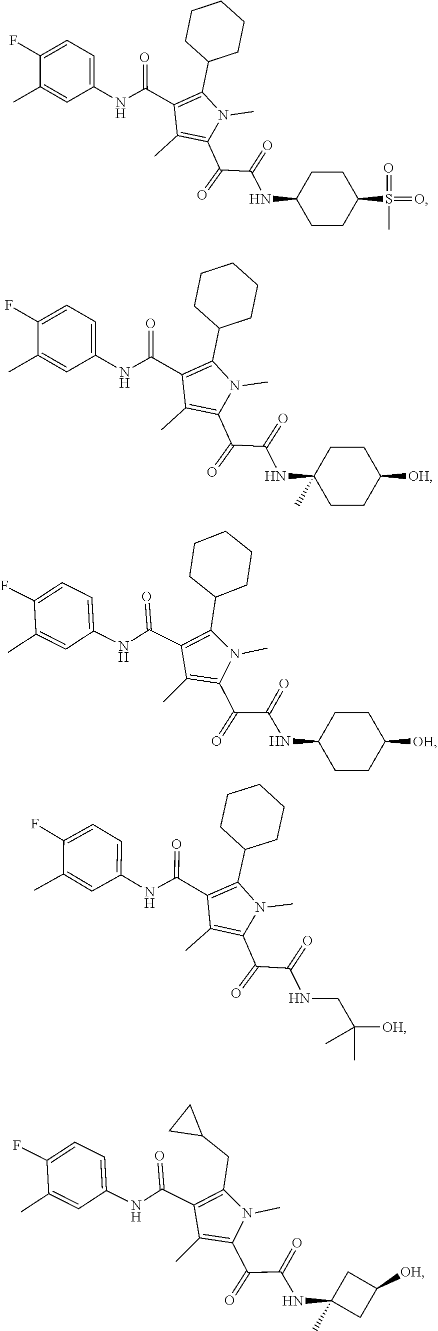 [SMILES: Cc1cc(NC(=O)c2c(C)c(C(=O)C(=O)N[C@H]3CC[C@@H](S(C)(=O)=O)CC3)n(C)c2C2CCCCC2)ccc1F ; Cc1cc(NC(=O)c2c(C)c(C(=O)C(=O)N[C@]3(C)CC[C@H](O)CC3)n(C)c2C2CCCCC2)ccc1F ; Cc1cc(NC(=O)c2c(C)c(C(=O)C(=O)N[C@H]3CC[C@@H](O)CC3)n(C)c2C2CCCCC2)ccc1F ; Cc1cc(NC(=O)c2c(C)c(C(=O)C(=O)NCC(C)(C)O)n(C)c2C2CCCCC2)ccc1F ; Cc1cc(NC(=O)c2c(C)c(C(=O)C(=O)N[C@]3(C)C[C@H](O)C3)n(C)c2CC2CC2)ccc1F]

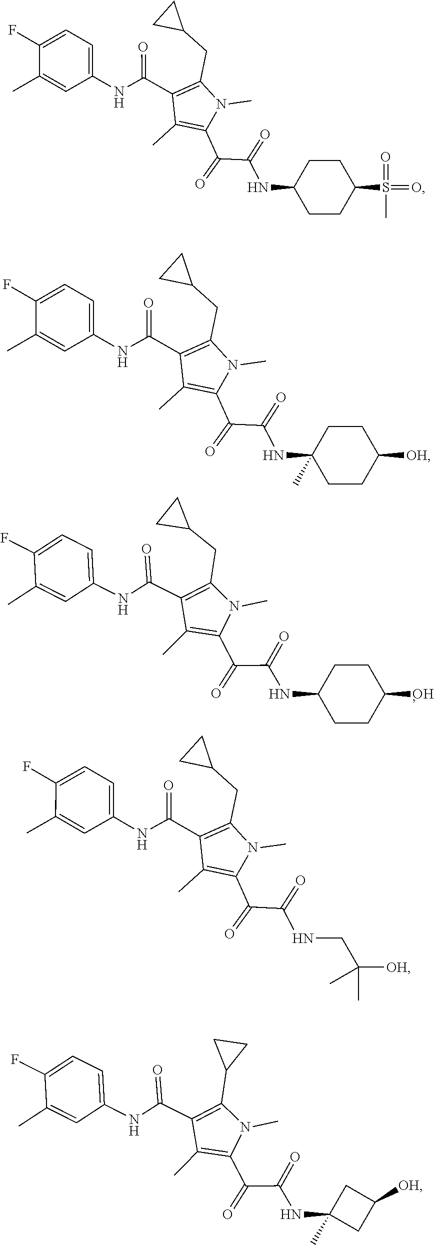

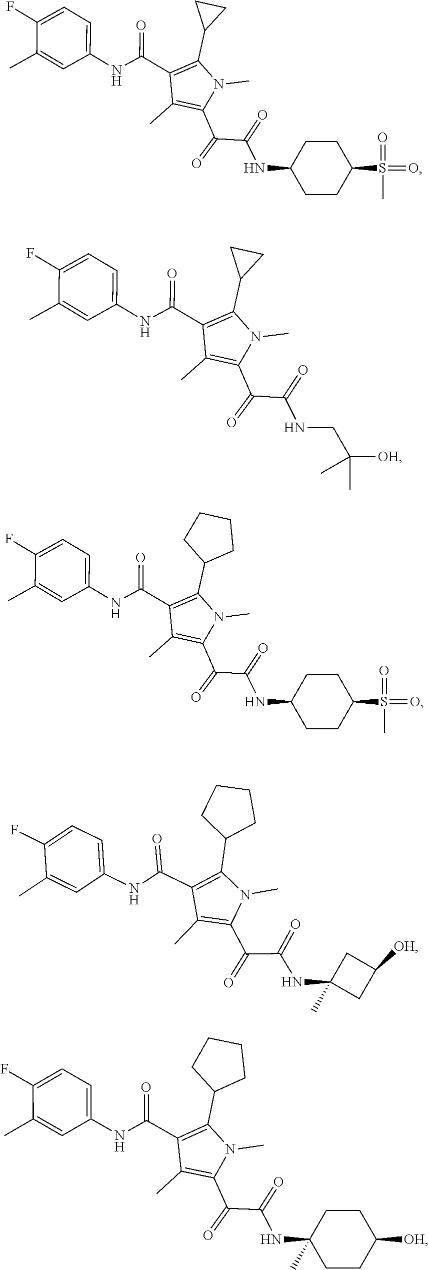 [SMILES: Cc1cc(NC(=O)c2c(C)c(C(=O)C(=O)N[C@H]3CC[C@@H](S(C)(=O)=O)CC3)n(C)c2C2CC2)ccc1F ; Cc1cc(NC(=O)c2c(C)c(C(=O)C(=O)NCC(C)(C)O)n(C)c2C2CC2)ccc1F ; Cc1cc(NC(=O)c2c(C)c(C(=O)C(=O)N[C@H]3CC[C@@H](S(C)(=O)=O)CC3)n(C)c2C2CCCC2)ccc1F ; Cc1cc(NC(=O)c2c(C)c(C(=O)C(=O)N[C@]3(C)C[C@H](O)C3)n(C)c2C2CCCC2)ccc1F ; Cc1cc(NC(=O)c2c(C)c(C(=O)C(=O)N[C@]3(C)CC[C@H](O)CC3)n(C)c2C2CCCC2)ccc1F]

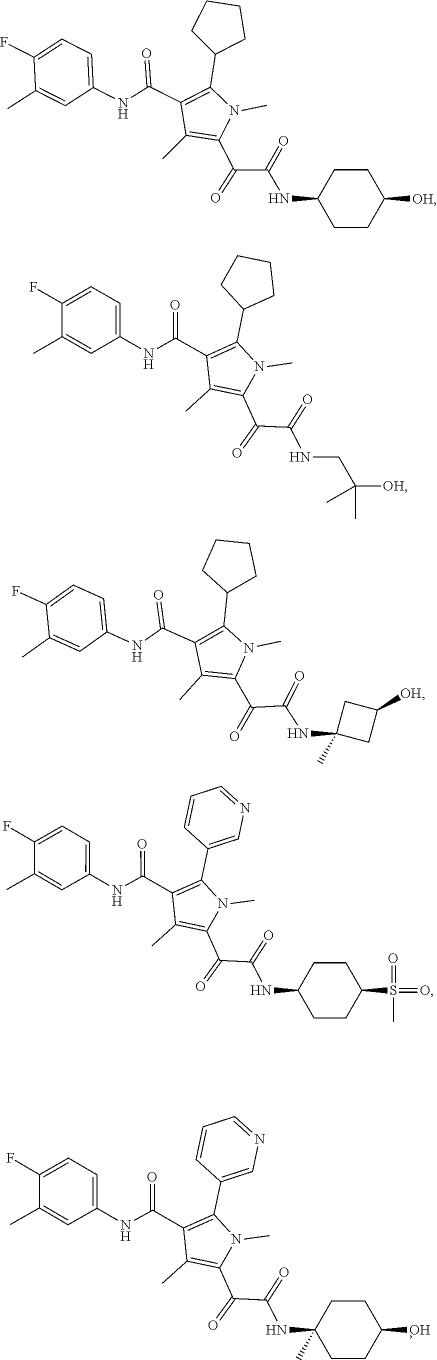 [SMILES: Cc1cc(NC(=O)c2c(C)c(C(=O)C(=O)N[C@H]3CC[C@@H](O)CC3)n(C)c2C2CCCC2)ccc1F ; Cc1cc(NC(=O)c2c(C)c(C(=O)C(=O)NCC(C)(C)O)n(C)c2C2CCCC2)ccc1F ; Cc1cc(NC(=O)c2c(C)c(C(=O)C(=O)N[C@]3(C)C[C@H](O)C3)n(C)c2C2CCCC2)ccc1F ; Cc1cc(NC(=O)c2c(C)c(C(=O)C(=O)N[C@H]3CC[C@@H](S(C)(=O)=O)CC3)n(C)c2-c2cccnc2)ccc1F ; Cc1cc(NC(=O)c2c(C)c(C(=O)C(=O)N[C@]3(C)CC[C@H](O)CC3)n(C)c2-c2cccnc2)ccc1F]

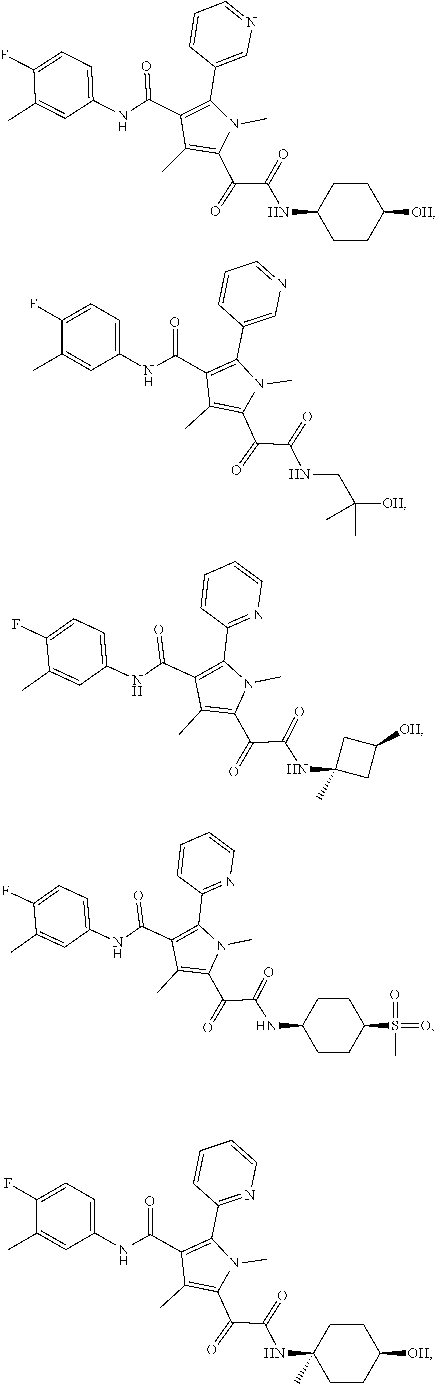 [SMILES: Cc1cc(NC(=O)c2c(C)c(C(=O)C(=O)N[C@H]3CC[C@@H](O)CC3)n(C)c2-c2cccnc2)ccc1F ; Cc1cc(NC(=O)c2c(C)c(C(=O)C(=O)NCC(C)(C)O)n(C)c2-c2cccnc2)ccc1F ; Cc1cc(NC(=O)c2c(C)c(C(=O)C(=O)N[C@]3(C)C[C@H](O)C3)n(C)c2-c2ccccn2)ccc1F ; Cc1cc(NC(=O)c2c(C)c(C(=O)C(=O)N[C@H]3CC[C@@H](S(C)(=O)=O)CC3)n(C)c2-c2ccccn2)ccc1F ; Cc1cc(NC(=O)c2c(C)c(C(=O)C(=O)N[C@]3(C)CC[C@H](O)CC3)n(C)c2-c2ccccn2)ccc1F]

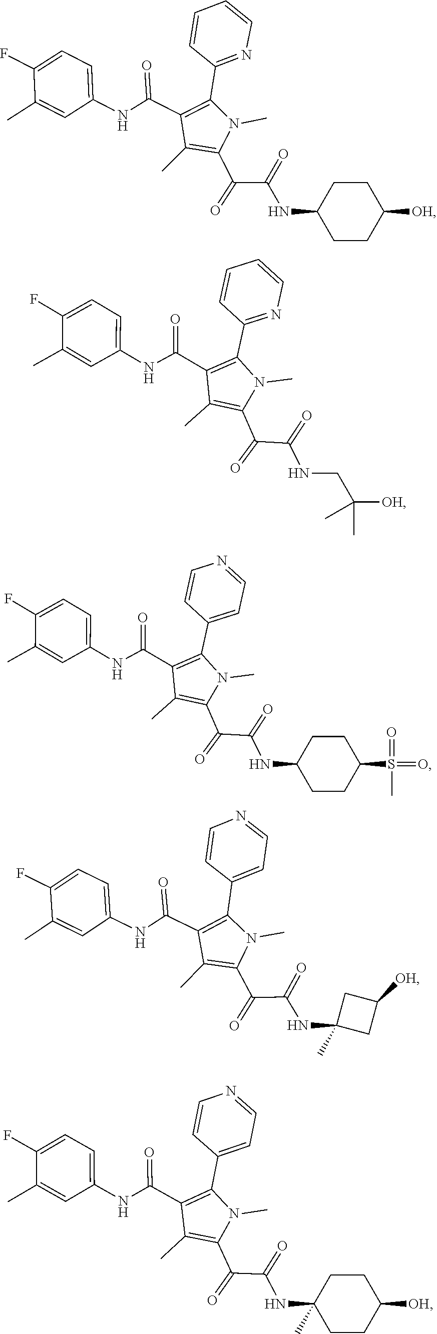 [SMILES: Cc1cc(NC(=O)c2c(C)c(C(=O)C(=O)N[C@H]3CC[C@@H](O)CC3)n(C)c2-c2ccccn2)ccc1F ; Cc1cc(NC(=O)c2c(C)c(C(=O)C(=O)NCC(C)(C)O)n(C)c2-c2ccccn2)ccc1F ; Cc1cc(NC(=O)c2c(C)c(C(=O)C(=O)N[C@H]3CC[C@@H](S(C)(=O)=O)CC3)n(C)c2-c2ccncc2)ccc1F ; Cc1cc(NC(=O)c2c(C)c(C(=O)C(=O)N[C@]3(C)C[C@H](O)C3)n(C)c2-c2ccncc2)ccc1F ; Cc1cc(NC(=O)c2c(C)c(C(=O)C(=O)N[C@]3(C)CC[C@H](O)CC3)n(C)c2-c2ccncc2)ccc1F]

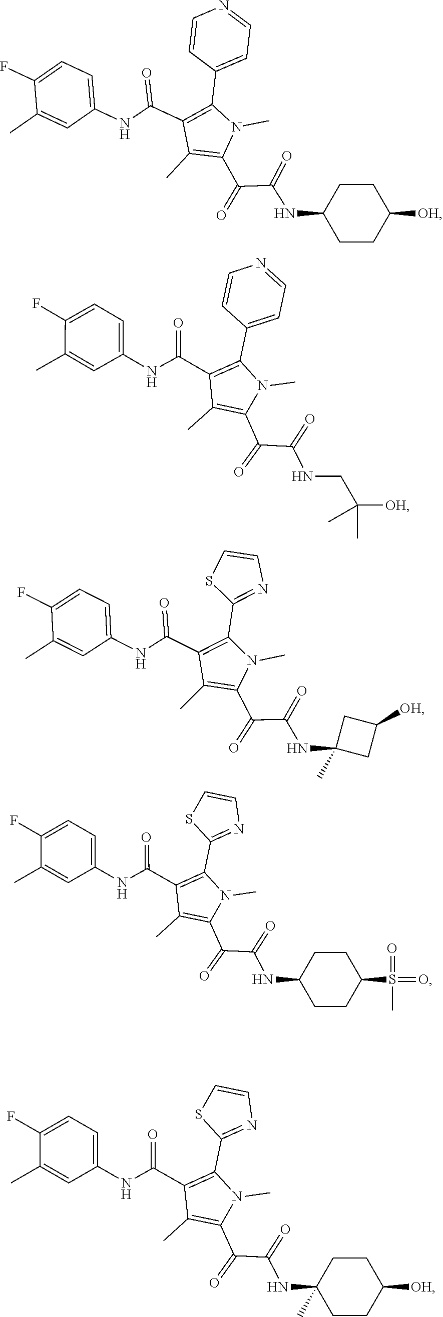 [SMILES: Cc1cc(NC(=O)c2c(C)c(C(=O)C(=O)N[C@H]3CC[C@@H](O)CC3)n(C)c2-c2ccncc2)ccc1F ; Cc1cc(NC(=O)c2c(C)c(C(=O)C(=O)NCC(C)(C)O)n(C)c2-c2ccncc2)ccc1F ; Cc1cc(NC(=O)c2c(C)c(C(=O)C(=O)N[C@]3(C)C[C@H](O)C3)n(C)c2-c2nccs2)ccc1F ; Cc1cc(NC(=O)c2c(C)c(C(=O)C(=O)N[C@H]3CC[C@@H](S(C)(=O)=O)CC3)n(C)c2-c2nccs2)ccc1F ; Cc1cc(NC(=O)c2c(C)c(C(=O)C(=O)N[C@]3(C)CC[C@H](O)CC3)n(C)c2-c2nccs2)ccc1F]

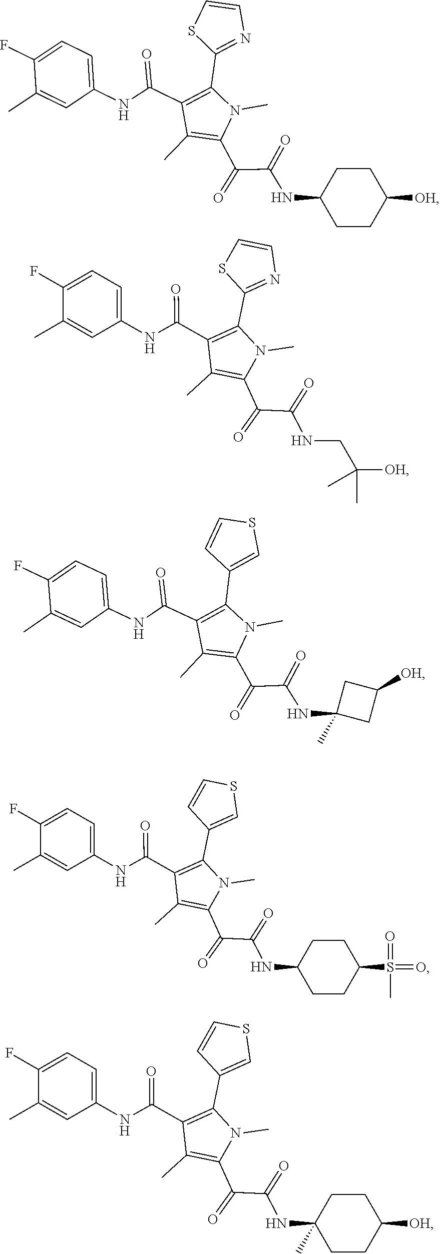

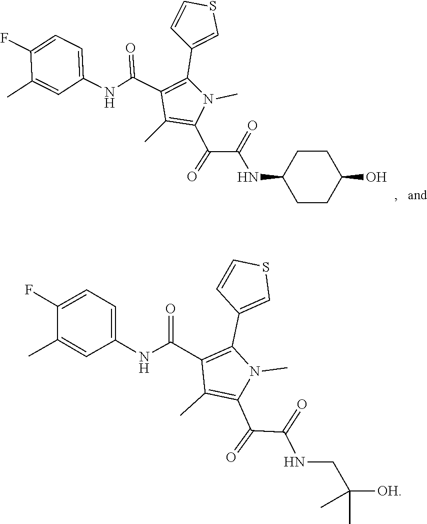 [SMILES: Cc1cc(NC(=O)c2c(C)c(C(=O)C(=O)N[C@H]3CC[C@@H](O)CC3)n(C)c2-c2ccsc2)ccc1F ; Cc1cc(NC(=O)c2c(C)c(C(=O)C(=O)NCC(C)(C)O)n(C)c2-c2ccsc2)ccc1F]

38. A compound, or a pharmaceutically acceptable salt, solvate, or stereoisomer thereof, selected from a compound of table 1.

39. A pharmaceutical composition comprising a compound of any one of claims 1-38, or a pharmaceutically acceptable salt, solvate, or stereoisomer thereof, and a pharmaceutically acceptable excipient.

40. A method of treating an infection in a subject, comprising administering to the subject a compound of any one of claims 1-38, or a pharmaceutically acceptable salt, solvate, or stereoisomer thereof.

41. A method of treating an infection in a subject, comprising administering to the subject a pharmaceutical composition of claim 1-38.

42. The method of claim 40 or 41, wherein the infection is a viral infection.

43. The method of any one of claims 40-42, wherein the infection is caused by the hepatitis B virus.

44. The method of any one of claims 40-43, wherein the infection is hepatitis B.

45. The method of any one of claims 41-44, further comprising administering an additional therapeutic agent useful for treating a chronic HBV infection.

46. The method of claim 45, wherein the additional therapeutic agent useful for treating a chronic HBV infection is a reverse transcriptase inhibitor; an HBV polymerase inhibitor, a capsid inhibitor; a cccDNA formation inhibitor; an RNA destabilizer; a checkpoint inhibitor (e.g., PD-1/PD-L1 inhibitor); a therapeutic vaccine; an RNA interference (RNAi) therapeutic; an antisense-based therapeutic, an HBV entry inhibitor; a TLR agonist; an RIG-I agonist, or an interferon.
